(19) United States
(12) Patent Application Publication
(10) Pub. No.: US 2009/0300781 A1
(43) Pub. Date: Dec. 3, 2009

(54) PREDICTION OF HETEROSIS AND OTHER TRAITS BY TRANSCRIPTOME ANALYSIS

(76) Inventors: Ian Bancroft, Norwich (GB); David Roger Stokes, Kent (GB); Colin Leslie Morgan, Norwich (GB); Fiona Patricia Fraser, Norwich (GB); Carmel Mary O'Neill, Norwich (GB)

Correspondence Address: KLARQUIST SPARKMAN, LLP, 121 SW SALMON STREET, SUITE 1600, PORTLAND, OR, 97204, US

(21) Appl. No.: 12/279,180

(22) PCT Filed: Mar. 30, 2007
(86) PCT No.: PCT/GB2007/001194

§ 371 Date: Feb. 16, 2009

Related U.S. Application Data
(60) Provisional application No. 60/787,877, filed on Mar. 31, 2006.

(30) Foreign Application Priority Data
Mar. 31, 2006 (GB) 0606583.3

(51) Int. Cl. C12N 15/82; A01H 5/00; A01K 67/027; C07H 21/04; G06N 5/02; G06N 3/12; G06F 15/18

(52) U.S. Cl. 800/13; 800/278; 800/281; 800/298; 536/23.1; 706/46; 706/13

(57) ABSTRACT

Transcriptome-based prediction of heterosis or hybrid vigour and other complex phenotypic traits. Analysis of transcript abundance in predictive gene sets, for predicting magnitude of heterosis or other complex traits in plants and animals. Transcriptome-based screening and selection of individuals with desired traits and/or good hybrid vigour.

This invention relates to methods of producing hybrid plants and hybrid non-human animals having high levels of hybrid vigour or heterosis and/or producing plants and non-human animals (e.g. hybrid, inbred or recombinant plants) having other traits such as desired flowering time, seed oil content and/or seed fatty acid ratios, and plants and non-human animals produced by these methods.

The invention relates to selection of suitable organisms, preferably plants or non-human animals, for use in producing hybrids and/or for use in breeding programmes, e.g. screening of germplasm collections for plants that may be suitable for inclusion in breeding programmes.

Many animal and plant species exhibit increased growth rates, reach larger sizes and, in the cases of crops [1,2] and farm animals [3,4], have higher yields and productivity when bred as hybrids, produced by crossing genetically dissimilar parents, a phenomenon known as hybrid vigour or heterosis [5]. The term heterosis can be applied to almost any aspect of biology in which a hybrid can be described as outperforming its parents.

The degree of heterosis observed varies a lot between different hybrids. The magnitude of heterosis can be described relative to the mean value of the parents (Mid-Parent Heterosis, MPH) or relative to the “better” of the parents (Best-Parent Heterosis, BPH).

Heterosis is of great importance in many agricultural crops and in plant and animal breeding, where it is clearly desirable to produce hybrids with high levels of heterosis. However, despite extensive genetic analysis in this area, the molecular mechanisms underlying heterosis remain poorly understood. Some progress has been made towards understanding the heterosis observed in simple traits controlled by single genes [6], but the mechanisms controlling more complex forms of heterosis, such as the vegetative vigour of hybrids, remain unknown [7, 8, 9].

Genetic analyses of heterosis have led to three, non-exclusive, genetic mechanisms being hypothesised to explain heterosis:

the “dominance” model, in which heterotic interactions are considered to be the cumulative effect of the phenotypic expression of dispersed dominant alleles, whereby deleterious alleles that are homozygous in the respective parents are complemented in the hybrids [2, 10];

the “overdominance” model, in which heterotic interactions are considered to be the result of heterozygous loci resulting in a phenotypic expression in excess of either parent, so that the heterozygosity per se produces heterosis [5, 11, 12];

the “epistatic” model, which includes other types of specific interactions between combinations of alleles at separate loci [13, 14].

Hypothetical models based on gene regulatory networks have been proposed to explain these types of interaction [15].

Whilst the hypothesised models attempt to explain in genetic terms at least a proportion of heterosis observed in hybrids, they do not provide a practical indicator that would enable breeders to predict quantitatively the level of heterosis for a given hybrid or to know which hybrid crosses are likely to perform well.

In allogamous crops, such as maize, heterotic groups have been established that enable the selection of inbreds that will show good heterosis when crossed. For example, Iowa Stiff Stalk vs. Non-Stiff Stalk lines [16]. Inter-group hybrids have greater genetic distance and heterosis than hybrids produced by crossing within an individual heterotic group [17] and it has been proposed that the level of genetic diversity may be a predictor of heterosis and yield [18]. However, this has not proven to be a reliable approach for the prediction of heterosis in crops [17]. Heterosis shows an inconsistent relationship with the degree of relatedness of the two parents, with an absence of correlation reported between heterosis and genetic distance in Arabidopsis thaliana [7, 19] and other species [20, 21, 22]. Thus, in general the level of heterosis observed in a hybrid does not depend solely upon the genetic distance between the two parents from which the hybrid was produced, nor does this variable, genetic distance, necessarily provide a good indicator of likely heterosis of hybrids.

At the gene transcript level, expression of alleles in a hybrid may represent the cumulative level of expression of the alleles inherited from each parent, or expression may be non-additive. Non-additive patterns of gene expression are believed to contribute to hybrid effects and therefore several studies have investigated non-additive gene expression in hybrids compared with their parents. Characteristics of the transcriptome (the contribution to the mRNA pool of each gene in the genome) have been analysed in heterotic hybrids of crop plants, and extensive differences in gene expression in the hybrids relative to the parents have been reported [23, 24, 25, 26, 27]. Hybrid transcriptomes were shown to be different from the transcriptomes of the parents. Quantitative changes were seen in the contribution to the mRNA pool of a subset of genes, when the transcriptomes of the hybrids were compared with the transcriptomes of their parents. These experiments were conducted with the expectation that differences in the transcriptomes of the hybrids, compared with their parents, contribute to the basis of heterosis.

Using differential display, Sun et al [24] identified differences in gene expression, of approximately 965 genes, between wheat seedling hybrids and their parents. The hybrids were generated from two single direction crosses, and represented one heterotic and one non-heterotic sample. Differences in gene expression were found between the hybrids and the parents, with some evidence provided of differences in response between the hybrids. In later experiments, Sun et al [28] used differential display techniques to identify changes in transcriptional remodelling for 2800 genes, between nine parental and 20 wheat hybrids. They found that around 30% of these genes showed some degree of remodelling. Broad trends in gene expression were assessed by random amplification. Gene expression differences were observed between the hybrid and both parents, between the hybrid and one parent only, and genes expressed only in the hybrid. The total number of non-additively expressed genes was found to correlate with some traits. The authors concluded that these differences in gene expression must be involved in developing a heterotic phenotype.

Guo et al. [29] reported allele-specific variation in transcript abundance in hybrids. Transcript abundance of 15 genes was analysed in maize hybrids, and transcript levels for the two alleles of each gene were compared. In 11 genes, the two alleles were found to be expressed unequally (bi-allelic expression), and in 4 genes just one allele was expressed (mono-allelic expression). Allele-specific differences in expression were observed between genetically different hybrids. Additionally, the two alleles in each hybrid were shown to respond differently to abiotic stress. Allele-specific differences may indicate different functions for the two parental alleles in hybrids, and this functional diversity of the two parental alleles in the hybrid was suggested to have an impact on heterosis.

Auger et al. [27] examined differences in transcript abundance between hybrids relative to their inbred parents. Several genes were found to be expressed at non-additive levels in the hybrids, but relevance to heterosis was not demonstrated.

Vuylsteke et al. [30] measured variations in transcript abundance between three inbred lines and two pairs of reciprocal F₁ hybrids of Arabidopsis. Non-additive levels of gene expression in the hybrids were used to estimate the proportion of genes expressed in a “dominance” fashion according to a genetic model of heterosis.

Microarray technology has also been used to study differences in transcript abundance across plant populations. For example, Kliebenstein et al. [31] used microarrays to quantify gene expression in seven Arabidopsis accessions, and found an average of 2234 genes to be significantly differentially expressed between any pair of accessions. The differences in gene expression were found to be related to sequence diversity in the accessions. Kirst et al. [32] examined transcript abundance in a pseudobackcross population of eucalyptus in order to compare transcript regulation in different genetic backgrounds of eucalyptus, and concluded that the genetic control of transcript levels was modulated by variation at different regulatory loci in different genetic backgrounds. Paux et al. [33] also conducted transcript profiling of eucalyptus genes, to examine gene expression during tension wood formation.

Another mechanism that has been proposed to explain heterosis is complementation of bottlenecks in metabolic systems [34]. It is possible that several different mechanisms are involved in heterosis, so that any one specific mechanism may only explain a proportion of heterosis observed.

Heterosis has been the subject of intense genetic analysis for almost a century, but no reliable and accurate basis for determining, predicting or influencing the degree of heterosis in a given hybrid has yet been identified. Thus, there has been a long-felt need to identify some basis on which parents may be selected in order to produce hybrids of increased vigour.

Attempts to produce hybrids with high levels of heterosis must currently be undertaken on the basis of trial and error, by experimentally crossing different parents and then waiting for the progeny to grow until it can be seen which of the new hybrids exhibit the most vigour. Breeding for new heterotic hybrids thus necessarily results in the co-production of significant numbers of under-performing hybrids with low hybrid vigour. The desired hybrids may not be obtained, or may only represent a fraction of the total number of hybrids produced overall. Additionally, hybrids must normally reach a certain age before their level of heterosis can be determined, which increases still further the time, cost and resources that must be invested in a breeding program, since it is necessary to continue to grow large numbers of hybrids even though many, or perhaps all, will not have the desired characteristics.

A method that could provide at least some measure of prediction of the level of heterosis likely to be exhibited by a given hybrid could result in significantly more effective breeding programs.

There are comparable needs to determine a basis on which plants or animals may be selected as parents for producing hybrids with further desirable multigenic traits, and for predicting which hybrid, inbred or recombinant plants or animals are likely to exhibit desired traits.

The invention disclosed herein is based on the unexpected finding that transcript abundance of certain genes is predictive of the degree of heterosis in a hybrid. Transcriptome analysis may be used to identify genes whose transcript abundance in hybrids correlates with heterosis. The abundance of those gene transcripts in a new hybrid can then be used to predict the degree of heterosis of the new hybrid. Moreover, transcriptome analysis may be used to identify genes whose transcript abundance in plants or animals correlates with heterosis in hybrids produced by crossing those plants or animals. Thus, transcriptome data from parents can be used to predict the magnitude of heterosis in hybrids which have yet to be produced.

We show herein that changes in transcript abundance in the transcriptome represent the majority of the basis of heterosis. Importantly, this means that predictions based on transcript abundance are close to the observed magnitude of heterosis, i.e. the invention allows quantitative prediction of the degree of heterosis in a hybrid. Transcriptome characteristics alone may thus be used to predict heterosis in hybrids and as a basis for selection of parents.

Thus, remarkably, we have solved a problem that has been unanswered for almost a century. By demonstrating that the basis of heterosis resides primarily at the level of the regulation of transcript abundance, we have provided a means of predicting heterosis in hybrids and thus selecting which hybrids to maintain. Furthermore, we were able to identify characteristics of parental transcriptomes that could be used successfully as markers to predict the magnitude of heterosis in untested hybrids, and we have thus also provided basis for identifying parents which can be crossed to produce heterotic hybrids.

This invention differs from previous studies involving transcriptome analysis of hybrids, since those earlier studies did not identify any relationship between the transcriptomes of hybrids and the degree of heterosis observed in those hybrids. As discussed above, earlier studies showed that transcript levels of some genes differ in hybrids compared with the parents from which those hybrids were derived, and differences between hybrid and parent transcriptome were suggested to contribute to phenotypic differences including heterosis. However, the previous investigators did not compare transcriptome remodelling in a range of non-heterotic hybrids and heterotic hybrids, and did not show whether transcriptome remodelling correlates with heterosis.

We have recognised that most differences in the hybrid transcriptome are due to hybrid formation, not heterosis. We found that, in fact, transcriptome remodelling involving transcript abundance fold-changes of 2 or more occurs to a similar extent in all hybrids relative to their parents, regardless of the degree of heterosis observed in the hybrids. Accordingly, the overall degree of transcriptome remodelling in a hybrid is not an indicator of the degree of heterosis in that hybrid.

Therefore, earlier studies involving limited numbers of hybrids were not able to identify genes whose transcript abundance correlated with heterosis. The vast majority of differences in transcript abundance observed in earlier studies would have been due only to hybrid formation itself, and would not show any correlation with heterosis. Nor was any such correlation even looked for in the prior art, since it was not recognised that a correlation might exist.

However, despite showing that the overall degree of transcriptome remodelling in a hybrid is not related to heterosis, we found that transcriptome analysis can nevertheless be used to reveal features of the hybrid transcriptome that are predictive of the degree of heterosis in a hybrid. Through transcriptome analysis of a wide range of hybrids we have unexpectedly shown that transcript abundance of a proportion of genes correlates with heterosis. As described herein, we studied 13 different heterotic hybrids of Arabidopsis thaliana, and identified features of the hybrid transcriptome that are characteristic of heterotic interactions. We identified 70 genes whose transcript abundance in the hybrid transcriptome correlated with the degree of heterosis in the Arabidopsis hybrids. We then successfully used the transcript abundance of that defined set of 70 genes to quantitatively predict the magnitude of heterosis observed in 3 untested hybrid combinations. Transcript abundance of two additional genes, At1g67500 and At5g45500, was also shown to have a significant negative correlation with heterosis. Transcript abundance of each of these genes successfully predicted heterosis in further hybrids.

Further, we identified a larger set of genes whose transcript abundance in the transcriptome of Arabidopsis inbred lines correlated with the degree of heterosis in hybrid progeny produced by crossing those lines. We successfully used the transcript abundance of that set of genes to quantitatively predict the magnitude of heterosis in 3 hybrids produced from those lines. Transcript abundance of At3g11220 was found to be negatively correlated with heterosis in a highly significant manner and transcript abundance of this gene in the parental transcriptome was found to be predictive of heterosis in hybrid offspring.

Heterosis in hybrids of Arabidopsis thaliana may be predicted on the basis of the transcript abundance of these identified Arabidopsis genes. Moreover, since heterosis is a widely observed phenomenon, and is not restricted to Arabidopsis or even to plants, but is also observed in animals, it is to be expected that many of the same genes whose transcript abundance correlates with heterosis in Arabidopsis will also correlate with heterosis in other organisms. Transcript abundance of orthologues of those genes in other species may thus correlate with heterosis.

However, prediction of heterosis need not be based on genes selected from the sets of genes disclosed herein, since one aspect of the invention is use of transcriptome analysis to identify the particular genes whose transcript abundance correlates with heterosis in any population of hybrids that is of interest. Once identified, those genes may then be used for prediction of heterosis or other trait in the particular hybrids of interest. Whilst the identified genes may include at least some genes, or orthologues thereof, from the set of genes identified in Arabidopsis, they need not do so.

The invention enables hybrids likely to exhibit high levels of heterosis to be identified and selected, while hybrids likely to exhibit lower degrees of heterosis may be discarded. Notably, the invention may be used to predict the level of heterosis in a hybrid at an early stage in the life of the hybrid, for example in a seedling, before it would be possible to directly observe differences between heterotic and non-heterotic hybrids. Thus, the invention may be used in a hybrid whose degree of heterosis is not yet determinable from its phenotype. The invention thus provides significant benefits to a breeder, since it allows a breeder to determine which particular hybrids in a potentially vast array of different hybrids should be retained and grown. For example, a breeder may use transcript abundance data from seedlings to decide which plant hybrids to grow or test in yield/performance trials.

Furthermore, we have shown that regulation of transcript abundance underlies not only heterosis but also other traits. These may include all genetically complex traits in hybrid, inbred or recombinant plants and animals, e.g. flowering time or seed composition in plants. Accordingly, the invention also relates to determining features of plant or non-human animal transcriptomes (e.g. transcriptomes of hybrids and/or inbred or recombinant plants or animals) for prediction of other traits in the plant or animal or offspring thereof. Where the invention relates to traits other than heterosis, the plant or animal may be a hybrid or alternatively it may be inbred or recombinant. Examples of traits that may be predicted using the invention are yield, flowering time, seed oil content and seed fatty acid ratios in plants, especially plant hybrids, e.g. accessions of A. thaliana. These and other traits may also be predicted in the plant or non-human animal (e.g. hybrid, inbred or recombinant plant or animal) before those traits are manifested in the phenotype. Thus, for example, we demonstrate herein that the invention allows seed oil content of inbred plants to be accurately predicted by analysis of plants that have not yet flowered. The invention thus confers significant predictive, cost and workload reductive advantages, particularly for traits manifested at a relatively late stage, since it means that it is not necessary to wait until a plant or animal reaches a particular (often late) stage of development before being able to know the magnitude or properties of the trait that will be exhibited by a given plant or animal.

Other aspects of the invention allow prediction of traits in plants or animals based on characteristics of their parents, and thus traits of plants or animals may be predicted and selected for even before those plants or animals are produced. As noted above, the trait may be heterosis in a plant or animal hybrid. Therefore, in accordance with the invention, features of plant or animal transcriptomes may be identified that allow the degree of heterosis of plants or animals produced by crossing those plants or animals to be predicted. The invention can be used to predict one or more traits, such as the degree of heterosis observed in plants or animals produced by crossing different combinations of parental germplasms. This is potentially as valuable or even more valuable than being able to predict heterosis and other traits in plants and animals that have already been produced, since it avoids producing under-performing plants or animals and therefore allows significant savings in logistics, costs and time. Particular plants or animals may thus be selected for breeding, with an increased chance that their progeny will be heterotic hybrids, or possess other traits, compared with if the parents were selected at random. Thus, the methods of the invention allow prediction in terms of the level of heterosis or of other traits produced by any particular cross between different parents, and allow particular parents to be selected accordingly. For example in agricultural crop plant breeding the invention reduces the need to make large numbers of different crosses in order to obtain new heterotic hybrids, since the invention can be used to identify in advance which particular crosses will be most productive.

Remarkably, methods of the invention may be used to predict traits based on transcript abundance in tissues in which the trait is not exhibited or which have no apparent relevance to the trait. For example, traits such as flowering time or seed composition may be predicted in plants based on transcript abundance data from non-flowering tissue, such as leaf tissue. Thus, the invention allows generation of statistical correlations between one or more traits and abundance of one or more gene transcripts. There is no requirement for the tissue sampled for transcriptome analysis to be the same as that used for trait measurement. It may be preferable that the tissue sampled for transcriptome analysis is, in terms of evolution, be a more ancient origin—hence the transcriptome in leaves can be used to predict more recently evolved characteristics of plants, such as flowering time or seed composition.

Based on the extensive transcriptome remodelling in hybrids of Arabidopsis thaliana disclosed herein, including some combinations that are heterotic for vegetative biomass and some combinations that are non-heterotic, it is evident that the methods of the invention may be applied to advantage in crops of economic importance.

Maize is currently bred as a hybrid crop, with its cultivation in the UK being for silage from the whole plant. Biomass yield is therefore paramount, and heterosis underpins this yield. In the USA maize is primarily grown for corn production, for which kernel weight represents the productive yield, and this yield is also dependent on heterosis. The ability to efficiently select for hybrid performance at an early stage of the hybrid parent breeding process provided by the method of this invention greatly accelerates the development of hybrid plant lines to increase yields and introduce a range of “sustainability” traits from exotic germplasm without loss of yield. Oilseed rape hybrids hold much potential, but their exploitation is limited as heterosis is often restricted to vegetative vigour, with little improvement in seed dry weight yield. The ability to select for specific performance traits at early stages of growth similarly accelerates the development of more productive and sustainable varieties. There is great potential for hybrid breeding of bread wheat (already a hexaploid, so benefits from some “fixed” heterosis) which, like oilseed rape, is supported by a breeding community based in the UK. In addition, hybrid varieties are important for a large number of vegetable species cultivated in the UK (such as cabbages, onions, carrots, peppers, tomatoes, melons), which are grown for enhancement of crop uniformity, appearance and general quality. Use of the invention to define a predictive marker for heterosis and other performance traits thus has the potential to revolutionise both the breeding process and the performance of crops for the farmer.

As demonstrated in the Examples, we identified relationships between gene expression in glasshouse-grown seedlings of maize inbreds and phenotypes (grain yield) in related plants at a later developmental stage and after growth under different environmental conditions.

In summary, the invention involves use of transcriptome analysis of plants or animals, e.g. hybrids and/or inbred or recombinant plants or animals, for:

(i) identifying genes involved in the manifestation of heterosis and other traits; and/or
(ii) predicting and producing plants or animals of improved heterosis and other traits by selecting plants or animals for breeding, wherein the plants or animals which exhibit enhanced transcriptome characteristics with respect to a selected set of genes relevant to the transcriptional regulatory networks present in potential parental breeding partners; and/or
(iii) predicting a range of trait characteristics for plants and animals based on transcriptome characteristics.

The invention also relates to plant and animal hybrids of improved heterosis, and to hybrids, inbreds or recombinants with improved traits as produced or predicted by the methods of the invention.

The results disclosed herein provide evidence for a link between heterosis and growth repression that is a consequence of stress tolerance mechanisms. We identified a number of genes which are highly predictive of heterosis, and which showed a significant negative correlation between gene expression and heterotic performance. As discussed in the Examples herein, these genes may represent key genetic loci that are downregulated in heterotic hybrids, leading to decreased expression of stress-avoidance genes and thus allowing better hybrid performance under favourable conditions. This raises the possibility that heterosis, at least for vegetative biomass, is at least partly a consequence of genetic interactions that lead to a reduction in repression of growth, rather than direct promotion of growth. However, whatever the molecular mechanism underlying heterosis, we have established that certain genes and sets of genes predictive of heterosis may be identified and successfully used in accordance with the present invention for predicting heterosis.

A hybrid is offspring of two parents of differing genetic composition. Thus, a hybrid is a cross between two differing parental germplasms. The parents may be plants or animals. A hybrid is typically produced by crossing a maternal parent with a different paternal parent. In plants, the maternal parent is usually, though not necessarily, impaired in male fertility and the paternal parent is a male fertile pollen donor. Parents may for example be inbred or recombinant.

An inbred plant or animal typically lacks heterozygosity. Inbred plants may be produced by recurrent self-pollination. Inbred animals may be produced by breeding between animals of closely related pedigree.

Recombinant plants or animals are neither hybrid nor inbred. Recombinants are themselves derived by the crossing of genetically dissimilar progenitors and may contain extensive heterozygosity and novel combinations of alleles. Most samples in germplasm collections of plant breeding programmes are recombinant.

The invention may be used with plants or animals. In some embodiments the invention preferably relates to plants. For example, the plants may be crop plants. The crop plants may be cotton, sugar beet, cereal plants (e.g. maize, wheat, barley, rice), oil-seed crops (e.g. soybeans, oilseed rape, sunflowers), fruit or vegetable crop plants (e.g. cabbages, onions, carrots, peppers, tomatoes, melons, legumes, leeks, brassicas e.g. broccoli) or salad crop plants e.g. lettuce [35]. The invention may be applied to hardwood timber trees or alder trees [36]. All species grown as crops could benefit from the invention, irrespective of whether they are currently cultivated extensively as hybrids.

Other embodiments relate to non-human animals e.g. mammals, birds and fish, including farm animals for example cattle, pigs, sheep, birds or poultry (e.g. chickens), goats, and farmed fish e.g. salmon, and other animals such as sports animals e.g. racehorses, racing pigeons, greyhounds or camels. Heterosis has been described in a variety of different animals including for example pigs [37], sheep [38, 39], goats [39], alpaca [39], Japanese quail [40] and salmon [41], and the invention may be applied to these and to other animals.

The invention can most conveniently be used in relation to organisms for which the genome sequence or extensive collections of Expressed Sequence Tags are available and in which microarrays are preferably also available and/or resources for transcriptome analysis have been developed.

In one aspect, the invention is a method comprising:

analysing the transcriptomes of plants or animals in a population of plants or animals;

measuring a trait of the plants or animals in the population; and

identifying a correlation between transcript abundance of one or more, preferably a set of, genes in the plant or animal transcriptomes and the trait in the plants or animals.

Thus the invention provides a method of identifying an indicator of a trait in a plant or animal.

The population may comprise e.g. at least 5, 10, 20, 30, 40, 50 or 100 plants or animals. Use of a large population to obtain trait measurements from many different plants or animals may allow increased accuracy of trait predictions based on correlations identified using the population.

The invention may thus be used to generate a model (e.g. a regression, as described in detail elsewhere herein) for predicting the trait based on transcript abundance of the one or more genes e.g. a set of genes.

One or more traits may be determined or measured, and thus correlations may be identified, and models may be generated, for a plurality of traits.

The plant or animal may be a hybrid, or it may be inbred or recombinant. In a preferred embodiment the plant or animal is a hybrid. A preferred trait is heterosis.

Plants or animals in a population may or may not be related to one another. The population may comprise plants or animals, e.g. hybrids, having different maternal and/or paternal parents. In some embodiments, all plants or animals, e.g. hybrids, in the population have the same maternal parent, but may have different paternal parents. In other embodiments, all plants or animals, e.g. hybrids, in the population have the same paternal parent, but may have different maternal parents. Parents may be inbred or recombinant, as explained elsewhere herein.

Methods for determining heterosis, for transcriptome analysis and for identifying statistical correlations are described in detail elsewhere herein.

Determining or measuring heterosis or other trait can be performed once the relevant phenotype is apparent e.g. once the heterosis can be calculated, or once the trait can be measured.

Transcriptome analysis may be performed at a time when the degree of heterosis or other trait of the plant or animal can be determined. Transcriptome analysis may be performed after, normally directly after, measurements are taken for determining or measuring heterosis or other trait in the plant or animal. This is suitable e.g. when measurements are taken for determining heterosis for fresh weight in hybrids.

However, we have demonstrated herein that it is possible to use transcriptome analysis of plants at a relatively early developmental stage, e.g. before flowering, to identify genes whose transcript abundance correlates with traits that only occur later in development, e.g. traits such as the time of flowering and aspects of the composition of seeds produced by plants. Accordingly, transcriptome analysis may be performed when the degree of heterosis or other trait is not yet determinable from the phenotype. This is suitable e.g. when measuring aspects of performance other than fresh weight, such as yield, for determining heterosis. For example, transcriptome analysis may be performed when plants are in vegetative phase or when animals are pre-adolescent, in order to predict heterosis for characteristics that are evident later in development, or to predict other traits that are evident later in development. For example, heterosis for seed or crop yields, or traits such as flowering time, seed or crop yields or seed composition, may be predicted using transcriptome data from vegetative phase plants.

Correlations between traits and transcript abundance represent models that may be used to predict traits in further plants or animals by determining transcript abundance in those plants or animals.

Thus, in another aspect, the invention is a method comprising:

determining transcript abundance of one or more, preferably a set of, genes in a plant or animal, wherein the transcript abundance of the one or more genes, or set of genes, in the transcriptome of the plant or animal correlates with a trait in the plant or animal; and

thereby predicting the trait in the plant or animal.

The analysis of transcript abundance is predictive of the trait in a plant or animal of the same genotype as the plant or animal in which transcript abundance was determined. Thus, in some embodiments the method may be used for the purpose of predicting a trait in the actual plant or animal whose transcript abundance is determined, and in other embodiments the method may be used for the purpose of predicting a trait in another plant or animal that is genetically identical to the plant or animal whose transcript abundance was sampled. For example the method may be used for predicting a trait in a genetically identical plant or animal that may be grown or produced subsequently, and indeed the decision whether to grow or produce the plant or animal may be informed by the trait prediction.

Methods of the invention may comprise determining transcript abundance of one or more genes, preferably a set of genes, in a plurality of plants or animals, and thus predicting one or more traits in the plurality of plants or animals. Thus, the invention may be used to predict a rank order for the trait in those plants or animals, which allows selection of plants or animals that are predicted to exhibit the highest or lowest trait (e.g. longest or shortest time to flowering, highest seed oil content, highest heterosis).

The plant or animal may be a hybrid, or it may be inbred or recombinant. In a preferred embodiment the plant or animal is a hybrid. A preferred trait is heterosis, and thus the method may be for predicting the magnitude of heterosis in a hybrid.

A method of the invention may comprise:

determining transcript abundance of one or more, preferably a set of, genes in a plant or animal, e.g. a hybrid, wherein transcript abundance of the one or more genes, or set of genes, correlates with a trait in a population of plants or animals, e.g. a population of hybrids; and

thereby predicting the trait in the plant or animal.

Plants or animals in the population may or may not be related to one another. The population typically comprises plants or animals, e.g. hybrids, having different maternal and/or paternal parents. In some embodiments, all plants or animals in the population have the same maternal parent, but may have different paternal parents. In other embodiments, all plants or animals in the population have the same paternal parent, but may have different maternal parents. Where plants or animals in the population share a common maternal parent or a common paternal parent, the plant or animal in which the trait is predicted may share the same common maternal or paternal parent, respectively.

The method may comprise, as an earlier step, a method of identifying an indicator of the trait in a plant or animal, as described above.

The plant or animal in which the indicator of the trait is identified may be the same genus and/or species as the plant or animal in which transcript abundance is determined for prediction of the trait. However, as discussed elsewhere herein, predictions of traits in one species may be performed based on correlations between transcript abundance and trait data obtained in other genus and/or species.

Thus, the invention may be used to predict one or more traits in a plant or animal, typically a previously untested plant or animal. As noted above, the method is useful for predicting heterosis or other trait in a plant or animal when heterosis or other trait is not yet determinable from the phenotype of the organism at the time, age or developmental stage at which the transcriptome is sampled. In a preferred embodiment the method comprises analysing the transcriptome of a plant prior to flowering.

Suitable methods of determining transcript abundance and of predicting heterosis or other traits based on transcript abundance are described in more detail elsewhere herein.

Once genes whose levels of transcript abundance are involved in heterosis or other traits have been identified for a given plant or animal species, further aspects of the invention may involve regulation of transcript abundance, regulation of expression of one or more of those genes, or regulation of one or more proteins encoded by those genes, in order to regulate, influence, increase or decrease heterosis or another trait in a plant or animal organism.

Thus, the invention may involve increasing or decreasing heterosis or other trait in an organism, by upregulating one or more genes or their encoded proteins, wherein transcript abundance of the one or more genes correlates positively with heterosis or other trait in the organism, or by down-regulating one or more genes or their encoded proteins in an organism, wherein transcript abundance of the one or more genes correlates negatively with heterosis or other trait in the organism. Thus, heterosis and other desirable traits in the organism may be increased using the invention. The invention also extends to plants and animals in which traits are up- or down-regulated using methods of the invention. The invention may comprise down-regulating one or more genes involved in stress avoidance or stress tolerance, wherein transcript abundance of the one or more genes is negatively correlated with heterosis, e.g. heterosis for biomass.

Examples of genes whose transcript abundance correlates positively with heterosis, and examples of genes whose transcript abundance correlates negatively with heterosis, are shown in Table 1 and Table 19. Additionally, transcript abundance of genes At1g67500 and At5g45500 correlates negatively with heterosis. In a preferred embodiment the one or more genes are selected from At1g67500 and At5g45500 and/or those shown in Table 1 and/or Table 19, or are orthologues of At1g67500 and/or At5g45500 and/or of one or more genes shown in Table 1 and/or Table 19.

The invention may involve increasing or decreasing a trait in an organism, by upregulating one or more genes whose transcript abundance correlates negatively with the trait in the organism, or by downregulating one or more genes whose transcript abundance correlates positively with the trait in hybrids. Thus, undesirable traits in organisms may be decreased using the invention.

Examples of genes whose transcript abundance correlates with particular traits are shown in Tables 3 to 17, Table 20 and Table 22. Preferred embodiments of the invention relate to one or more of those traits, and preferably to one or more of the listed genes for which transcript abundance is shown to correlate with those traits, as discussed elsewhere herein. Thus, the one or more genes may be selected from the genes shown in the relevant tables, or may be orthologues of those genes. For example, flowering time (e.g. as represented by leaf number at bolting) may be delayed (time to flowering increased, e.g. leaf number at bolting increased) by upregulating expression of one or more genes in Table 3A or Table 4A. Flowering time may be accelerated (time to flowering decreased, e.g. leaf number at bolting decreased) by downregulating expression of one or more genes in Table 3B or Table 4B.

A trait may be increased by upregulating a gene for which transcript abundance correlates positively with the trait or by downregulating a gene for which transcript abundance correlates negatively with the trait. A trait may be decreased by downregulating a gene for which transcript abundance correlates positively with the trait or by upregulating a gene for which transcript abundance correlates positively with the trait.

Upregulation of a gene involves increasing its level of transcription or expression, and thus increasing the transcript abundance of that gene. Upregulation of a gene may comprise expressing the gene from a strong and/or constitutive promoter such as 35S CaMV promoter. Upregulation may comprise increasing expression of an endogenous gene. Alternatively, upregulation may comprise expressing a heterologous gene in a plant or animal, e.g. from a strong and/or constitutive promoter. Heterologous genes may be introduced into plant or animal cells by any suitable method, and methods of transformation are well known in the art. A plant or animal cell may for example be transformed or transfected with an expression vector comprising the gene operably linked to a promoter e.g. a strong and/or constitutive promoter, for expression in the cell. The vector may integrate into the cell genome, or may remain extra-chromosomal.

By “promoter” is meant a sequence of nucleotides from which transcription may be initiated of DNA operably linked downstream (i.e. in the 3′ direction on the sense strand of double-stranded DNA).

“Operably linked” means joined as part of the same nucleic acid molecule, suitably positioned and oriented for transcription to be initiated from the promoter. DNA operably linked to a promoter is under transcriptional initiation regulation of the promoter.

Downregulation of a gene involves decreasing its level of transcription or expression, and thus decreasing the transcript abundance of that gene. Downregulation may be achieved for example by antisense or RNAi, using RNA complementary to messenger RNA (mRNA) transcribed from the gene.

Anti-sense oligonucleotides may be designed to hybridise to the complementary sequence of nucleic acid, pre-mRNA or mature mRNA, interfering with the production of polypeptide encoded by a given DNA sequence (e.g. either native polypeptide or a mutant form thereof), so that its expression is reduce or prevented altogether. Anti-sense techniques may be used to target a coding sequence, a control sequence of a gene, e.g. in the 5′ flanking sequence, whereby the antisense oligonucleotides can interfere with control sequences. Anti-sense oligonucleotides may be DNA or RNA and may be of around 14-23 nucleotides, particularly around 15-18 nucleotides, in length. The construction of antisense sequences and their use is described in refs. [42] and [43].

Small RNA molecules may be employed to regulate gene expression. These include targeted degradation of mRNAs by small interfering RNAs (siRNAs), post transcriptional gene silencing (PTGs), developmentally regulated sequence-specific translational repression of mRNA by micro-RNAs (miRNAs) and targeted transcriptional gene silencing.

A role for the RNAi machinery and small RNAs in targeting of heterochromatin complexes and epigenetic gene silencing at specific chromosomal loci has also been demonstrated. Double-stranded RNA (dsRNA)-dependent post transcriptional silencing, also known as RNA interference (RNAi), is a phenomenon in which dsRNA complexes can target specific genes of homology for silencing in a short period of time. It acts as a signal to promote degradation of mRNA with sequence identity. A 20-nt siRNA is generally long enough to induce gene-specific silencing, but short enough to evade host response. The decrease in expression of targeted gene products can be extensive with 90% silencing induced by a few molecules of siRNA.

In the art, these RNA sequences are termed “short or small interfering RNAs” (siRNAs) or “microRNAs” (miRNAs) depending in their origin. Both types of sequence may be used to down-regulate gene expression by binding to complimentary RNAs and either triggering mRNA elimination (RNAi) or arresting mRNA translation into protein. siRNA are derived by processing of long double stranded RNAs and when found in nature are typically of exogenous origin. Micro-interfering RNAs (miRNA) are endogenously encoded small non-coding RNAs, derived by processing of short hairpins. Both siRNA and miRNA can inhibit the translation of mRNAs bearing partially complimentary target sequences without RNA cleavage and degrade mRNAs bearing fully complementary sequences.

The siRNA ligands are typically double stranded and, in order to optimise the effectiveness of RNA mediated down-regulation of the function of a target gene, it is preferred that the length of the siRNA molecule is chosen to ensure correct recognition of the siRNA by the RISC complex that mediates the recognition by the siRNA of the mRNA target and so that the siRNA is short enough to reduce a host response.

miRNA ligands are typically single stranded and have regions that are partially complementary enabling the ligands to form a hairpin. miRNAs are RNA genes which are transcribed from DNA, but are not translated into protein. A DNA sequence that codes for a miRNA gene is longer than the miRNA. This DNA sequence includes the miRNA sequence and an approximate reverse complement. When this DNA sequence is transcribed into a single-stranded RNA molecule, the miRNA sequence and its reverse-complement base pair to form a partially double stranded RNA segment. The design of microRNA sequences is discussed in ref. [44].

Typically, the RNA ligands intended to mimic the effects of siRNA or miRNA have between 10 and 40 ribonucleotides (or synthetic analogues thereof), more preferably between 17 and 30 ribonucleotides, more preferably between 19 and 25 ribonucleotides and most preferably between 21 and 23 ribonucleotides. In some embodiments of the invention employing double-stranded siRNA, the molecule may have symmetric 3′ overhangs, e.g. of one or two (ribo)nucleotides, typically a UU of dTdT 3′ overhang. Based on the disclosure provided herein, the skilled person can readily design of suitable siRNA and miRNA sequences, for example using resources such as Ambion's siRNA finder, see http://www.ambion.com/techlib/misc/siRNA_finder.html. siRNA and miRNA sequences can be synthetically produced and added exogenously to cause gene downregulation or produced using expression systems (e.g. vectors). In a preferred embodiment the siRNA is synthesized synthetically.

Longer double stranded RNAs may be processed in the cell to produce siRNAs (see for example ref. [45]). The longer dsRNA molecule may have symmetric 3′ or 5′ overhangs, e.g. of one or two (ribo)nucleotides, or may have blunt ends. The longer dsRNA molecules may be 25 nucleotides or longer. Preferably, the longer dsRNA molecules are between 25 and 30 nucleotides long. More preferably, the longer dsRNA molecules are between 25 and 27 nucleotides long. Most preferably, the longer dsRNA molecules are 27 nucleotides in length. dsRNAs 30 nucleotides or more in length may be expressed using the vector pDECAP [46].

Another alternative is the expression of a short hairpin RNA molecule (shRNA) in the cell. shRNAs are more stable than synthetic siRNAs. A shRNA consists of short inverted repeats separated by a small loop sequence. One inverted repeat is complimentary to the gene target. In the cell the shRNA is processed by DICER into a siRNA which degrades the target gene mRNA and suppresses expression. In a preferred embodiment the shRNA is produced endogenously (within a cell) by transcription from a vector. shRNAs may be produced within a cell by transfecting the cell with a vector encoding the shRNA sequence under control of a RNA polymerase III promoter such as the human H1 or 7SK promoter or a RNA polymerase II promoter. Alternatively, the shRNA may be synthesised exogenously (in vitro) by transcription from a vector. The shRNA may then be introduced directly into the cell. Preferably, the shRNA molecule comprises a partial sequence of the gene to be down-regulated. Preferably, the shRNA sequence is between 40 and 100 bases in length, more preferably between 40 and 70 bases in length. The stem of the hairpin is preferably between 19 and 30 base pairs in length. The stem may contain G-U pairings to stabilise the hairpin structure.

siRNA molecules, longer dsRNA molecules or miRNA molecules may be made recombinantly by transcription of a nucleic acid sequence, preferably contained within a vector. Preferably, the siRNA molecule, longer dsRNA molecule or miRNA molecule comprises a partial sequence of the gene to be down-regulated.

In one embodiment, the siRNA, longer dsRNA or miRNA is produced endogenously (within a cell) by transcription from a vector. The vector may be introduced into the cell in any of the ways known in the art. Optionally, expression of the RNA sequence can be regulated using a tissue specific promoter. In a further embodiment, the siRNA, longer dsRNA or miRNA is produced exogenously (in vitro) by transcription from a vector.

In one embodiment, the vector may comprise a nucleic acid sequence according to the invention in both the sense and antisense orientation, such that when expressed as RNA the sense and antisense sections will associate to form a double stranded RNA. In another embodiment, the sense and antisense sequences are provided on different vectors.

Alternatively, siRNA molecules may be synthesized using standard solid or solution phase synthesis techniques which are known in the art. Linkages between nucleotides may be phosphodiester bonds or alternatives, for example, linking groups of the formula P(O)S, (thioate); P(S)S, (dithioate); P(O)NR′2; P(O)R′; P(O)OR6; CO; or CONR′2 wherein R is H (or a salt) or alkyl (1-12C) and R6 is alkyl (1-9C) is joined to adjacent nucleotides through —O— or —S—.

Modified nucleotide bases can be used in addition to the naturally occurring bases, and may confer advantageous properties on siRNA molecules containing them.

For example, modified bases may increase the stability of the siRNA molecule, thereby reducing the amount required for silencing. The provision of modified bases may also provide siRNA molecules which are more, or less, stable than unmodified siRNA.

The term ‘modified nucleotide base’ encompasses nucleotides with a covalently modified base and/or sugar. For example, modified nucleotides include nucleotides having sugars which are covalently attached to low molecular weight organic groups other than a hydroxyl group at the 3′position and other than a phosphate group at the 5′position. Thus modified nucleotides may also include 2′substituted sugars such as 2′-O-methyl-; 2-O-alkyl; 2-O-allyl; 2′-S-alkyl; 2′-S-allyl; 2′-fluoro-; 2′-halo or 2; azido-ribose, carbocyclic sugar analogues a-anomeric sugars; epimeric sugars such as arabinose, xyloses or lyxoses, pyranose sugars, furanose sugars, and sedoheptulose.

Modified nucleotides are known in the art and include alkylated purines and pyrimidines, acylated purines and pyrimidines, and other heterocycles. These classes of pyrimidines and purines are known in the art and include pseudoisocytosine, N4,N4-ethanocytosine, 8-hydroxy-N-6-methyladenine, 4-acetylcytosine, 5-(carboxyhydroxylmethyl) uracil, 5 fluorouracil, 5-bromouracil, 5-carboxymethylaminomethyl-2-thiouracil, 5-carboxymethylaminomethyl uracil, dihydrouracil, inosine, N6-isopentyl-adenine, 1-methyladenine, 1-methylpseudouracil, 1-methylguanine, 2,2-dimethylguanine, 2-methyladenine, 2-methylguanine, 3-methylcytosine, 5-methylcytosine, N6-methyladenine, 7-methylguanine, 5-methylaminomethyl uracil, 5-methoxy amino methyl-2-thiouracil, -D-mannosylqueosine, 5-methoxycarbonylmethyluracil, 5-methoxyuracil, 2 methylthio-N6-isopentenyladenine, uracil-5-oxyacetic acid methyl ester, psueouracil, 2-thiocytosine, 5-methyl-2 thiouracil, 2-thiouracil, 4-thiouracil, 5-methyluracil, N-uracil-5-oxyacetic acid methylester, uracil 5-oxyacetic acid, queosine, 2-thiocytosine, 5-propyluracil, 5-propylcytosine, 5-ethyluracil, 5-ethylcytosine, 5-butyluracil, 5-pentyluracil, 5-pentylcytosine, and 2,6,diaminopurine, methylpsuedouracil, 1-methylguanine, 1-methylcytosine.

Methods relating to the use of RNAi to silence genes in C. elegans, Drosophila, plants, and mammals are known in the art [47, 48, 49, 50, 51, 52, 53, 54, 55, 56, 57, 58, 59].

Other approaches to specific down-regulation of genes are well known, including the use of ribozymes designed to cleave specific nucleic acid sequences. Ribozymes are nucleic acid molecules, actually RNA, which specifically cleave single-stranded RNA, such as mRNA, at defined sequences, and their specificity can be engineered. Hammerhead ribozymes may be preferred because they recognise base sequences of about 11-18 bases in length, and so have greater specificity than ribozymes of the Tetrahymena type which recognise sequences of about 4 bases in length, though the latter type of ribozymes are useful in certain circumstances. References on the use of ribozymes include refs. [60] and [61].

The plant or animal in which the gene is upregulated or downregulated may be hybrid, recombinant or inbred. Thus, in some embodiments the invention may involve over-expressing genes correlated with one or more traits, in order to improve vigour or other characteristics of the transformed derivatives of inbred plants and animals.

In a further aspect, the invention is a method comprising:

analysing transcriptomes of parental plants or animals in a population of parental plants or animals;

measuring heterosis or other trait in a population of hybrids, wherein each hybrid in the population is a cross between a first plant or animal and a plant or animal selected from the population of parental plants or animals;

and

identifying a correlation between transcript abundance of one or more genes, preferably a set of genes, in the population of parental plants or animals and heterosis or other trait in the population of hybrids.

Thus, the invention provides a method of identifying an indicator of heterosis or other trait in a hybrid.

The plants or animals in the population whose transcriptomes are analysed are thus parents of the hybrids. These parents may be inbred or recombinant.

All hybrids in the population of hybrids used for developing each predictive model are the result of crossing one common parent with an array of different parents. Normally, all hybrids in the population share one common parent, which may be either the maternal parent or the paternal parent. Thus, the paternal parent of the all the hybrids in the population may be the “first parent plant or animal”, or the maternal parent of all the hybrids in the population may be the “first parent plant or animal”. For plants, a first female parent is normally crossed to a population of different male parents. For animals, a first male parent may preferably be crossed with a population of different females.

Suitable methods of determining or measuring heterosis in hybrids, of transcriptome analysis and of identifying correlations are discussed elsewhere herein.

Correlations between traits and transcript abundance represent models that may be used to predict traits in further plants or animals by determining transcript abundance in those plants or animals. The invention may thus be used to generate a model (e.g. a regression, as described in detail elsewhere herein) for predicting the trait based on transcript abundance of the one or more genes e.g. a set of genes.

Accordingly, in another aspect, the invention is a method of predicting heterosis or other trait in a hybrid, wherein the hybrid is a cross between a first plant or animal and a second plant or animal; comprising

determining the transcript abundance of one or more genes, preferably a set of genes, in the second plant or animal, wherein the transcript abundance of those one or more genes, or of the set of genes, in a population of parental plants or animals correlates with heterosis or other trait in a population of hybrids produced by crossing the first plant or animal with a plant or animal from the population of parental plants and animals; and

thereby predicting heterosis or other trait in the hybrid.

The invention may be used to predict one or more traits in hybrid offspring of parental plants or animals, based on transcript abundance in one of the parents. The parental plants or animals may be inbred or recombinant. Plants or animals may be referred to as “parents” or “parental plants or animals” even where they have not yet been crossed to produce a hybrid, since the invention may be used to predict traits in hybrids before those hybrids are produced. This is a particular advantage of the invention, in that methods of the invention may be used to predict heterosis or other trait in a potential hybrid, without needing to produce that hybrid in order to determine its heterosis or traits.

A plurality of plants or animals may be tested by determining transcript abundance using the method of the invention, each plant or animal representing the second parent for crossing to produce a hybrid, in order to identify a suitable plant or animal to use for breeding to produce a hybrid with a desired trait. A parent may then be selected for breeding based on the predicted trait for a hybrid produced by crossing that parent. Thus, in one example a germplasm collection, which may comprise a population of recombinants, may be screened for plants that may be suitable for inclusion in breeding programmes.

Following prediction of the trait in the hybrid, the inbred or recombinant plant or animal may be selected for breeding to produce a hybrid, e.g. as discussed further below. Alternatively, if the hybrid for which the trait is predicted has already been produced, that hybrid may be selected e.g. for further cultivation.

The method of predicting the trait may comprise, as an earlier step, a method of identifying an indicator of the trait in a hybrid, as described above.

When the method is used for predicting heterosis in hybrids based upon parental transcriptome data, for example data from inbred plants or animals, the one or more genes may comprise At3g112200 and/or one or more of the genes shown in Table 2, or one or more orthologues thereof.

When the method is used for predicting yield, e.g. grain yield, in hybrids based on parental transcriptome data, for example data from inbred plants or animals, e.g. maize, the one or more genes may comprise one or more of the genes shown in Table 22, or one or more orthologues thereof. For example, transcript abundance of one or more genes, e.g. a set of genes, from Table 22 may be determined in a maize plant and used for predicting yield in a hybrid cross between that maize line and B73.

Genes with transcript abundance correlating with other traits are shown in Tables 3 to 17 and Table 20, and transcript abundance of one or more of those genes in parental plants or animals may be used to predict those traits in accordance with hybrid offspring of those plants or animals, in accordance with this aspect of the invention. Alternatively, the invention may be used to identify other genes with transcript abundance in parental plants or animals correlating with those traits in their hybrid offspring.

By predicting heterosis and other traits in hybrids produced by crossing parental germplasm, whether they be inbred or recombinant, the invention allows selection of inbred or recombinant plants and animals that can be crossed to produce hybrids with high or improved levels of heterosis and desirable or improved levels of other traits.

Inbred or recombinant plants and animals may thus be selected on the basis of heterosis or other trait predicted in hybrids produced by crossing those plants and animals.

Accordingly, one aspect of the invention is a method comprising:

determining transcript abundance of one or more genes, preferably a set of genes, in parental plants or animals, wherein the transcript abundance of the one or more genes in a population of parental plants or animals correlates with heterosis or other trait in hybrid crosses between a first parental plant or animal and plants or animals from the population of parental plants or animals;

selecting one of the parental plants or animals; and

producing a hybrid by crossing the selected plant or animal and a different plant or animal, e.g. by crossing the selected plant or animal and the first plant or animal.

Thus, one or more traits may be predicted for hybrid crosses between the parental plants or animals, and then a parental plant or animal predicted to produce a hybrid with a desired trait e.g. late flowering, high heterosis, and/or high yield, and/or with a reduced undesirable trait, may be selected. Methods for predicting traits are discussed in more detail elsewhere herein.

Genes whose transcript abundance correlates with heterosis or other trait in hybrids produced by crossing a first plant or animal and other plants or animals are referred to elsewhere herein, and may be At3g112200 and/or one or more genes selected from the genes in Table 2, or orthologues thereof. Genes with transcript abundance correlating with other traits are shown in Tables 3 to 17 and Table 20, as described elsewhere herein.

Hybrids produced by methods of the invention may be raised or cultivated, e.g. to maturity or breeding age. The invention also extends to hybrids produced using methods of the invention.

The invention may be applied to any trait of interest. For example, traits to which the invention applies include, but are not limited to, heterosis, flowering time or time to flowering, seed oil content, seed fatty acid ratios, and yield. Examples genes whose transcript abundance correlates with certain traits are shown in the appended Tables. For animals, preferred traits are heterosis, yield and productivity. Traits such as yield may be underpinned by heterosis, and the invention may relate to modelling and/or predicting yield and other traits, and/or modelling and/or predicting heterosis for yield and other traits, based on transcript abundances of genes.

Genes in Tables shown herein are identified by AGI numbers, Affymetrix Probe identifier numbers and/or GenBank database accession numbers. AGI numbers can be used to identify the gene from TAIR (The Arabidopsis Information Resource), available on-line at http://www.arabidopsis.org/index.jsp, or findable by searching for “TAIR” and/or “Arabidopsis information resource” using an internet search engine. Affymetrix Probe identifier numbers can be used to identify sequences from Netaffx, available on-line at http://www.affymetrix.com/analysis/index.affx, or findable by searching for “netaffx” and/or “Affymetrix” using an internet search engine. It is now possible to convert between the two identifier formats using the converter, from Toronto university, currently available at http://bbc.botany.utoronto.ca/ntools/cgi-bin/ntools_{—}agi_converter.cgi, or findable by searching for “agi converter” using an internet search engine. GenBank accession numbers can be used to obtain the corresponding sequence from GenBank, available at http://www.ncbi.nlm.nih.gov/Genbank/index.html or findable using any internet search engine.

A set of genes may comprise a set of genes selected from the genes shown in a table herein.

In methods of the invention relating to heterosis, the one or more genes may comprise one or more of the 70 genes listed in Table 1 or one or more orthologues thereof, and/or may comprise one or more of the genes listed in Table 19 or one or more orthologues thereof.

In methods relating to traits other than heterosis, the trait may for example be a trait referred for Tables 3 to 17, Table 20 or Table 22, and the one or more genes may comprise one or more of the genes shown in the relevant tables, or one or more orthologues thereof. Preferably, the genes in Tables 3 to 17, 20 and/or 22 are used for predicting or influencing (increasing or decreasing) traits in inbred plants or animals. However, the genes may also be used for predicting, increasing or decreasing traits in recombinants and/or hybrids.

When the trait is flowering time, or time to flowering, in plants, e.g. as represented by leaf number at bolting, the one or more genes may comprise one or more genes shown in Table 3 or Table 4, or orthologues thereof. Table 3 shows genes for which transcript abundance was shown to correlate with flowering time in vernalised plants, and Table 4 shows genes for which transcript abundance was shown to correlate with flowering time in unvernalised plants. These may be used for predicting flowering time in vernalised or unvernalised plants, respectively. However, as discussed elsewhere herein, transcript abundance of genes which correlates with a trait in vernalised plants may also correlate (normally according to a different model or equation) with the trait in unvernalised plants. Thus, transcript abundance of genes in either Table 3 or Table 4 may be used to predict flowering time in either vernalised or unvernalised plants, using the appropriate correlation for vernalised or unvernalised plants respectively.

Whilst the transcript abundance data of the genes listed in many of the Tables herein were used in our example for predicting traits in vernalised plants, these data could also be used to predict traits in unvernalised plants. Thus, a first correlation may be identified between transcript abundance and the trait in vernalised plants, and a second correlation may be identified between transcript abundance and the trait in unvernalised plants. The appropriate model may then be used to predict the trait in vernalised or unvernalised plants respectively, based on transcript abundance of one or more of those genes, or orthologues thereof.

Oil content is a useful trait to measure in plants. This is one of the measures used to determine seed quality, e.g. in oilseed rape.

When the trait is oil content of seeds, e.g. as represented by % dry weight, the one or more genes may comprise one or more genes shown in Table 6, or orthologues thereof.

Seed quality may also be represented by the proportion, percentage weight or ratio of certain fatty acids.

Normally, seed traits are predicted for vernalised plants, e.g. oilseed rape in the UK is grown as a Winter crop and will therefore be vernalised at the time of trait expression (seed production in this example). However, predictions may be for either vernalised or unvernalised plants.

When the trait is ratio of 18:2/18:1 fatty acids in seed oil, the one or more genes may comprise one or more genes selected from the genes shown in Table 7, or orthologues thereof.

When the trait is ratio of 18:3/18:1 fatty acids in seed oil, the one or more genes may comprise one or more genes selected from the genes shown in Table 8, or orthologues thereof.

When the trait is ratio of 18:3/18:2 fatty acids in seed oil, the one or more genes may comprise one or more genes selected from the genes shown in Table 9, or orthologues thereof.

When the trait is ratio of 20C+22C/16C+18C fatty acids in seed oil, the one or more genes may comprise one or more genes selected from the genes shown in Table 10, or orthologues thereof.

When the trait is ratio of polyunsaturated/monounsaturated+saturated 18C fatty acids in seed oil, the one or more genes may comprise one or more genes selected from the genes shown in Table 12, or orthologues thereof.

When the trait is % 16:0 fatty acid in seed oil, the one or more genes may comprise one or more genes selected from the genes shown in Table 14, or orthologues thereof.

When the trait is % 18:1 fatty acid in seed oil, the one or more genes may comprise one or more genes selected from the genes shown in Table 15, or orthologues thereof.

When the trait is % 18:2 fatty acid in seed oil, the one or more genes may comprise one or more genes selected from the genes shown in Table 16, or orthologues thereof.

When the trait is % 18:3 fatty acid in seed oil, the one or more genes may comprise one or more genes selected from the genes shown in Table 17, or orthologues thereof.

It may be desirable to predict responsiveness of a plant trait to vernalisation, and this may be measured for example as the ratio of a trait measurement in vernalised plants to the trait measurement in unvernalised plants.

For example, responsiveness of flowering time to vernalisation may be measured as the ratio of leaf number at bolting in vernalised plants to leaf number at bolting in unvernalised plants. Genes whose transcript abundance correlates with this ratio are shown in Table 5. Thus, in embodiments of the invention where the trait is responsiveness of plant flowering time to vernalisation, the one or more genes may comprise one or more genes shown in Table 5, or orthologues thereof.

Responsiveness to vernalisation of the ratio of 20C+22C/16C+18C fatty acids in seed oil may be measured as the ratio of (ratio of 20C+22C/16C+18C fatty acids in seed oil in vernalised plants) to (ratio of 20C+22C/16C+18C fatty acids in seed oil in unvernalised plants). Genes whose transcript abundance correlates with this ratio are shown in Table 11. Thus, in embodiments of the invention where the trait is responsiveness of this ratio to vernalisation, the one or more genes may comprise one or more genes shown in Table 11, or orthologues thereof.

Responsiveness to vernalisation of the ratio of polyunsaturated/monounsaturated+saturated 18C fatty acids in seed oil may be measured as the ratio of (ratio of polyunsaturated/monounsaturated+saturated 18C fatty acids in seed oil in vernalised plants) to (ratio of polyunsaturated/monounsaturated+saturated 18C fatty acids in seed oil in unvernalised plants). Genes whose transcript abundance correlates with this ratio are shown in Table 13. Thus, in embodiments of the invention where the trait is responsiveness of this ratio to vernalisation, the one or more genes may comprise one or more genes shown in Table 13, or orthologues thereof.

When the trait is yield, the one or more genes may comprise one or more of the genes shown in Table 20 or Table 22, or orthologues thereof.

Genes in Tables 1 to 17 are from Arabidopsis thaliana, and may be used in embodiments of the invention relating to A. thaliana or to another organism, such as for predicting or increasing heterosis in a plant or animal (genes of Tables 1 and 2, or orthologues thereof), or for predicting, increasing or decreasing another trait in A. thaliana or other plant. Genes in Tables 19, and 22 are from maize, and may be used in embodiments of the invention relating to maize or to another organism, such as for predicting or increasing heterosis in a plant or animal (genes of Table 19 or orthologues thereof) or for predicting, increasing or decreasing another trait in maize or other plant.

We have demonstrated that transcript abundance in plants of genes shown in Tables 1, 3 to 17, 20 and 22 is predictive of the described traits in those plants. In some embodiments of the invention relating to use of parental transcriptome data for prediction of traits in hybrids, transcript abundance in plants of genes shown in Tables 1, 3 to 17, 20 and 22 or orthologues thereof may be used to predict the described traits in hybrid offspring of those plants.

Preferably, in embodiments of the invention relating to use of parental transcriptome data for prediction of heterosis in hybrids, transcript abundance in plants of At3g112200 and/or of genes shown in Table 2, or orthologues thereof, is used to predict the magnitude of heterosis in hybrid offspring of those plants.

In embodiments of the invention relating to use of parental transcriptome data for prediction of yield, e.g. grain yield, in hybrids, transcript abundance in plants of one or more genes shown in Table 22 is used to predict the yield in hybrid offspring of those plants.

Heterosis or other trait is normally determined quantitatively. As noted above, heterosis may be described relative to the mean value of the parents (Mid-Parent Heterosis, MPH) or relative to the “better” of the parents (Best-Parent Heterosis, BPH).

Heterosis may be determined on any suitable measurement, e.g. size, fresh or dry weight at a given age, or growth rate over a given time period, or in terms of some measure of yield or quality. Heterosis may be determined using historical data from the parental and/or hybrid lines.

Heterosis may be calculated based on size, for which size measurements may for example be taken of the maximum length and width of the plant or animal, or of a part of the plant or animal, e.g. using electronic callipers. For plants, heterosis may be calculated based on total aerial fresh weight of the plants, which may be determined by cutting off all above soil plant material, quickly removing any soil attached, and weighing.

In preferred embodiments, heterosis is heterosis for yield (e.g. in plants or animals, yield of harvestable product), or heterosis for fresh weight (e.g. fresh weight of aerial parts of a plant).

The magnitude of heterosis may thus be determined, and is normally expressed as a % value. For example, mid parent heterosis for fresh weight can be presented as a percentage figure calculated as (weight of the hybrid−mean weight of the parents)/mean weight of the parents. Best parent heterosis for fresh weight can be presented as a percentage figure calculated as (weight of the hybrid−weight of the heaviest parent)/weight of the heaviest parent.

For other traits, an appropriate measurement can be determined by the skilled person. Some traits can be directly recorded as a magnitude, e.g. seed oil content, weight of plant or animal, or yield. Other traits would be determined with reference to another indicator, e.g. flowering time may be represented by leaf number at bolting. The skilled person is able to select an appropriate way to quantify a particular trait, e.g. as a magnitude, ratio, degree, volume, time or rate, and to measure suitable factors representative of the relevant trait.

A transcript is messenger RNA transcribed from a gene. The transcriptome is the contribution of each gene in the genome to the mRNA pool. The transcriptome may be analysed and/or defined with reference to a particular tissue, as discussed elsewhere herein. Analysis of the transcriptome may thus be determination of transcript abundance of one or more genes, or a set of genes.

Transcriptome analysis or determination of transcript abundance is normally performed on tissue samples from the plants or animals. Any part of the plant or animal containing RNA transcripts may be used for transcriptome analysis. Where an organism is a plant, the tissue is preferably from one or more, preferably all, aerial parts of the plant, preferably when the plant is in the vegetative phase before flowering occurs. In some embodiments, transcriptome analysis may be performed on seeds. Methods of the invention may involve taking tissue samples from the plants or animals. In methods of predicting the heterosis or other trait, the sampled organism may remain viable after the tissue sample has been taken. Where prediction is to be performed for genetically identical plants or animals, which may be grown on a different occasion, tissues may include all parts or all aerial plants or a whole seed (for plants) or the whole embryo (for animals). Where prediction is to be performed for the exact plant sampled, a subset of the leaves of the plant may be sampled. However, there is no requirement for the organism to remain viable, since sampling of one or more individuals for transcriptome analysis that results in loss of viability may be used for the prediction of heterosis or other traits in hybrid, inbred or recombinant organisms of similar or identical genetic composition grown on either the same or a different occasion and under the same or different environmental conditions.

Typically, transcriptome analysis is performed on RNA extracted from the plant or animal. The invention may comprise extracting RNA from a tissue sample of the hybrid or inbred plant or animal. Any suitable methods of RNA extraction may be used, e.g. see the protocol set out in the Examples.

Transcriptome analysis comprises determining the abundance of an array of RNA transcripts in the transcriptome. Where oligonucleotide chips are used for transcriptome analysis, the numbers of genes potentially used for model development are the numbers of probes on the GeneChips—ca. 23,000 for Arabidopsis and ca. 18,000 for the present maize Chip. Thus, while in some embodiments, the transcript abundance of each gene in the genome is assessed, normally transcript abundance of a selected array of genes in the genome is assessed.

Various techniques are available for transcriptome analysis, and any suitable technique may be used in the invention. For example, transcriptome analysis may be performed by bringing an RNA sample into contact with an oligonucleotide array or oligonucleotide chip, and detecting hybridisation of RNA transcripts to oligonucleotides on the array or chip. The degree of hybridisation to each oligonucleotide on the chip may be detected. Suitable chips are available for various species, or may be produced. For example, Affymetrix GeneChip array hybridisation may be used, for example using protocols described in the Affymetrix Expression Analysis Technical Manual II (currently available at http://www.affymetrix.com/support/technical/manuals.affx. or findable using any internet search engine). For detailed examples of transcriptome analysis, please see the Examples below.

Transcript abundance of one or more genes, e.g. a set of genes, may be determined, and any of the techniques above may be employed. Alternatively, reverse transcriptase may be used to synthesise double stranded DNA from the RNA transcript, and quantitative polymerase chain reaction (PCR) may be used for determining abundance of the transcript.

Transcript abundance of a set of genes may be determined. A set of genes is a plurality of genes, e.g. at least 10, 20, 30, 40, 50, 60, 70, 80, 90 or 100 genes. The set may comprise genes correlating positively with a trait and/or genes correlating negatively with the trait. As noted below, preferably, the set of genes is one for which transcript abundance of that set of genes allows prediction of heterosis or other trait. The skilled person may use methods of the invention to determine which genes are most useful for predicting heterosis or other traits in hybrids, and therefore to determine which genes can most usefully be assessed for transcript abundance in accordance with the invention. Additionally, examples of sets of genes for prediction of heterosis and other traits are shown herein.

Preferably, analysis of transcript abundance is performed in the same way for the plants or animals used to generate a model or correlation with a trait “model organism” as for the plants or animals in which the trait is predicted based on that model “test organism”. Preferably, the model and test organisms are raised under identical conditions and transcriptome analysis is performed on both the model and test organisms at the same age, time of day and in the same environment, in order to maximise the predictive value of the model based on transcriptome data from the model organisms.

Accordingly, predicting a trait in a test plant or animal may comprise determining transcript abundance of one or more genes in the test plant or animal at a particular age, wherein transcript abundance of the one or more genes in the transcriptome of model plants or animals at that age conditions correlates with the trait. Thus, preferably transcript abundance in the organism (i.e. plant or non-human animal) is determined when the organism is at the same age as the organisms in the population on which the correlation between transcript abundance and heterosis or other trait was determined. Thus, predicting the degree of a trait in an organism may comprise determining the abundance of transcripts of one or more genes, preferably a set of genes, in the organism at a selected age, and determining the transcript abundance of one or more genes, preferably a set of genes, wherein the transcript abundance of those one or more genes or set of genes in the transcriptome of organisms at the said age correlates with heterosis or other trait in the organism.

As noted elsewhere herein, the age at which transcript abundance is determined may be earlier than the age at which the trait is expressed, e.g. where the trait is flowering time the transcriptome analysis may be performed when plants are in vegetative phase.

Preferably, transcriptome analysis and determination of transcript abundance is determined on plant or animal material sampled at a particular time of day. For example, plant tissue samples may be taken at the middle of the photoperiod (or as close as practicable). Thus, when predicting a trait by determining the transcript abundance of one or more genes (e.g. set of genes) whose transcript abundance correlates with that trait, the transcript abundance data for making the prediction are preferably determined at the same time of day as the transcript abundance data used to generate the correlation.

Some aspects of the invention relate to plants, such as cereals, that require vernalisation before flowering. Vernalisation is a period of exposure to cold, which promotes subsequent flowering. Plants requiring vernalisation do not flower the same year when sown in Spring, but continue to grow vegetatively. Such plants (“winter varieties”) require vernalisation over Winter, and so are planted in the Autumn to flower the following year. In the present invention, plants may be vernalised or unvernalised.

Transcriptome data may be obtained from plants when vernalised or unvernalised, and those data may be used to identify a correlation between transcript abundance and a trait measured in vernalised plants and/or a correlation between transcript abundance and the trait measured in unvernalised plants. Thus, surprisingly, we have shown that transcriptome data from vernalised plants can be used to develop a model for predicting traits in unvernalised plants, as well as being useful to develop a model for predicting traits in vernalised plants.

In methods of the invention, comparisons and predictions are preferably between plants or animals of the same genus and/or species. Thus, methods of predicting heterosis or other trait in a plant or animal may be based on correlations obtained in a population of hybrids, inbreds or recombinants of that species of plant or animal. However, as discussed elsewhere herein, correlations obtained in one species may be applied to other species, e.g. to other plants or other animals in general, or to both plants and animals, especially where the other species exhibit similar traits. Thus, the test organism in which the trait is predicted need not be of the same species as the model organisms in which the correlation for prediction of the trait was developed.

Determination of transcript abundance for prediction of a trait is normally performed on the same type of tissue as that in which the correlation between the trait and transcript abundance was determined. Thus, predicting the degree of heterosis in a hybrid may comprise determining transcript abundance in tissue in or from the hybrid, and determining the transcript abundance of one or more genes, preferably a set of genes, wherein the transcript abundance of those one or more genes in the transcriptome of the said tissue in hybrids correlates with heterosis or other trait in hybrids.

Data may be compiled, the data comprising:

(i) a value representing the magnitude of heterosis or other trait in each plant or animal;
(ii) transcriptome analysis data in each plant or animal, wherein the transcriptome analysis data represents the abundance of each of an array of gene transcripts.

For determination of a correlation, data should be obtained from a plurality of plants or animals. In methods of the invention it is thus preferable that transcriptome analyses are performed and traits are determined for at least three plants or animals, more preferably at least five, e.g. at least ten. Use of more plants or animals, e.g. in a population, can lead to more reliable correlations and thus increase the quantitative accuracy of predictions according to the invention.

Any suitable statistical analysis may be employed to identify a correlation between transcript abundance of one or more genes in the transcriptomes of the plants or animals and the magnitude of heterosis or other trait. The correlation may be positive or negative. For example, it may be found that some transcripts have an abundance correlating positively with heterosis or other trait, while other transcripts have an abundance correlating negatively with heterosis or other trait.

Data from each plant or animal may be recorded in relation to heterosis and/or multiple other traits. Accordingly, the invention may be used to identify which genes have a transcript abundance correlating with which traits in the organism. Thus, a detailed profile may be compiled for the relationship between transcript abundance and heterosis and other traits in the population of organisms.

Typically, an analysis is performed using linear regression to identify the relationship between transcript abundance and the magnitude of heterosis (MPH and/or BPH) or other trait. An F-value may then be calculated. The F value is a standard statistic for regression. It tests the overall significance of the regression model. Specifically, it tests the null hypothesis that all of the regression coefficients are equal to zero. The F value is the ratio of the mean regression sum of squares divided by the mean error sum of squares with values that range from zero upward. From this we get the F Prob (the probability that the null hypothesis that there is no relationship is true). A low value implies that at least some of the regression parameters are not zero and that the regression equation does have some validity in fitting the data, indicating that the variables (gene expression level) are not purely random with respect to the dependent variable (trait value at that point).

Preferably a correlation identified using the invention is a statistically significant correlation. Significance levels may be determined as F statistics from the regression Mean Square in the analysis of variance tables of the linear regression analysis. Statistical significance may be indicated for example by F<0.05, or <0.001.

Other potential relationships exist between gene expression and plant phenotype, besides simple linear relationships. For example, relationships may fall on a logistic curve. A computer model (e.g. GenStat) may be used to fit the data to a logistic curve.

Non-linear modelling covers those expression patterns that form any part of a sigmoidal curve, from exponential-type patterns, to threshold and plateau type patterns. Non-linear methods may also cover many linear patterns, and thus may preferentially be used in some embodiments of the invention.

Normally a computer program is used to identify the correlation or correlations. For example, as described in more detail in the Examples below, linear regression analysis may be performed using GenStat, e.g. Program 3 below is an example of a linear regression programme to identify linear regressions between the hybrid transcriptome and MPH.

More generally, each of the methods of the above aspects may be implemented in whole or in part by a computer program which, when executed by a computer, performs some or all of the method steps involved. The computer program may be capable of performing more than one of the methods of the above aspects.

Another aspect of the invention provides a computer program product containing one or more such computer programs, exemplified by a data carrier such as a compact disk, DVD, memory storage device or other non-volatile storage medium onto which the computer program(s) is/are recorded.

A further aspect of the invention is a computer system having a processor and a display, wherein the processor is operably configured to perform the whole or part of the method of one or more of the above aspects, for example by means of a suitable computer program, and to display one or more results of those methods on the display. Typically the computer will be a general purpose computer and the display will be a monitor. Other output devices may be used instead of or in addition to the display including, but not limited to, printers.

Preferably, a set of genes, e.g. less than 1000, 500, 250 or 100 genes, is identified for which transcript abundance correlates with heterosis or other trait, wherein transcript abundance of that set of genes allows prediction of heterosis or other trait. A smaller set of genes that remains predictive of the trait may then be identified by iterative testing of the precision of predictions by progressively reducing the numbers of genes in the models, preferentially retaining those with the best correlation of transcript abundance with heterosis or the other trait, e.g. genes with the most significant (e.g. p<0.001) correlations between transcript abundance and traits. Thus, methods of the invention may comprise identifying a correlation between a trait and transcript abundance of a set of genes in transcriptomes, and then identifying a smaller set or sub-set of genes from within that set, wherein transcript abundance of the smaller set of genes is predictive of the trait. Preferably the smaller set of genes retains most of the predictive power of the set of genes.

The magnitude of heterosis or other trait may be predicted from transcript abundance of one or more genes, preferably of a set of genes as noted above, based on a correlation of the transcript abundance with heterosis or other trait (e.g. a linear regression as described above).

Thus, the equation of the linear regression line (linear or non-linear) for each of the gene transcripts showing a correlation with magnitude of heterosis or other trait may be used to calculate the expected magnitude of heterosis or other trait from the transcript abundance of that gene. The aggregate of the predicted contributions for each gene is then used to calculate the trait value (e.g. as the sum of the contribution from each gene transcript, normalised by the coefficient of determination, r².

### DRAWINGS

FIG. 1: Workflows for the analysis of expression data for the investigation of heterosis. a) Standard protocols; b) Recommended Prediction Protocol; c) Alternative ‘Basic’ Prediction Protocol; d) Transcription Remodelling Protocol

### LIST OF TABLES

Table 1: Genes in Arabidopsis thaliana hybrids, transcripts of which correlate with magnitude of heterosis in the hybrids

Table 2: Genes in Arabidopsis thaliana inbred lines, transcripts of which correlate with magnitude of heterosis in hybrids produced by crossing those lines with Ler ms1. (A: positive correlation; B: negative correlation)

Table 3: Genes in Arabidopsis thaliana inbred lines, showing correlation in transcript abundance with leaf number at bolting in vernalised plants (A: positive correlation; B: negative correlation)

Table 4: Genes in Arabidopsis thaliana inbred lines showing correlation in transcript abundance with leaf number at bolting in unvernalised plants (A: positive correlation; B: negative correlation)

Table 5: Genes in Arabidopsis thaliana inbred lines showing correlation in transcript abundance with ratio of leaf number at bolting (vernalised plants)/leaf number at bolting (unvernalised plants). (A: positive correlation; B: negative correlation)

Table 6: Genes in Arabidopsis thaliana inbred lines showing correlation between transcript abundance and oil content of seeds, % dry weight in vernalised plants (A: positive correlation; B: negative correlation)

Table 7: Genes in Arabidopsis thaliana inbred lines showing correlation between transcript abundance and ratio of 18:2/18:1 fatty acids in seed oil in vernalised plants (A: positive correlation; B: negative correlation)

Table 8: Genes in Arabidopsis thaliana inbred lines showing correlation between transcript abundance and ratio of 18:3/18:1 fatty acids in seed oil in vernalised plants (A: positive correlation; B: negative correlation)

Table 9: Genes in Arabidopsis thaliana inbred lines showing correlation between transcript abundance and ratio of 18:3/18:2 fatty acids in seed oil in vernalised plants (A: positive correlation; B: negative correlation)

Table 10: Genes in Arabidopsis thaliana inbred lines showing correlation between transcript abundance and ratio of 20C+22C/16C+18C fatty acids in seed oil in vernalised plants (A: positive correlation; B: negative correlation)

Table 11: Genes in Arabidopsis thaliana inbred lines showing correlation between transcript abundance and ratio of (ratio of 20C+22C/16C+18C fatty acids in seed oil (vernalised plants))/(ratio of 20C+22C/16C+18C fatty acids in seed oil (unvernalised plants)) (A: positive correlation; B: negative correlation)

Table 12: Genes in Arabidopsis thaliana inbred lines showing correlation between transcript abundance and ratio of polyunsaturated/monounsaturated+saturated 18C fatty acids in seed oil in vernalised plants (A: positive correlation; B: negative correlation)

Table 13: Genes in Arabidopsis thaliana inbred lines showing correlation between transcript abundance and ratio of (ratio of polyunsaturated/monounsaturated+saturated 18C fatty acids in seed oil (vernalised plants))/(ratio of polyunsaturated/monounsaturated+saturated 18C fatty acids in seed oil (unvernalised plants)) (A: positive correlation; B: negative correlation)

Table 14: Genes in Arabidopsis thaliana inbred lines showing correlation between transcript abundance and % 16:0 fatty acid in seed oil in vernalised plants (A: positive correlation; B: negative correlation)

Table 15: Genes in Arabidopsis thaliana inbred lines showing correlation between transcript abundance and % 18:1 fatty acid in seed oil (vernalised plants)

(A: positive correlation; B: negative correlation)

Table 16: Genes in Arabidopsis thaliana Inbred Lines Showing correlation between transcript abundance and % 18:2 fatty acid in seed oil (vernalised plants) (A: positive correlation; B: negative correlation)

Table 17: Genes in Arabidopsis thaliana inbred lines showing correlation between transcript abundance and % 18:3 fatty acid in seed oil (vernalised plants) (A: positive correlation; B: negative correlation)

Table 18: Prediction of complex traits in inbred lines (accessions) using models based on accession transcriptome data

Table 19: Genes in maize for prediction of heterosis for plant height. Data were obtained in plants at CLY location only (model from 13 hybrids). Representative public ID shows GenBank accession numbers. (A: positive correlation; B: negative correlation)

Table 20: Genes in maize for prediction of average yield. Data were obtained in plants across 2 sites, MO and L (model from 12 hybrids to predict 3). Representative public ID shows GenBank accession numbers. (A: positive correlation; B: negative correlation)

Table 21: Pedigree and seedling growth characteristics of maize inbred lines used in Example 6a

Table 22: Maize genes for which transcript abundance in inbred lines of the training dataset is correlated (P<0.00001) with plot yield of hybrids with line B73. A negative value for the slope indicates a negative correlation between abundance of the transcript and yield, and a positive value indicates a positive correlation.

Table 23: Maize plot yield data for Example 6a.

### EXAMPLES

### Example 1

### Transcriptome Remodelling in Arabidopsis Hybrids

Our initial studies employed Arabidopsis thaliana. We conducted all of our heterosis analyses in F1 hybrids between accessions of A. thaliana, which can be considered inbred lines due to their lack of heterozygosity. The genome sequence of A. thaliana is available [62] and resources for transcriptome analysis in this species are well developed [63]. A. thaliana also shows a wide range of magnitude of hybrid vigour [7, 64, 65].

The null hypothesis is that all parental alleles contribute to the transcriptome in an additive manner, i.e. if alleles differ in their contribution to transcript abundance, the observed value in the hybrid will be the mean of the parent values. There are six patterns of transcript abundance in hybrids that depart from this expected additive effect of contrasting parental alleles [28]:

(i) transcript abundance in the hybrid is higher than either parent;
(ii) transcript abundance in the hybrid is lower than either parent;
(iii) transcript abundance in the hybrid is similar to the maternal parent and both are higher than the paternal parent;
(iv) transcript abundance in the hybrid is similar to the paternal parent and both are higher than the maternal parent;
(v) transcript abundance in the hybrid is similar to the maternal parent and both are lower than the paternal parent;
(vi) transcript abundance in the hybrid is similar to the paternal parent and both are lower than the maternal parent.

When using quantitative analytical methods, the terms “higher than”, “lower than” and “similar to” can be defined by specific fold-difference criteria. Although differences in the contributions to the transcriptome of divergent alleles in maize hybrids has been reported as common [29, 66] the lack of absolute quantitative analysis of transcript abundance in parental inbred lines means that it is not possible to determine whether the observed effects are due to allelic interaction in the hybrid or simply the expected additive effects of alleles with differing transcript abundance characteristics. We would not consider such additive effects as components of transcriptome remodelling.

We produced reciprocal hybrids between A. thaliana accessions Kondara and Br-0, and between Landsberg er ms1 and Kondara, Mz-0, Ag-0, Ct-1 and Gy-0, with Landsberg er ms1 as the maternal parent. Hybrids and parents were grown under identical environmental conditions and heterosis calculated for the fresh weight of the aerial parts of the plants after 3 weeks growth (see Materials and Methods). The heterosis observed for each combination was recorded (BPH (%) and MPH (%))

RNA was extracted from the same material and the transcriptome was analysed using ATH1 GeneChips. Plants were grown in three replicates on three successive occasions. RNA was pooled from the three replicates for analysis of gene expression levels on each occasion.

Transcript abundance values in A. thaliana hybrids were compared over all experimental occasions and genes showing differences, at defined fold-levels from 1.5 to 3.0, corresponding to the six patterns indicative of transcriptome remodelling, were identified. Genes with transcript abundance differing between the parents by the same defined fold-level were also identified. The number of genes that appeared consistently in each of these 8 categories across all 3 experimental occasions was counted. To assess whether the number of genes classified into each category differed from that expected by chance, permutation analysis (bootstrapping) was used to calculate an expected value under the null hypothesis of no remodelling.

The significance of the experimental results was assessed, for each category independently, using Chi square tests. The results of the analysis, summarised in Table 1 for 2-fold differences, show that transcriptome remodelling occurred in all of the hybrids analysed, with most individual observations showing highly significant (p<0.001) divergence from the null hypothesis. Similar analyses were conducted for 1.5- and 3-fold differences, with extensive remodelling also being identified. Based on the analysis of gene ontology information, there were no obvious functional relationships of the remodelled genes in the hybrids.

Further analysis of selected genes from these categories were conducted using additional GeneChip hybridisation experiments and by quantitative RT-PCR, and confirmed the transcript abundance patterns. GeneChip hybridization was also performed using genomic DNA from accessions Kondara, Br-0 and Landsberg er ms1, to assess the proportion of differences between parental transcriptomes attributable to sequence polymorphisms that would prevent accurate reporting of transcript abundance by the arrays. We found that ca. 20% of the differences between parental transcriptomes may be attributable to sequence variation. However, this does not affect the remodelling analysis, as additivity of allelic contributions to the mRNA pool in hybrids where one parental allele failed to report accurately on the array would result in intermediate signal strength, so would not be assigned to any of the remodelled classes.

The relationship of transcriptome remodelling with hybrid vigour was assessed by carrying out linear regression of the number of genes remodelled in each hybrid combination, at the 1.5, 2 and 3-fold levels, on the magnitude of heterosis observed. This revealed a strong relationship between heterosis and the transcriptome remodelling at the 1.5-fold level (r+0.738, coefficient of determination r²=0.544 for MPH; r=+0.736, r²=0.542 for BPH). The correlation was more modest between heterosis and the transcriptome remodelling involving higher fold level changes (r²=0.213 and 0.270 for MPH and BPH, respectively, for 2-fold changes; r²=0.300 and 0.359 for MPH and BPH, respectively, for 3-fold changes). There was extensive remodelling, at all fold changes, even in the hybrid combinations showing the least heterosis. Consequently, the majority of remodelling events identified that result in transcript abundance changes of 2-fold or greater, even in strongly heterotic hybrids, are likely to be unrelated to heterosis. The most highly enriched class in heterotic hybrids is those genes showing 1.5-fold differential abundance, which is below the threshold usually set in transcriptome analysis experiments.

Heterosis shows an inconsistent relationship with the degree of relatedness of parental lines, with an absence of correlation reported between heterosis and genetic distance in A. thaliana [7]. We estimated the genetic distance between the accessions used in the hybrid combinations we have analysed, and these are shown in Table 1. To assess the relationship of transcriptome remodelling with genetic distance, we regressed the number of genes classified as having remodelled transcript abundance in each hybrid combination against genetic distance. We found that transcriptome remodelling is associated with genetic distance in the higher-fold remodelling classes (r²=0.351 and 0.281 for 2 and 3-fold changes respectively), but not for 1.5-fold remodelling (r²=0.030). We found no relationship between heterosis and genetic distance, in accordance with previous reports in A. thaliana (r²=0.024 and 0.005 for MPH and BPH, respectively, against relative genetic distance). We conclude that the formation of hybrids between divergent inbred lines results in transcriptome remodelling, with the extent of remodelling increasing with the degree of genetic divergence of those lines. This result is consistent with the expected effects of allelic variation on transcriptional regulatory networks. The relationship between transcriptome remodelling and heterosis can be interpreted as meaning that heterosis is likely to require transcriptome remodelling to occur, but that much of this involves low magnitude remodelling of the transcript abundance of a large number of genes.

The results of the above experiments indicate that the conventional approach to the analysis of the transcriptome in the hybrid, i.e. studying one or very few hybrid combinations, is unlikely to result in the identification of genes involved specifically in heterosis.

### Example 2

### Transcript Abundance in Hybrid Transcriptomes

We carried out an analysis using linear regression to identify the relationship between transcript abundance in a range of hybrids and the strength of heterosis (both MPH and BPH) shown by those hybrids. Significance levels were determined as F statistics from the regression Mean Square in the analysis of variance tables of the linear regression analysis. For this, we used the heterosis measurements and hybrid transcriptome data from the combinations described above with Landsberg er ms1 as the maternal parent, and from additional hybrids between Landsberg er ms1, as the maternal parent, and Columbia, Wt-1, Cvi-0, Sorbo, Br-0, Ts-5, Nok3 and Ga-0. Transcriptome data from 32 GeneChips, representing between 1 and 3 replicates from each of these 13 hybrid combinations of accessions, were used in this study. Nine genes were identified that showed highly significant (F<0.001) regressions (all positive) of transcript abundance in the hybrid on the magnitude of both MPH and BPH. Thirty-four genes showed highly significant regressions (F<0.001; 22 positive, 12 negative) of transcript abundance in the hybrid on MPH and significant regressions (F<0.05) on BPH. Twenty-seven genes showed highly significant regressions (F<0.001; 23 positive, 4 negative) of transcript abundance in the hybrid on magnitude of BPH and significant (F<0.05) regression on MPH. The genes are shown in Table 1 below. Based on gene ontology information, there are no obvious functional relationships between these 70 genes and no excess representation of genes involved in transcription.

The ability to identify a set of genes that show highly significant correlation of transcript abundance and magnitude of heterosis across 13 hybrids indicates that transcriptome-level events are predominant in the manifestation of heterosis. To confirm that this is correct, and that the genes we have identified are indicative of the transcript abundance characteristics that are important in heterosis, we utilized these discoveries to predict the strength of heterosis in new hybrid combinations based on the transcript abundance of the 70 defined genes. We built a mathematical model using the equations of the linear regression lines recalculated for each of the 70 genes against both MPH and BPH, to calculate the expected heterosis as the sum of the contribution from each gene, normalised by the coefficient of determination, r². The model operates as a Microsoft Excel spreadsheet, which is available as supplementary materials on Science Online. The spreadsheet also contained the normalised transcriptome data for the 70 genes from each of the hybrids studied. The model was validated by “predicting” the heterosis in the training set of 32 hybrids from which transcriptome data were used for its construction. It predicted heterosis across the full range of magnitude observed, for both MPH and BPH, with a very high correlation between predicted and observed values for individual samples (r²=0.768 for MPH, r²=0.738 for BPH). Three new hybrid combinations were produced, between the maternal parent Landsberg er ms1 and accessions Shakdara, Kas-1 and Ll-0. These were grown, in a “blind” experiment, under the same environmental conditions as the training set for the model, heterosis for fresh weight was measured and the transcriptomes analysed. The transcript abundance data for the 70 genes of the model were extracted for each of the new hybrids and entered into the heterosis prediction model. The results, as summarised below, confirmed that the model produced excellent quantitative predictions of heterosis, particularly MPH, confirming that transcriptome-level events were, indeed, predominant in the manifestation of heterosis.

### Prediction of Heterosis Using a Model Based on Hybrid Transcriptome Data

| | Mid-Parent | | Best-Parent | |
|---|---|---|---|---|
| | Heterosis % | | Heterosis % | |
| Hybrid | Predicted | Observed | Predicted | Observed |
| Landsberg er ms1 × | 43 | 34 | 15 | 22 |
| Shakdara | | | | |
| Landsberg er ms1 × | 46 | 57 | 16 | 24 |
| Kas-1 | | | | |
| Landsberg er ms1 × | 66 | 69 | 33 | 67 |
| Ll-0 | | | | |

Mid parent heterosis for fresh weight is presented as a percentage figure calculated as (weight of the hybrid−mean weight of the parents)/mean weight of the parents.

Best parent heterosis for fresh weight is presented as a percentage figure calculated as (weight of the hybrid−weight of the heaviest parent)/weight of the heaviest parent.

### Example 2a

### Highly Significant and Specific Correlation Between Heterosis and Transcript Abundance of At1g67500 and At5g45500 in Hybrids

In a further experiment to identify specific genes that show transcript abundance (gene expression) patterns in hybrids correlated with heterosis, we conducted an additional analysis based upon linear regression. For this we used a “training” dataset consisting of hybrid combinations between Landsberg er ms1 and Ct-1, Cvi-0, Ga-0, Gy-0, Kondara, Mz-0, Nok-3, Ts-5, Wt-5, Br-0, Col-0 and Sorbo. For each individual gene represented on the array, the transcript abundance in hybrids was regressed on the magnitude of heterosis exhibited by those hybrids. Twenty one genes showed highly significant (p<0.001) correlation, but this is no more than is expected by chance, as data for almost 23,000 genes were analysed. However, the exceptionally high significance for the two genes showing the greatest correlation (r²=0.457, P=6.0×10⁻⁶ for gene At1g67500; r²=0.453, P=6.9×10⁻⁶ for gene At5g45500) is highly unlikely to have occurred by chance. In both cases the correlation was negative, i.e. expression is lower in more strongly heterotic hybrids.

We tested whether the expression characteristics of these genes could be used for the prediction of heterosis. This was conducted by removing one hybrid from the dataset, formulating the regression line and using this relationship to predict the expected heterosis corresponding to the gene expression measured for the hybrid that had been removed. The analysis was repeated by the removal and prediction of heterosis in each of the 12 hybrids in turn. Three untested hybrids were developed (Landsberg er ms1 crossed with Ll-0, Kas-1 and Shakdara) as a “test” dataset, grown and assessed for heterosis as for the lines of the training dataset, and their transcriptomes analysed using ATH1 GeneChips. Using formulae derived by regression using all 12 hybrids in the training dataset, the expression data for genes At1g67500 and At5g45500 in the hybrids of the test dataset were used to predict the heterosis in these test hybrids. Both showed very high correlation between predicted and measured heterosis. Overall, predicted heterosis based on the expression of At1g67500 are better correlated with measured heterosis (r²=0.708) than those based on the expression of At5g45500 (r²=0.594). However, removal of one anomalous prediction in the training dataset (that of the heterosis shown by the hybrid Landsberg er ms1×Nok-3) improves the latter to r²=0.773. Nevertheless, the predictions of heterosis in all three hybrids of the test dataset based on the expression of At5g45500, in particular, are remarkably accurate.

Hybrids that show greater heterosis tend to be heavier than hybrids that show little heterosis. As expected, we identified such a correlation between the magnitude of heterosis we measured and weight for the 15 hybrids of our training and test datasets (r²=0.492). In order to assess whether the expression of genes At1g67500 and At5g45500 are specifically predicting heterosis, we assessed the possibility of correlation between gene expression and the weight of the plants in which expression is being measured. For this, we used the plant weight and gene expression data from the 12 parental lines in the training dataset. We found the expression of At1g67500 to show weak negative correlation with the weight of the plants (r²=0.321), but there was no correlation for At5g45500 (r²<0.001). We conclude that the transcript abundance of At5g45500 is indicative specifically of heterosis, but that of At1g67500 is likely to be influenced also by the weight of hybrid plants. This conclusion is consistent with the errors in prediction of heterosis in the test dataset using the expression of At1g67500: the prediction of heterosis in the hybrid Landsberg er ms1×Kas-1 (which is unusually heavy for the heterosis it shows) is over-estimated, whereas the prediction of heterosis in the hybrid Landsberg er ms1×Ll-0 (which is unusually light for the heterosis it shows) is underestimated.

Gene At5g45500 is annotated as encoding “unknown protein”, so its functions in the process of heterosis cannot be deduced based upon homology. The function of gene At1g67500 is known: it encodes the catalytic subunit of DNA polymerase zeta and the locus has been named AtREV3 due to the homology of the corresponding protein with that of yeast REV3 [67]. REV3 is important in resistance to UV-B and other stresses that result in DNA damage as its function is in translesion synthesis, which is required to repair forms of damage to DNA that blocks replication. Studies have shown no differential expression for At1g67500 in response to UV-B or other stresses [68]. However, the expression of At5g45500 is increased in aerial parts that were subjected to UV-B, genotoxic and osmotic stresses [68]. Thus both of the genes with expression correlated with heterosis in hybrid plants have potential roles in stress resistance. As the expressions of both are negatively correlated with heterosis, one hypothesis is that greater expression of these genes might be related to increased resilience to specific stresses, but this has a repressive effect on growth under favourable conditions. This resembles the situation where biomass and seed yield penalties were found to be associated with R-gene-mediated pathogen resistance to Pseudomonas syringae [69]. Heterosis, at least for vegetative biomass, may therefore be the consequence of genetic interactions that lead to a reduction in repression of growth, rather than direct promotion of growth.

### Example 3

### Transcript Abundance in Transcriptomes of Inbred Lines

We carried out separate analyses using linear regression to identify the relationship between transcript abundance in the parental lines and the strength of MPH shown by their respective hybrids with Landsberg er ms1. Significance levels were determined as F statistics from the regression Mean Square in the analysis of variance tables of the linear regression analysis.

In total, 272 genes were identified that showed highly significant (F<0.00) regressions of transcript abundance in the parent on the magnitude of MPH. See Table 2 below. Based on gene ontology information, there are no obvious functional relationships between these genes and no excess representation of genes involved in transcription.

The invention permits use of transcriptome characteristics of inbred lines as “markers” to predict the magnitude of heterosis in new hybrid combinations.

We built mathematical models, using the equations of the linear regression lines for each of the genes, to calculate the expected heterosis. These models operate as programmes within the Genstat statistical analysis package [70]. The results, as summarised in the table below, confirmed that the model successfully predicted the heterosis observed in the untested combinations using transcriptome characteristics of the inbred parents as markers.

### Prediction of Heterosis Using a Model Based on Parental Transcriptome Data

Mid-Parent
Heterosis % (44)

| Hybrid | Predicted | Observed |
|---|---|---|
| Landsberg er ms1 × | 34 | 34 |
| Shakdara | | |
| Landsberg er ms1 × Kas-1 | 46 | 57 |
| Landsberg er ms1 × Ll-0 | 50 | 69 |

### Example 3a

### Highly Significant Correlation Between Heterosis and Transcript Abundance of At3g11220 in Inbred Parents

We conducted an additional analysis based upon linear regression to identify genes that show expression patterns in inbred parents correlated with heterosis shown by the hybrids. For each individual gene represented on the array, transcript abundance in paternal parent lines was regressed on the magnitude of heterosis exhibited by the corresponding hybrids with accession Landsberg er ms1 in the training dataset.

The expression of one gene, At3g11220, showed an exceptionally high correlation (r²=0.649; P=2.7×10⁻⁸). The correlation was negative, i.e. expression is lower in parental lines that produce more strongly heterotic hybrids. We assessed the utility of using the expression of this gene in parental lines to predict the heterosis that would be shown by the corresponding hybrids with accession Landsberg er ms1. This was conducted for both training and test datasets, as for the predictions based on the expression of At1g67500 and At5g45500 in hybrids. The heterosis predicted was well correlated with the measured heterosis (r²=0.719) and the predicted values for two of the three hybrids in the test dataset were very accurate. However, heterosis was substantially overestimated for the hybrid Landsberg er ms1×Kas-1, despite there being no correlation between the expression of At3g11220 in parental accessions and the weight of those accessions (r²<0.001).

Gene At3g11220 is annotated as encoding “unknown protein”, so its function in the process of heterosis cannot be deduced based upon homology.

### Example 4

### Transcriptome Analysis for Prediction of Other Traits

We used the methodology as described for the prediction of heterosis using parental transcriptome data to develop models for the prediction of additional traits in accessions. The transcriptome data set used for the construction of the models was that obtained for 11 accessions: Br-0, Kondara, Mz-0, Ag-0, Ct-1, Gy-0, Columbia, Wt-1, Cvi-0, Ts-5 and Nok3, as previously described. Trait data had previously been obtained from these, and accessions Ga-0 and Sorbo. Transcriptome data from accessions Ga-0 and Sorbo were used for trait prediction in these accessions. The lists of genes incorporated into the models relating to the 15 measured traits are listed in Tables 3 to 17. The predicted trait values for Ga-0 and Sorbo were compared with measured trait values for these accessions, to assess the performance of the models.

As the models developed for the prediction of additional traits were developed using only 11 accessions, we expected them to contain some false components. These would tend to shift trait predictions towards the average value of the trait across the set of accessions used for the construction of the models. Therefore, our criterion for success of each model was whether or not it ranked the accessions Ga-0 and Sorbo correctly. The results, as summarised in Table 18, show that the models were able to successfully predict flowering time, seed oil content and seed fatty acid ratios. As expected, the values produced by the models were between the measured value for the trait in the respective accessions and the average value of the trait across all accessions. Only the models to predict the absolute seed content of a subset of specific fatty acids were unsuccessful. This lack of success in the experiment we conducted may have been due to the relative lack of precision of the data for these traits and/or insufficient numbers of genes with transcript abundance correlated with the trait to overcome the effects of false components in the models developed using the data sets available at the time. We believe that models based on more extensive data sets would be able to successfully predict these traits.

The ability to use transcriptome data from an early stage of plant growth under specific environmental conditions (i.e. aerial parts of vegetative-phase plants after 3 weeks growth in a controlled environment room under 8 hour photoperiod) to predict characteristics that appear later in the development of plants grown in different environmental conditions (flowering time, details of seed composition and vernalisation responses of plants grown in a glasshouse under 16 hour photoperiod) is remarkable. We interpret this as evidence of extensive interconnection and multiplicity of gene function, regulated, as for heterosis, largely at the level of transcript abundance. The results presented here indicate that our methodology will allow the use of specific characteristics of the transcriptomes of organisms, including both plants and animals, early in their life cycle as “markers” to predict many complex traits later in their life cycle, and to increase our understanding of the underlying biological processes.

### Example 5

### Methods and Materials

### Accessions Used

The accessions used for the studies underlying this disclosure were obtained from the Nottingham Arabidopsis Stock Centre (NASC): Kondara, Cvi-0, Sorbo, Ag-0, Br-0, Col-0, Ct-1, Ga-0, Gy-0, Mz-0, Nok-3, Ts-5, Wt-5 (catalogue numbers N916, N902, N931, N936, N994, N1092, N194, N1180, N1216, N1382, N1404, N1558 and N1612, respectively). A male sterile mutant of Landsberg erecta (Ler ms1) was also obtained from NASC (catalogue number N75).

### Growth Conditions

Seeds of parental accessions and hybrids were sown into pots containing A. thaliana soil mix (as described in O'Neill et al [71]) and Intercept (Intercept 5GR). The pot was then watered, and sealed to retain moisture, before being placed at 4° C. for 6 weeks to partially normalize flowering time. At the end of this time period the pot was placed in a controlled environment room (heated at 22° C. and lit for 8 hours per day). Gradually the seal was removed in order to acclimatise the plants to the reduced air moisture. When the first true leaves appeared the plants were transplanted to individual pots, which were again sealed and returned to the controlled environment rooms. Again the seal was gradually removed over the next few days. The positions of A. thaliana plants in controlled environment rooms was determined using a complete randomised block design, with the trays of plants being regularly rotated and moved in order to reduce environmental effects.

### The Production of Hybrid Seeds

Hybrids were produced by crossing accessions Kondara and Br-0 by selecting a raceme of the maternal plant, removing all branches and siliques, leaving only the inflorescence. All immature and open buds were removed, along with the apical meristem, leaving 5-6 mature closed buds. From these buds the sepals, petals, and stamens were removed leaving only a complete pistil. For crosses involving Ler ms1 as the maternal parent, only enough tissue was removed, from unopened buds, to allow access to the stigma. Buds of all plants were then pollinated by removing a stamen from the pollen donor plant, and rubbing the anther against the stigma. This was repeated until the stigma was well coated with pollen when viewed under the microscope. The pollinated buds were then protected from additional pollination by being enclosed in a ‘bubble’ of Clingfilm, which was removed after 2-3 days.

### Trait Measurements

The total aerial fresh weight of the plants was determined by cutting off all above soil plant material, quickly removing any soil attached, and weighing on electronic scales (Ohaus Corp. New. Jersey. USA). The plant material was then frozen in liquid nitrogen. All plant harvesting and weight measurements were taken as close as practicable to the middle of the photoperiod. Where trait data were combined for replicate sets of plants grown at different time, the data were weighted to correct for differences in absolute growth rates between the replicates caused by environmental effects. The mean weight for each of the 14 parent accessions and 13 hybrids was calculated for each of the three growth replicates. These were then normalised to the first replicate mean, to take account of any between-occasion variation in the growth conditions. This was done by dividing each replicate mean by the first replicate mean and then multiplying by itself (for example [a/b]*b) in order to obtain the adjusted mean.

RNA Extraction and Hybridisation

200 mg of plant tissue were ground to a fine powder using liquid nitrogen in a baked pre-cooled mortar, and using a chilled spatula, transferred to labelled chilled 1.5 ml tube. To these tubes 1 ml of TRI Reagent (Sigma-Aldrich, Saint Louis USA) was added, then shaken to suspend the tissue. After a 5 minute incubation at room temperature 0.2 ml of chloroform was added, and thoroughly mixed with the TRI Reagent by inverting the tubes for around 15 seconds, followed by 2-3 minutes incubation at room temperature. The tubes were centrifuged at 12000 rpm for 15 minutes and the upper aqueous phase transferred to a clean, labelled tube. 0.5 ml of isopropanol was then added to the tubes, which were inverted repeatedly for 30 seconds to precipitate the RNA, followed by a10 minutes incubation at room temperature. The tubes were then were centrifuged at 12000 rpm for 10 minutes at 4° C., revealing a white pellet on the side of the tube. The supernatant was poured off of the pellet, and the lip of the tube gently blotted with tissue paper. 1 ml 75% ethanol was added and the tubes shaken to detach the pellet from the side of the tube, followed by centrifugation at 7500 rpm for 5 minutes. Again the supernatant was poured off of the pellet, which was quickly spun down again and any remaining liquid removed using a pipette. The pellet was then dried in a laminar flow hood, before 50 μl DEPC treated water (Severn Biotech Ltd. Kidderminster, UK) was added to dissolve the pellet.

Sample concentrations were determined using an Eppendorf BioPhotometer (Eppendorf UK Limited. Cambridge. UK), and RNA quality was determined by running out 111 on a 1% agarose gel for 1 hour. RNA from replicated plants were then pooled according concentration in order to ensure an equal contribution of each replicate.

The pooled samples were then cleaned using Qiagen Rneasy columns (Qiagen Sciences. Maryland. USA) following the protocol on page 79 of the Rneasy Mini Handbook (06/2001), before again determining the concentrations using an Eppendorf BioPhotometer, and running out 111 on a 1% agarose gel.

Affymetrix GeneChip array hybridisation was carried out at the John Innes Genome Lab (http://www.jicgenomelab.co.uk). All protocols described can be found in the Affymetrix Expression Analysis Technical Manual II (Affymetrix Manual II http://www.affymetrix.com/support/technical/manual.affx.)

Following clean up, RNA samples, with a minimum concentration of 1 μg, μl-1, were assessed by running 1 μl of each RNA sample on Agilent RNA6000 nano LabChips® (Agilent Technology 2100 Bioanalyzer Version A.01.20 SI211). First strand cDNA synthesis was performed according to the Affymetrix Manual II, using 10 μg of total RNA. Second strand cDNA synthesis was performed according to the Affymetrix Manual II with the following minor modifications: cDNA termini were not blunt ended and the reaction was not terminated using EDTA. Instead Double-stranded cDNA products were immediately purified following the “Cleanup of Double-Stranded cDNA” protocol (Affymetrix Manual II). cDNA was resuspended in 22 μl of RNase free water.

cRNA production was performed according to the Affymetrix Manual II with the following modifications: 11 μl of cDNA was used as a template to produce biotinylated cRNA using half the recommended volumes of the ENZO BioArray High Yield RNA Transcript Labelling Kit. Labelled cRNAs were purified following the “Cleanup and Quantification of Biotin-Labelled cRNA” protocol (Affymetrix Manual II). cRNA quality was assessed by on Agilent RNA6000 nano LabChips® (Agilent Technology 2100 Bioanalyzer Version A.01.20 SI211). 20 μg of cRNA was fragmented according to the Affymetrix Manual II.

High-density oligonucleotide arrays (either Arabidopsis ATH1 arrays, or AT Genomel arrays, Affymetrix, Santa Clara, Calif.) were used for gene expression detection. Hybridisation overnight at 45° C. and 60 RPM (Hybridisation Oven 640), washing and staining (GeneChip® Fluidics Station 450, using the EukGEws2_{—}450 Antibody amplification protocol) and scanning (GeneArray® 2500) was carried out according to the Affymetrix Manual II.

Microarray suite 5.0 (Affymetrix) was used for image analysis and to determine probe signal levels. The average intensity of all probe sets was used for normalization and scaled to 100 in the absolute analysis for each probe array. Data from MAS 5.0 was analysed in GeneSpring® software version 5.1 (Silicon Genetics, Redwood City, Calif.).

Identification of Genes with Non-Additive Transcript Abundance in Hybrids

Analysis of the normalised transcript abundance data was performed using GenStat [70]. This was undertaken using a script of directives programmed in the GenStat command language (see below), and used to identify the set of defined patterns of transcript abundance. Briefly, each hybrid transcript abundance data set was compared to its appropriate parental data sets, for each gene, for each of the particular expression patterns of interest. Those genes showing a particular pattern in each data set were given a test value. Once completed all of these values were added together and only those data sets with a combined test value equal to a given a critical value (equivalent to the value if all data sets displayed that pattern) were counted. Once this had been completed for the experimental data, the results were checked by hand against the source data.

Program 1 below is an example of the pattern recognition programme. This example identifies patterns in the KoBr hybrid and its parents, for three replicates of each at the two-fold threshold criteria.

### Permutation Analysis to Calculate Expected Values for Non-Additive Transcript Abundance in Hybrids

Due to the relatively limited replication within the experiment and the large number of genes assayed on the GeneChips it is expected that a proportion of the genes displaying defined patterns will have occurred by chance. It is therefore essential to use appropriate statistical analysis of the data to determine the significance of the results. In order to determine this, random permutation analysis (bootstrapping) was used to generate expected values for random occurrences of defined abundance patterns of the data. Pseudoreplicate data sets were generated by randomly sampling the original data within individual arrays, and using a rotating ‘seed number’ in order to create random data sets of the same size, and variance, as the original. The same pattern recognition directives were then used for this random data set as were used on the original data and the resulting numbers of probes were recorded.

In order to get a statistically significant number of randomized replicates, this randomization and analysis of the data was repeated 250 times. The average numbers of probes identified for each pattern were then used as the value that would be expected to arise by random chance for that pattern. It was determined that 250 cycles was a sufficiently large random data set, for this experiment by comparing the expected random averages of the defined patterns at 1.5 fold, at 50 cycles and at 250 cycles. Comparisons between higher numbers of cycles (500-1000 cycles) exhibited very little difference between the means except that the longer runs served to reduce the standard errors. A Wilcoxon matched-pairs two-tailed t-test on the means of the two repetition levels (50 cycles and 250 cycles) gave a P-value of 0.674, suggesting very strongly that the means are not statistically different from each other. Based on this it was assumed that the average random values will not change significantly with increased replication, and that 250 cycles is a significantly large number of replicates to generate this mean random value in this case.

Program 2 below is an example of the bootstrapping programme. This example bootstraps the KoBr hybrid at the two-fold threshold criteria, for 250 repetitions.

### Chi2 Tests for Significance of Transcriptome Remodelling

Fold changes in themselves are not statistical tests, and cannot be used alone to designate a confidence level of the reported differences in expression. The average numbers of probes identified for each pattern after permutation analysis represent the number expected to arise by random chance for that pattern. Once this expected value has been determined it can be used in a maximum likelihood Chi square test, under the null hypothesis of no difference between observed and expected, in order to determine whether the observed patterns differ significantly from random chance. This was undertaken using the “Chi-Square goodness of fit” option of GenStat, and testing the difference between the mean number of genes observed fitting a given expression pattern, and the mean number of genes expected to fit that same pattern (as calculated above), with a single degree of freedom. Significant relationships, fitting the alternative hypotheses of significant differences between the two mean values, were considered to be those exhibiting P values of 0.05 or less.

### Normalisation of Transcriptome Remodelling

Transcriptome remodelling was calculated, normalised for the divergence of the transcriptomes of the parental accessions, using the equation:

NT=R_{T}/(Rₚ/Rₚₘ)

Where NT=normalised level of transcriptome remodelling of a cross
R_{T}=total number of genes summed across all 6 classes indicative of remodelling for the specific hybrid, at the appropriate fold-level
Rₚ=total number of genes with transcript abundance differing between the parental accessions of the specific hybrid, at the appropriate fold-level.
Rₚₘ=Mean number of genes with transcript abundance differing between the parental accessions across all combinations analysed, at the appropriate fold-level.

### Estimation of Relative Genetic Distance

In order to develop a measure of the Relative Genetic Distance (RGD) between accession Ler and the 13 accessions crossed with it to produce hybrids the following method was used. A set of 216 loci were selected that were polymorphic for the 14 main accessions studied in this thesis. These were downloaded from the web site of the NSF 2010 project DEB-0115062 (http://walnut.usc.edu/2010/). Loci were selected to cover the genome by defining 500 kb intervals throughout the genome, starting at base pair 1 on each chromosome, and selecting the polymorphic locus with the lowest base pair coordinate that has a complete set of sequence data for all 14 accessions, if any, in each interval. The number of polymorphisms across these 216 loci between each accession and Ler were determined and normalised relative to the polymorphism rate observed between Ler and Columbia (with 45 polymorphisms, the most similar to Ler) to give the RGD.

Regression Analysis to Identify Genes with Transcript Abundance in Hybrid Lines Correlated with the Strength of Heterosis

In order to identify genes showing a significant linear relationship between strength of heterosis and transcript abundance in hybrid lines, regression analysis was undertaken using a script of directives programmed in the GenStat command language. This programme conducted a linear regression, for the transcript abundance of each probe, against the phenotypic value for 32 GeneChips. There were three replicate GeneChips for each of the hybrids LaAg, LaCt, LaCv, LaGy, LaKo, and LaMz, and two replicates each for LaBr, LaCo, LaGa, LaNo, LaSo, LaTs, and LaWt, each representing the pooled RNA of three individual hybrid plants. The results of these regressions were presented as F-values. Once this had been completed for the experimental data, significant results were checked by hand against the source data.

Program 3 below is an example of the linear regression programme. This example identifies linear regressions between the hybrid transcriptome and MPH.

Once this had been completed for the transcription data, permutation analysis was used to determine how often particular regression line would arise by random chance. The data was randomised within individual arrays, using a rotating ‘seed number’ and the regression analyses were repeated for this random data, using the same directives used for the original data. In order to get a statistically significant number of random replicates, this randomisation and analysis of the data was repeated 1000 times. Following this, the 1000 regression values for each gene were ranked according to the probability of a relationship between the phenotypic values and random expression values, and the F values of the first, tenth and fiftieth values (corresponding to the 0.1%, 1% and 5% significance values) were recorded. The probabilities of the actual and randomised samples were then compared and only those genes where the probability of occurring randomly is less than in the actual data at one of the three significance values were counted as showing a significant relationship.

Program 4 below is an example of the linear regression bootstrapping programme. This example randomises linear regressions between the hybrid transcriptome and MPH. Due to the size of the outputs, the files are saved into intermediary files that can be read by the computer but not opened visually.
Program 5 below is an example of the programme written to extract the significant values out of the bootstrapping intermediary data files, into a file that can be manipulated in excel. Again this example handles linear regression data between the hybrid transcriptome and MPH.
Regression Analysis to Identify Genes with Transcript Abundance in Parental Lines Correlated with the Strength Of Heterosis

In order to identify genes showing a significant linear relationship between strength of heterosis and transcript abundance in parental lines, regression analysis was undertaken as described for the identification of genes with transcript abundance in hybrids correlated with the strength of heterosis.

### Example 6

### A Transcriptomic Approach to Modelling and Prediction of Hybrid Vigour and Other Complex Traits in Maize

### Modelling and Prediction of Heterosis in Maize

The experimental design uses a series of 15 different hybrid maize lines, all with line B73 as the maternal parent. The hybrids and parental lines were grown in replicated trials at three locations (two in North Carolina and one in Missouri) in 2005, and data were collected for heterosis and a range of other traits, as listed below. All 31 lines (15 hybrids and 16 parents) were grown for 3 weeks and aerial tissues cut, weighed and frozen in liquid nitrogen. RNA was prepared and Affymetrix maize GeneChips were used to analyse the transcriptome in 2 replicates of each. The methods successfully developed in Arabidopsis, as described above, were used to (i) identify genes with transcript abundance correlated with the magnitude of heterosis, (ii) develop predictive models using the transcriptome data from 12 or 13 hybrids and the corresponding parents and (iii) test the ability of the models to “predict” the performance of additional hybrids, based only upon their transcriptome characteristics.

Genes whose transcript abundance was shown to correlate with heterosis in maize are shown in Table 19. Heterosis was calculated for plant height, for plants at CLY location (Clayton, N.C.) only (model from 13 hybrids).

These data were used to develop a model for prediction of heterosis in two further hybrids. All of the genes used in producing the calibration line were have been used in the prediction, both for the model development and the further “test” plants.

Prediction of Heterosis for Plant Height, CLY Location Only (Model from 13 Hybrids to Predict 2):

MPH PH CLY

| Location | | Hybrids | |
|---|---|---|---|
| CLY | | B73 × Ki3 | B73 × OH43 |
| | Actual | 149.19 | 134.88 |
| | Value | | |
| | Predicted | 144.59 | 141.45 |
| | | No. of correlated | 370 |
| | | genes: | |

The same procedures can be used to develop predictive models for each of the additional traits for which complete data sets are available. For maize, the data from 14 inbred lines (used as parents of the hybrids described above) can be used to develop models for prediction of traits in further inbred lines.

The following traits may be measured in maize: yield; grain moisture; plant height; flowering time; ear height; ear length; ear diameter; cob diameter; seed length; seed width; 50 kernel weight; 50 kernel volume.

Genes with transcript abundance correlating with yield, measured as harvestable product, are shown in Table 20. Average yield was calculated for 12 plants across 2 sites, MO and L.

These genes were used to develop a model for prediction of yield in three further hybrids. All of the genes used in producing the calibration line were have been used in the prediction, both for the model development and the further “test” plants.

Rank order of yield was successfully predicted in these hybrids, and the magnitude was accurate for 2 out of the 3 hybrids, shown below. With improved trait data, accurate predictions would be expected for all hybrids.

Prediction of Average Yield Across 2 Sites, MO and L (Model from 12 Hybrids to Predict 3)

Weight
Mo&L

| Location | | Hybrids | | |
|---|---|---|---|---|
| MO & L | | B73 × | B73 × CML247 | B73 × Mo18W |
| | | M37W | | |
| | Actual | 9.70 | 11.87 | 11.81 |
| | Value | | | |
| | Predicted | 9.63 | 11.38 | 10.90 |
| | | | No. of correlated | 419 |
| | | | genes: | |

### Example 6a

### Prediction of Plot Yield in Maize Hybrids Using Parental Transcriptome Data

We used linear regression to identify genes for which expression levels in a training dataset of 20 genetically diverse inbred lines (B97, CML52, CML69, CML228, CML247, CML277, CML322, CML333, IL14H, Ki11, Ky21, M37W, Mo17, Mo18W, NC350, NC358, Oh43, P39, Tx303, Tzi8) was correlated with the plot yield of the corresponding hybrids with line B73. Pedigrees and phylogenetic grouping 72 of the maize lines used in our studies are summarised in Table 21.

Using a stringent cut-off for significance (P<0.00001), correlations (0.288<r²<0.648) were identified for 186 genes. These are listed in Table 22. In the majority of cases (129), gene expression in the inbred lines was negatively correlated with yield of the hybrids. We were able to discount the possibility that these correlations were artefacts of differing proportions of cell types in different sizes of plants, which may have arisen if the sizes of the inbred seedlings were indicative of the performance of the corresponding hybrids, as we found no correlation between plot yield and either the weight (r²=0.039) or the height (r²=0.001) of the sampled seedlings of the corresponding parental lines.

To assess whether gene expression characteristics may be used successfully for the prediction of yield, each hybrid in turn was removed from the training dataset and models developed based upon a regression conducted with the remaining lines. This was conducted as for A. thaliana, except that the mean of the predictions for all of the genes with highly significant correlation (P<0.00001) was used as the overall prediction of heterosis for the excluded line. The numbers of genes exceeding this significance threshold varied from 84 (with P39 excluded) to 262 (with NC350 excluded). Gene expression data for a test dataset of four additional inbred lines (CML103, Hp301, Ki3, OH7B) was then used to predict the heterosis that would be shown by the corresponding hybrids with B73, by averaging the predictions from each of the 186 genes identified by regression analysis using the complete training dataset. The results showed that the predicted plot yield is strongly correlated with the measured plot yield (r²=0.707), demonstrating that gene expression characteristics can, indeed, be used for the prediction of heterosis, as quantified by yield. Although the relationship was non-linear, with reduced ability to quantitatively predict yields at the higher end of the range studied, the method was able to correctly resolve the two highest yielding hybrids in the test dataset from the two lowest yielding hybrids. The poor yield performance of hybrids including the popcorn (HP301) and the two sweet corns (IL14H and P39) were correctly predicted, but the exceptionally high yield of the hybrid NC350×B73 was not predicted. We conclude that maternal effects are minor, as the analysis was based on a mixture of crosses with B73 as the maternal parent (15 hybrids) and as the paternal parent (9 hybrids).

### Growth and Trait Analysis of Maize Plants

Plants used for transcriptome analysis were grown from seeds for 2 weeks. Maize seeds were first imbibed in distilled water for 2 days in glasshouse conditions to break dormancy, before transfer to peat and sand P7 pots. They were grown in long day glass house conditions (16 hours photoperiod) at 22° C. Aerial parts above the coleoptiles were excised, weighed and frozen in liquid nitrogen. All plant harvesting and weight measurements were taken as close as practicable to the middle of the photoperiod. Plants for yield trials were grown in field conditions in Clayton, N.C. in 2005. Forty plants of each hybrid were grown in duplicate 0.0007 hectare plots. Yield was calculated as pounds of grain harvested per plot, corrected to 15% moisture, as shown in Table 23.

### Example 7

### A Transcriptomic Approach to Modelling and Prediction of Hybrid Vigour and Other Complex Traits in Oilseed Rape

### Modelling and Prediction of Heterosis in Oilseed Rape

The experimental design uses a series of 14 different hybrid oilseed rape restorer lines, all with line MSL 007 C (which is a male sterile winter line and has been used for commercial hybrid production) as the maternal parent. The hybrids and parental lines were grown in Hohenlieth and Hovedissen in Germany and Wuhan in China in 2004/5, and data for heterosis and a range of other traits, as listed below, were collected. All 29 lines (14 hybrids and 15 parents) are grown for 3 weeks and aerial tissues cut, weighed and frozen in liquid nitrogen. RNA is prepared and Affymetrix Brassica GeneChips are used to analyse the transcriptome in 3 replicates of each. The methods successfully developed in Arabidopsis are used to (i) identify genes with transcript abundance correlated with the magnitude of heterosis, (ii) predictive models are developed using the transcriptome data from 12 hybrids and the corresponding parents and (iii) the ability of the models to “predict” the performance of the 2 additional hybrids, based only upon their transcriptome characteristics, is demonstrated.

Traits measured in oilseed rape: Seed yield, seed weight, seed oil content, seed protein content; seed glucosinolates; establishment; Winter hardiness; Spring development; flowering time; plant height; standing ability.

### Modelling and Prediction of Additional Traits

Upon completion of heterosis modelling, the same procedures are used to develop predictive models for each of the additional traits for which complete data sets are available. For oilseed rape, the data from 12 inbred lines (used as parents of the hybrids described above) is used to develop models, which is used to “predict” the traits in 2 further inbred lines. The performance of the models is validated.

### Example 8

### Further Data Modelling Techniques

### Improvement of the Models

The models developed in Arabidopsis utilize linear regression approaches. However, non-linear approaches may enable the identification of more comprehensive gene sets and, hence, more precise models. Non-linear approaches are therefore incorporated into the model development protocols. Additional opportunities for refinement include weighting of the contribution of individual genes and data transformations.

### Development of Reduced Representation Models

Although approaches based on the use of GeneChips or microarrays may continue to be the preferred analytical platform for commercialization, there are other methods available for the quantitative determination of transcript abundance. Quantitative PCR methods can be reliable and are amenable to some automation. However, when such approaches are to be used, it is desirable to identify a subset of genes (ideally under 10) that retain most of the predictive power of the sets of genes used to date in the models (70 for prediction of heterosis based on hybrid transcriptomes, typically >150 for prediction of heterosis or other traits based on inbred transcriptomes). Therefore, a limited set of genes is identified by iterative testing of the precision of predictions by progressively reducing the numbers of genes in the models, preferentially retaining those with the best correlation of transcript abundance with the trait.

### Example 9

### Standard Operating Instruction for the Analysis of Gene Expression Data

This section provides detailed guidance for development and use of predictive models using the program GenStat [70].

### List of Programmes

The following GenStat programmes may be used in accordance with the invention and are suitable for analysing any Affymetrix based expression data.

### GenStat Programme 1˜Basic Regression Programme˜Method 4

### GenStat Programme 2˜Basic Prediction Regression Programme˜Method 5

### GenStat Programme 3˜Prediction Extraction Programme˜Method 5

### GenStat Programme 4˜Basic Best Predictor Programme˜Method 7

### GenStat Programme 5˜Basic Linear Regression Bootstrapping Programme˜Method 9

### GenStat Programme 6˜Basic Linear Regression Bootstrapping Data Extraction Programme˜Method 9

### GenStat Programme 7˜Basic Transcriptome Remodelling Programme˜Method 10

### GenStat Programme 8˜Dominance Pattern Programme˜Method 11

### GenStat Programme 9˜Dominance Permutation Programme˜Method 11

### GenStat Programme 10˜Transcriptome Remodelling Bootstrap Programme˜Method 12

### Introduction

These standard operating procedures are designed to enable the undertaking of gene expression analysis studies, from RNA extraction through to advanced prediction.

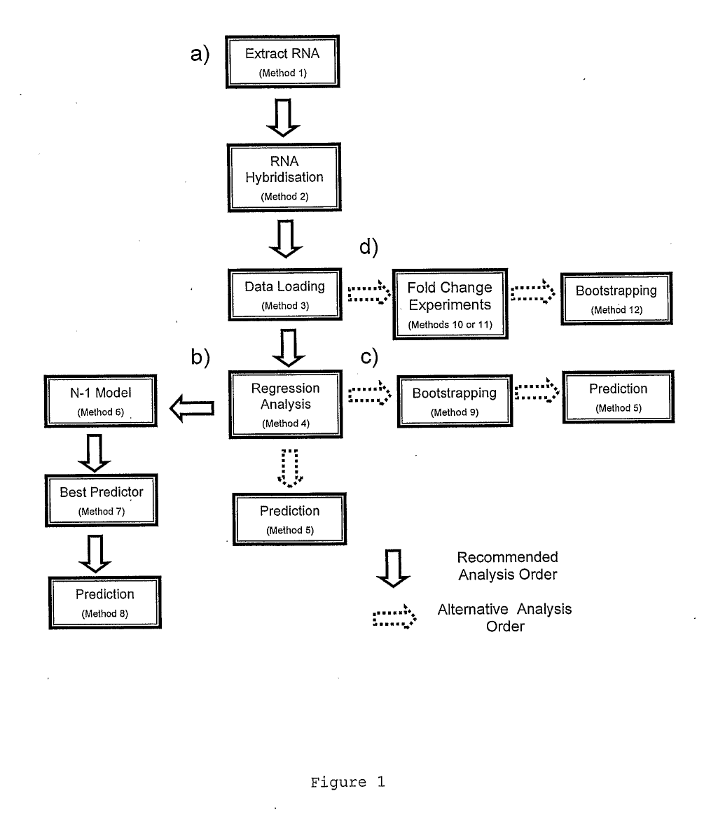

The procedures are divided into 4 workflows, depending on the type of analyses you wish to undertake. See FIG. 1.

Workflow a) follows the basic first steps, common to all analyses (methods 1-3), to the stage of predicting traits based upon transcription profiles.

Workflow b) follows the recommended analysis procedure (based on the latest analysis developments). It culminates in the prediction of traits based on a subset of best predictor genes.

Workflow c) follows an alternative analysis procedure, used to generate the prediction reported in my thesis, and includes a bootstrapping step.

Workflow d) describes to methods for analysing the degree of transcriptome remodelling between hybrids and their parent lines.

All of these workflows are designed to be ‘worked through’ and contain step-by-step instruction on how to complete the analysis.

### a) Standard Protocols

### Method 1, Extract RNA

This stage results in the production of good quality total RNA at a concentration of between 0.2-1 μg μl⁻¹ for hybridisation to Affymetrix GeneChips. These methods are the same for both Arabidopsis and Maize chips, for other species, contact Affymetrix for their recommended methods.

### 1.1 Trizol RNA Extraction

200 mg of plant tissue were ground to a fine powder using liquid nitrogen in a baked pre-cooled mortar, and using a chilled spatula, transferred to labelled chilled capped tube. To these tubes 1 ml of TRI REAGENT (Sigma-Aldrich, Saint-Louis USA) was added and shaken to suspend the tissue. After a 5 minute incubation at room temperature 0.2 ml of chloroform was added, and thoroughly mixed with the TRI REAGENT by inverting the tubes for around 15 seconds, followed by 2-3 minutes incubation at room temperature. The tubes were centrifuged at 12000 rpm for 15 minutes and the upper aqueous phase transferred to a clean, labelled tube.

0.5 ml of isopropanol was then added to the tubes, which were inverted repeatedly for 30 seconds to precipitate the RNA, followed by 10 minutes incubation at room temperature. The tubes were then centrifuged at 12000 rpm for 10 minutes at 4° C., revealing a white pellet on the side of the tube. The supernatant was poured off the pellet, and the lip of the tube gently blotted with tissue paper. 1 ml 75% ethanol was added and the tubes shaken to detach the pellet from the side of the tube, followed by centrifugation at 7500 rpm for 5 minutes. Again the supernatant was poured off the pellet, which was quickly spun down again and any remaining liquid removed using a pipette. The pellet was then dried in a laminar flow-hood; before 50 μl DEPC treated water (Severn Biotech Ltd. Kidderminster, UK) was added to dissolve the pellet.

### 1.2 RNA Clean-Up

RNA samples were cleaned up using RNeasy® mini columns (Qiagen Ltd, Crawly, UK), according to the protocol given in the RNeasy® Mini Handbook (3^{rd} edition 06/2001 pages 79-81). Due to the maximum binding capacity, no more than 100 μg of RNA could be loaded on to each column. In order to obtain as high a concentration as possible during the elution step, 40 μl was used and the elute run through the column twice. This was followed by a second 40 μl volume of DEPC treated water in order to remove any remaining RNA, which could be used to increase the amount of clean RNA available, should further concentration be required.

### 1.3 Concentration of RNA Samples

If the concentration of the clean RNA was less than 1 μg μl⁻¹ a further precipitation and dissolution can be performed using an Affymetrix recommended method which can be found in the Affymetrix Expression Analysis Technical Manual II (http://www.affymetrix.com/support/technical/manuals.affx).

5 μl 3 M NaOAc, pH 5.2 (or one tenth of the volume of the RNA sample) was added to the RNA sample requiring concentrating, together with 250 μl of 100% ethanol (or two and a half volumes of the RNA sample). These were mixed and incubated at −20° C. for at least 1 hour. The samples were centrifuged at 12000 rpm in a micro-centrifuge (MSE, Montana, USA) for 20 minutes at 4° C., and the supernatant poured off leaving a white pellet. This pellet was washed twice with 80% ethanol (made up with DEPC treated water), and air-dried in a laminar flow hood. Finally the pellet was re-suspended in DEPC treated water, to a volume appropriate to the required concentration.

### Method 2, RNA Hybridisation

### 2.1 Hybridisation to GeneChips

Affymetrix GeneChip array hybridisation was carried out at the John Innes Genome Lab (http://www.jicgenomelab.co.uk). All protocols described can be found in the Affymetrix Expression Analysis Technical Manual II (Affymetrix Manual II http://www.affymetrix.com/support/technical/manuals.affx.)

Following clean up, RNA samples, with a concentration of between 0.2-1 μg, μl⁻¹, were assessed by running 1 μl of each RNA sample on Agilent RNA6000 nano LabChips® (Agilent Technology 2100 Bioanalyzer Version A.01.20 SI211). First strand cDNA synthesis was performed according to the Affymetrix Manual II, using 10 μg of total RNA. Second strand cDNA synthesis was performed according to the Affymetrix Manual II with the following minor modifications:

cDNA termini were not blunt ended and the reaction was not terminated using EDTA. Instead Double-stranded cDNA products were immediately purified following the “Cleanup of Double-Stranded cDNA” protocol (Affymetrix Manual II). cDNA was re-suspended in 22 μl of RNase free water.

cRNA production was performed according to the Affymetrix Manual II with the following modifications:

11 μl of cDNA was used as a template to produce biotinylated cRNA using half the recommended volumes of the ENZO BioArray High Yield RNA Transcript Labelling Kit. Labelled cRNAs were purified following the “Cleanup and Quantification of Biotin-Labelled cRNA” protocol (Affymetrix Manual II). cRNA quality was assessed by on Agilent RNA6000 nano LabChips® (Agilent Technology 2100 Bioanalyzer Version A.01.20 SI211). 20 μg of cRNA was fragmented according to the Affymetrix Manual II.

High-density oligonucleotide arrays were used for gene expression detection. Hybridisation overnight at 45° C. and 60 RPM (Hybridisation Oven 640), washing and staining (GeneChip® Fluidics Station 450, using the EukGEws2_{—}450 Antibody amplification protocol) and scanning (GeneArray® 2500) was carried out according to the Affymetrix Manual II.

Microarray suite 5.0 (Affymetrix) was used for image analysis and to determine probe signal levels. The average intensity of all probe sets was used for normalization and scaled to 100 in the absolute analysis for each probe array. Data from MAS 5.0 was analysed in GeneSpring® software version 5.1 (Silicon Genetics, Redwood City, Calif.).

Files were saved as .txt files, for further analysis.

### Method 3, Data Loading

This section describes the methods used to load the expression data into GeneSpring, how to normalise the data, and how to save it in excel for further analysis. These instructions are best followed while carrying out the analysis. A GeneSpring course is recommended if further analysis is required using this programme.

3.1 Loading Data into GeneSpring
Open GeneSpring, >File>Import data>select the first of the data files you wish to load>click Open
Choose file format—Affy pivot table
(Create new genome—if you don't want to go into an existing one)
Select genome—Arabidopsis, Maize, etc, or create a new genome following instructions on screen
Import data: selected files—select any remaining files you want to analyse
Import data: sample attributes—this is where you can enter the MIAME info
Import data: create experiment—yes. Save new experiment—give it a name, it will appear in the experiment folder in the navigator toolbar.

### 3.2 New Experiment Checklist

These 4 factors should be completed in turn, to ensure that the data is properly normalised. This will impact upon all of the subsequent analyses. Generally the defaults or recommended orders should be used.

### Define Normalisations

Click on ‘use recommended order’ and check that the following is included:

Data transformation: measurements less than 0.01 to 0.01 Per chip: 50th %
Per gene: normalise to median, cut off=10 in raw signal

### Define Parameters

Here we define the names of the expression data. Depending upon the labelling of the expression files, changes may not be required here. If changes are required:

Click on ‘New custom’ Type the name of each sample.
Delete other parameters to avoid confusion.

### Save

### Define Default Interpretation

No changes needed for this experiment
Define Error model
No changes needed for this experiment

### 3.3 Transfer Data in to Excel

Once the data is normalised it can be transferred into an excel spreadsheet.

To do this, click on the relevant data in the experiment tree (on the far left of the main GeneSpring screen)

Click View>view as spreadsheet
select all>copy all>paste into Excel spreadsheet.

### Save.

This forms the master Excel chart.

### Method 4, Regression Analysis

These instructions describe the basic regression method. This regression forms the basis of the subsequent prediction methods.

### 4.1 Create Data File

To create a data file for use in GenStat. Open the master Excel file (with normalised expression data from GeneSpring)>Copy the relevant data columns (the data for those accessions that will form the ‘training data set’ from which significant predictive genes will be selected) into a new chart>add a column of “:” at the far end>save chart as .txt file>close file

Open the text file in GenStat>Enclose any title names in speech marks (“ ”), this should have the effect of turning the titles green>Find and replace (ctrl R)* with blanks>Replace all>Save file again

### 4.2 Regression Programme

Open ‘basic regression programme’ (GenStat Programme 1˜Basic Regression Programme) in GenStat
Check that the input data filename is correct, and is opening to channel 2
Check that the output data file is going to the correct destination and is opening to channel 3. These input and output file names should be RED
Check that the phenotypic trait data are correct for the trait under investigation. Use “\” to go on to new lines, these backslashes will turn GREEN.
Check that the number of genes to be investigated is set to the correct value (usually 22810 for Arabidopsis, or 17734 for Maize).

If the R², Slope, and Intercept are required remove the “ ” from the appropriate analysis section, and from the print command, both will turn BLACK from green.

### 4.3 Running the Programme

To run the programme, ensure that both the programme window and output windows are open (to tile horizontally Alt+Shift+F4). Select the programme window and press Ctrl+W. This will set the programme running, check that the GenStat server icon (histogram symbol, in taskbar at bottom right-hand corner of the screen) has changed colour to red.

To cancel the programme right click on the server icon and choose interrupt

Once complete the GenStat icon will change colour back to green

### 4.4 Analysing the Output

To analyse the data, first open it in Excel, select “delimited”>next>tick the “Tab” and “Space”>Finish
Add a new row at the far left-hand side of the sheet, and label the appropriate columns “value” “Df” and “R square” “Slope” and “Intercept” if these were included in the analysis
Add a new column to the beginning and label it “ID”
Fill the remaining cells of the ID column with a series 1-22810 for Arabidopsis or 1-17734 for Maize (edit>fill>series>OK)
Delete the column “Df”
Select all of the data columns>Data>Sort>P value ascending
Select all of the rows where the P value are less than or equal to 0.05. Colour these cells using the “paint” option, and record the number in this list. These are the genes significant at the 5% level
Select all of the rows where the P value are less than or equal to 0.01. Colour these cells an alternative colour using the “paint” option, and record the number in this list. These are the genes significant at the 1% level
Select all of the rows where the P value are less than or equal to 0.001. Colour these cells a third colour using the “paint” option, and record the number in this list. These are the genes significant at the 0.1% level
These three values are the number of OBSERVED significant probes in the data set

These observed significant probes, can be used as ‘prediction probes’ for the prediction of traits in other accessions, or hybrid combinations.

### Method 5, Prediction

These instructions describe the basic prediction method. All subsequent prediction methods are a variation on this.

### 5.1 Producing the Prediction Calibration Lines

Using the list of identified prediction probes; create a specific prediction sub-set gene list. This can be done by copying your ID and P-value columns (sorted by ID to return the data to its original order) in to a new excel sheet along with the expression data of your training line accessions. You can then sort by P-value and delete those genes that do not appear in the relevant significance (usually 0.1%) list. Remember to sort by ID again to return the file to its correct order, then delete the ID and Sig0.1% columns you added. Save this file under a new file name as a .txt file (for example trainingsetdata.txt).

### Open the ‘Basic Prediction Regression Programmer’ (GenStat Programme 2)

Check that the input file is the one that you have just created
Check that the output file is named correctly (calibration output file)
Check that the number of genes is correct (for example the 0.1% significant genes)
Check that the bin values are appropriate for the trait data. These values should cover the range of the data and a little way either side.
Save the file and run the programme (Ctrl+W)

### 5.2 Making the Test Expression File

To make the predictions use the identified prediction probes, and the expression data of the ‘unknown lines’ for which we are making the prediction of heterosis. Using the list of identified prediction probes, create a specific prediction sub-set gene list, as was done when generating the file for the calibration curves (section 5.1). This can be done by copying your ID and P-value columns (sorted by ID to return the data to its original order) in to a new excel sheet along with the expression data of your training line accessions. You can then sort by P-value and delete those genes that do not appear in the relevant significance (usually 0.1%) list. Remember to sort by ID again to return the file to its correct order, then delete the ID and Sig0.1% columns you added. Save this file under a new file name as an Excel spread sheet.

In this file add two blank columns between each of the data columns. In the first column, next to the first unknown line's expression measurement, insert a number series from 1 to however long the list on gene measurements is. In the next column, list the identifier for those measurements (the best identifier would be the parent name, for instance Kas, B73 etc.).

In the first column next to the second data list type the command “=B2+0.0” Then copy this down the column. This will have the effect of giving a number series that is 0.01 greater than its equivalent for the first parent. In the next column, list the identifier for those measurements again

Repeat this process for any remaining parent data sets. Each number series should always be 0.01 greater than its equivalent in the previous series.

Starting with the second set of data columns, cut all of the genes, number series and identifies, and add them to the bottom of first set of data columns. Be sure to use Edit>Paste Special>Values so as not to upset your commands. Repeat this for the remaining columns. You should now have three long columns with all of the data in.

Select all of the data. Click Data>Sort>Column B (or whichever is the column with the number sequence in). After sorting, you should have all of your parental data mixed together, with all of the same genes next to each other (for example, with three parents your number sequence should read 1, 1.1, 1.2, 2, 2.1, 2.2 etc. and the identifier column should read Kas, Sha, Ll-0, Kas, Sha, Ll-0 etc. or equivalent) save the file. This is your identifier file.

Copy only the column with the expression data into a new work book. Delete all headings and add a column of colons “:”. Save the file as a .txt file. This is your ‘Tester’ data file. Ensure that you close this file, as GenStat will not recognise the file if open in Excel.

Open this file in GenStat press Ctrl+R and in the ‘Find What’ box type * leave the ‘Replace With’ box blank. Click ‘Replace All’ then save this file. This is your test expression file.

### 5.3 Running the Prediction File

### Open the ‘Prediction Extraction Programmer’ (GenStat Programme 3

Check the variate “mpadv” these are the X-axis values for the calibration lines. Ensure that these are the same as the bin values entered earlier (section 5.1).

Check the first input file. This should be the expression data of your Tester lines (section 5.2).

Check the second input file. This should be the output file from your calibration line (calibration output file—section 5.1).

Check that the “ntimes” command is the number of test genes multiplied by the number of parents, therefore the total number of genes in your test expression file.

Check that the “calc Z=Z+3” command is correct for your number of Tester lines, for example, for four Tester lines this should read “calc Z=Z+4”.

Check that your “if (estimate)” commands are appropriate for the range of your trait data. This is for the ‘capped’ prediction. These should be set at 2 ‘bin sizes’ beyond and below the bin range, if appropriate.

Run the programme (Ctrl+W). This programme prints to the output window, which should be saved as an output (.out) file.

Note it is normal for there to be error messages, if all of the previous steps have been followed ignore these.

### 5.4 Analysing the Output

Open your saved output file in Excel. Choose Delimited>Next and tick the Tab and Space buttons.

Delete the writing found in the file until you reach the first data point. Usually the first 60 lines.

Name the columns “No.” “Cap” “Raw”

Scroll to the bottom and delete all of the messages you see there.

Select all and sort by “No” ascending.

Check that you have the correct number of rows remaining. This should equal the ntimes value from the Prediction Extraction Programme (the number of prediction genes you have generated, multiplied by the number of Tester lines you are predicting for). Scroll to the bottom and delete all of the non-relevant information you see there (for example “regvr=regms/resms” “code CA” etc)

Delete any remaining warning messages, to the left and right of the ‘useful data.’

Open the identifier .xls file you generated earlier. Copy the Number series and Identifier columns in to your output file.

Select all (Ctrl+A) and sort by Identifier, this should separate the data by parent name.

Cut and paste all of the parents into neighbouring columns (so that they are next to each other).

Scroll to the bottom of the list under the cap column enter the command “=AVERAGE(B2:B203)” (Note, this command is based on 202 predictive genes, you should adjust this command to cover the number of predictions for your gene set).

Copy this command to the bottom of all of your lists. You should now have two predictions for each of your Tester lines, the CAPPED and RAW prediction values.

These predictions can be used individually, or they can be averaged between replicates of the same accessions.

### b) Recommended Prediction Protocol

### Method 6, N-1 Model

These instructions describe the first steps of the recommended prediction protocol. The N-1 model is a modification to the basic regression method, and using the same GenStat programme, however this regression is repeated for each accession in the training set.

### 6.1 Running the N-1 Model

To undertake the N-1 model, prepare an expression file containing all of the accessions you wish to use in your training set.

Run a basic regression (GenStat Programme 1-Basic Regression Programme) using all but one of these accessions. If you have multiple replicates of the same accession, ensure that all are removed.

Using the genes identified from this experiment, undertake a prediction as described in Method 5, using the removed accession as the tester line. Record the ID list of the predictive genes (section 4.4), and the results of the RAW prediction for each gene (as listed in section 5.4) for each replicate.

Repeat this process for all of the accession in the training set, until you have predicted each accession against a training set containing all of the other accessions. These data can be used to assess the overall accuracy of these predictions by plotting the ACTUAL trait values against the predicted, or they can be used for the later ‘Best Predictor’ prediction method.

### Method 7, Best Predictor

This programme calculates which genes consistently predict well over a wide range of accessions and phenotypes. You can also use the output to investigate the frequency of genes appearing in the predictive lists, and thereby identify many noise genes.

### 7.1 Creating the Data File

To create the data file first open a new Excel spreadsheet. In the first column, paste the list of predictive gene IDs (the numbers assigned at the regressions stage) from the first of the N-1 accessions (section 6.1). In the next column paste the list of predictions for these genes for this accession, as generated in the prediction stage for that accession in the N-1 model. In the third column at each stage paste the accession name, repeated next to each gene in the list. In the fourth column type the replicate number for that accession, if there is only one replicate type 1. In the fifth type the actual trait value for that accession.

### 7.2 Running the Prediction File

Open the ‘Basic Best Predictor Programme’ (GenStat Programme 4) Check that the names of the accessions are correctly listed.

Check that the number of replicates is correct (note these should be written [values=‘chip 1’,‘chip 2’] and so on for however many replicates there are).

Check that the Input file name is correct.

Run the programme (Ctrl+W). This programme prints to the output window, which should be saved as an output (.out) file.

### 7.3 Generating a Best Predictor File

Open your saved output file in Excel. Choose Delimited>Next and tick the Tab and Space buttons.

Delete the copy of the programme in the output (first 31 lines or so) at the top of the file, and the programme information at the bottom of the file (last 8 lines).

Only the first 4 columns (gene, number, Delta, and se_delta) are at the top of the file. Scroll half way down the sheet; there are 3 further columns (a repeat of gene, Ratio, and se_ratio) copy these columns next to the 4 columns at the top of the sheet.

Ensure that the column names are gene, number, Delta, and se_delta, gene, Ratio, se_ratio; respectively.

Delete the second ‘gene’ column.

Save the file. This file is your Best Predictor file

### 7.4 Using the Best Predictor File

The information in the Best Predictor file is:

Gene Gene is the gene ID list of the predictive genes (section 4.4).

Number The number of occasions that each gene occurs in the predictive gene lists of the N-1 model. Using this we can quickly understand the distribution of this gene between gene lists from the N-1 model (section 6.1). This information can be used to quickly identify ‘noise genes’ by their low frequency in gene lists.

Delta The Absolute Difference (AD) is the mean of the differences between actual trait values and the values predicted for each line in the model. The closer the AD to 0 the closer the predictions are, on average, to the actual value. This value gives a good ‘feel’ for how close a prediction is to the actual, in relation to the trait of interest. For example, an AD of 4 might seem good if the trait was height in cm, and seem a fair tolerance for a prediction, however if the trait was plot yield in Kg, this value might be rather large.

se_delta The standard error of the Absolute Difference (seAD). This value gives a measure of the variability of the prediction, the smaller this value is the smaller the variability of the AD. An ideal predictive gene will have a small AD and seAD.

Ratio Ratio of the Difference (RD). This is the mean of the Ratio between actual trait values and the values predicted for each line in the model. This value is a more universal measure of AD, as all values are normalised to 1 (1 being a perfect match between prediction and actual), and the closer to 1 a gene is the better the gene appears to be for prediction. In theory this should allow the predictive ability of a gene can be assigned, independently of the trait value. For example, a particular gene might have an AD of −0.12 for yield weight, but an RD of 0.98. Saying that the gene is on average a 98% accurate predictor is perhaps an easier concept to understand.

se_ratio The standard error of the Ratio of the Difference (seRD). This value gives a measure of the variability of the ratio of the prediction, the smaller this value is the smaller the variability of the RD. An ideal predictive gene will have an RD close to 1 and a small seRD.

Using these parameters it is possible to generate more accurate gene list for the prediction of heterosis. This is a trial and error process at present, experimenting with different combinations of parameters will identify the best combination of genes for that trait. At present the most consistent combination of parameters for a good analysis has been a gene frequency of ALL MODELS (the predictive gene must appear in all N-1 models), and a Ratio (or RD) of >0.98 and <1.02.

In order to the gene combination with the parameters of gene frequency of all models, and an RD of >0.98 and <1.02, firstly sort (data>sort) the Best Predictor file by ‘number’ with the data descending. Before pressing ‘OK’ use the ‘THEN BY’ function to sort the data by Ratio ascending. Press OK.

This will bring all of the most consistent genes to the top of the worksheet. Select all of the genes that display an RD of between 0.98 and 1.02.

To test whether this is a good predictor list, calculate the average prediction for each accession and replicate for this best predictor gene list, and plot these predictions against the actual values for that trait.

An R² value between 0.5 and 1 suggests that gene list contains genes that are good markers for predictions of that trait.

### Method 8, Best Predictor-Prediction

### 8.1 Best Predictor Prediction

This method is a variation on the standard predictive method (method 5), and uses the same GenStat programmes.

The only variation of this programme is to use the best predictor gene list in place of the 0.1% P-valve list, for generating the training and tester files.

### c) Alternative “Basic” Prediction Protocol

### Method 9, Bootstrapping

These instructions describe the first steps of the alternative prediction protocol. These methods are an addition to the basic regression method, and using the same GenStat programmes for the early stages. This Bootstrapping follows on directly from the basic regression (method 4), but prior to the prediction, and acts as an alternative method for identifying significant ‘marker’ genes. It works by generating a ‘customised T-table’ that is specific for the experiment in question.

### 9.1 Regression Bootstrapping

### Open the ‘Basic Linear Regression Bootstrapping Programme’(GenStat Programme 5) in GenStat

Check that the input data filename is correct, and is opening to channel 2. This input file will be the same expression data file used for the initial regression (section 4.1)
Check that the output data files are going to the correct destinations and are opening to channels 2, 3, 4, and 5
Check that the numbers of genes to be analysed are correct for each output file (for Arabidopsis ATH-1 GeneChips this will be three files with 6000 genes and one with 4810), and that the print directives are pointing to the correct channels

To run the programme, ensure that both the programme window and output windows are open. Select the programme window and press Ctrl+W. This will set the programme running, check that the GenStat server icon (bottom right-hand corner of the screen) has changed colour to red.

To cancel the programme right click on the server icon and choose interrupt.

Once complete the GenStat icon will change colour back to green. This programme can take many days to run due to the large number calculations, and produces output files totaling up to 430 Mb, so plenty of disk space would be required. Once generated, the data for this programme needs to be extracted.

### 9.2 Data Extraction Programme

Open the ‘Basic Linear Regression Bootstrapping Data Extraction Programme’ (GenStat Programme 6) in GenStat

Check that the input files are correct (the output files from the bootstrapping programme)
Run the programme (Ctrl-W)

This programme prints to the Output window. Save this window as an .out file.

### 9.3 Analysing the Output

To analyse the data, first open it in Excel, select “delimited”>next>tick the “Tab” and “Space”>Finish Delete the first 32 rows, all of the gaps (after 6000, 12000, and 18000 probes), and all the text at the end of the data file. The data should be the same length as the regression file (for Arabidopsis 22810 lines long).

Add a new row, and label the columns “boot@5%” “boot@1%” and “boot@0.1%”
Add a new column to the beginning and label it “ID”
Fill the remaining cells of the ID column with a series 1-22810 (edit>fill>series>OK)
Copy all of these columns into the same sheet as the Observed significant probes data set, generated from the initial regression (section 4.4) with a one column gap
Leaving another single column gap label three further columns “sig@5%” “sig@1%” and “sig@0.1%”. In the first cell in the column “sig@5%” type “=E2−$B2”. Copy this to all of the cells in the three new columns.

### 9.4 Calculating Significance

Select all of the data columns>Data>Sort>Sig@5% descending Select all of the cells in this row where the value is positive. Colour these cells using the “paint” option, and record the number in this list. These are the genes significant at the 5% level
Select all of the data columns>Data>Sort>Sig@1% descending
Select all of the cells in this row where the value is positive. Colour these cells using the “paint” option, and record the number in this list. These are the genes significant at the 1% level
Select all of the data columns>Data>Sort>Sig@0.1% descending
Select all of the cells in this row where the value is positive. Colour these cells using the “paint” option, and record the number in this list. These are the genes significant at the 0.1% level

These results indicate whether or not the OBSERVED values differ significantly from random chance. These lists of significant genes can be used as markers, for the prediction of this trait as described in Method 5.

### d) Transcription Remodelling Protocol

These analyses are designed to investigate the degree of difference in the transcriptome profiles between the hybrid and parental lines. There are two methods, investigating the transcriptome remodelling, and investigating the degree of dominance.

### Method 10, Transcriptome Remodelling Fold-Change Experiments

This analysis is designed to investigating the transcriptome remodelling between hybrid and parental transcriptomes.

### 10.1 Create Data File

To create a data file for use in GenStat. Open master normalised expression Excel file>Copy the relevant data columns (in the order 3 hybrid files, 3 paternal files, 3 maternal files) into a new chart>add a colon “:” at the very end of the last row>save chart as .txt file>close file
Open the text file in GenStat>Enclose any title names in speech marks (“ ”), this should have the effect of turning the titles green>Find and replace (Ctrl+R)* with blanks>Save file again

### 10.2 Fold Change Analysis Programme

### Open the ‘Basic Transcriptome Remodelling Programme’ (GenStat Programme 7) in GenStat

Check that the input data filename is correct, and is opening to channel 2
Check that the output data file is going to the correct destination and is opening to channel 3
Check that the ratios are set correctly for the ratio comparison under investigation.

For example, for

“if ((elem(i;k).gt.0.5).and.(elem(i;k).lt.2))”
This is set for a 2-fold ratio
For 3 fold the values would be 0.33 and 3
For 1.5 fold the values would be 0.66 and 1.5
The values are entered 3 times in the programme
Check that the ratios are set correctly for the fold change comparison under investigation. This is undertaken for all of the sections and should be set simply to the relevant fold level

To run the programme, ensure that both the programme window and output windows are open. Select the programme window and press ctrl>W. This will set the programme running, check that the GenStat server icon (bottom right-hand corner of the screen) has changed colour to red.

To cancel the programme right click on the server icon and choose interrupt
Once complete the GenStat icon will change colour back to green

### 10.3 Analysing the Output

To analyse the data, first open it in Excel, select “delimited”>next>tick the “Tab” and “Space”>Finish

Delete the first 266 rows in Excel, until you reach the column headers. Then delete bottom line beyond the data output

At the bottom of each column calculate the total number of significant patterns in that list. This can be done by using the directive “=SUM(C2:C22811)” in the first column and copying this into the remaining columns, ensuring that the correct data is selected.

The initial analysis is now complete. These values represent the OBSERVED data in the further analysis, following bootstrapping to generate the expected values.

### Method 11, Transcriptome Remodelling Dominance Experiments

This analysis is designed to investigating dominance type transcriptome remodelling between hybrid and parental transcriptomes. Significance is calculated by comparing observed values to the expected generated from random data. Note, this programme is in its early stages, and is not easy to modify.

### 11.1 Create Data File

This experiment compares the expression of the profile of the hybrid against the mean of it parents. To do this we must first calculate these mean values.

Open a new Excel worksheet. Paste in the parent expression data (both maternal and paternal) for the first replicate of the first accession.

Calculate the mean value for each gene. This can be done using typing the equation=AVERAGE(A2:B2) into the next cell along. Copy this equation all the way down this column.

Open another worksheet and paste in the expression data of the first hybrid, copy the newly generated mean parental expression value and Edit>Paste Special>Values in to the next column. Repeat this for all of the replicates and accessions. Note that this programme is designed to analyse 3 replicates of each hybrid, a total of 6 columns per accession.

Once this is complete, save the file as .txt. Open the file in GenStat>enclose the titles in “ ” which should change their colour to green. Save the file again. This is the input file.

### 11.2 Running the Dominance Pattern Recognition Programme

### Open the ‘Dominance Pattern Programme’ (GenStat Programme 8) in GenStat

Check the accession names (first scalar command) are correct. If you are investigating less than 8 accessions, you will need to change the numbers of these identifiers throughout the programme. Should you not wish to do this, running ‘pseudo-data’ in the remaining columns will not affect the output and can be ignored at the analysis stage.

Check the number of columns (second scalar command) is correct. It should be a 6× the number of accessions used (default is 48). Check that the out put file is correctly named and addressed.

Check that the input file is correct.

Check that the fold level is correct for the analysis you wish to under take. These values a recorded for 2 fold as

if (ratio.ge.0.5).and.(ratio.le.2) “calculates flags”
calc heqmp=1
elsif (ratio.gt.2)
calc hgtmp=1
elsif (ratio.lt.0.5)
calc hltmp=1

For other fold levels change the 0.5 and 2 values to the appropriate value for that fold level.

For 3 fold the values would be 0.33 and 3
For 1.5 fold the values would be 0.66 and 1.5
Run the file by pressing Ctrl+W.

### 11.3 Analysing the Pattern Recognition Output

To analyse the output file, first open it in Excel, select “delimited”>next>tick the “Tab” and “Space”>Finish

You will see a file filled with ‘1s’ and ‘0s.’ Scroll to the bottom of this file. Underneath the first filled column write the equation “=SUM(B1:B22810)” (ensuring that all of the data in that column is filled). Copy this equation to all of the columns.

Each set of three ‘sum values’ represent the data output for a single accession (3 replicates), in the order that the data was loaded into the programme. These values represent

Column 1=The number of genes who's hybrid expression falls within the fold level criterion of the mid-parent value, for ALL 3 replicates.

Column 2=The number of genes who's hybrid expression is greater than that of the mid-parent value, by at least the fold level criterion, for ALL 3 replicates.

Column 3=The number of genes who's hybrid expression is lower than that of the mid-parent value, by at least the fold level criterion, for ALL 3 replicates.

Record these values, as the OBSERVED for these data.

11.4 Generating the EXPECTED value.
The expected data set is generated using the ‘Dominance Permutation Programme’ (GenStat Programme 9)

Check the number of columns (second scalar command) is correct. It should be a 6× the number of accessions used (default is 48).

Check that the out put file is correctly named and addressed.

Check that the input file is correct. This is the same input file as generated previously.

Check that the fold level is correct for the analysis you wish to under take. These values a recorded for 2 fold as before (section 11.1)

Check the number in the permutation loop is correct for then number of permutations you require. A minimum of 100 is recommended (although 1000 is ideal).

Run the file by pressing Ctrl+W.

This programme may take a few days to run, depending upon how many permutations are added.

### 11.5 Analysing the Pattern Recognition Permutation Output

To analyse the output file, first open it in Excel, select “delimited”>next>tick the “Tab” and “Space”>Finish

You will see a file filled with numbers. Scroll to the bottom of this file. Underneath the first filled column write the equation “=SUM(B1:B123)” (ensuring that all of the data in that column is filled). Copy this equation to all of the columns.

Each set of three ‘sum values’ represent the permuted data output for a single accession (3 replicates), in the order that the data was loaded into the programme. The three values represent the ‘expected by random chance’ versions of the values calculated in section 11.3.

The calculated values at the bottom of the columns are the EXPECTED values required for this analysis. As these data are effectively random it is acceptable to combine these for comparison, if time is limiting.

### 11.6 Analysing the Significance

The level of significance is calculated by chi square analysis, using the observed and expected data generated previously, and 1 degree of freedom.

### Method 12, Transcriptome Remodelling Fold-Change Bootstrapping

This analysis is designed to assess the significance of fold change experiments described in Method 10. Significance is calculated by comparing observed values to expected generated from random data

### 12.1 Fold Change Bootstrapping

### Open ‘Transcriptome Remodelling Bootstrap Programme’ (GenStat Programme 10) in GenStat

Check that the input data filename is correct, and is opening to channel 2. This will be the same input file as created in section 10.1.

Check that the output data files is going to the correct destinations and is opening to channels 3

Check that the number of randomisations is set to the desired value. As few as 50 randomisations are sufficient to give valid estimates of random chance, however 1000 would be ideal, but this can take many days to obtain.

Check that the ratios are set correctly for the ratio comparison under investigation.

For example:

“if ((elem(i;k).gt.0.5).and.(elem(i;k).lt.2))”
This is set for a 2-fold ratio
For 3 fold the values would be 0.33 and 3
For 1.5 fold the values would be 0.66 and 1.

To run the programme, ensure that both the programme window and output windows are open. Select the programme window and press Ctrl>W. This will set the programme running, check that the GenStat server icon (bottom right-hand corner of the screen) has changed colour to red.

To cancel the programme right click on the server icon and choose interrupt
Once complete the GenStat icon will change colour back to green

### 12.2 Analysing the Output

To analyse the data, first open it in Excel, select “delimited”>next>tick the “Tab” and “Space”>Finish
Delete the first 281 rows in Excel, until you reach the first row of data. Then delete bottom line beyond the data output
Select the whole sheet and go to data>sort>sort by “Column B”. This will remove the empty rows from the data.

At the bottom of each column calculate the mean number of significant patterns in that list. This can be done by using the directive “=AVERAGE(B2:B22811)” in the first column and copying this into the remaining columns, ensuring that the correct data is selected.

This will give the EXPECTED mean value, expected by random chance in the data

### 12.3 Calculating Significance

Calculating the significance of the observed patterns requires the use of a maximum likelihood chi square test
Firstly open GenStat>Stats>Statistical Tests>Chi-Square Goodness of Fit
Click on “Observed data create table”>Spreadsheet
Name the table OBS>Change rows and columns to 1>OK and ignore the error message
In the new table cell type the number of the first OBSERVED column sum value
Click on “expected frequencies create table”>Spreadsheet Name the table EXP>leave rows and columns as 1>OK and ignore the error message
In the new table cell type the number of the first Expected mean column mean value
On the Chi-Square window put 1 into the degrees of freedom box and click Run

Record the Chi-Square and P value that appears in the Output window.

Type the next OBSERVED value into the OBS box and click onto the output window
Type the next EXPECTED value into the EXP box and click onto the output window
On the Chi-Square window click Run, and record the new Chi-Square and P value that appears in the Output window

This should then be undertaken for all of the remaining OBSERVED and EXPECTED values.

These results indicate whether or not the OBSERVED values differ significantly from random chance.

### Troubleshooting

This section describes some of the most common problems that can occur while running these programmes. Many of these problems/solutions apply to most of the programmes and as a result this section has not been divided up along programme lines. This list is not exhaustive, but should cover the majority of problems encountered. It should be noted that the ‘fault codes’ given are only for illustration, often many fault codes can result from the same root problem.

General GenStat problems

One common method of solving general problems is to ensure that all of the input files are closed prior to running the programme. This is achieved by typing (to close channel 2) “close ch=2” and then running this directive. By repeating this for channels 3-5, you can ensure that all of the channels are closed before running your programme, and thus avoiding conflicts.

Fault 16, code VA 11, statement 4 in for loop Command: fit [print=*]mpadv Invalid or incompatible type(s) Structure mpadv is not of the required type.

Remove comma from the end of the variate list.

Fault 29, code VA 11, statement 4 in for loop Command: fit [print=*]mpadv Invalid or incompatible type(s) Structure mpadv is not of the required type
Problem with the trait-data identifier. Possibly a different or missing identifier following the trait data variates (X-axis data)

### Failure to Run Problems

### —Too Many Values

Fault # code VA 5, statement 2 in for loop Command: read [ch=2; print=*; serial=n]exp Too many values

1) Ensure that the width parameter is large enough, set to a large enough value (400 is standard)
2) Ensure that if titles are included in the data file, that they are ‘greened out’ and not being read as data
3) Ensure that the “Unit” number (at the beginning of the programme) and the number of trait “variate”s are the same
—Too Few values
Fault 13, code VA 6, statement 4 in for loop Command: fit [print=*]mpadv Too few values (including null subset from RESTRICT) Structure mpadv has 37 values, whereas it should have 38
Ensure that the “Unit” number (at the beginning of the programme) and the number of trait “variate” are the same
Warning 6, code VA 6, statement 2 in for loop Command: read [ch=2; print=*; serial=n]exp Too few values (including null subset from RESTRICT)
Ensure that the “ntimes=” number and the number of probes in the data file are the same

### File Opening Failure

Fault #, code IO 25, statement 2 in for loop Command: read [ch=2; print=*; serial=n]exp Channel for input or output has not been opened, or has been terminated Input File on Channel 2
1) Input file name is incorrect
2) Input file address is incorrect
Fault 32, code IO 25, statement 12 in for loop Command: print [ch=3; iprint=*; clprint=*; rlprint=*]bin Channel for input or output has not been opened, or has been terminated Output File on Channel 3

Output file address is incorrect.

### Very Slow Running of Bootstrapping

Check that the programme is not having conflicts with anti-virus software. This should be solved by the computing department, but results from anti-virus software scanning the file each time it makes a write-to-disk operation. This can often be easily changed by modifying the scanning settings.

### If all Else Fails

Check that the file C:\Temp\Genstat is not filled. This can result from too many temp (.tmp) files being generated as a result of bootstrapping programmes. Deleting these files may improve the running of the programme.

Finally VSN (GenStat providers) can be contacted at ‘support@vsn-intl.com’

### Data Analysis Problems

### Missing or Very High F-Problems

Ensure that the data has not ‘shifted’ at very low f-probabilities. At the regression stage (section 4.4), before creating the ID column, add an extra column to the beginning of the file. Insert the ID column, and sort by DF, if the data has shifted, this should become apparent here.

TABLE 1
Genes showing correlation of transcript abundance in
hybrids with the magnitude of heterosis exhibited by those
hybrids

| | | |
|---|---|---|
| Affymetrix | AGI Code | Description |

Genes with transcript abundance in hybrids correlated with
strength of heterosis F < 0.001 MPH and F < 0.001 BPH

| Positive correlation | | |
|---|---|---|
| 251222_at | AT3G62580 | expressed protein |
| 257635_at | AT3G26280 | cytochrome P450 family protein |
| 250900_at | AT5G03470 | serine/threonine protein phosphatase 2A (PP2A) regulatory |
| 252637_at | AT3G44530 | transducin family protein/WD-40 repeat family protein |
| 253415_at | AT4G33060 | peptidyl-prolyl cis-trans isomerase cyclophilin-type family protein |
| 265226_at | AT2G28430 | expressed protein |
| 259770_s_at | AT1G07780 | phosphoribosylanthranilate isomerase 1 (PAI1) |
| 261075_at | AT1G07280 | expressed protein |
| 252501_at | AT3G46880 | expressed protein |

Genes with transcript abundance in hybrids correlated with
strength of heterosis F < 0.001 MPH and F < 0.01 BPH

| Positive correlation | | |
|---|---|---|
| 265217_s_at | AT4G20720 | dentin sialophosphoprotein-related |
| 253236_at | AT4G34370 | IBR domain-containing protein |
| 246592_at | AT5G14890 | NHL repeat-containing protein |
| 266018_at | AT2G18710 | preprotein translocase secY subunit, chloroplast (CpSecY) |
| 250755_at | AT5G05750 | DNAJ heat shock N-terminal domain-containing protein |
| 261555_s_at | AT1G63230 | pentatricopeptide (PPR) repeat-containing protein |
| 262321_at | AT1G27570 | phosphatidylinositol 3- and 4-kinase family protein |
| 246649_at | AT5G35150 | CACTA-like transposase family (Ptta/En/Spm) |
| 264214_s_at | AT1G65330 | MADS-box family protein |
| 261326_s_at | AT1G44180 | aminoacylase, putative/N-acyl-L-amino-acid amidohydrolase, |
| 255007_at | AT4G10020 | short-chain dehydrogenase/reductase (SDR) family protein |
| 246450_at | AT5G16820 | heat shock factor protein 3 (HSF3)/heat shock transcription factor |
| Negative correlation | | |
| 251608_at | AT3G57860 | expressed protein |
| 260595_at | AT1G55890 | pentatricopeptide (PPR) repeat-containing protein |
| 248940_at | AT5G45400 | replication protein, putative |
| 254958_at | AT4G11010 | nucleoside diphosphate kinase 3, mitochondrial (NDK3) |
| 257020_at | AT3G19590 | WD-40 repeat family protein/mitotic checkpoint protein, putative |

Genes with transcript abundance in hybrids correlated with
strength of heterosis F < 0.001 MPH and F < 0.05 BPH

| Positive correlation | | |
|---|---|---|
| 254431_at | AT4G20840 | FAD-binding domain-containing protein |
| 248941_s_at | AT5G45460 | expressed protein |
| 256770_at | AT3G13710 | prenylated rab acceptor (PRA1) family protein |
| 247443_at | AT5G62720 | integral membrane HPP family protein |
| 258059_at | AT3G29035 | no apical meristem (NAM) family protein |
| 246259_at | AT1G31830 | amino acid permease family protein |
| 262844_at | AT1G14890 | invertase/pectin methylesterase inhibitor family protein |
| 246602_at | AT1G31710 | copper amine oxidase, putative |
| 247092_at | AT5G66380 | mitochondrial substrate carrier family protein |
| 264986_at | AT1G27130 | glutathione S-transferase, putative |
| Negative correlation | | |
| 258747_at | AT3G05810 | expressed protein |
| 266427_at | AT2G07170 | expressed protein |
| 263908_at | AT2G36480 | zinc finger (C2H2-type) family protein |
| 250924_at | AT5G03440 | expressed protein |
| 249690_at | AT5G36210 | expressed protein |
| 245447_at | AT4G16820 | lipase class 3 family protein |
| 260383_s_at | AT1G74060 | 60S ribosomal protein L6 (RPL6B) |

Genes with transcript abundance in hybrids correlated with
strength of heterosis F < 0.001 BPH and F < 0.01 MPH

| Positive correlation | | |
|---|---|---|
| 260260_at | AT1G68540 | oxidoreductase family protein |
| 252502_at | AT3G46900 | copper transporter, putative |
| 256680_at | AT3G52230 | expressed protein |
| 254651_at | AT4G18160 | outward rectifying potassium channel, putative (KCO6) |
| 264973_at | AT1G27040 | nitrate transporter, putative |
| 256813_at | AT3G21360 | expressed protein |
| 248697_at | AT5G48370 | thioesterase family protein |
| 267071_at | AT2G40980 | expressed protein |
| 246835_at | AT5G26640 | hypothetical protein |
| 252205_at | AT3G50350 | expressed protein |

Genes with transcript abundance in hybrids correlated with
strength of heterosis F < 0.001 BPH and F < 0.05 MPH

| Positive correlation | | |
|---|---|---|
| 266879_at | AT2G44590 | dynamin-like protein D (DL1D) |
| 253999_at | AT4G26200 | 1-aminocyclopropane-1-carboxylate synthase, putative/ACC |
| 266268_at | AT2G29510 | expressed protein |
| 264565_at | AT1G05280 | fringe-related protein |
| 255408_at | AT4G03490 | ankyrin repeat family protein |
| 261166_s_at | AT1G34570 | expressed protein |
| 252375_at | AT3G48040 | Rac-like GTP-binding protein (ARAC8) |
| 264192_at | AT1G54710 | expressed protein |
| 259886_at | AT1G76370 | protein kinase, putative |
| 251255_at | AT3G62280 | GDSL-motif lipase/hydrolase family protein |
| 260197_at | AT1G67623 | F-box family protein |
| 253645_at | AT4G29830 | transducin family protein/WD-40 repeat family protein |
| 245621_at | AT4G14070 | AMP-binding protein, putative |
| Negative correlation | | |
| 246053_at | AT5G08340 | riboflavin biosynthesis protein-related |
| 264341_at | At1G70270 | unknown protein |
| 250349_at | AT5G12000 | protein kinase family protein |
| 256412_at | AT3G11220 | Paxneb protein-related |

TABLE 2
List of genes showing a correlation between
transcript abundance in parents with the magnitude of MPH
exhibited by their hybrids with Landsberg er msl.
2A: Genes showing positive correlation between transcript
abundance and trait value

| | | | |
|---|---|---|---|
| AT5G10140 | AT2G32340 | AT4G04960 | AT3G58010 |
| AT1G03710 | AT2G07717 | AT3G06640 | AT5G65520 |
| AT3G29035 | AT1G03620 | AT1G02180 | AT3G03590 |
| AT5G24480 | AT2G41650 | AT4G25280 | AT5G46770 |
| AT3G47750 | AT1G13980 | AT5G20410 | AT1G68540 |
| AT1G65370 | AT1G22090 | AT4G01897 | AT2G26500 |
| AT5G66310 | AT1G65310 | AT1G31360 | AT5G53540 |
| AT1G70890 | AT2G39680 | AT2G21195 | AT5G18150 |
| AT2G06460 | AT3G28750 | AT5G13730 | AT5G54095 |
| AT4G19470 | AT2G47780 | AT5G43720 | AT1G54780 |
| AT1G54923 | AT4G11760 | AT3G59680 | AT5G55190 |
| AT5G60610 | AT3G51000 | AT2G27490 | AT1G80600 |
| AT5G46750 | AT1G09540 | AT2G16860 | AT3G57040 |
| AT1G27030 | AT5G63080 | AT2G20350 | AT5G59400 |
| AT4G18330 | AT4G14410 | AT2G13610 | AT5G58960 |
| AT5G61290 | AT1G51360 | AT4G00530 | AT2G41890 |
| AT3G23760 | AT1G44180 | AT1G14150 | AT1G78790 |
| AT3G47220 | AT3G51530 | AT2G14520 | AT1G70760 |
| AT3G05540 | AT4G20720 | AT1G72650 | AT2G32400 |
| AT3G47250 | AT3G27400 | AT1G64810 | AT2G36440 |
| AT3G22940 | AT5G48340 | AT4G24660 | AT5G16610 |
| AT3G23570 | AT1G34460 | AT5G38360 | AT5G05700 |
| AT5G25220 | AT5G38790 | AT5G03010 | AT2G31820 |
| AT5G28560 | AT1G15000 | AT3G21360 | AT1G05190 |
| AT1G14890 | AT1G58080 | AT3G56140 | AT5G64350 |
| AT5G27270 | AT3G26130 | AT3G17880 | AT2G35795 |
| AT4G10380 | AT1G67910 | AT1G60830 | AT4G00420 |
| AT2G07671 | AT1G80130 | AT1G79880 | AT1G04830 |
| AT2G16980 | AT4G16170 | AT2G42450 | AT5G04410 |
| AT2G45830 | AT2G44480 | AT2G36350 | AT1G68550 |
| AT3G09160 | orf107f | AT5G04900 | |
| AT2G29710 | AT1G21770 | AT4G15545 | |
| AT5G17790 | AT5G58130 | AT4G21280 | |
| AT4G20860 | AT2G35690 | AT2G22905 | |
| AT1G04660 | AT2G24040 | AT2G32650 | |
| AT5G66380 | AT1G18990 | AT4G16470 | |
| nad9 | AT4G10030 | AT1G70480 | |
| AT5G56870 | AT3G20270 | AT2G36370 | |
| AT5G24310 | ycf9 | AT5G64280 | |
| AT5G06530 | AT4G20830 | AT3G10750 | |
| AT1G29410 | AT1G71480 | AT3G61070 | |
| AT1G67600 | AT3G14560 | AT5G11840 | |
| AT3G44120 | AT5G66960 | AT5G40960 | |
| AT3G58350 | AT1G26230 | AT1G76080 | |
| AT4G10410 | AT4G28100 | AT3G23540 | |
| AT1G70870 | AT3G50810 | AT1G34620 | |
| psbI | AT5G37540 | AT3G12010 | |
| AT1G33910 | AT1G03300 | AT1G45050 | |
| AT3G10450 | AT1G65070 | AT4G17740 | |

2B: Genes showing negative correlation between transcript
abundance and trait value

| | |
|---|---|
| AT1G50120 | AT4G22753 |
| AT4G30890 | AT5G66750 |
| AT5G11560 | AT3G53170 |
| AT3G07170 | AT5G28460 |
| AT3G50000 | AT3G22310 |
| AT5G26100 | AT3G47530 |
| AT1G12310 | AT3G02230 |
| AT3G03070 | AT4G37870 |
| AT5G63220 | AT3G30867 |
| AT2G14835 | AT1G25230 |
| AT1G61770 | AT2G14890 |
| AT1G74050 | AT1G47210 |
| AT1G42480 | AT4G19040 |
| AT5G50000 | AT5G10390 |
| AT1G13900 | AT1G71880 |
| AT2G40290 | AT3G52500 |
| AT2G03220 | AT1G04040 |
| AT5G57870 | AT5G06265 |
| AT2G26140 | AT4G34710 |
| AT4G04910 | AT3G60450 |
| AT1G48140 | AT4G21480 |
| AT2G38970 | AT3G23560 |
| AT5G63400 | AT5G45270 |
| AT2G42910 | AT2G34840 |
| AT4G03550 | AT5G11580 |
| AT2G41110 | AT3G23080 |
| AT2G33845 | AT3G09270 |
| AT2G30530 | AT5G40370 |
| AT3G55360 | AT4G23570 |
| AT3G45770 | AT5G53940 |
| AT5G20280 | AT4G36680 |
| AT3G51550 | AT1G64450 |
| AT4G00860 | AT3G19590 |
| AT5G27120 | AT5G45550 |
| AT3G49310 | AT2G32190 |
| AT4G27430 | AT2G37340 |
| AT5G19320 | AT3G11220 |
| AT1G21830 | AT2G32190 |
| AT2G17440 | AT4G27590 |
| AT5G54100 | AT2G22470 |
| AT2G15000 | AT1G31550 |
| AT4G13270 | AT2G22200 |
| AT1G55890 | AT5G45510 |
| AT5G40890 | AT5G45500 |
| AT3G62960 | AT1G59930 |
| AT3G58180 | AT4G21650 |
| AT4G31630 | |
| AT3G57550 | |
| AT4G24370 | |

TABLE 3
Genes used for prediction of leaf number at bolting in
vernalised plants; Transcript ID (AGI code)

3A: Genes showing positive correlation
between transcript abundance and
trait value
At1g02620
At1g09575
At1g10740
At1g16460
At1g27210
At1g27590
At1g29440
At1g29610
At1g30970
At1g32150
At1g32740
At1g35660
At1g36160
At1g43730
At1g45474
At1g52870
At1g52990
At1g53170
At1g55130
At1g55300
At1g57760
At1g58470
At1g67690
At1967960
At1968330
At1g68840
At1g70730
At1g70830
At1g75490
At1g77490
At2g02750
At2g03330
At2g03760
At2g06220
At2g07050
At2g15810
At2g16650
At2g19010
At2g20550
At2g22440
At2g23180
At2g23480
At2g23560
At2g24660
At2g24790
At2g25850
At2g27190
At2g27220
At2g30990
At2g31800
At2g32020
At2g34020
At2g40420
At2g40940
At2g42380
At2g42590
At2g43320
At2g44800
At3g02180
At3g05750
At3g09470
At3g10810
At3g11100
At3g11750
At3g13120
At3g13222
At3g14000
At3g14250
At3g14440
At3g15190
At3g18050
At3g19170
At3g19850
At3g20020
At3g21210
At3g22710
At3g27020
At3g27325
At3g27770
At3g30220
At3g44410
At3g44720
At3g45580
At3g45780
At3g45840
At3g48730
At3g51560
At3g53680
At3g55560
At3g57780
At3g60260
At3g60290
At3g60430
At3g61530
At3g62430
At4g02610
At4g08680
At4g10550
At4g10925
At4g12510
At4g13800
At4g14920
At4g17240
At4g17260
At4g17560
At4g18460
At4g18820
At4g19140
At4g19240
At4g19985
At4g23290
At4g23300
At4g27050
At4g27990
At4g29420
At4g31030
At4g32000
At4g32250
At4g32410
At4g32810
At4g35760
At4g35930
At4g39390
At4g39560
At5g04190
At5g14340
At5g14800
At5g16010
At5g16800
At5g17210
At5g17570
At5g38310
At5g40290
At5g41870
At5g44860
At5g45320
At5g45390
At5g47390
At5g48900
At5g49730
At5g51080
At5g51230
At5g52780
At5g52900
At5g53130
At5g55750
At5g56520
At5g57345
At5g59650
At5g63360
At5g63800
At5g67430
ndhA
ndhH
psbM
rpl33
3B: Genes showing negative correlation
between transcript abundance and
trait value
At1g01230
At1g03710
At1g03820
At1g03960
At1g07070
At1g13090
At1g13680
At1g14930
At1g15200
At1g18250
At1g18850
At1g19340
At1g20070
At1g22340
At1g24070
At1g24100
At1g24260
At1g29050
At1g29310
At1g29850
At1g32770
At1g51380
At1g51460
At1g52040
At1g52760
At1g52930
At1g53160
At1g59670
At1g61570
At1g62560
At1g63540
At1g64900
At1g68990
At1g69440
At1g69750
At1g69760
At1g74660
At1g75390
At1g77540
At1g77600
At1g78050
At1g78780
At1g79520
At1g80170
At2g01520
At2g01610
At2g04740
At2g14120
At2g17670
At2g18040
At2g18600
At2g18740
At2g19480
At2g19750
At2g19850
At2g20450
At2g22240
At2g22920
At2g23700
At2g25670
At2g27360
At2g28450
At2g29070
At2g34570
At2g35150
At2g36170
At2g37020
At2g40435
At2g41140
At2g45660
At2g45930
At2g47640
At3g02310
At3g02800
At3g03610
At3g05230
At3g09310
At3g09720
At3g12520
At3g13570
At3g14120
At3g15270
At3g16080
At3g18280
At3g19370
At3g20100
At3g20430
At3g22370
At3g22540
At3g25220
At3g28500
At3g49600
At3g51780
At3g52590
At3g53140
At3g56900
At4g02290
At4g03156
At4g08150
At4g11160
At4g14010
At4g14350
At4g14850
At4g15910
At4g17770
At4g18470
At4g18780
At4g19850
At4g21090
At4g29230
At4g29550
At4g35940
At4g39320
At5g01730
At5g01890
At5g02030
At5g03840
At5g04850
At5g04950
At5g05280
At5g06190
At5g07370
At5g08370
At5g11630
At5g15800
At5g16040
At5g17370
At5g17420
At5g20740
At5g22460
At5g22630
At5g37260
At5g40380
At5g42180
At5g43860
At5g44620
At5g45010
At5g47540
At5g50110
At5g50350
At5g50915
At5g52040
At5g53770
At5g54250
At5g55560
At5g57920
At5g58710
At5g59305
At5g59310
At5g59460
At5g60490
At5g60690
At5g60910
At5g61310
At5g62290

TABLE 4
Genes used for prediction of leaf number at bolting in
unvernalised plants; Transcript ID (AGI code)

4A. Genes showing positive correlation
between transcript abundance and
trait value
At1g02813
At1g02910
At1g03840
At1g08750
At1g13810
At1g15530
At1g16280
At1g18530
At1g20370
At1g21070
At1g24390
At1g24735
At1g28430
At1g28610
At1g31500
At1g31660
At1g33265
At1g34480
At1g42690
At1g45616
At1g47230
At1g47980
At1g48040
At1g50230
At1g51340
At1g52290
At1g52600
At1g53500
At1g55370
At1g56500
At1g59510
At1g59720
At1g61280
At1g62630
At1g63150
At1g63680
At1g66070
At1g66850
At1g68600
At1g69680
At1g70870
At1g74700
At1g74800
At1g76380
At1g76880
At1g77140
At1g77870
At1g78070
At1g78720
At1g78930
At2g01860
At2g01890
At2g02050
At2g03420
At2g03460
At2g03480
At2g04840
At2g07734
At2g12400
At2g13690
At2g17250
At2g17870
At2g20200
At2g23610
At2g28620
At2g30390
At2g30460
At2g35400
At2g38650
At2g41770
At2g42120
At2g44820
At3g01040
At3g01110
At3g01250
At3g01440
At3g01790
At3g02350
At3g03230
At3g03780
At3g07040
At3g11980
At3g13280
At3g15400
At3g16100
At3g17170
At3g17710
At3g17840
At3g17990
At3g18000
At3g18130
At3g18700
At3g20140
At3g20320
At3g21950
At3g23310
At3g24150
At3g25140
At3g25805
At3g25960
At3g27240
At3g27360
At3g27780
At3g28007
At3g29660
At3g51680
At3g55510
At3g59780
At4g00640
At4g01970
At4g02820
At4g04790
At4g05640
At4g08140
At4g08250
At4g12460
At4g14605
At4g16120
At4g17615
At4g18030
At4g18070
At4g18720
At4g21890
At4g22040
At4g22800
At4g23740
At4g26310
At4g26360
At4g30720
At4g31590
At4g33070
At4g33770
At4g38050
At4g38760
At5g05450
At5g05840
At5g07630
At5g07720
At5g08180
At5g10020
At5g10250
At5g10950
At5g11240
At5g11270
At5g16690
At5g20680
At5g25070
At5g26780
At5g27330
At5g36120
At5g40830
At5g41480
At5g42700
At5g46330
At5g46690
At5g47435
At5g51050
At5g51100
At5g53070
At5g56280
At5g57310
At5g59350
At5g59530
At5g63040
At5g63150
At5g63440
At5g64480
accD
nad4L
orf121b
orf294
rps12.1
rps2
ycf4
4B. Genes showing negative correlation
between transcript abundance and
trait value
At1g02360
At1g04300
At1g04810
At1g04850
At1g06200
At1g08450
At1g10290
At1g12360
At1g15920
At1g18700
At1g18880
At1g21000
At1g22190
At1g22930
At1g23050
At1g23950
At1g24340
At1g30720
At1g33990
At1g34300
At1g34370
At1g48090
At1g50570
At1g54250
At1g54360
At1g59590
At1g59960
At1g60710
At1g60940
At1g61560
At1g65980
At1g66080
At1g68920
At1g70090
At1g70590
At1g72300
At1g72890
At1g75400
At1g78420
At1g78870
At1g78970
At1g79380
At1g79840
At1g80630
At2g01060
At2g02390
At2g05070
At2g15080
At2g21180
At2g22800
At2g25080
At2g26300
At2g28070
At2g29120
At2g30140
At2g31350
At2g32850
At2g35900
At2g41640
At2g41870
At2g42270
At2g43000
At2g44130
At2g45600
At2g47250
At2g47800
At2g48020
At3g01650
At3g01770
At3g04070
At3g06130
At3g07690
At3g08650
At3g09735
At3g09840
At3g10500
At3g11410
At3g12480
At3g13062
At3g15900
At3g17770
At3g18370
At3g20250
At3g21640
At3g23600
At3g26520
At3g29180
At3g43520
At3g44880
At3g46960
At3g48410
At3g48760
At3g51010
At3g51890
At3g52550
At3g55005
At3g56310
At3g59950
At3g60245
At3g60980
At3g62590
At4g02470
At4g07950
At4g09800
At4g15420
At4g15620
At4g16760
At4g16830
At4g16845
At4g16990
At4g17040
At4g17340
At4g17600
At4g18260
At4g20110
At4g22190
At4g23880
At4g28160
At4g29735
At4g29900
At4g31985
At4g33300
At4g35060
At5g01650
At5g03455
At5g05680
At5g06960
At5g12250
At5g14240
At5g15880
At5g18900
At5g21070
At5g22450
At5g24450
At5g25120
At5g25440
At5g25490
At5g25560
At5g25880
At5g38850
At5g39610
At5g39950
At5g40250
At5g40330
At5g42310
At5g42560
At5g43460
At5g44390
At5g45050
At5g45420
At5g45430
At5g45500
At5g45510
At5g48180
At5g49000
At5g49500
At5g52240
At5g57160
At5g57340
At5g58220
At5g58350
At5g59150
At5g66810
At5g67380

TABLE 5
Genes used for prediction of ratio of leaf number at
bolting (vernalised plants)/leaf number at bolting
(unvernalised plants); Transcript ID (AGI code)

5A. Genes showing positive correlation
between transcript abundance and trait value
At1g01550
At1g02360
At1g02390
At1g02740
At1g02930
At1g03210
At1g03430
At1g07000
At1g07090
At1g08050
At1g08450
At1g09560
At1g10340
At1g10660
At1g12360
At1g13100
At1g13340
At1g14070
At1g14870
At1g15520
At1g15790
At1g15880
At1g15890
At1g18570
At1g19250
At1g19960
At1g21240
At1g21570
At1g22890
At1g22930
At1g22985
At1g23780
At1g23830
At1g23840
At1g26380
At1g26390
At1g28130
At1g28280
At1g28340
At1g28670
At1g30900
At1g32700
At1g32740
At1g32940
At1g34300
At1g34540
At1g35230
At1g35320
At1g35560
At1g43910
At1g45145
At1g48320
At1g49050
At1g50420
At1g50430
At1g50570
At1g51280
At1g51890
At1g53170
At1g54320
At1g54360
At1g55730
At1g57650
At1g57790
At1g58470
At1g61740
At1g62763
At1g66090
At1g66100
At1g66240
At1g66880
At1g67330
At1g67850
At1g68300
At1g68920
At1g69930
At1g71070
At1g71090
At1g72060
At1g72280
At1g72900
At1g73260
At1g73805
At1g75130
At1g75400
At1g78410
At1g79840
At1g80460
At2g02390
At2g02930
At2g03070
At2g03870
At2g03980
At2g05520
At2g06470
At2g11520
At2g13810
At2g14560
At2g14610
At2g15390
At2g16790
At2g17040
At2g17120
At2g17650
At2g17790
At2g18680
At2g18690
At2g20145
At2g22170
At2g22690
At2g22800
At2g23810
At2g24160
At2g24850
At2g25625
At2g26240
At2g26400
At2g26600
At2g26630
At2g28210
At2g28940
At2g29350
At2g29470
At2g30500
At2g30520
At2g30550
At2g30750
At2g30770
At2g31880
At2g31945
At2g32140
At2g33220
At2g33770
At2g34500
At2g35980
At2g39210
At2g39310
At2g40410
At2g40600
At2g40610
At2g41100
At2g42390
At2g43000
At2g43570
At2g44380
At2g45760
At2g46020
At2g46150
At2g46330
At2g46400
At2g46450
At2g46600
At2g47710
At3g01080
At3g03560
At3g04070
At3g04210
At3g04720
At3g08650
At3g08690
At3g08940
At3g09020
At3g09735
At3g09940
At3g10640
At3g10720
At3g11010
At3g11820
At3g11840
At3g12040
At3g13100
At3g13270
At3g13370
At3g13610
At3g13772
At3g13950
At3g13980
At3g14210
At3g14470
At3g16990
At3g18250
At3g18490
At3g18860
At3g18870
At3g20250
At3g22060
At3g22231
At3g22240
At3g22600
At3g22970
At3g23050
At3g23080
At3g23110
At3g25070
At3g25610
At3g26170
At3g26210
At3g26220
At3g26230
At3g26450
At3g26470
At3g28180
At3g28450
At3g28510
At3g43210
At3g44630
At3g45240
At3g45780
At3g47050
At3g47480
At3g48090
At3g48640
At3g50290
At3g50770
At3g50930
At3g51010
At3g51330
At3g51430
At3g51440
At3g51890
At3g52240
At3g52400
At3g52430
At3g53410
At3g56310
At3g56400
At3g56710
At3g57260
At3g57330
At3g60420
At3g60980
At3g61010
At3g61540
At4g00330
At4g00355
At4g00700
At4g00955
At4g01010
At4g01700
At4g02380
At4g02420
At4g02540
At4g03450
At4g04220
At4g05040
At4g05050
At4g08480
At4g10500
At4g11890
At4g11960
At4g12010
At4g12510
At4g12720
At4g13560
At4g14365
At4g14610
At4g15420
At4g15620
At4g16260
At4g16750
At4g16845
At4g16850
At4g16870
At4g16880
At4g16890
At4g16950
At4g16990
At4g17250
At4g17270
At4g17900
At4g19660
At4g21830
At4g22560
At4g22670
At4g23140
At4g23150
At4g23180
At4g23220
At4g23260
At4g23310
At4g25900
At4g26070
At4g26410
At4g27280
At4g29050
At4g29740
At4g29900
At4g33300
At4g34135
At4g34215
At4g35750
At4g36990
At4g37010
At5g04720
At5g05460
At5g06330
At5g06960
At5g07150
At5g08240
At5g10380
At5g10740
At5g10760
At5g11910
At5g11920
At5g13320
At5g14430
At5g18060
At5g18780
At5g21070
At5g22570
At5g24530
At5g25260
At5g25440
At5g26920
At5g27420
At5g35200
At5g37070
At5g37930
At5g38850
At5g38900
At5g39030
At5g39520
At5g39670
At5g40170
At5g40780
At5g40910
At5g41150
At5g42050
At5g42090
At5g42250
At5g42560
At5g43440
At5g43460
At5g43750
At5g44570
At5g44980
At5g45050
At5g45110
At5g45420
At5g45500
At5g45510
At5g48810
At5g51640
At5g51740
At5g52240
At5g52760
At5g53050
At5g53130
At5g53870
At5g54290
At5g54610
At5g55450
At5g55640
At5g57220
At5g58220
At5g59420
At5g60280
At5g60950
At5g61900
At5g62150
At5g62950
At5g63180
At5g64000
At5g66590
At5g67340
At5g67590
5B. Genes showing negative correlation between
transcript abundance and trait value
At1g03820
At1g05480
At1g06020
At1g06470
At1g07370
At1g18100
At1g20750
At1g28610
At1g31660
At1g44790
At1g47230
At1g49740
At1g51340
At1g52290
At1g61280
At1g63130
At1g63680
At1g64100
At1g66140
At1g67720
At1g69420
At1g69700
At1g71920
At1g74800
At1g76270
At1g77680
At1g78720
At1g78930
At2g01890
At2g03480
At2g13920
At2g14530
At2g17280
At2g18890
At2g20470
At2g22870
At2g33330
At2g36230
At2g36930
At2g37860
At2g39220
At2g39830
At2g40160
At2g44310
At3g05030
At3g05940
At3g06200
At3g10450
At3g10840
At3g13560
At3g13640
At3g15400
At3g17990
At3g18000
At3g18070
At3g19790
At3g20240
At3g21510
At3g24470
At3g27180
At3g28270
At3g45930
At3g47510
At3g49750
At3g50810
At3g52370
At3g54250
At3g54820
At3g57000
At4g04790
At4g08140
At4g10280
At4g10320
At4g12430
At4g14420
At4g16700
At4g17180
At4g19100
At4g23720
At4g23750
At4g24670
At4g26140
At4g31210
At4g31540
At4g34740
At4g35990
At4g38050
At4g38760
At5g02050
At5g02180
At5g02590
At5g02740
At5g06050
At5g07800
At5g08180
At5g14370
At5g15050
At5g19920
At5g20240
At5g22430
At5g22790
At5g23570
At5g27330
At5g27660
At5g41480
At5g43880
At5g49555
At5g51050
At5g51350
At5g53760
At5g53770
At5g55400
At5g55710
At5g56620
At5g57960
At5g59350
At5g61770
At5g62575
orf121b

TABLE 6
Genes for prediction of oil content of seeds, % dry
weight (vernalised plants); Transcript ID (AGI code)

6A. Genes showing positive correlation
between transcript abundance and
trait value
At1g02640
At1g02750
At1g02890
At1g04170
At1g05550
At1g05720
At1g08110
At1g08560
At1g09200
At1g09575
At1g10170
At1g10590
At1g13250
At1g15260
At1g17590
At1g18650
At1g23370
At1g27590
At1g29180
At1g31020
At1g34030
At1g42480
At1g48140
At1g49660
At1g51950
At1g52800
At1g54850
At1g55300
At1g60010
At1g60230
At1g61810
At1g63780
At1g64105
At1g64450
At1g65260
At1g66130
At1g66180
At1g67350
At1g69690
At1g70730
At1g71970
At1g74670
At1g74690
At2g01090
At2g14890
At2g17650
At2g18400
At2g18550
At2g18990
At2g20210
At2g20220
At2g20840
At2g21860
At2g25170
At2g25900
At2g27260
At2g29550
At2g30050
At2g30530
At2g31120
At2g31640
At2g31955
At2g32440
At2g36490
At2g37050
At2g37410
At2g38120
At2g38720
At2g39850
At2g39870
At2g39990
At2g40040
At2g40570
At2g41370
At2g42300
At2g42590
At2g42740
At2g44130
At2g44530
At2g45190
At3g02500
At3g03310
At3g03380
At3g05410
At3g06470
At3g07080
At3g14240
At3g15550
At3g17850
At3g18390
At3g19170
At3g24660
At3g28345
At3g51150
At3g53110
At3g53170
At3g55480
At3g55610
At3g57340
At3g57490
At3g57860
At3g60390
At3g60520
At3g61180
At3g62720
At3g63000
At4g00180
At4g00600
At4g00860
At4g00930
At4g01120
At4g01460
At4g02440
At4g02700
At4g03050
At4g03070
At4g07400
At4g11790
At4g12600
At4g12880
At4g14550
At4g15780
At4g16490
At4g17560
At4g20070
At4g21650
At4g27830
At4g29750
At4g32760
At4g34250
At4g38670
At5g02770
At5g04600
At5g07000
At5g07030
At5g07300
At5g07640
At5g07840
At5g08330
At5g08500
At5g09330
At5g10390
At5g15390
At5g17100
At5g19530
At5g22290
At5g23420
At5g24210
At5g25180
At5g25760
At5g26270
At5g27360
At5g32470
At5g36210
At5g36900
At5g37510
At5g38140
At5g40150
At5g41650
At5g44860
At5g45260
At5g45270
At5g46160
At5g47030
At5g47760
At5g48900
At5g50230
At5g51660
At5g52110
At5g52250
At5g54190
At5g54580
At5g55670
At5g55900
At5g57660
At5g58600
At5g60850
At5g62530
At5g62550
At5g63860
At5g65650
6B. Genes showing negative correlation
between transcript abundance and
trait value
At1g01790
At1g03710
At1g04220
At1g04960
At1g04985
At1g06550
At1g06780
At1g10550
At1g11070
At1g11280
At1g11630
At1g12550
At1g15310
At1g16060
At1g16540
At1g16880
At1g18830
At1g22480
At1g23120
At1g27440
At1g29700
At1g31580
At1g34040
At1g34210
At1g47410
At1g47960
At1g49710
At1g50580
At1g51070
At1g51440
At1g51580
At1g51805
At1g53690
At1g54560
At1g55850
At1g61667
At1g62860
At1g63320
At1g64950
At1g65480
At1g66930
At1g69750
At1g70250
At1g70270
At1g72800
At1g73177
At1g74590
At1g74650
At1g75690
At1g77000
At1g77380
At1g78450
At1g78740
At1g78750
At1g79950
At1g80130
At1g80170
At2g02960
At2g11690
At2g13770
At2g19570
At2g19850
At2g20410
At2g20500
At2g21630
At2g22920
At2g23340
At2g26170
At2g27760
At2g30020
At2g31450
At2g31820
At2g32490
At2g33480
At2g37970
At2g37975
At2g44850
At2g47570
At2g47640
At3g01720
At3g01970
At3g05210
At3g05540
At3g09410
At3g09480
At3g14395
At3g14720
At3g16520
At3g17800
At3g18980
At3g19320
At3g19710
At3g20270
At3g22370
At3g22740
At3g23170
At3g24400
At3g25120
At3g26130
At3g27960
At3g28050
At3g29787
At3g30720
At3g42840
At3g43240
At3g45070
At3g45270
At3g46500
At3g47320
At3g49360
At3g50810
At3g51030
At3g51580
At3g53690
At3g57630
At3g57680
At3g57760
At3g60170
At3g62390
At3g62400
At3g62410
At4g00960
At4g01070
At4g01080
At4g02450
At4g03060
At4g03260
At4g03400
At4g03500
At4g03640
At4g04900
At4g09680
At4g10150
At4g12020
At4g13050
At4g13180
At4g14040
At4g17390
At4g18210
At4g18780
At4g19980
At4g20840
At4g21400
At4g22790
At4g24130
At4g24940
At4g25040
At4g25890
At4g26610
At4g28350
At4g32240
At4g32690
At4g33040
At4g34240
At4g37150
At4g39780
At5g02820
At5g05420
At5g08600
At5g08750
At5g10180
At5g11600
At5g15600
At5g16520
At5g17060
At5g17420
At5g17790
At5g20180
At5g23010
At5g24510
At5g24850
At5g25640
At5g25830
At5g26665
At5g28560
At5g35400
At5g35520
At5g37300
At5g38780
At5g38980
At5g39550
At5g39940
At5g42180
At5g43480
At5g43500
At5g44030
At5g44740
At5g45170
At5g46490
At5g47050
At5g47630
At5g48110
At5g48340
At5g49530
At5g49540
At5g52380
At5g53090
At5g53350
At5g54660
At5g54690
At5g56030
At5g56700
At5g58980
At5g59305
At5g59690
At5g60160
At5g61640
At5g63590
At5g64816

TABLE 7
Genes with transcript abundance correlating with ratio
of 18:2/18:1 fatty acids in seed oil (vernalised plants);
Transcript ID (AGI code)

7A. Genes showing positive correlation
between transcript abundance and
trait value
At1g01730
At1g15490
At1g16060
At1g16540
At1g23120
At1g26730
At1g34220
At1g35260
At1g50580
At1g54560
At1g59620
At1g61400
At1g62860
At1g67550
At1g74650
At1g76690
At1g77380
At1g77590
At1g78450
At1g78750
At1g79950
At1g80170
At2g01120
At2g02960
At2g03680
At2g13770
At2g17220
At2g20410
At2g21630
At2g27090
At2g34440
At2g37975
At2g38010
At2g44850
At2g44910
At3g01720
At3g05210
At3g05270
At3g05320
At3g11880
At3g13840
At3g14450
At3g16520
At3g19930
At3g22690
At3g24400
At3g42840
At3g45640
At3g48580
At3g49360
At3g57760
At4g02450
At4g03060
At4g04650
At4g10150
At4g12020
At4g13050
At4g13180
At4g15260
At4g17390
At4g24920
At4g24940
At4g32240
At5g06730
At5g06810
At5g08750
At5g13890
At5g17060
At5g19560
At5g20180
At5g23010
At5g28500
At5g28560
At5g38980
At5g43330
At5g44740
At5g47050
At5g49540
At5g56910
At5g60160
At5g64816
7B. Genes showing negative correlation
between transcript abundance and
trait value
At1g02050
At1g04170
At1g04790
At1g06580
At1g08110
At1g13250
At1g14700
At1g15280
At1g18650
At1g26920
At1g29180
At1g29950
At1g33055
At1g35720
At1g49660
At1g51950
At1g52800
At1g52810
At1g54450
At1g60190
At1g60390
At1g60800
At1g62500
At1g62510
At1g63780
At1g64105
At1g66180
At1g66250
At1g66900
At1g67590
At1g67830
At1g69690
At1g75710
At1g76320
At2g04700
At2g14900
At2g16800
At2g18990
At2g20210
At2g20220
At2g20360
At2g21860
At2g25900
At2g27970
At2g31120
At2g34560
At2g36490
At2g37410
At2g38120
At2g39450
At2g39870
At2g40040
At2g40570
At2g42740
At2g44860
At3g02500
At3g07200
At3g08000
At3g11420
At3g11760
At3g14240
At3g24660
At3g26310
At3g27420
At3g44010
At3g47060
At3g53230
At3g55480
At3g55610
At3g56060
At3g57860
At3g60520
At3g60530
At3g61830
At3g62430
At3g62460
At4g00600
At4g00930
At4g03050
At4g03070
At4g12600
At4g13980
At4g14550
At4g15780
At4g16920
At4g17560
At4g22160
At4g25150
At4g26555
At4g36140
At4g36740
At5g07000
At5g07030
At5g10390
At5g15120
At5g17020
At5g17100
At5g17220
At5g18070
At5g25590
At5g26270
At5g37510
At5g40150
At5g43280
At5g46160
At5g47760
At5g51080
At5g51660
At5g52230
At5g54190
At5g55670
At5g57660
At5g63860
At5g65390
At5g65650
At5g65880
18:2 = linoleic acid
18:1 = oleic acid

TABLE 8
Genes for prediction of ratio of 18:3/18:1 fatty
acids in seed oil (vernalised plants); Transcript ID (AGI code)

8A. Genes showing positive correlation
between transcript abundance and
trait value
At1g11940
At1g15490
At1g22200
At1g23890
At1g28030
At1g33560
At1g49030
At1g51430
At1g59265
At1g62610
At1g64190
At1g69450
At1g71140
At1g78210
At2g07050
At2g31770
At2g35736
At2g46640
At3g14780
At3g16700
At3g26430
At3g46540
At3g49360
At3g51580
At4g01690
At4g08240
At4g11900
At4g12300
At4g18593
At4g23300
At4g24940
At4g38930
At4g39390
At5g03290
At5g05750
At5g08590
At5g11270
At5g13890
At5g14700
At5g16250
At5g17880
At5g18400
At5g20180
At5g22860
At5g23510
At5g27760
At5g28940
At5g44240
At5g44290
At5g44520
At5g46630
At5g47410
At5g49540
At5g49630
At5g54970
At5g55760
At5g55930
At5g64110
8B. Genes showing negative correlation
between transcript abundance and
trait value
At1g05550
At1g06500
At1g06580
At1g10320
At1g10980
At1g16170
At1g21080
At1g24070
At1g29180
At1g30880
At1g32310
At1g33055
At1g59900
At1g61810
At1g63780
At1g63850
At1g65560
At1g66130
At1g67830
At1g70430
At1g72260
At1g76720
At2g01090
At2g17550
At2g18100
At2g20490
At2g20515
At2g20585
At2g21090
At2g21860
At2g31840
At2g32160
At2g36570
At3g06470
At3g07080
At3g11410
At3g14150
At3g15900
At3g18940
At3g22210
At3g23325
At3g24660
At3g26240
At3g44600
At3g44890
At3g50380
At3g51780
At3g52090
At3g53110
At3g53390
At3g54290
At3g57860
At3g62080
At3g62860
At4g01330
At4g02210
At4g03070
At4g05450
At4g10320
At4g14870
At4g14890
At4g14960
At4g16830
At4g17410
At4g18975
At4g23870
At4g26170
At4g35240
At4g35880
At4g36380
At5g07640
At5g08540
At5g11310
At5g13970
At5g17010
At5g17100
At5g19830
At5g22290
At5g23330
At5g25120
At5g25180
At5g26270
At5g41970
At5g47550
At5g47760
At5g48580
At5g48760
At5g49190
At5g49500
At5g50950
At5g51660
At5g64650
At5g65010
18:3 = linolenic acid
18:1 = oleic acid

TABLE 9
Genes with transcript abundance correlating with ratio
of 18:3/18:2 fatty acids in seed oil (vernalised plants);
Transcript ID (AGI code)

9A. Genes showing positive correlation
between transcript abundance and
trait value
At1g01370
At1g01530
At1g02300
At1g02710
At1g03420
At1g05650
At1g08170
At1g11940
At1g13280
At1g13810
At1g15050
At1g20810
At1g20980
At1g21710
At1g22200
At1g23670
At1g23890
At1g27210
At1g33880
At1g44960
At1g51430
At1g51980
At1g57760
At1g57780
At1g59740
At1g60300
At1g60560
At1g62630
At1g62770
At1g66520
At1g66620
At1g70830
At1g71690
At1g77490
At1g79000
At1g79060
At2g02590
At2g02770
At2g07050
At2g07702
At2g11270
At2g15790
At2g18115
At2g19310
At2g28100
At2g28160
At2g32330
At2g34310
At2g35890
At2g38140
At2g39700
At2g41600
At2g43320
At2g44100
At2g45150
At2g45710
At2g45920
At2g46640
At2g47600
At3g05520
At3g09140
At3g10810
At3g11090
At3g12920
At3g14780
At3g16370
At3g18060
At3g18270
At3g22710
At3g22850
At3g22880
At3g27325
At3g28090
At3g29770
At3g31415
At3g43960
At3g45440
At3g46670
At3g48730
At3g59860
At3g61160
At3g61170
At3g62430
At4g01350
At4g07420
At4g11835
At4g12300
At4g12510
At4g17650
At4g18460
At4g18593
At4g18820
At4g20140
At4g23300
At4g25570
At4g31870
At4g32960
At4g33160
At4g35530
At4g37220
At4g39390
At5g03730
At5g05840
At5g05890
At5g07250
At5g08280
At5g17210
At5g18390
At5g20590
At5g22500
At5g22860
At5g26140
At5g26180
At5g28620
At5g28940
At5g35490
At5g38120
At5g40230
At5g43070
At5g45120
At5g45320
At5g46630
At5g47400
At5g49630
At5g51080
At5g51230
At5g51960
At5g56370
At5g57345
At5g59660
At5g62030
At5g64110
At5g64970
At5g65100
At5g66985
cox1
orf154
9B. Genes showing negative correlation
between transcript abundance and
trait value
At1g02500
At1g02780
At1g03710
At1g06500
At1g06520
At1g12750
At1g13090
At1g14930
At1g14990
At1g15200
At1g19340
At1g22500
At1g22630
At1g26170
At1g28060
At1g29850
At1g30530
At1g31340
At1g32310
At1g47480
At1g50140
At1g52040
At1g53590
At1g54250
At1g59670
At1g59900
At1g60710
At1g62560
At1g63540
At1g64140
At1g64900
At1g66690
At1g67860
At1g72510
At1g73177
At1g74880
At1g76260
At1g76560
At1g76890
At1g77540
At1g77600
At1g78080
At1g78750
At1g78780
At1g79430
At1g80170
At2g15630
At2g19740
At2g19850
At2g20490
At2g21640
At2g22920
At2g25670
At2g25970
At2g27360
At2g28200
At2g28450
At2g29070
At2g29120
At2g30000
At2g36750
At2g37585
At2g39910
At2g40010
At2g45930
At2g47250
At2g48020
At3g01860
At3g03610
At3g06110
At3g06790
At3g07230
At3g09480
At3g11410
At3g12090
At3g13490
At3g13800
At3g15900
At3g16080
At3g17770
At3g18940
At3g21250
At3g22210
At3g23325
At3g25220
At3g25740
At3g28700
At3g31910
At3g44890
At3g46490
At3g47320
At3g48860
At3g51780
At3g53390
At3g53500
At3g53630
At3g53890
At3g54260
At3g55005
At3g55630
At3g56900
At3g57180
At3g59810
At3g61100
At3g61980
At3g62040
At4g02075
At4g03240
At4g04620
At4g05450
At4g10120
At4g13195
At4g14020
At4g14350
At4g14615
At4g15230
At4g17410
At4g18330
At4g18780
At4g19850
At4g21090
At4g22380
At4g25890
At4g29230
At4g29550
At4g30220
At4g30290
At4g30760
At4g31310
At4g31985
At4g32240
At4g35240
At4g37150
At5g02610
At5g02670
At5g03455
At5g03540
At5g04420
At5g04850
At5g05680
At5g07370
At5g07690
At5g08535
At5g08540
At5g13970
At5g16040
At5g17930
At5g25120
At5g28080
At5g28500
At5g39550
At5g40540
At5g45840
At5g47050
At5g47540
At5g48110
At5g48580
At5g49530
At5g50915
At5g50940
At5g50950
At5g51010
At5g51820
At5g55560
At5g57160
At5g58520
At5g59460
At5g61450
At5g61830
At5g62290
At5g63590
At5g64140
At5g64190
At5g66530
18:3 = linolenic acid
18:2 = linoleic acid

TABLE 10
Genes with transcript abundance correlating with ratio
of 20C + 22C/16C + 18C fatty acids in seed oil (vernalised
plants); Transcript ID (AGI code)

10A. Genes showing positive correlation
between transcript abundance and
trait value
At1g01370
At1g03420
At1g04790
At1g06730
At1g09850
At1g11800
At1g21690
At1g43650
At1g49200
At1g50660
At1g53460
At1g53850
At1g55120
At1g60390
At1g62150
At1g69670
At1g79060
At1g79460
At1g79970
At2g25450
At2g35155
At2g40070
At2g40480
At2g45710
At2g46710
At2g47380
At3g04680
At3g09710
At3g10650
At3g14240
At3g26090
At3g26310
At3g26380
At3g29770
At3g44500
At3g56060
At3g57880
At4g13360
At4g14090
At4g24390
At4g26555
At4g31570
At4g35900
At5g05230
At5g05370
At5g10400
At5g17210
At5g23940
At5g24280
At5g24520
At5g25940
At5g37290
At5g38630
At5g40880
At5g47320
At5g52410
At5g54860
At5g55810
10B. Genes showing negative correlation
between transcript abundance and
trait value
At1g02410
At1g02475
At1g02500
At1g05350
At1g05360
At1g07260
At1g17310
At1g17970
At1g21110
At1g21190
At1g21350
At1g22520
At1g22910
At1g27000
At1g32050
At1g32070
At1g32310
At1g33330
At1g33600
At1g34580
At1g35650
At1g44750
At1g47480
At1g47920
At1g49240
At1g50630
At1g51940
At1g53650
At1g58300
At1g59900
At1g60810
At1g60970
At1g61400
At1g62090
At1g64150
At1g66540
At1g66645
At1g72920
At1g73120
At1g73250
At1g73940
At1g74620
At1g77590
At1g77960
At1g77970
At1g78750
At1g79890
At1g80640
At1g80700
At2g02500
At2g02960
At2g05950
At2g14170
At2g15560
At2g15930
At2g16750
At2g17265
At2g19800
At2g19950
At2g21070
At2g22570
At2g23360
At2g24610
At2g28850
At2g28930
At2g29680
At2g30000
At2g30270
At2g32160
At2g34690
At2g35520
At2g38220
At2g40010
At2g41830
At2g45740
At2g46730
At3g01520
At3g01860
At3g04610
At3g06100
At3g06110
At3g08990
At3g09530
At3g11400
At3g11500
At3g11780
At3g13450
At3g15150
At3g17690
At3g19515
At3g22690
At3g24030
At3g27050
At3g27920
At3g42120
At3g44020
At3g44890
At3g45430
At3g46370
At3g46770
At3g46840
At3g48720
At3g48860
At3g50050
At3g55005
At3g59180
At3g61950
At3g63310
At3g63330
At4g00030
At4g00234
At4g00950
At4g01410
At4g02500
At4g02790
At4g02850
At4g02960
At4g04110
At4g05460
At4g11820
At4g12310
At4g14100
At4g19100
At4g19490
At4g19500
At4g19520
At4g19550
At4g21410
At4g22330
At4g24950
At4g29380
At4g31720
At4g32240
At4g33330
At4g34265
At4g38240
At4g38980
At5g01970
At5g02010
At5g02610
At5g03090
At5g03220
At5g05060
At5g08535
At5g08540
At5g14680
At5g16980
At5g25530
At5g27410
At5g33250
At5g35260
At5g35740
At5g36890
At5g37330
At5g42310
At5g43330
At5g44880
At5g44910
At5g45490
At5g45550
At5g45680
At5g46540
At5g49080
At5g50130
At5g51010
At5g51820
At5g52070
At5g52430
At5g58120
At5g60710
16C fatty acid = palmitic
18C fatty acids = oleic, stearic, linoleic, linolenic
20C fatty acids = eicosenoic
22C fatty acids = erucic

TABLE 11
Genes with transcript abundance showing correlation
with ratio of (ratio of 20C + 22C/16C + 18C fatty acids in seed
oil (vernalised plants))/(ratio of 20C + 22C/16C + 18C fatty
acids in seed oil (unvernalised plants)); transcript ID (AGI
code)

11A. Genes showing positive
correlation between transcript
abundance and trait value
At1g01230
At1g02190
At1g02500
At1g02780
At1g02840
At1g03710
At1g06500
At1g06520
At1g06530
At1g10360
At1g11070
At1g12750
At1g13090
At1g13680
At1g14930
At1g15200
At1g17100
At1g19340
At1g22160
At1g22480
At1g22500
At1g23390
At1g26170
At1g27980
At1g28060
At1g29050
At1g29850
At1g30490
At1g30530
At1g31340
At1g31580
At1g32310
At1g32770
At1g37826
At1g52040
At1g52690
At1g52760
At1g53280
At1g53590
At1g54250
At1g55950
At1g56075
At1g59660
At1g59670
At1g59900
At1g60710
At1g62250
At1g62560
At1g63540
At1g64140
At1g64270
At1g64360
At1g64370
At1g64900
At1g66690
At1g67860
At1g68440
At1g69510
At1g69750
At1g70480
At1g72510
At1g73177
At1g73640
At1g74590
At1g74880
At1g76260
At1g76560
At1g76890
At1g77540
At1g77590
At1g77600
At1g78080
At1g78750
At1g78780
At1g79430
At1g80020
At1g80170
At2g01520
At2g01610
At2g06480
At2g14120
At2g14730
At2g15630
At2g18600
At2g19850
At2g19930
At2g20490
At2g21290
At2g21640
At2g21890
At2g22920
At2g25670
At2g25970
At2g27360
At2g28110
At2g28200
At2g28450
At2g29070
At2g29120
At2g32860
At2g33990
At2g36130
At2g36750
At2g36850
At2g37430
At2g37585
At2g38080
At2g38600
At2g39910
At2g40010
At2g44850
At2g45930
At2g47250
At2g47640
At2g48020
At3g01860
At3g02800
At3g03610
At3g04630
At3g06110
At3g06720
At3g06790
At3g07230
At3g07590
At3g08030
At3g09310
At3g09410
At3g09480
At3g10340
At3g11410
At3g12090
At3g13490
At3g13800
At3g14120
At3g15352
At3g15900
At3g16080
At3g16920
At3g17770
At3g18940
At3g20100
At3g20430
At3g21250
At3g22210
At3g22220
At3g22370
At3g22540
At3g22740
At3g25220
At3g25740
At3g26130
At3g28700
At3g29180
At3g29787
At3g31910
At3g44890
At3g45270
At3g46490
At3g46590
At3g47320
At3g47990
At3g48860
At3g49600
At3g50380
At3g51780
At3g52590
At3g53390
At3g53630
At3g53890
At3g54260
At3g54290
At3g55005
At3g55630
At3g56730
At3g56900
At3g57180
At3g57320
At3g59810
At3g60170
At3g60245
At3g60650
At3g61100
At3g61980
At3g62040
At4g00390
At4g02020
At4g02075
At4g03156
At4g04620
At4g04900
At4g05450
At4g09480
At4g10120
At4g12470
At4g13180
At4g13195
At4g14020
At4g14060
At4g14350
At4g14615
At4g15230
At4g15490
At4g15660
At4g17410
At4g18330
At4g18780
At4g19850
At4g21090
At4g21590
At4g22350
At4g22380
At4g22760
At4g24130
At4g25890
At4g27580
At4g29230
At4g29550
At4g30110
At4g30220
At4g30290
At4g31310
At4g31985
At4g32240
At4g32710
At4g35240
At4g35940
At4g36190
At4g37150
At4g37470
At4g37970
At4g39320
At5g01360
At5g02610
At5g03455
At5g03540
At5g03590
At5g04420
At5g04850
At5g05680
At5g06710
At5g07370
At5g07690
At5g08100
At5g08535
At5g08540
At5g08600
At5g09480
At5g10210
At5g10550
At5g11630
At5g13970
At5g16040
At5g17420
At5g17930
At5g18880
At5g20740
At5g24290
At5g25120
At5g28080
At5g28500
At5g28910
At5g29090
At5g39550
At5g40540
At5g40930
At5g42180
At5g42980
At5g43860
At5g45010
At5g45840
At5g47050
At5g47540
At5g48110
At5g48870
At5g49250
At5g49530
At5g50915
At5g50940
At5g50950
At5g51010
At5g51820
At5g52040
At5g53460
At5g54250
At5g55560
At5g57160
At5g58520
At5g58710
At5g59460
At5g59780
At5g60490
At5g61310
At5g61830
At5g62290
At5g63320
At5g63590
At5g64190
At5g65530
At5g66530
11B. Genes showing negative
correlation between transcript
abundance and trait value
At1g02300
At1g02710
At1g03420
At1g05650
At1g08170
At1g08770
At1g11940
At1g13280
At1g13810
At1g15050
At1g20810
At1g20980
At1g21710
At1g22200
At1g27210
At1g33880
At1g44960
At1g51430
At1g51980
At1g55130
At1g57760
At1g57780
At1g59520
At1g59740
At1g60560
At1g62050
At1g62630
At1g66620
At1g69450
At1g70830
At1g71690
At1g77490
At1g79000
At1g79060
At2g02770
At2g07050
At2g07702
At2g15790
At2g15810
At2g19310
At2g23180
At2g23560
At2g28100
At2g28160
At2g32330
At2g33540
At2g34310
At2g35780
At2g35890
At2g38140
At2g39700
At2g41600
At2g42590
At2g43130
At2g43320
At2g44100
At2g45150
At2g45710
At2g46640
At2g47600
At3g02290
At3g05520
At3g05750
At3g06710
At3g10810
At3g11090
At3g12920
At3g14780
At3g16370
At3g18060
At3g18270
At3g22710
At3g22850
At3g22880
At3g22990
At3g27325
At3g28090
At3g29770
At3g43510
At3g43960
At3g46510
At3g46670
At3g48730
At3g61160
At3g61170
At3g62430
At4g00860
At4g01350
At4g02610
At4g04750
At4g10780
At4g11835
At4g11900
At4g12300
At4g12510
At4g17650
At4g18460
At4g18593
At4g18820
At4g20140
At4g23300
At4g25570
At4g28740
At4g31870
At4g32960
At4g35530
At4g39390
At5g03730
At5g05840
At5g05890
At5g07250
At5g08280
At5g14800
At5g17210
At5g17570
At5g18390
At5g20590
At5g22860
At5g26180
At5g28940
At5g35490
At5g38120
At5g38310
At5g40230
At5g43070
At5g45320
At5g46630
At5g47400
At5g49630
At5g51080
At5g51230
At5g51960
At5g53580
At5g57345
At5g59660
At5g62030
At5g64110
orf154
16C fatty acid = palmitic
18C fatty acids = oleic, stearic, linoleic, linolenic
20C fatty acids = eicosenoic
22C fatty acids = erucic

TABLE 12
Genes with transcript abundance correlating with ratio
of polyunsaturated/monounsaturated + saturated 18C fatty acids
in seed oil (vernalised plants)

12A. Genes showing positive correlation
between transcript abundance and
trait value
At1g15490
At1g33560
At1g34220
At1g49030
At1g59620
At1g74650
At1g78210
At2g03680
At2g27090
At2g35736
At2g38010
At3g01720
At3g05210
At3g13840
At3g16520
At3g19930
At3g49360
At3g51580
At3g59660
At4g02450
At4g10150
At4g12020
At4g13050
At4g17390
At4g22840
At4g24940
At5g13890
At5g17060
At5g18400
At5g20180
At5g38980
At5g49540
At5g58910
12B. Genes showing negative correlation
between transcript abundance and
trait value
At1g02050
At1g05550
At1g06580
At1g08560
At1g10980
At1g13250
At1g15280
At1g29180
At1g33055
At1g34030
At1g51950
At1g52800
At1g52810
At1g60190
At1g60390
At1g60800
At1g61810
At1g62500
At1g63780
At1g64105
At1g65560
At1g66180
At1g66900
At1g67590
At1g67830
At1g69690
At1g76320
At2g20360
At2g20585
At2g21860
At2g25900
At2g27970
At2g36490
At2g39450
At2g39870
At2g40570
At2g41370
At2g44860
At3g02500
At3g07200
At3g07270
At3g11420
At3g14150
At3g14240
At3g24660
At3g27420
At3g44010
At3g44600
At3g53110
At3g53230
At3g55610
At3g57860
At3g60520
At4g00600
At4g00930
At4g03050
At4g03070
At4g12600
At4g12880
At4g15780
At4g17560
At4g20070
At4g21650
At4g22160
At4g26170
At4g36380
At4g36740
At5g07000
At5g07030
At5g09630
At5g17100
At5g18070
At5g25180
At5g25590
At5g26230
At5g26270
At5g40150
At5g46160
At5g47760
At5g48760
At5g49190
At5g51660
At5g52230
At5g54190
At5g63860
Polyunsaturated 18C fatty acids = linoleic, linolenic
Monounsaturated 18C fatty acid = oleic
Saturated 18C fatty acid = stearic

TABLE 13
Genes with transcript abundance showing correlation
with ratio of (ratio of polyunsaturated/monounsaturated +
saturated 18C fatty acids in seed oil (vernalised plants))/
(ratio of polyunsaturated/monounsaturated + saturated 18C fatty
acids in seed oil (unvernalised plants)); Transcript ID (AGI
code)

13A. Genes showing positive correlation
between transcript abundance and
trait value
At1g05040
At1g06225
At1g06650
At1g07640
At1g09740
At1g14340
At1g15410
At1g23130
At1g23880
At1g24490
At1g24530
At1g29410
At1g31240
At1g33265
At1g33790
At1g33900
At1g34400
At1g45180
At1g52590
At1g56270
At1g61090
At1g61180
At1g62540
At1g64190
At1g65330
At1g67910
At1g70870
At1g71140
At1g73630
At1g77070
At1g77310
At1g78720
At1g79460
At1g79640
At1g80190
At2g01350
At2g02080
At2g04520
At2g07550
At2g13570
At2g15040
At2g17600
At2g19110
At2g23560
At2g30695
At2g39750
At2g40313
At2g40980
At2g44740
At2g47300
At2g47340
At3g01510
At3g03780
At3g05165
At3g06060
At3g16190
At3g16500
At3g19490
At3g20390
At3g20950
At3g22850
At3g23570
At3g47750
At3g48730
At3g52750
At3g58830
At3g61160
At3g62580
At4g07960
At4g10470
At4g10920
At4g11560
At4g13050
At4g15440
At4g17180
At4g18810
At4g19470
At4g19770
At4g19985
At4g23920
At4g24940
At4g31920
At4g34480
At4g39560
At4g39660
At5g01690
At5g04740
At5g04750
At5g07580
At5g07630
At5g10140
At5g16140
At5g17210
At5g24230
At5g28410
At5g38360
At5g39080
At5g40670
At5g43830
At5g46030
At5g48800
At5g50250
At5g50970
At5g54095
At5g56185
At5g63020
At5g63150
At5g63370
At5g64630
At5g64830
At5g67060
orf107g
13B. Genes showing negative correlation
between transcript abundance and
trait value
At1g02500
At1g03430
At1g18570
At1g23750
At1g28670
At1g30530
At1g32310
At1g52550
At1g59840
At1g59900
At1g66970
At1g68560
At1g78970
At2g04550
At2g21830
At2g22425
At2g2g120
At2g29320
At2g29570
At2g35950
At3g01560
At3g01740
At3g01850
At3g04670
At3g09310
At3g10930
At3g17890
At3g17940
At3g19520
At3g20480
At3g23880
At3g26470
At3g27340
At3g44890
At3g45240
At3g46590
At3g47990
At3g50000
At3g50380
At3g51610
At3g52310
At3g53390
At3g55005
At3g58460
At3g61100
At3g62860
At4g01330
At4g01400
At4g02420
At4g02500
At4g02530
At4g05460
At4g08470
At4g10710
At4g14350
At4g15420
At4g15620
At4g16760
At4g18260
At4g19530
At4g23880
At5g01650
At5g04380
At5g23420
At5g24450
At5g25020
At5g25120
At5g40450
At5g42310
At5g42720
At5g44450
At5g45490
At5g45800
At5g49500
At5g50350
At5g57160
Polyunsaturated 18C fatty acids = linoleic, linolenic
Monounsaturated 18C fatty acid = oleic
Saturated 18C fatty acid = stearic

TABLE 14
Genes with transcript abundance showing correlation
with % 16:0 fatty acid in seed oil (vernalised plants);
Transcript ID (AGI code)
14A. Genes showing positive correlation between
transcript abundance and trait value

| | | | | |
|---|---|---|---|---|
| At1g03300 | At1g74170 | At2g41760 | At3g60350 | At5g10820 |
| At1g03420 | At1g74180 | At2g42750 | At3g60980 | At5g13740 |
| At1g04640 | At1g75490 | At2g43180 | At3g61160 | At5g15680 |
| At1g08170 | At1g78460 | At2g45050 | At3g61200 | At5g17210 |
| At1g13980 | At1g79000 | At2g48100 | At3g61600 | At5g19050 |
| At1g20640 | At1g80600 | At3g01330 | At3g63440 | At5g20150 |
| At1g22200 | At1g80660 | At3g02700 | At4g00500 | At5g22000 |
| At1g24420 | At1g80920 | At3g04350 | At4g00730 | At5g22700 |
| At1g25260 | At2g05540 | At3g04800 | At4g02970 | At5g24410 |
| At1g27210 | At2g05980 | At3g05250 | At4g03970 | At5g25040 |
| At1g28960 | At2g07240 | At3g11210 | At4g04870 | At5g27400 |
| At1g33170 | At2g07675 | At3g11760 | At4g10020 | At5g35330 |
| At1g33880 | At2g07687 | At3g12820 | At4g11530 | At5g38080 |
| At1g34110 | At2g07702 | At3g14750 | At4g12300 | At5g38310 |
| At1g35340 | At2g07741 | At3g15095 | At4g13800 | At5g38895 |
| At1g35420 | At2g11270 | At3g15120 | At4g16960 | At5g38930 |
| At1g36060 | At2g15040 | At3g15290 | At4g18593 | At5g39020 |
| At1g47330 | At2g15230 | At3g15840 | At4g18600 | At5g41850 |
| At1g47750 | At2g15880 | At3g16750 | At4g20360 | At5g41870 |
| At1g48380 | At2g18115 | At3g17280 | At4g26200 | At5g42030 |
| At1g52420 | At2g18190 | At3g18215 | At4g28130 | At5g44240 |
| At1g52920 | At2g19310 | At3g20090 | At4g30993 | At5g47410 |
| At1g52990 | At2g19340 | At3g20930 | At4g32960 | At5g50565 |
| At1g53290 | At2g22170 | At3g21420 | At4g33500 | At5g50600 |
| At1g54710 | At2g23170 | At3g22880 | At4g33570 | At5g51080 |
| At1g56150 | At2g23560 | At3g25900 | At4g35530 | At5g51980 |
| At1g61730 | At2g25850 | At3g26040 | At4g37590 | At5g53430 |
| At1g63690 | At2g27190 | At3g26380 | At4g40050 | At5g54730 |
| At1g64230 | At2g27620 | At3g27990 | At5g01670 | At5g55540 |
| At1g65950 | At2g29860 | At3g29650 | At5g02540 | At5g55870 |
| At1g66570 | At2g35155 | At3g46900 | At5g03730 | At5g65250 |
| At1g66980 | At2g35690 | At3g49210 | At5g05080 | At5g65380 |
| At1g67960 | At2g37120 | At3g53800 | At5g05290 | At5g66040 |
| At1g70300 | At2g38180 | At3g55850 | At5g05690 | ndhG |
| At1g71000 | At2g40070 | At3g57270 | At5g05700 | ndhJ |
| At1g72650 | At2g40970 | At3g57470 | At5g05750 | orf111d |
| At1g73480 | At2g41340 | At3g60040 | At5g05890 | orf262 |
| At1g73680 | At2g41430 | At3g60290 | At5g06130 | petD |

14B. Genes showing negative correlation between
transcript abundance and trait value

| | | | | |
|---|---|---|---|---|
| At1g02500 | At1g66200 | At2g36880 | At3g48130 | At5g20110 |
| At1g04040 | At1g69250 | At2g37020 | At3g48720 | At5g22630 |
| At1g05760 | At1g69700 | At2g37110 | At3g49720 | At5g23540 |
| At1g06410 | At1g72450 | At2g37400 | At3g51780 | At5g23750 |
| At1g08580 | At1g75390 | At2g39560 | At3g52500 | At5g25920 |
| At1g12310 | At1g75590 | At2g40010 | At3g52900 | At5g26330 |
| At1g14780 | At1g75780 | At2g40230 | At3g54430 | At5g27990 |
| At1g17620 | At1g75840 | At2g40660 | At3g54980 | At5g36890 |
| At1g22710 | At1g76260 | At2g41830 | At3g63200 | At5g37330 |
| At1g27000 | At1g76550 | At2g43290 | At4g01100 | At5g40770 |
| At1g27700 | At1g77970 | At2g44745 | At4g05530 | At5g42150 |
| At1g29310 | At1g77990 | At2g46730 | At4g14350 | At5g45550 |
| At1g30510 | At1g78090 | At3g05020 | At4g18570 | At5g45650 |
| At1g30690 | At2g04780 | At3g05230 | At4g20120 | At5g46280 |
| At1g31340 | At2g15860 | At3g05490 | At4g20410 | At5g47210 |
| At1g31660 | At2g16280 | At3g06160 | At4g21090 | At5g47540 |
| At1g32050 | At2g17670 | At3g06510 | At4g28780 | At5g49510 |
| At1g32450 | At2g19540 | At3g06930 | At4g31480 | At5g50740 |
| At1g35670 | At2g20270 | At3g08990 | At4g34870 | At5g54900 |
| At1g44800 | At2g21580 | At3g12370 | At4g35510 | At5g56350 |
| At1g48830 | At2g22470 | At3g15150 | At4g37190 | At5g56950 |
| At1g50010 | At2g22475 | At3g15260 | At4g39280 | At5g58030 |
| At1g50500 | At2g28510 | At3g16340 | At5g02740 | At5g59290 |
| At1g52040 | At2g28760 | At3g16760 | At5g06160 | At5g61660 |
| At1g52910 | At2g29070 | At3g17780 | At5g06190 | At5g62165 |
| At1g54830 | At2g29540 | At3g19590 | At5g11630 | At5g65710 |
| At1g56170 | At2g33430 | At3g21020 | At5g14680 | |
| At1g57620 | At2g33620 | At3g23620 | At5g18280 | |
| At1g63000 | At2g35120 | At3g25220 | At5g18690 | |
| At1g65010 | At2g36620 | At3g27200 | At5g19910 | |

16:0 = palmitic acid

TABLE 15
Genes with transcript abundance correlating with %
18:1 fatty acid in seed oil (vernalised plants); Transcript ID
(AGI code)
15A. Genes showing positive correlation between
transcript abundance and trait value

| | | | | |
|---|---|---|---|---|
| At1g05550 | At1g67830 | At3g14150 | At4g20030 | At5g18070 |
| At1g06580 | At1g69690 | At3g19590 | At4g20070 | At5g19830 |
| At1g08560 | At1g70430 | At3g24450 | At4g21650 | At5g23420 |
| At1g10320 | At1g72260 | At3g24660 | At4g22620 | At5g25180 |
| At1g10980 | At1g74690 | At3g26240 | At4g23870 | At5g25920 |
| At1g13250 | At1g75110 | At3g28345 | At4g28040 | At5g26230 |
| At1g15280 | At2g01090 | At3g44010 | At4g30910 | At5g26270 |
| At1g21080 | At2g17550 | At3g44600 | At4g32130 | At5g40150 |
| At1g23750 | At2g19370 | At3g48130 | At4g35880 | At5g41970 |
| At1g29180 | At2g20360 | At3g53110 | At4g36380 | At5g47550 |
| At1g33055 | At2g20585 | At3g53170 | At4g36740 | At5g47760 |
| At1g34030 | At2g21860 | At3g54680 | At5g06160 | At5g48470 |
| At1g51950 | At2g25900 | At3g57860 | At5g06190 | At5g48760 |
| At1g52800 | At2g32160 | At3g60880 | At5g07000 | At5g49190 |
| At1g52810 | At2g36490 | At3g62860 | At5g07030 | At5g49500 |
| At1g61810 | At2g37050 | At4g00600 | At5g07640 | At5g50950 |
| At1g62500 | At2g39870 | At4g01330 | At5g08540 | At5g51660 |
| At1g63780 | At2g41370 | At4g03050 | At5g10390 | At5g54190 |
| At1g64105 | At2g44230 | At4g03070 | At5g11310 | At5g58300 |
| At1g65560 | At3g02500 | At4g12600 | At5g13970 | At5g63860 |
| At1g66130 | At3g06470 | At4g12880 | At5g14070 | At5g64650 |
| At1g67590 | At3g08680 | At4g15070 | At5g17100 | At5g65010 |

15B. Genes showing negative correlation between
transcript abundance and trait value

| | | | | |
|---|---|---|---|---|
| At1g04985 | At2g27090 | At3g51580 | At5g05750 | At5g39940 |
| At1g15490 | At2g35736 | At3g59660 | At5g08590 | At5g44290 |
| At1g26530 | At2g38010 | At4g02450 | At5g11270 | At5g47580 |
| At1g28030 | At3g01930 | At4g12020 | At5g13890 | At5g49540 |
| At1g33560 | At3g05210 | At4g12300 | At5g16250 | At5g55760 |
| At1g49030 | At3g16520 | At4g13050 | At5g18400 | |
| At1g59620 | At3g17300 | At4g17390 | At5g20180 | |
| At1g76520 | At3g20900 | At4g24940 | At5g23010 | |
| At1g78210 | At3g49360 | At4g32870 | At5g27760 | |

18:1 = oleic acid

TABLE 16
Genes with transcript abundance correlating with %
18:2 fatty acid in seed oil (vernalised plants); Transcript ID
(AGI code)
16A. Genes showing positive correlation between
transcript abundance and trait value

| | | | | |
|---|---|---|---|---|
| At1g02500 | At1g65000 | At2g44850 | At3g54260 | At5g04420 |
| At1g06500 | At1g67860 | At2g46730 | At3g54420 | At5g06730 |
| At1g10460 | At1g72510 | At3g01860 | At3g55005 | At5g07370 |
| At1g11880 | At1g73177 | At3g02800 | At3g55630 | At5g08535 |
| At1g13090 | At1g73940 | At3g03360 | At3g57180 | At5g08540 |
| At1g13750 | At1g74590 | At3g05320 | At3g61980 | At5g08600 |
| At1g14780 | At1g76890 | At3g06110 | At4g00030 | At5g09480 |
| At1g14990 | At1g77590 | At3g07230 | At4g01190 | At5g11600 |
| At1g19340 | At1g77600 | At3g08990 | At4g01410 | At5g16040 |
| At1g21100 | At1g78750 | At3g09410 | At4g02960 | At5g16980 |
| At1g21110 | At1g79890 | At3g09870 | At4g03240 | At5g19560 |
| At1g21190 | At1g79950 | At3g10525 | At4g04620 | At5g27410 |
| At1g22520 | At1g80170 | At3g11400 | At4g09900 | At5g28500 |
| At1g23120 | At1g80700 | At3g15150 | At4g10120 | At5g38530 |
| At1g26170 | At2g01120 | At3g15352 | At4g10955 | At5g38980 |
| At1g30530 | At2g02500 | At3g17690 | At4g11820 | At5g39550 |
| At1g32050 | At2g02960 | At3g19515 | At4g12310 | At5g42310 |
| At1g32450 | At2g05950 | At3g20430 | At4g13180 | At5g43330 |
| At1g33600 | At2g13750 | At3g22690 | At4g14615 | At5g45190 |
| At1g34210 | At2g13770 | At3g22930 | At4g15230 | At5g47050 |
| At1g34740 | At2g15560 | At3g24050 | At4g15260 | At5g47540 |
| At1g35143 | At2g15650 | At3g27610 | At4g18780 | At5g48110 |
| At1g35650 | At2g17265 | At3g27920 | At4g19100 | At5g50940 |
| At1g42705 | At2g21640 | At3g28700 | At4g19850 | At5g51010 |
| At1g47480 | At2g22920 | At3g30720 | At4g21090 | At5g51820 |
| At1g47870 | At2g27360 | At3g30810 | At4g25890 | At5g53360 |
| At1g50630 | At2g28200 | At3g31910 | At4g27580 | At5g55560 |
| At1g52040 | At2g28450 | At3g44890 | At4g29230 | At5g56700 |
| At1g52760 | At2g29070 | At3g46840 | At4g32240 | At5g57160 |
| At1g54250 | At2g30000 | At3g48720 | At4g34120 | At5g57300 |
| At1g55850 | At2g35585 | At3g48860 | At4g37150 | t5g61450 |
| At1g59670 | At2g37585 | At3g48920 | At5g01360 | At5g61830 |
| At1g59900 | At2g37970 | At3g50050 | At5g02010 | At5g64816 |
| At1g60710 | At2g37975 | At3g53630 | At5g02610 | At5g66380 |
| At1g62860 | At2g40010 | At3g53650 | At5g03090 | At5g66530 |
| At1g63540 | At2g41830 | At3g53720 | At5g03540 | |

16B. Genes showing negative correlation between
transcript abundance and trait value

| | | | | |
|---|---|---|---|---|
| At1g01370 | At1g66250 | At2g34560 | At3g56060 | At5g05370 |
| At1g02300 | At1g66520 | At2g39700 | At3g57830 | At5g08280 |
| At1g02710 | At1g68810 | At2g40070 | At3g57880 | At5g17210 |
| At1g03420 | At1g70830 | At2g41600 | At3g60350 | At5g17220 |
| At1g04790 | At1g71690 | At2g43130 | At3g61160 | At5g18390 |
| At1g06730 | At1g79000 | At2g44740 | At3g62430 | At5g22700 |
| At1g11800 | At1g79060 | At2g44760 | At4g00340 | At5g24280 |
| At1g12250 | At1g79460 | At2g45710 | At4g01350 | At5g24760 |
| At1g15050 | At1g80530 | At3g05520 | At4g12300 | At5g26110 |
| At1g20930 | At2g04700 | At3g07200 | At4g12510 | At5g26180 |
| At1g20980 | At2g06255 | At3g11090 | At4g13360 | At5g28940 |
| At1g21690 | At2g07702 | At3g11760 | At4g13980 | At5g35490 |
| At1g21710 | At2g15790 | At3g14240 | At4g17560 | At5g38120 |
| At1g22200 | At2g17450 | At3g18060 | At4g17650 | At5g45320 |
| At1g28440 | At2g18990 | At3g22850 | At4g24390 | At5g51080 |
| At1g47750 | At2g23560 | At3g26070 | At4g26555 | At5g52230 |
| At1g50660 | At2g28100 | At3g26310 | At4g31870 | At5g55810 |
| At1g53460 | At2g29995 | At3g26990 | At4g32960 | At5g59130 |
| At1g55130 | At2g32990 | At3g29770 | At4g35900 | At5g59330 |
| At1g57760 | At2g33540 | At3g48040 | At4g39230 | At5g63180 |
| At1g62050 | At2g34310 | At3g55480 | At5g05230 | At5g64110 |

18:2 = linoleic acid

TABLE 17
Genes with transcript abundance correlating with %
18:3 fatty acid in seed oil (vernalised plants); Transcript ID
(AGI code)
17A. Genes showing positive correlation between
transcript abundance and trait value

| | | | | |
|---|---|---|---|---|
| At1g05060 | At1g64230 | At3g11090 | At4g15960 | At5g28940 |
| At1g08170 | At1g69450 | At3g14780 | At4g18460 | At5g35350 |
| At1g13280 | At1g71800 | At3g17840 | At4g18593 | At5g38310 |
| At1g13580 | At1g74290 | At3g18270 | At4g18820 | At5g38460 |
| At1g13810 | At1g77140 | At3g18650 | At4g23300 | At5g39790 |
| At1g14660 | At1g77490 | At3g20230 | At4g25570 | At5g40230 |
| At1g15330 | At1g79000 | At3g22710 | At4g26870 | At5g44240 |
| At1g20370 | At2g02360 | At3g22850 | At4g27900 | At5g44290 |
| At1g20810 | At2g02770 | At3g22880 | At4g31150 | At5g44520 |
| At1g20980 | At2g07050 | At3g26430 | At4g31870 | At5g46270 |
| At1g21710 | At2g16090 | At3g30140 | At4g39390 | At5g46630 |
| At1g22200 | At2g18115 | At3g43790 | At4g39920 | At5g47400 |
| At1g23890 | At2g32330 | At3g48730 | At4g39930 | At5g47410 |
| At1g33265 | At2g35890 | At3g53680 | At5g03290 | At5g49630 |
| At1g33880 | At2g41600 | At3g53900 | At5g05840 | At5g51960 |
| At1g51430 | At2g43180 | At3g56590 | At5g05890 | At5g55760 |
| At1g51980 | At2g43320 | At3g61480 | At5g07250 | At5g59660 |
| At1g57780 | At2g44690 | At4g01690 | At5g08280 | At5g63370 |
| At1g59780 | At2g45150 | At4g01970 | At5g17210 | At5g63740 |
| At1g61830 | At2g45560 | At4g11835 | At5g17520 | At5g64110 |
| At1g63200 | At2g46640 | At4g11900 | At5g18400 | orf114 |
| At1g64190 | At3g05520 | At4g12300 | At5g22860 | ycf4 |

17B. Genes showing negative correlation between
transcript abundance and trait value

| | | | | |
|---|---|---|---|---|
| At1g02500 | At1g76560 | At3g09310 | At4g02290 | At5g07640 |
| At1g05550 | At1g76720 | At3g10340 | At4g03156 | At5g08540 |
| At1g06500 | At1g77600 | At3g11410 | At4g04620 | At5g09760 |
| At1g06520 | At1g78080 | At3g12110 | At4g05450 | At5g13970 |
| At1g06530 | At1g78780 | At3g12520 | At4g09760 | At5g16040 |
| At1g07470 | At1g78970 | At3g13490 | At4g10120 | At5g16470 |
| At1g09660 | At1g79430 | At3g14150 | At4g10320 | At5g18790 |
| At1g10980 | At2g01520 | At3g15900 | At4g12490 | At5g19830 |
| At1g13090 | At2g15620 | At3g16080 | At4g13195 | At5g24740 |
| At1g13680 | At2g18100 | At3g20100 | At4g14010 | At5g25120 |
| At1g14930 | At2g18650 | At3g21250 | At4g14020 | At5g25180 |
| At1g15200 | At2g19740 | At3g22210 | At4g14320 | At5g27720 |
| At1g18810 | At2g20450 | At3g22230 | At4g14350 | At5g35240 |
| At1g18880 | At2g20490 | At3g23325 | At4g14615 | At5g40250 |
| At1g21080 | At2g20515 | At3g25220 | At4g16830 | At5g42720 |
| At1g23950 | At2g20820 | At3g25740 | At4g17410 | At5g45010 |
| At1g24070 | At2g21290 | At3g26240 | At4g18750 | At5g45840 |
| At1g26170 | At2g21640 | At3g29180 | At4g21590 | At5g47540 |
| At1g28060 | At2g21890 | At3g46490 | At4g22380 | At5g47550 |
| At1g29180 | At2g23090 | At3g47370 | At4g23870 | At5g47760 |
| At1g29850 | At2g25670 | At3g47990 | At4g25890 | At5g48580 |
| At1g30530 | At2g25970 | At3g48130 | At4g26230 | At5g49190 |
| At1g33055 | At2g26460 | At3g49600 | At4g26790 | At5g49500 |
| At1g50140 | At2g27360 | At3g50380 | At4g29230 | At5g49970 |
| At1g52040 | At2g28450 | At3g51780 | At4g29550 | At5g50915 |
| At1g52690 | At2g29070 | At3g52590 | At4g30220 | At5g50950 |
| At1g53030 | At2g29120 | At3g53260 | At4g30290 | At5g51390 |
| At1g54250 | At2g36170 | At3g53390 | At4g31985 | At5g51660 |
| At1g59840 | At2g36570 | At3g53500 | At4g35240 | At5g52040 |
| At1g59900 | At2g41560 | At3g53630 | At4g35880 | At5g53460 |
| At1g61570 | At2g41790 | At3g53890 | At4g35940 | At5g57160 |
| At1g61810 | At2g47250 | At3g54290 | At4g36190 | At5g58520 |
| At1g63020 | At2g47790 | At3g55005 | At4g36380 | At5g59460 |
| At1g63540 | At3g03610 | At3g56900 | At4g37250 | At5g61830 |
| At1g64900 | At3g04670 | At3g58840 | At4g39200 | At5g64190 |
| At1g66080 | At3g05530 | At3g59540 | At5g01890 | At5g64650 |
| At1g66920 | At3g06110 | At3g62080 | At5g03455 | At5g65050 |
| At1g72260 | At3g06130 | At3g62860 | At5g04420 | At5g65530 |
| At1g74250 | At3g06310 | At4g02075 | At5g04850 | At5g65890 |
| At1g74270 | At3g06790 | At4g02210 | At5g05680 | |
| At1g74880 | At3g08030 | At4g02230 | At5g06710 | |

18:3 = linolenic acid

TABLE 18
Prediction of complex traits using models based on
accession transcriptome data

| | No. genes | Accession: Ga-0 | | Accession: Sorbo | | |
|---|---|---|---|---|---|---|
| Trait | in model | Measured | Predicted | Measured | Predicted | Ranking |

Flowering time

| | | | | | | |
|---|---|---|---|---|---|---|
| Leaf number - | 311 | 12.00 | 11.53 | 9.00 | 10.36 | correct |
| vernalised | | | | | | |
| Leaf number - | 339 | 16.10 | 18.87 | 24.20 | 20.33 | correct |
| unvernalised | | | | | | |
| Leaf number - | 485 | 0.75 | 0.71 | 0.37 | 0.61 | correct |
| vern/unvern ratio | | | | | | |

Seed oil content

| | | | | | | |
|---|---|---|---|---|---|---|
| Oil content % - | 390 | 42.18 | 40.71 | 38.65 | 39.55 | correct |
| vernalised | | | | | | |

Seed fatty acid ratios

| | | | | | | |
|---|---|---|---|---|---|---|
| Chain length ratio - | 228 | 0.21 | 0.21 | 0.14 | 0.18 | correct |
| vernalised | | | | | | |
| Chain length ratio - | 438 | 1.37 | 1.35 | 1.58 | 1.47 | correct |
| vern/unvern | | | | | | |
| Desaturation ratio - | 118 | 3.69 | 3.88 | 4.25 | 4.28 | correct |
| vernalised | | | | | | |
| Desaturation ratio - | 188 | 1.08 | 1.08 | 0.92 | 1.07 | correct |
| vern/unvern | | | | | | |
| 18:3/18:1 ratio - | 151 | 1.98 | 2.15 | 1.91 | 2.07 | correct |
| vernalised | | | | | | |
| 18:3/18:2 ratio - | 311 | 0.73 | 0.76 | 0.64 | 0.70 | correct |
| vernalised | | | | | | |
| 18:2/18:1 ratio - | 197 | 2.72 | 2.86 | 3.01 | 3.37 | correct |
| vernalised | | | | | | |

Seed fatty acid absolute content

| | | | | | | |
|---|---|---|---|---|---|---|
| %16:0 - vernalised | 337 | 9.29 | 10.34 | 8.37 | 9.90 | correct |
| %18:1 - vernalised | 151 | 11.97 | 11.83 | 13.14 | 11.18 | not |
| | | | | | | correct |
| %18:2 - vernalised | 288 | 32.40 | 32.31 | 38.38 | 34.85 | correct |
| %18:3 - vernalised | 313 | 23.81 | 24.36 | 24.10 | 24.06 | not |
| | | | | | | correct |

TABLE 19
Maize genes with transcript abundance in hybrids
correlating with heterosis

| | Probe Set ID | Representative Public ID |
|---|---|---|
| 19A. Positive | Zm.18469.1.S1_at | BM378527 |
| Correlation | ZmAffx.448.1.S1_at | AI677105 |
| | Zm.5324.1.A1_at | AI619250 |
| | Zm.886.5.S1_a_at | BU499802 |
| | Zm.5494.1.A1_at | AI622241 |
| | Zm.17363.1.S1_at | CK370960 |
| | Zm.1234.1.A1_at | BM073436 |
| | Zm.11688.1.A1_at | CK347476 |
| | Zm.695.1.A1_at | U37285.1 |
| | Zm.12561.1.A1_at | AI834417 |
| | Zm.17443.1.A1_at | CK347379 |
| | Zm.11579.2.S1_a_at | CF629377 |
| | Zm.342.2.A1_at | U65948.1 |
| | Zm.8950.1.A1_at | AY109015.1 |
| | Zm.18417.1.A1_at | CO528437 |
| | Zm.2553.1.A1_a_at | BQ619023 |
| | Zm.13487.1.A1_at | AY108830.1 |
| | Zm.13746.1.S1_at | CD998898 |
| | Zm.8742.1.A1_at | BM075443 |
| | Zm.17701.1.S1_at | CK370965 |
| | Zm.2147.1.A1_a_at | BM380613 |
| | Zm.10826.1.S1_at | BQ619411 |
| | ZmAffx.501.1.S1_at | AI691747 |
| | Zm.17970.1.A1_at | CK827393 |
| | Zm.12592.1.S1_at | CA830809 |
| | Zm.13810.1.S1_at | AB042267.1 |
| | Zm.4669.1.S1_at | AI737897 |
| | ZmAffx.351.1.S1_at | AI670538 |
| | Zm.5233.1.A1_at | CF626276 |
| | Zm.9738.1.S1_at | BM337426 |
| | Zm.8102.1.A1_at | CF005906 |
| | Zm.6393.4.A1_at | BQ048072 |
| | Zm.15120.1.A1_at | BM078520 |
| | Zm.17342.1.S1_at | CK370507 |
| | Zm.2674.1.A1_at | CF045775 |
| | Zm.4191.2.S1_a_at | BQ547780 |
| | Zm.14504.1.A1_at | AY107583.1 |
| | Zm.6049.3.A1_a_at | AI734480 |
| | Zm.2100.1.A1_at | CD001187 |
| | Zm.13795.2.S1_a_at | CF042915 |
| | Zm.5351.1.S1_at | AI619365 |
| | Zm.5939.1.A1_s_at | AI738346 |
| | Zm.2626.1.S1_at | AY112337.1 |
| | Zm.15454.1.A1_at | CD448347 |
| | Zm.4692.1.A1_at | AI738236 |
| | Zm.5502.1.A1_at | BM378399 |
| | Zm.2758.1.A1_at | AW067110 |
| | ZmAffx.752.1.S1_at | AI712129 |
| | Zm.14994.1.A1_at | BQ538997 |
| | Zm.12748.1.S1_at | AW066809 |
| | Zm.18006.1.A1_at | AW400144 |
| | ZmAffx.601.1.A1_at | AI715029 |
| | Zm.6045.7.A1_at | CK347781 |
| | Zm.81.1.S1_at | AY106090.1 |
| | ZmAffx.292.1.S1_at | AI670425 |
| | Zm.17917.1.A1_at | CF629332 |
| | ZmAffx.424.1.S1_at | AI676856 |
| | Zm.6371.1.A1_at | AY122273.1 |
| | Zm.1125.1.A1_at | BI993208 |
| | Zm.4758.1.S1_at | AY111436.1 |
| | Zm.17779.1.S1_at | CK370643 |
| | Zm.2964.1.S1_s_at | AY106674.1 |
| | Zm.17937.1.A1_at | CO529646 |
| | Zm.7162.1.A1_at | BM074641 |
| | Zm.13402.1.S1_at | AF457950.1 |
| | Zm.18189.1.S1_at | CN844773 |
| | Zm.4312.1.A1_at | BM266520 |
| | Zm.2141.1.A1_at | BM347927 |
| | Zm.19317.1.S1_at | CO521190 |
| | Zm.4164.2.A1_at | CF627018 |
| | Zm.8307.2.A1_a_at | CF635305 |
| | Zm.16805.2.A1_at | CF635679 |
| | Zm.19080.1.A1_at | CO522397 |
| | Zm.1489.1.A1_at | CO519391 |
| | Zm.13462.1.A1_at | CO522224 |
| | ZmAffx.191.1.S1_at | AI668423 |
| | Zm.19037.1.S1_at | CA404446 |
| | Zm.4109.1.A1_at | CD441071 |
| | Zm.2588.1.S1_at | AI714899 |
| | Zm.10920.1.A1_at | CA399553 |
| | Zm.1710.1.S1_at | AY106827.1 |
| | Zm.16301.1.S1_at | CK787019 |
| | Zm.4665.1.A1_at | CK370646 |
| | Zm.7336.1.A1_at | AF371263.1 |
| | Zm.16501.1.S1_at | AY108566.1 |
| | Zm.10223.1.S1_at | BM078528 |
| | Zm.3030.1.A1_at | CA402193 |
| | Zm.14027.1.A1_at | AW499409 |
| | Zm.8796.1.A1_at | BG841012 |
| | Zm.13732.1.S1_at | AY106236.1 |
| | Zm.4870.1.A1_a_at | CK985786 |
| | ZmAffx.555.1.A1_x_at | AI714437 |
| | Zm.7327.1.A1_at | AF289256.1 |
| | Zm.2933.1.A1_at | AW091233 |
| | Zm.949.1.A1_s_at | CF624182 |
| | Zm.15510.1.A1_at | CD441066 |
| | Zm.8375.1.A1_at | BM080176 |
| | Zm.4824.6.S1_a_at | AI665566 |
| | Zm.612.1.A1_at | AF326500.1 |
| | Zm.12881.1.A1_at | CA401025 |
| | Zm.7687.1.A1_at | BM072867 |
| | Zm.10587.1.A1_at | AY107155.1 |
| | Zm.17807.1.S1_at | CK371584 |
| | Zm.3947.1.S1_at | BE510702 |
| | Zm.6626.1.A1_at | AI491257 |
| | Zm.1527.2.A1_a_at | BM078218 |
| | Zm.6856.1.A1_at | AI065480 |
| | ZmAffx.1477.1.S1_at | 40794996-104 |
| | Zm.12588.1.S1_at | CO530559 |
| | Zm.15817.1.A1_at | D87044.1 |
| | Zm.16278.1 A1_at | CO532740 |
| | Zm.18877.1.A1_at | CO529651 |
| | Zm.2090.1.A1_at | AI691653 |
| | Zm.5160.1.A1_at | CD995815 |
| | Zm.17651.1.A1_at | CF043781 |
| | Zm.15722.2.A1_at | CA404232 |
| | Zm.5456.1.A1_at | AI622004 |
| | Zm.13992.1.A1_at | CK827024 |
| | Zm.3105.1.S1_at | AY108981.1 |
| | ZmAffx.941.1.S1_at | AI820356 |
| | Zm.3913.1.A1_at | CF000034 |
| | Zm.1657.1.A1_at | BG842419 |
| | Zm.13200.1.A1_at | CF635119 |
| | Zm.18789.1.S1_at | CO525842 |
| | Zm.10090.1.A1_at | BM382713 |
| | Zm.312.1.A1_at | S72425.1 |
| | Zm.9118.1.A1_at | BM336433 |
| | Zm.9117.1.A1_at | CF636944 |
| | Zm.610.1.A1_at | AF326498.1 |
| | Zm.5725.1.A1_at | CK986059 |
| | Zm.6805.1.S1_a_at | BG266504 |
| | Zm.1621.1.S1_at | AY107628.1 |
| | Zm.1997.1.A1_at | BM075855 |
| | ZmAffx.1086.1.S1_at | AW018229 |
| | Zm.17377.1.A1_at | CK144565 |
| | Zm.15822.1.S1_at | AY313901.1 |
| | Zm.5486.1.A1_at | AI629867 |
| | Zm.4469.1.S1_at | AI734281 |
| | Zm.8620.1.S1_at | BM073355 |
| | Zm.18031.1.A1_at | CK985574 |
| | Zm.13597.1.A1_at | CF630886 |
| | Zm.75.2.S1_at | CK371662 |
| | Zm.4327.1.S1_at | BI993026 |
| | Zm.17157.1.A1_at | BM074525 |
| | Zm.7342.1.A1_at | AF371279.1 |
| | Zm.2781.1.S1_at | CF007960 |
| | Zm.3944.1.S1_at | M29411.1 |
| | Zm.98.1.S1_at | AY106729.1 |
| | Zm.3892.6.A1_x_at | CD441708 |
| | Zm.12051.1.A1_at | AI947869 |
| | Zm.4193.1.A1_at | AY106195.1 |
| | Zm.2197.1.S1_a_at | AF007785.1 |
| | Zm.12164.1.A1_at | CO521714 |
| | Zm.15998.1.A1_at | CA403811 |
| | ZmAffx.1186.1.A1_at | AY110093.1 |
| | Zm.19149.1.S1_at | CO526376 |
| | Zm.14820.1.S1_at | AY106101.1 |
| | Zm.15789.1.A1_a_at | CD440056 |
| | ZmAffx.655.1.A1_at | AI715083 |
| | Zm.19077.1.A1_at | CO526103 |
| | Zm.698.1.A1_at | AY112103.1 |
| | Zm.10332.1.A1_at | BQ048110 |
| | Zm.10642.1.A1_at | BQ539388 |
| | Zm.11901.1.A1_at | BM381636 |
| | ZmAffx.1494.1.S1_s_at | 40794996-111 |
| | ZmAffx.871.1.A1_at | AI770769 |
| | Zm.13463.1.S1_at | AY109103.1 |
| | Zm.18502.1.A1_at | CF623953 |
| | Zm.2171.1.A1_at | BG841205 |
| | Zm.14069.2.A1_at | AY110342.1 |
| | Zm.6036.1.S1_at | AY110222.1 |
| | Zm.17638.1.S1_at | CK368502 |
| | Zm.813.1.S1_at | AF244683.1 |
| | Zm.8376.1.S1_at | BM073880 |
| | Zm.16922.1.A1_a_at | CD998944 |
| | Zm.16913.1.S1_at | BQ619268 |
| | Zm.12851.1.A1_at | CA400703 |
| | Zm.3225.1.S1_at | BE512131 |
| | Zm.13628.1.S1_at | CD437947 |
| | Zm.9998.1.A1_at | BM335619 |
| | Zm.15967.1.S1_at | CA404149 |
| | Zm.6366.2.A1_at | CA398774 |
| | Zm.1784.1.S1_at | BF728627 |
| | Zm.19031.1.A1_at | BU051425 |
| | Zm.6170.1.A1_a_at | AY107283.1 |
| | Zm.3789.1.S1_at | AW438148 |
| | Zm.4310.1.A1_at | BM078907 |
| | Zm.3892.10.A1_at | AI691846 |
| | RPTR-Zm-U47295-1_at | RPTR-Zm-U47295-1 |
| | Zm.15469.1.S1_at | CD438450 |
| | Zm.7515.1.A1_at | BM078765 |
| | Zm.6728.1.A1_at | CN844413 |
| | Zm.16798.2.A1_a_at | CF633780 |
| | Zm.455.1.S1_a_at | AF135014.1 |
| | Zm.10134.1.A1_at | BQ619055 |
| 19B. Negative | Zm.10492.1.S1_at | CA826941 |
| Correlation | Zm.5113.2.A1_a_at | CF633388 |
| | Zm.3533.1.A1_at | AY110439.1 |
| | ZmAffx.674.1.S1_at | AI734487 |
| | ZmAffx.1060.1.S1_at | AI881420 |
| | ZmAffx.361.1.A1_at | AI670571 |
| | Zm.10190.1.S1_at | CF041516 |
| | Zm.12256.1.S1_at | BU049042 |
| | ZmAffx.1529.1.S1_at | 40794996-124 |
| | Zm.19120.1.A1_at | CO523709 |
| | Zm.2614.2.A1_at | CD436098 |
| | Zm.10429.1.S1_at | BQ528642 |
| | Zm.13457.1.S1_at | AY109190.1 |
| | Zm.4040.1.A1_at | AI834032 |
| | Zm.5083.2.S1_at | AY109962.1 |
| | Zm.5704.1.A1_at | AI637031 |
| | Zm.3934.1.S1_at | AI947382 |
| | Zm.6478.1.S1_at | AI692059 |
| | Zm.1161.1.S1_at | BE511616 |
| | Zm.12135.1.A1_at | BM334402 |
| | Zm.4878.1.A1_at | AW288995 |
| | Zm.18825.1.A1_at | CO527281 |
| | Zm.4087.1.A1_at | AI834529 |
| | Zm.9321.1.A1_at | AY108492.1 |
| | Zm.9121.1.A1_at | CF631233 |
| | Zm.7797.1.A1_at | BM079946 |
| | Zm.1228.1.S1_at | CF006184 |
| | Zm.1118.1.S1_at | CF631214 |
| | Zm.3612.1.A1_at | AY103746.1 |
| | Zm.17612.1.S1_at | CK368134 |
| | Zm.7082.1.S1_at | CF637101 |
| | Zm.6188.2.A1_at | AY108898.1 |
| | Zm.6798.1.A1_at | CA400889 |
| | Zm.6205.1.A1_at | CK985870 |
| | Zm.582.1.S1_at | AF186234.2 |
| | Zm.5798.1.A1_at | BM072971 |
| | Zm.8598.1.A1_at | BM075029 |
| | Zm.15207.1.A1_at | BM268677 |
| | Zm.4164.3.A1_s_at | CF636517 |
| | Zm.1802.1.A1_at | BM078736 |
| | Zm.13583.1.S1_at | AY108161.1 |
| | ZmAffx.513.1.A1_at | AI692067 |
| | ZmAffx.853.1.A1_at | AI770653 |
| | Zm.2128.1.S1_at | AY105930.1 |
| | Zm.18488.1.A1_at | BM269253 |
| | Zm.10471.1.A1_at | CA399504 |
| | ZmAffx.716.1.S1_at | AI739804 |
| | Zm.10756.1.S1_at | CD975109 |
| | Zm.1482.5.S1_at | AI714961 |
| | ZmAffx.494.1.S1_at | AI770346 |
| | Zm.5688.1.A1_at | AY105372.1 |
| | Zm.4673.2.A1_a_at | CA400524 |
| | Zm.9542.1.A1_at | CF624708 |
| | Zm.10557.2.A1_at | BQ538273 |
| | ZmAffx.1051.1.A1_at | AI881809 |
| | Zm.3724.1.A1_x_at | CF627032 |
| | Zm.6575.1.A1_at | AI737943 |
| | Zm.18046.1.A1_at | BI993031 |
| | Zm.4990.1.A1_at | AI586885 |
| | ZmAffx.891.1.A1_at | AI770848 |
| | Zm.10750.1.A1_at | AY104853.1 |
| | Zm.6358.1.S1_at | CA402045 |
| | Zm.2150.1.A1_a_at | CD977294 |
| | Zm.4068.2.A1_at | BQ619512 |
| | Zm.1327.1.A1_at | BE643637 |
| | Zm.3699.1.S1_at | U92045.1 |
| | ZmAffx.175.1.S1_at | AI668276 |
| | Zm.311.1.A1_at | BM268583 |
| | Zm.19326.1.A1_at | CO530193 |
| | Zm.728.1.A1_at | BM338202 |
| | ZmAffx.963.1.A1_at | AI833792 |
| | Zm.5155.1.S1_at | CD433333 |
| | Zm.3186.1.S1_a_at | CK827152 |
| | ZmAffx.1164.1.A1_at | AW455679 |
| | Zm.10069.1.A1_at | AY108373.1 |
| | Zm.17869.1.S1_at | CK701080 |
| | Zm.1670.1.A1_at | AY109012.1 |
| | Zm.737.1.A1_at | D45403.1 |
| | Zm.9947.1.A1_at | BM349454 |
| | Zm.3553.1.S1_at | AY112170.1 |
| | Zm.11794.1.A1_at | BM380817 |
| | ZmAffx.139.1.S1_at | AI667769 |
| | Zm.5328.2.A1_at | AW258090 |
| | Zm.534.1.A1_x_at | AF276086.1 |
| | Zm.17724.3.S1_x_at | CK370253 |
| | Zm.13806.1.S1_at | AY104790.1 |
| | Zm.8710.1.A1_at | BM333560 |
| | Zm.14397.1.A1_at | BM351246 |
| | Zm.5495.1.S1_at | AY103870.1 |
| | Zm.4338.3.S1_at | AW000126 |
| | Zm.9199.1.A1_at | CO522770 |
| | Zm.15839.1.A1_at | AY109200.1 |
| | Zm.12386.1.A1_at | CF630849 |
| | Zm.7495.1.A1_at | CF636496 |
| | Zm.2181.1.S1_at | BF727788 |
| | ZmAffx.144.1.S1_at | AI667795 |
| | Zm.4449.1.A1_at | BM074466 |
| | Zm.8111.1.S1_at | CD972041 |
| | Zm.17784.1.S1_at | CK370703 |
| | Zm.16247.1.S1_at | AY181209.1 |
| | Zm.3699.5.S1_a_at | AY107222.1 |
| | Zm.7823.1.S1_at | BM078187 |
| | Zm.5866.1.S1_at | CF044154 |
| | Zm.6469.1.S1_at | BE345306 |
| | Zm.10434.1.S1_at | BQ577392 |
| | Zm.16929.1.S1_at | AW055615 |
| | Zm.7572.1.S1_at | CO521006 |
| | Zm.6726.1.S1_x_at | AI395973 |
| | ZmAffx.387.1.S1_at | AI673971 |
| | Zm.9543.1.A1_at | CK370330 |
| | Zm.1632.1.S1_at | AY104990.1 |
| | Zm.8897.1.S1_at | BM079371 |
| | Zm.14869.1.A1_at | AI586666 |
| | Zm.1059.2.A1_a_at | CO518029 |
| | Zm.4611.1.A1_s_at | BG842817 |
| | ZmAffx.1172.1.S1_at | AW787638 |
| | Zm.8751.1.A1_at | BM348137 |
| | Zm.1066.1.S1_a_at | AY104986.1 |
| | Zm.13931.1.S1_x_at | Z35302.1 |
| | Zm.9916.1.A1_at | BM348997 |
| | ZmAffx.1203.1.A1_at | BE128869 |
| | Zm.9468.1.S1_at | AY108678.1 |
| | Zm.4049.1.A1_at | AI834098 |
| | Zm.14325.1.S1_at | AY104177.1 |
| | Zm.9281.1.A1_at | BM267756 |
| | Zm.229.1.S1_at | L33912.1 |
| | Zm.2244.1.S1_a_at | CF348841 |
| | Zm.4587.1.A1_at | CO528135 |
| | Zm.9604.1.A1_at | BM333654 |
| | Zm.7831.1.A1_at | BM080062 |
| | Zm.648.1.S1_at | AF144079.1 |
| | Zm.5018.3.A1_at | AI668145 |
| | ZmAffx.962.1.A1_at | AI833777 |
| | Zm.11663.1.A1_at | CO531620 |
| | Zm.19167.2.A1_x_at | CF636656 |
| | ZmAffx.776.1.A1_at | AI746212 |
| | Zm.4736.1.A1_at | AY108189.1 |
| | ZmAffx.1053.1.A1_at | AI881846 |
| | Zm.4248.1.A1_at | AY110118.1 |
| | ZmAffx.1523.1.S1_at | 40794996-120 |
| | Zm.4922.1.A1_at | AI586404 |
| | Zm.6601.2.A1_a_at | BM078978 |
| | Zm.18355.1.A1_at | CO532040 |
| | Zm.16351.1.A1_at | CF623648 |
| | Zm.12150.1.S1_at | AY106576.1 |
| | ZmAffx.1428.1.S1_at | 11990232-13 |
| | Zm.11468.1.A1_at | BM382262 |
| | Zm.11550.1.A1_at | BG320003 |
| | Zm.12235.1.A1_at | CF972364 |
| | Zm.10911.1.A1_x_at | BM340657 |
| | Zm.1497.1.S1_at | AF050631.1 |
| | Zm.2440.1.A1_a_at | BM347886 |
| | Zm.6638.1.A1_at | AI619165 |
| | ZmAffx.840.1.S1_at | AI770592 |
| | Zm.15800.2.A1_at | CD998623 |
| | Zm.2220.4.S1_at | AY110053.1 |
| | Zm.5791.1.A1_at | AY103953.1 |
| | Zm.9435.1.A1_at | BM268868 |
| | Zm.2565.1.S1_at | AY112147.1 |
| | ZmAffx.964.1.A1_at | AI833796 |
| | Zm.3134.1.A1_at | AY112040.1 |
| | Zm.8549.1.A1_at | BM339103 |
| | Zm.10807.2.A1_at | CD970321 |
| | Zm.3286.1.A1_at | BG265986 |
| | Zm.11983.1.A1_at | BM382368 |
| | ZmAffx.841.1.A1_at | AI770596 |
| | Zm.2950.1.A1_at | AI649878 |
| | Zm.900.1.S1_at | BF728342 |
| | Zm.8147.1.A1_at | BM073080 |
| | Zm.18430.1.S1_at | CO524429 |
| | Zm.15859.1.A1_at | D14578.1 |
| | Zm.17164.1.S1_at | AY188756.1 |
| | Zm.1204.1.S1_at | BE519063 |
| | Zm.17968.1.A1_at | CK827143 |

TABLE 20
Maize genes with transcript abundance in hybrids used
for prediction of average yield in hybrids

| | Probe Set ID | Representative Public ID |
|---|---|---|
| 20A. Positive | Zm.4900.2.A1_at | AY105715.1 |
| Correlation | Zm.6390.1.S1_at | BU098381 |
| | Zm.17314.1.S1_at | CK369303 |
| | Zm.8720.1.S1_at | AY303682.1 |
| | ZmAffx.435.1.A1_at | AI676952 |
| | Zm.4807.1.A1_at | CO518291 |
| | Zm.16794.1.A1_at | AF330034.1 |
| | Zm.19357.1.A1_at | CO533449 |
| | Zm.13190.1.A1_at | CD433968 |
| | Zm.16025.1.A1_at | BM340438 |
| | AFFX-r2-TagC_at | AFFX-r2-TagC |
| | ZmAffx.844.1.S1_at | AI770609 |
| | Zm.6342.1.S1_at | AW052791 |
| | Zm.9453.1.A1_at | CO521132 |
| | Zm.13708.1.A1_at | AY106587.1 |
| | Zm.10609.1.A1_at | BQ538614 |
| | Zm.6589.1.A1_at | AI622544 |
| | ZmAffx.1308.1.S1_s_at | 11990232-76 |
| | Zm.4024.1.S1_at | AY105692.1 |
| | Zm.16805.4.A1_at | AI795617 |
| | Zm.10032.1.S1_at | CN844905 |
| | Zm.4943.1.A1_at | BG320867 |
| | Zm.6970.1.A1_a_at | AY111674.1 |
| | Zm.8150.1.A1_at | BM073089 |
| | Zm.4696.1.S1_at | BG266403 |
| | ZmAffx.994.1.A1_at | AI855283 |
| | Zm.11585.1.A1_at | BM379130 |
| | ZmAffx.45.1.S1_at | AI664925 |
| | Zm.6214.1.A1_a_at | BQ538548 |
| | Zm.9102.1.A1_at | BM333481 |
| | Zm.4909.1.A1_at | AY111633.1 |
| | Zm.13916.1.S1_at | AF037027.1 |
| | Zm.17317.1.S1_at | CK370700 |
| | Zm.5684.1.A1_at | BM334571 |
| | AFFX-r2-TagJ-3_at | AFFX-r2-TagJ-3 |
| | Zm.2232.1.S1_at | BM380334 |
| | Zm.15667.1.S1_at | CD437700 |
| | Zm.1996.1.S1_at | CK347826 |
| | Zm.9642.1.A1_at | BM338826 |
| | Zm.12716.1.S1_at | AY112283.1 |
| | Zm.6556.1.A1_at | AY109683.1 |
| | ZmAffx.54.1.S1_at | AI665038 |
| | Zm.5099.1.S1_at | AI600819 |
| | Zm.5550.1.S1_at | AI622648 |
| | Zm.1352.1.A1_at | AY106566.1 |
| | Zm.4312.3.S1_at | CF075294 |
| | Zm.2202.1.A1_at | AY105037.1 |
| | Zm.14089.1.S1_at | AW324724 |
| | Zm.13601.1.S1_at | AY107674.1 |
| | Zm.4.1.S1_a_at | CD434423 |
| | ZmAffx.219.1.S1_at | AI670227 |
| | ZmAffx.122.1.S1_at | AI665696 |
| | ZmAffx.109.1.S1_at | AI665560 |
| | ZmAffx.331.1.A1_at | AI670513 |
| | Zm.4118.1.A1_at | AY105314.1 |
| | Zm.6369.3.A1_at | AI881634 |
| | Zm.15323.1.A1_at | BM349667 |
| | Zm.3050.3.A1_at | CF630494 |
| | Zm.2957.1.A1_at | CK371564 |
| | ZmAffx.439.1.A1_at | AI676966 |
| | Zm.4860.2.A1_at | AI770577 |
| | Zm.19141.1.A1_at | CF625022 |
| | Zm.5268.1.S1_at | CF626642 |
| | Zm.5791.2.A1_a_at | AW438331 |
| | Zm.4616.1.A1_x_at | BQ538201 |
| | Zm.12940.1.S1_at | AY104675.1 |
| | Zm.4265.1.A1_at | CA402796 |
| | Zm.8412.1.A1_at | AY108596.1 |
| | Zm.18041.1.A1_at | BQ620926 |
| | Zm.13365.1.A1_at | CK827054 |
| | Zm.2734.2.S1_at | BF727671 |
| | Zm.16299.2.A1_a_at | BM336250 |
| | Zm.13007.1.S1_at | CO532826 |
| | Zm.12716.1.A1_at | AY112283.1 |
| | Zm.11827.1.A1_at | BM381077 |
| | Zm.14824.1.S1_at | AJ430693.1 |
| | Zm.15083.2.A1_at | AY107613.1 |
| | Zm.445.2.A1_at | AF457968.1 |
| | Zm.5834.1.A1_a_at | BM335098 |
| | ZmAffx.823.1.S1_at | AI770503 |
| | Zm.8924.1.A1_at | BM381215 |
| | Zm.722.1.A1_at | AW288498 |
| | Zm.13341.1.S1_at | CF044863 |
| | Zm.12037.1.S1_at | BI894209 |
| | Zm.2557.1.S1_at | CF649649 |
| | ZmAffx.1152.1.A1_at | AW424633 |
| | Zm.5423.1.S1_at | CD997936 |
| | ZmAffx.243.1.S1_at | AI670255 |
| | Zm.17696.1.A1_at | BM073027 |
| | Zm.13194.2.A1_at | AY108895.1 |
| | Zm.13059.1.S1_at | AB112938.1 |
| | Zm.3255.2.A1_a_at | BM073865 |
| | ZmAffx.57.1.A1_at | AI665066 |
| | Zm.18764.1.A1_at | CO519979 |
| 20B. Negative | Zm.4875.1.S1_at | AI691556 |
| Correlation | Zm.5980.2.A1_a_at | AI666161 |
| | Zm.6045.2.A1_a_at | BM337093 |
| | Zm.14497.15.A1_x_at | CF016873 |
| | Zm.281.1.S1_at | U06831.1 |
| | Zm.2376.1.A1_x_at | AF001634.1 |
| | Zm.6007.1.S1_at | AI666154 |
| | ZmAffx.316.1.A1_at | AI670498 |
| | Zm.17786.1.S1_at | CF623596 |
| | Zm.18419.1.A1_at | CF631047 |
| | Zm.16237.1.A1_at | CF624893 |
| | Zm.6594.1.A1_at | CF972362 |
| | Zm.18998.1.S1_at | BF727820 |
| | ZmAffx.421.1.S1_at | AI676853 |
| | Zm.3198.2.A1_a_at | CN844169 |
| | Zm.1551.1.A1_at | BM339714 |
| | Zm.936.1.A1_at | CF052340 |
| | Zm.6194.1.A1_at | AW519914 |
| | AFFX-ThrX-M_at | AFFX-ThrX-M |
| | Zm.4304.1.S1_at | AI834719 |
| | Zm.3616.1.A1_at | BM380107 |
| | Zm.16207.1.A1_at | AW355980 |
| | Zm.5917.2.A1_at | BM379236 |
| | ZmAffx.914.1.A1_at | AI770970 |
| | Zm.18260.1.A1_at | CF602623 |
| | Zm.16879.1.A1_at | CF645954 |
| | Zm.19203.1.S1_at | CO520849 |
| | Zm.17500.1.A1_at | CK371009 |
| | Zm.5705.1.S1_at | AI637038 |
| | Zm.7892.1.A1_at | CO520489 |
| | ZmAffx.586.1.A1_at | AI715014 |
| | Zm.11783.1.A1_at | BM380733 |
| | Zm.18254.2.A1_at | CF632979 |
| | Zm.4258.1.A1_at | BM348441 |
| | Zm.13790.1.S1_at | AY105115.1 |
| | Zm.14428.1.S1_at | AY106109.1 |
| | Zm.13947.2.A1_at | AI737859 |
| | Zm.12517.1.A1_at | CF624446 |
| | Zm.5507.1.S1_at | CN071496 |
| | Zm.11055.1.A1_at | BM336314 |
| | Zm.13417.1.A1_at | CA400681 |
| | Zm.12101.2.S1_at | AI833552 |
| | Zm.10202.1.A1_at | AY112463.1 |
| | ZmAffx.273.1.A1_at | AI670401 |
| | Zm.784.1.A1_at | CF005849 |
| | Zm.7858.1.A1_at | AY108500.1 |
| | Zm.9839.1.A1_at | BM339393 |
| | ZmAffx.1198.1.S1_at | BE056195 |
| | Zm.4326.1.A1_at | AI711615 |
| | Zm.9735.1.A1_at | BM336891 |
| | Zm.3634.1.A1_at | CF638013 |
| | Zm.1408.1.A1_at | CN845023 |
| | Zm.16848.1.A1_at | CK369421 |
| | Zm.8114.1.A1_at | BM072985 |
| | ZmAffx.138.1.A1_at | AI667759 |
| | Zm.5803.1.A1_at | AI691266 |
| | Zm.10681.1.A1_at | BQ538977 |
| | Zm.9867.1.A1_at | AY106142.1 |
| | Zm.1511.1.S1_at | CO532736 |
| | Zm.7150.1.A1_x_at | AY103659.1 |
| | Zm.9614.1.A1_at | BM335440 |
| | Zm.1338.1.S1_at | W49442 |
| | Zm.8900.1.A1_at | CK827399 |
| | ZmAffx.721.1.A1_at | AI665110 |
| | Zm.7596.1.A1_at | BM079087 |
| | Zm.19034.1.S1_at | BQ833817 |
| | Zm.8959.1.A1_at | BM335622 |
| | Zm.2243.1.A1_at | BM349368 |
| | Zm.13403.1.S1_x_at | AF457949.1 |
| | AFFX-Zm-r2-Ec-bioB-3_at | AFFX-Zm-r2-Ec-bioB-3 |
| | Zm.3633.1.A1_at | U33816.1 |
| | Zm.17529.1.S1_at | CK394827 |
| | Zm.18275.1.A1_at | CO526155 |
| | Zm.7056.6.A1_at | CF051906 |
| | Zm.5796.1.A1_at | BM332299 |
| | ZmAffx.1106.1.S1_at | AW216267 |
| | Zm.12965.1.A1_at | CA402509 |
| | Zm.13845.1.A1_at | AY103950.1 |
| | Zm.12765.1.A1_at | AI745814 |
| | ZmAffx.1500.1.S1_at | 40794996-117 |
| | Zm.10867.1.A1_at | BM073190 |
| | Zm.19144.1.A1_at | CO518283 |
| | ZmAffx.262.1.A1_s_at | AI670379 |
| | Zm.7012.9.A1_at | BE123180 |
| | ZmAffx.1295.1.S1_s_at | 40794996-25 |
| | Zm.4682.1.S1_at | AI737946 |
| | Zm.2367.1.S1_at | AW497505 |
| | Zm.8847.1.A1_at | BM075896 |
| | Zm.2813.1.A1_at | BM381379 |
| | ZmAffx.586.1.S1_at | AI715014 |
| | Zm.14450.1.A1_at | AI391911 |
| | Zm.1454.1.A1_at | BG841866 |
| | Zm.18933.2.S1_at | AI734652 |
| | Zm.1118.1.S1_at | CF631214 |
| | Zm.18416.1.A1_at | CO524449 |
| | ZmAffx.939.1.S1_at | AI820322 |
| | Zm.16251.1.A1_at | AI711812 |
| | Zm.18427.1.S1_at | CO523584 |
| | Zm.10053.1.A1_at | CO523900 |
| | Zm.18439.1.A1_at | BM267666 |
| | Zm.12356.1.S1_at | BQ547740 |
| | ZmAffx.507.1.A1_at | AI691932 |
| | Zm.10718.1.A1_at | BM339638 |
| | Zm.15796.1.S1_at | BE640285 |
| | ZmAffx.270.1.A1_at | AI670398 |
| | Zm.54.1.S1_at | L25805.1 |
| | Zm.8391.1.A1_at | BM347365 |
| | Zm.9238.1.A1_at | CO533275 |
| | Zm.3633.2.S1_x_at | CF634876 |
| | Zm.4505.1.S1_at | AY111153.1 |
| | Zm.12070.1.A1_at | BM418472 |
| | Zm.17977.1.A1_s_at | CK827616 |
| | Zm.5789.3.S1_at | X83696.1 |
| | ZmAffx.771.1.A1_at | AI746147 |
| | Zm.11620.1.A1_at | BM379366 |
| | Zm.5571.2.A1_a_at | AY107402.1 |
| | Zm.12192.1.A1_at | BM380585 |
| | Zm.19243.1.A1_at | AW181224 |
| | Zm.12382.1.S1_at | BU097491 |
| | Zm.7538.1.A1_at | BM337034 |
| | Zm.1738.2.A1_at | CF630684 |
| | Zm.1313.1.A1_s_at | BM078737 |
| | Zm.9389.2.A1_x_at | BQ538340 |
| | ZmAffx.678.1.A1_at | AI734611 |
| | Zm.18105.1.S1_at | CO527288 |
| | Zm.19042.1.A1_at | CO521963 |
| | ZmAffx.782.1.A1_at | AI759014 |
| | Zm.5957.1.S1_at | AY105442.1 |
| | Zm.18908.1.S1_at | CO531963 |
| | Zm.1004.1.S1_at | BE511241 |
| | Zm.6743.1.S1_at | AF494284.1 |
| | Zm.8118.1.A1_at | AY107915.1 |
| | ZmAffx.960.1.S1_at | AI833639 |
| | Zm.17425.1.S1_at | CK145186 |
| | Zm.8106.1.S1_at | BM079856 |
| | ZmAffx.277.1.S1_at | AI670405 |
| | Zm.13686.1.A1_at | AY106861.1 |
| | Zm.1068.1.S1_at | BM381276 |
| | Zm.778.1.A1_a_at | CO529433 |
| | Zm.11834.1.S1_at | BM381120 |
| | Zm.16324.1.A1_at | CF032268 |
| | Zm.18774.1.S1_at | CO524725 |
| | Zm.14811.1.S1_at | CF629330 |
| | Zm.6654.1.A1_at | CF038689 |
| | Zm.17243.1.S1_at | CK786707 |
| | Zm.6000.1.S1_at | BG265807 |
| | Zm.17212.1.A1_at | CO529021 |
| | Zm.8233.2.S1_a_at | BM381462 |
| | Zm.138842.A1_at | AF099414.1 |
| | ZmAffx.1362.1.S1_at | 11990232-90 |
| | Zm.7904.1.A1_at | BM080363 |
| | Zm.16742.1.A1_at | AW499330 |
| | Zm.5119.1.A1_a_at | CF634150 |
| | Zm.152.1.S1_at | J04550.1 |
| | Zm.15451.1.S1_at | CD439729 |
| | Zm.5492.1.A1_at | AI622235 |
| | Zm.2710.1.S1_at | CO520765 |
| | Zm.8937.1.A1_at | BM080734 |
| | Zm.14283.4.S1_at | BG841525 |
| | Zm.6437.1.A1_a_at | CA402215 |
| | Zm.10175.1.A1_at | BM379420 |
| | Zm.6228.1.A1_at | AI739920 |
| | Zm.5558.1.A1_at | AY072298.1 |
| | Zm.10269.1.S1_at | BM660878 |
| | Zm.1894.2.S1_at | CK371174 |
| | Zm.12875.1.A1_at | CA400938 |
| | Zm.3138.1.A1_a_at | AI621861 |
| | Zm.15984.1.A1_at | CD441218 |
| | ZmAffx.1073.1.A1_at | AI947671 |
| | Zm.8489.1.A1_at | BQ538173 |
| | Zm.14962.1.A1_at | BM268018 |
| | Zm.9799.1.A1_at | AY111917.1 |
| | Zm.3833.1.A1_at | AW288806 |
| | Zm.15467.1.A1_at | CD219385 |
| | Zm.4316.1.S1_a_at | AI881448 |
| | Zm.4246.1.A1_at | AI438854 |
| | Zm.9521.1.A1_x_at | CF624102 |
| | Zm.17356.1.A1_at | CF634567 |
| | Zm.17913.1.S1_at | CF625344 |
| | Zm.17630.1.A1_at | CK348094 |
| | Zm.3350.1.A1_x_at | BM266649 |
| | Zm.2031.1.S1_at | AY103664.1 |
| | Zm.5623.1.A1_at | BG840990 |
| | Zm.16338.1.A1_at | CF348862 |
| | Zm.6430.1.A1_at | AY111839.1 |
| | Zm.10210.1.A1_at | CF627510 |
| | Zm.4418.1.A1_at | BM378152 |
| | ZmAffx.791.1.A1_at | AI759133 |
| | Zm.9048.1.A1_at | CF024226 |
| | Zm.2542.1.A1_at | CF636373 |
| | Zm.19011.2.A1_at | AY108328.1 |
| | Zm.9650.1.S1_at | BM380250 |
| | Zm.7804.1.S1_at | AF453836.1 |
| | Zm.17656.1.S1_at | CK369512 |
| | Zm.7860.1.A1_at | BM333940 |
| | Zm.3395.1.A1_at | AY103867.1 |
| | Zm.14505.2.A1_at | CF059379 |
| | Zm.3099.1.S1_at | CO522746 |
| | Zm.12133.1.S1_at | CF636936 |
| | Zm.4999.1.S1_at | AI600285 |
| | Zm.16080.1.A1_at | AY108583.1 |
| | Zm.2715.1.A1_at | AW066985 |
| | Zm.5797.1.S1_at | CF012679 |
| | ZmAffx.844.1.A1_at | AI770609 |
| | Zm.13263.1.A1_at | AY109418.1 |
| | Zm.3852.1.S1_at | CD998914 |
| | Zm.12391.1.S1_at | CF349132 |
| | Zm.6624.1.S1_at | AI491254 |
| | Zm.13961.1.S1_at | AY540745.1 |
| | Zm.8632.1.A1_at | BM268513 |
| | Zm.15102.1.A1_at | AI065586 |
| | Zm.11831.1.S1_a_at | CA401860 |
| | Zm.4460.1.A1_at | AI714963 |
| | Zm.4546.1.A1_at | BG266283 |
| | RPTR-Zm-U55943-1_at | RPTR-Zm-U55943-1 |
| | Zm.7915.1.A1_at | BM080414 |
| | ZmAffx.188.1.S1_at | AI668391 |
| | Zm.3889.5.A1_x_at | AI737901 |
| | Zm.2078.1.A1_at | CF675000 |
| | Zm.7648.1.A1_at | CO517814 |
| | Zm.3167.1.S1_s_at | U89342.1 |
| | Zm.19347.1.S1_at | AI902024 |
| | Zm.1881.1.A1_at | AY110751.1 |
| | Zm.6982.1.S1_at | AY105052.1 |
| | Zm.4187.1.S1_at | AY105088.1 |
| | Zm.6298.1.A1_at | CD444675 |
| | Zm.9529.1.A1_at | CA399003 |
| | Zm.1383.1.A1_at | BG873830 |
| | Zm.9339.1.A1_at | BM332063 |
| | Zm.6318.1.A1_at | BM073937 |
| | Zm.16926.1.S1_at | CO522465 |
| | ZmAffx.485.1.S1_at | AI691349 |
| | Zm.3795.1.A1_at | BM335144 |
| | Zm.5367.1.A1_at | CF638282 |
| | Zm.2040.2.S1_a_at | CB331475 |
| | Zm.7056.12.S1_at | AI746152 |
| | Zm.5656.1.A1_at | BG837879 |
| | Zm.1212.1.S1_at | CF011510 |
| | Zm.9098.1.A1_a_at | BM336161 |
| | Zm.3805.1.S1_at | AY112434.1 |
| | Zm.6645.1.S1_at | CF637989 |
| | Zm.9250.1.S1_at | CF016507 |
| | Zm.2656.2.S1_s_at | AY111594.1 |
| | Zm.13585.1.S1_at | AY107846.1 |
| | ZmAffx.261.1.S1_at | AI670366 |
| | Zm.1056.1.S1_a_at | AW120162 |
| | ZmAffx.474.1.S1_at | AI677507 |
| | Zm.2225.1.S1_at | BF728179 |
| | Zm.8292.1.S1_at | AY106611.1 |
| | Zm.6569.9.A1_x_at | AW091447 |
| | Zm.4230.1.S1_at | CO523811 |
| | RPTR-Zm-J01636-4_at | RPTR-Zm-J01636-4 |
| | Zm.13326.1.S1_at | CF042397 |
| | ZmAffx.728.1.A1_at | AI740010 |
| | Zm.6048.2.S1_at | AI745933 |
| | Zm.9513.1.A1_at | BM349310 |
| | Zm.5944.1.A1_at | BG874229 |
| | ZmAffx.1059.1.A1_at | AI881930 |
| | Zm.14352.2.S1_at | AY104356.1 |
| | ZmAffx.607.1.S1_at | AI715035 |
| | Zm.2199.2.S1_at | CA404051 |
| | Zm.9169.2.S1_at | CO521754 |
| | ZmAffx.630.1.S1_at | AI715058 |
| | Zm.16285.1.S1_at | CD970925 |
| | Zm.9747.1.S1_at | BM337726 |
| | Zm.9783.1.A1_at | BM347856 |
| | ZmAffx.827.1.A1_at | AI770520 |
| | Zm.3133.1.S1_at | CK371248 |
| | Zm.15512.1.S1_at | CD436002 |
| | Zm.4531.1.A1_at | AI734623 |
| | Zm.12810.1.A1_at | CA399348 |
| | Zm.17498.1.A1_at | CK144816 |
| | ZmAffx.821.1.A1_at | AI770497 |
| | Zm.5723.1.A1_at | BM079835 |
| | Zm.16535.2.A1_s_at | CF062633 |
| | Zm.14502.1.S1_at | CO531791 |
| | Zm.10792.1.A1_at | AY106092.1 |
| | Zm.14170.1.A1_a_at | BG841910 |
| | ZmAffx.1005.1.A1_at | AI881362 |
| | Zm.5048.6.A1_at | BM380925 |
| | Zm.8270.1.A1_at | AY649984.1 |
| | Zm.1899.1.A1_at | BM333426 |
| | Zm.17843.1.A1_at | BM380806 |
| | Zm.7005.1.A1_at | BM333037 |
| | Zm.15576.1.A1_a_at | CK827910 |
| | Zm.13930.1.A1_x_at | Z35298.1 |
| | Zm.12433.1.S1_at | AY105016.1 |
| | ZmAffx.1031.1.A1_at | AI881675 |
| | ZmAffx.237.1.S1_at | AI670249 |
| | Zm.13103.1.S1_at | CO534624 |
| | Zm.16538.1.S1_at | BM337996 |
| | Zm.10271.1.S1_at | CA452443 |
| | Zm.6625.2.S1_at | BM347999 |
| | Zm.8756.1.A1_at | BM333012 |
| | Zm.885.1.S1_at | BM080781 |
| | ZmAffx.1077.1.A1_at | AI948123 |
| | Zm.14463.1.A1_at | BM336602 |
| | ZmAffx.58.1.S1_at | AI665082 |
| | Zm.5112.1.A1_at | AI600906 |
| | Zm.14076.2.A1_a_at | CO526265 |
| | Zm.3077.2.S1_x_at | CF061929 |
| | Zm.9814.1.A1_at | BM351590 |
| | Zm.161.2.S1_x_at | X70153.1 |
| | Zm.16266.1.S1_at | CF243553 |
| | Zm.17657.1.A1_at | CK369553 |
| | Zm.19019.1.A1_at | BM080703 |
| | Zm.10514.1.S1_at | BQ485919 |
| | Zm.2473.1.S1_at | AY104610.1 |
| | Zm.13720.1.S1_s_at | AY106348.1 |
| | Zm.2266.1.A1_at | AW330883 |
| | Zm.5228.1.A1_at | AW061845 |
| | AFFX-Zm-r2-Ec-bioC-3_at | AFFX-Zm-r2-Ec-bioC-3 |
| | Zm.13858.1.S1_at | CO524282 |
| | Zm.5847.1.A1_at | BM078382 |
| | Zm.9056.1.A1_at | BM334642 |
| | Zm.4894.1.A1_at | BM076024 |
| | ZmAffx.1032.1.S1_at | AI881679 |
| | Zm.9757.1.A1_at | BM338070 |
| | Zm.4616.1.A1_a_at | BQ538201 |
| | Zm.4287.1.A1_at | BG266567 |
| | Zm.5988.1.A1_at | AI666062 |
| | Zm.4187.1.A1_at | AY105088.1 |
| | Zm.8665.1.A1_at | BM075117 |
| | Zm.5080.1.A1_at | AI600750 |
| | Zm.5930.1.S1_at | CF018694 |

TABLE 21
Pedigree and seedling growth characteristics of the
maize inbred lines used in Example 6a

| | | | | Seedling characteristics | |
|---|---|---|---|---|---|
| | | Group | Subgroup | after 2 weeks' growth | |
| Line | Pedigree [72] | [72] | [72] | Weight/g | Height/mm |

Parent in all crosses

| | | | | | |
|---|---|---|---|---|---|
| B73 | lowa Stiff Stalk Synthetic | SS | B73 | 1.62 | 204 |
| | C5 | | | | |

Training dataset

| | | | | | |
|---|---|---|---|---|---|
| B97 | derived from BSCB1(R)C9 | NSS | NSS-mixed | 1.30 | 204 |
| CML52 | Pop. 79? | TS | TZI | 2.18 | 262 |
| CML69 | Pop. 36 = Cogollero | TS | Suwan | 2.56 | 273 |
| | (Caribbean) | | | | |
| CML228 | Suwan-1/SR | TS | Suwan | 0.88 | 159 |
| CML247 | Pool 24 (Tuxpeño) | TS | CML-early | 2.11 | 227 |
| CML277 | Pop. 43 = La Posta (Tux.) | TS | CML-P | 1.26 | 205 |
| CML322 | Recyc. US + Mex | TS | CML-early | 1.29 | 173 |
| CML333 | Pop. 590 = ? | TS | CML-P | 1.46 | 184 |
| II14H | White Narrow Grain | Sweet | | 1.68 | 264 |
| | Evergreen | corn | | | |
| Ki11 | Suwan 1 | TS | Suwan | 2.04 | 174 |
| Ky21 | Boone County White | NSS | K64W | 1.40 | 191 |
| M37W | AUSTRALIA/JELLICORSE | Mixed | | 1.12 | 204 |
| Mo17 | C.I.187-2*C103 | NSS | CO109:Mo17 | 2.39 | 231 |
| Mo18W | Wf9*Mo22(2) | Mixed | | 1.12 | 197 |
| NC350 | H5*PX105A/H101 | TS | NC | 1.49 | 206 |
| NC358 | TROPHY SYN | TS | TZI | 1.12 | 161 |
| Oh43 | Oh40B*W8 | NSS | M14:Oh43 | 3.13 | 293 |
| P39 | Purdue Bantam | Sweet | | 0.49 | 146 |
| | | corn | | | |
| Tx303 | Yellow Surcropper | Mixed | | 1.10 | 179 |
| Tzi8 | TZB × TZSR | TS | TZI | 1.22 | 206 |

Test dataset

| | | | | | |
|---|---|---|---|---|---|
| CML103 | Pop. 44 | TS | CML-late | 1.52 | 199 |
| HP301 | Supergold | Popcorn | | 1.02 | 240 |
| Ki3 | Suwan-1 lines | TS | Suwan | 1.79 | 230 |
| Oh7B | Oh07B = [(Oh07*38- | Mixed | | 0.72 | 149 |
| | 11)Oh07] | | | | |

TABLE 22
Maize genes for which transcript abundance in inbred
lines of the training dataset is correlated (P < 0.00001) with plot
yield of hybrids with line B73

| Systematic Name | P value | R2 | Slope | Intercept | GenBank entry |
|---|---|---|---|---|---|
| Zm.3907.1.S1_at | 0 | 0.648 | −0.1182 | 1.773 | gb: L81162.2 |
| | | | | | DB_XREF = gi: 50957230 |
| Zm.18118.1.S1_at | 0 | 0.5906 | −0.3374 | 5.653 | gb: CN844890 |
| | | | | | DB_XREF = gi: 47962181 |
| Zm.2741.1.A1_at | 1.13E−12 | 0.585 | −0.3268 | 5.597 | gb: CB603857 |
| | | | | | DB_XREF = gi: 29543461 |
| Zm.13075.1.A1_at | 4.58E−12 | 0.5647 | −0.8445 | 12.26 | gb: CA403748 |
| | | | | | DB_XREF = gi: 24768619 |
| Zm.11896.1.A1_at | 4.62E−12 | 0.5646 | −0.523 | 7.705 | gb: CO530711 |
| | | | | | DB_XREF = gi: 50335585 |
| Zm.8790.1.A1_at | 3.76E−11 | 0.5324 | −0.1699 | 3.336 | gb: CF005102 |
| | | | | | DB_XREF = gi: 32865420 |
| Zm.14547.1.S1_a_at | 4.19E−11 | 0.5307 | −0.2015 | 2.891 | gb: BG840169 |
| | | | | | DB_XREF = gi: 14243004 |
| Zm.17578.1.A1_at | 5.68E−11 | 0.5258 | −3.303 | 48.37 | gb: CK368635 |
| | | | | | DB_XREF = gi: 40334565 |
| ZmAffx.1036.1.S1_at | 8.13E−11 | 0.52 | −0.1258 | 1.934 | gb: AI881726 |
| | | | | | DB_XREF = gi: 5566710 |
| Zm.6469.1.S1_at | 8.45E−11 | 0.5194 | 0.0888 | −0.1612 | gb: BE345306 |
| | | | | | DB_XREF = gi: 9254838 |
| ZmAffx.1211.1.A1_at | 9.65E−11 | 0.5172 | −0.5151 | 8.386 | gb: BG842238 |
| | | | | | DB_XREF = gi: 14244259 |
| Zm.17743.1.S1_at | 1.06E−10 | 0.5156 | −0.8687 | 12.7 | gb: CK370833 |
| | | | | | DB_XREF = gi: 40336763 |
| Zm.11126.1.S1_at | 3.41E−10 | 0.496 | 0.103 | −0.3613 | gb: AA979835 |
| | | | | | DB_XREF = gi: 3157213 |
| Zm.17115.1.S1_at | 4.19E−10 | 0.4925 | −0.395 | 6.294 | gb: CN844978 |
| | | | | | DB_XREF = gi: 47962269 |
| Zm.1465.1.A1_at | 1.08E−09 | 0.476 | −1.141 | 17.41 | gb: BG840947 |
| | | | | | DB_XREF = gi: 14243198 |
| ZmAffx.175.1.A1_at | 1.58E−09 | 0.4692 | −0.7394 | 11.35 | gb: AI668276 |
| | | | | | DB_XREF = gi: 4827584 |
| Zm.7407.1.A1_a_at | 1.77E−09 | 0.4672 | −0.1588 | 3.222 | gb: BM074289 |
| | | | | | DB_XREF = gi: 16919636 |
| Zm.12072.1.S1_at | 1.86E−09 | 0.4663 | −0.2694 | 3.894 | gb: BM417375 |
| | | | | | DB_XREF = gi: 18384175 |
| Zm.17209.1.A1_at | 2.01E−09 | 0.4648 | 0.07619 | −0.06023 | gb: BM073068 |
| | | | | | DB_XREF = gi: 16916971 |
| Zm.1615.1.S1_at | 2.37E−09 | 0.4618 | −0.1839 | 3.377 | gb: AY106014.1 |
| | | | | | DB_XREF = gi: 21209092 |
| Zm.1835.2.A1_at | 2.76E−09 | 0.459 | −0.1609 | 2.806 | gb: CK985959 |
| | | | | | DB_XREF = gi: 45568216 |
| Zm.5605.1.S1_at | 3.21E−09 | 0.4563 | −0.1728 | 3.327 | gb: CO528780 |
| | | | | | DB_XREF = gi: 50333654 |
| Zm.17923.1.A1_at | 3.99E−09 | 0.4523 | −0.2692 | 4.808 | gb: AY110526.1 |
| | | | | | DB_XREF = gi: 21214935 |
| Zm.7407.1.A1_x_at | 4.46E−09 | 0.4502 | −0.1987 | 3.798 | gb: BM074289 |
| | | | | | DB_XREF = gi: 16919636 |
| Zm.1143.1.S1_at | 4.54E−09 | 0.4499 | −0.166 | 3.287 | gb: CD443909 |
| | | | | | DB_XREF = gi: 31359552 |
| Zm.5656.1.A1_at | 5.20E−09 | 0.4473 | 0.1137 | −0.4548 | gb: BG837879 |
| | | | | | DB_XREF = gi: 14204202 |
| Zm.7397.1.A1_at | 5.31E−09 | 0.4469 | 0.168 | −1.328 | gb: BQ539216 |
| | | | | | DB_XREF = gi: 28984830 |
| Zm.11141.1.S1_at | 7.30E−09 | 0.441 | −0.1185 | 2.511 | gb: AY106810.1 |
| | | | | | DB_XREF = gi: 21209888 |
| Zm.6221.1.S1_at | 7.80E−09 | 0.4397 | −0.06997 | 1.969 | gb: AW585256 |
| | | | | | DB_XREF = gi: 7262313 |
| Zm.4741.1.A1_a_at | 8.01E−09 | 0.4392 | −0.2734 | 4.707 | gb: AI600480 |
| | | | | | DB_XREF = gi: 4609641 |
| Zm.8535.1.A1_at | 1.06E−08 | 0.4338 | −0.1364 | 2.904 | gb: AY104401.1 |
| | | | | | DB_XREF = gi: 21207479 |
| Zm.14547.1.S1_at | 1.39E−08 | 0.4287 | −0.2202 | 3.814 | gb: BG840169 |
| | | | | | DB_XREF = gi: 14243004 |
| Zm.16839.1.A1_at | 1.67E−08 | 0.4251 | 0.0764 | 0.004757 | gb: CF630748 |
| | | | | | DB_XREF = gi: 37387111 |
| Zm.19172.1.A1_at | 1.90E−08 | 0.4226 | −0.1808 | 3.45 | gb: CO528850 |
| | | | | | DB_XREF = gi: 50333724 |
| Zm.5170.1.S1_at | 2.20E−08 | 0.4197 | 0.11 | −0.4471 | gb: CF349172 |
| | | | | | DB_XREF = gi: 33942572 |
| Zm.5851.11.A1_x_at | 2.71E−08 | 0.4156 | −0.7137 | 11.37 | gb: CO527835 |
| | | | | | DB_XREF = gi: 50332709 |
| Zm.7006.2.A1_at | 2.84E−08 | 0.4147 | 0.07037 | 0.09825 | gb: AW225324 |
| | | | | | DB_XREF = gi: 6540662 |
| Zm.8914.1.S1_at | 2.95E−08 | 0.414 | 0.0947 | −0.2888 | gb: BM073720 |
| | | | | | DB_XREF = gi: 16918380 |
| Zm.1974.1.A1_at | 3.19E−08 | 0.4124 | −0.3785 | 6.334 | gb: CF920129 |
| | | | | | DB_XREF = gi: 38229816 |
| Zm.13497.1.S1_at | 3.62E−08 | 0.4099 | 0.08851 | −0.1197 | gb: CK368613 |
| | | | | | DB_XREF = gi: 40334543 |
| Zm.10640.1.S1_at | 3.96E−08 | 0.4081 | −0.08601 | 2.231 | gb: AY107547.1 |
| | | | | | DB_XREF = gi: 21210625 |
| Zm.19062.1.S1_at | 4.74E−08 | 0.4045 | −0.08075 | 2.065 | gb: CO531568 |
| | | | | | DB_XREF = gi: 50336442 |
| Zm.18060.1.A1_at | 4.79E−08 | 0.4043 | −0.2694 | 4.583 | gb: CK985812 |
| | | | | | DB_XREF = gi: 45567918 |
| Zm.878.1.S1_x_at | 5.24E−08 | 0.4025 | 0.1231 | −0.4754 | gb: AI855310 |
| | | | | | DB_XREF = gi: 5499443 |
| Zm.5159.1.A1_at | 6.20E−08 | 0.3991 | 0.0685 | 0.06159 | gb: CA403363 |
| | | | | | DB_XREF = gi: 24768234 |
| Zm.4632.1.A1_at | 6.24E−08 | 0.399 | −0.1062 | 2.425 | gb: AI737439 |
| | | | | | DB_XREF = gi: 5058963 |
| Zm.11189.1.A1_at | 6.86E−08 | 0.3971 | −0.08985 | 1.381 | gb: BM339882 |
| | | | | | DB_XREF = gi: 18170042 |
| Zm.1541.2.S1_at | 8.18E−08 | 0.3935 | 0.09864 | −0.363 | gb: CF650678 |
| | | | | | DB_XREF = gi: 37425858 |
| Zm.15307.1.A1_at | 8.20E−08 | 0.3934 | −4.65 | 68.91 | gb: CF014037 |
| | | | | | DB_XREF = gi: 32909225 |
| Zm.12775.1.A1_x_at | 8.37E−08 | 0.393 | −0.1098 | 1.876 | gb: CA398576 |
| | | | | | DB_XREF = gi: 24763400 |
| Zm.5086.1.A1_at | 1.03E−07 | 0.3887 | 0.05381 | 0.329 | gb: CF625592 |
| | | | | | DB_XREF = gi: 37377894 |
| Zm.5851.9.S1_at | 1.15E−07 | 0.3865 | −0.2305 | 3.44 | gb: AY105349.1 |
| | | | | | DB_XREF = gi: 21208427 |
| Zm.3182.1.A1_at | 1.31E−07 | 0.3838 | −0.06838 | 1.868 | gb: CK827062 |
| | | | | | DB_XREF = gi: 44900517 |
| Zm.5415.1.A1_at | 1.32E−07 | 0.3837 | −0.3297 | 5.269 | gb: BM074945 |
| | | | | | DB_XREF = gi: 16921022 |
| Zm.16855.1.A1_at | 1.34E−07 | 0.3833 | −0.1675 | 2.758 | gb: AF036949.1 |
| | | | | | DB_XREF = gi: 2865393 |
| Zm.5851.11.A1_a_at | 1.35E−07 | 0.3832 | −2.667 | 40.08 | gb: CO527835 |
| | | | | | DB_XREF = gi: 50332709 |
| ZmAffx.106.1.A1_at | 1.42E−07 | 0.3822 | −0.317 | 5.565 | gb: AI665540 |
| | | | | | DB_XREF = gi: 4776537 |
| Zm.5688.2.A1_at | 1.73E−07 | 0.3781 | −0.733 | 12.07 | gb: BM338540 |
| | | | | | DB_XREF = gi: 18168700 |
| Zm.9294.1.A1_at | 1.99E−07 | 0.3751 | −0.4105 | 6.62 | gb: BM335301 |
| | | | | | DB_XREF = gi: 18165462 |
| Zm.11189.1.A1_x_at | 2.14E−07 | 0.3736 | −0.1475 | 2.193 | gb: BM339882 |
| | | | | | DB_XREF = gi: 18170042 |
| Zm.8904.1.A1_at | 2.24E−07 | 0.3726 | −0.2324 | 3.566 | gb: CK371274 |
| | | | | | DB_XREF = gi: 40337204 |
| Zm.9631.1.A1_at | 2.37E−07 | 0.3714 | −0.1776 | 2.7 | gb: BM336220 |
| | | | | | DB_XREF = gi: 18166381 |
| Zm.2106.1.S1_at | 2.38E−07 | 0.3713 | −0.2349 | 4.515 | gb: CK786800 |
| | | | | | DB_XREF = gi: 44681752 |
| Zm.552.1.A1_at | 2.74E−07 | 0.3683 | 0.1283 | −0.6816 | gb: AF244691.1 |
| | | | | | DB_XREF = gi: 11385502 |
| Zm.9371.1.A1_x_at | 3.1E−07 | 0.3657 | −0.1302 | 2.806 | gb: BM350310 |
| | | | | | DB_XREF = gi: 18174922 |
| Zm.16747.1.A1_at | 3.18E−07 | 0.3652 | 0.06149 | 0.2381 | gb: BM335125 |
| | | | | | DB_XREF = gi: 18165286 |
| Zm.878.1.S1_at | 3.2E−07 | 0.365 | 0.2286 | −1.663 | gb: AI855310 |
| | | | | | DB_XREF = gi: 5499443 |
| Zm.12188.1.A1_at | 3.43E−07 | 0.3636 | −0.08906 | 1.631 | gb: BM382754 |
| | | | | | DB_XREF = gi: 18181544 |
| Zm.4452.1.A1_at | 3.5E−07 | 0.3631 | −0.1109 | 2.573 | gb: AI691174 |
| | | | | | DB_XREF = gi: 4938761 |
| Zm.17790.1.S1_at | 3.51E−07 | 0.363 | 0.1348 | −0.6063 | gb: CK370971 |
| | | | | | DB_XREF = gi: 40336901 |
| Zm.13843.1.A1_at | 3.79E−07 | 0.3614 | 0.06967 | 0.1099 | gb: AY104026.1 |
| | | | | | DB_XREF = gi: 21207104 |
| Zm.4271.4.A1_at | 3.88E−07 | 0.3609 | 0.05597 | 0.2215 | gb: BG316519 |
| | | | | | DB_XREF = gi: 13126069 |
| Zm.8922.1.S1_at | 3.95E−07 | 0.3605 | −0.1195 | 2.683 | gb: BM080861 |
| | | | | | DB_XREF = gi: 16927792 |
| Zm.6092.1.S1_at | 4.22E−07 | 0.3591 | 0.07163 | 0.03375 | gb: CB885460 |
| | | | | | DB_XREF = gi: 30087252 |
| Zm.5851.6.S1_x_at | 4.64E−07 | 0.3571 | −1.814 | 27.33 | gb: L46399.1 |
| | | | | | DB_XREF = gi: 939782 |
| Zm.3467.1.A1_at | 4.7E−07 | 0.3568 | −0.11 | 2.537 | gb: CF626421 |
| | | | | | DB_XREF = gi: 37379355 |
| Zm.495.1.A1_at | 5.15E−07 | 0.3548 | 0.05399 | 0.3248 | gb: AF236369.1 |
| | | | | | DB_XREF = gi: 7716457 |
| Zm.446.1.S1_at | 5.28E−07 | 0.3543 | −0.764 | 12.28 | gb: AF529266.1 |
| | | | | | DB_XREF = gi: 27544873 |
| Zm.5960.1.A1_at | 5.32E−07 | 0.3541 | −0.215 | 3.564 | gb: AI665953 |
| | | | | | DB_XREF = gi: 4804087 |
| Zm.4213.1.A1_at | 5.5E−07 | 0.3534 | −0.1478 | 3.071 | gb: BG841480 |
| | | | | | DB_XREF = gi: 14243777 |
| Zm.4728.1.A1_at | 5.59E−07 | 0.3531 | −0.1074 | 2.592 | gb: AI855200 |
| | | | | | DB_XREF = gi: 5499333 |
| Zm.9580.1.A1_at | 5.62E−07 | 0.3529 | −0.2372 | 4.381 | gb: BM332976 |
| | | | | | DB_XREF = gi: 18163137 |
| Zm.13808.1.S1_at | 5.75E−07 | 0.3524 | −0.105 | 2.492 | gb: AY104740.1 |
| | | | | | DB_XREF = gi: 21207818 |
| Zm.2626.1.A1_at | 6.12E−07 | 0.3511 | −0.05262 | 1.708 | gb: AY112337.1 |
| | | | | | DB_XREF = gi: 21216927 |
| Zm.15868.1.A1_at | 6.23E−07 | 0.3507 | 0.1032 | −0.2451 | gb: BM336226 |
| | | | | | DB_XREF = gi: 18166387 |
| Zm.4180.1.S1_at | 6.88E−07 | 0.3485 | 0.1176 | −0.5887 | gb: CD964540 |
| | | | | | DB_XREF = gi: 32824818 |
| Zm.5851.15.A1_x_at | 7.11E−07 | 0.3478 | −0.3181 | 5.392 | gb: AI759130 |
| | | | | | DB_XREF = gi: 5152832 |
| Zm.1739.1.A1_at | 7.48E−07 | 0.3467 | 0.1393 | −0.8398 | gb: BM337820 |
| | | | | | DB_XREF = gi: 18167980 |
| Zm.5390.1.A1_at | 7.81E−07 | 0.3458 | −0.1602 | 3.31 | gb: BM078263 |
| | | | | | DB_XREF = gi: 16925195 |
| Zm.3097.1.A1_at | 7.87E−07 | 0.3456 | 0.1663 | −0.8862 | gb: AY103827.1 |
| | | | | | DB_XREF = gi: 21206905 |
| Zm.6736.1.S1_at | 8.55E−07 | 0.3438 | −0.1797 | 3.458 | gb: AY108079.1 |
| | | | | | DB_XREF = gi: 21211157 |
| Zm.2910.1.S1_at | 8.67E−07 | 0.3435 | 0.09427 | −0.2644 | gb: CK145276 |
| | | | | | DB_XREF = gi: 38688245 |
| Zm.8697.1.A1_at | 8.83E−07 | 0.3431 | −0.1124 | 2.472 | gb: BM079294 |
| | | | | | DB_XREF = gi: 16926226 |
| Zm.4046.1.S1_at | 8.85E−07 | 0.343 | 0.1288 | −0.7911 | gb: CA400292 |
| | | | | | DB_XREF = gi: 24765132 |
| Zm.1285.1.A1_at | 9.43E−07 | 0.3416 | 0.05565 | 0.2897 | gb: AY111542.1 |
| | | | | | DB_XREF = gi: 21216132 |
| Zm.2563.1.A1_at | 9.52E−07 | 0.3414 | −0.05074 | 1.192 | gb: BE638571 |
| | | | | | DB_XREF = gi: 9951988 |
| Zm.17952.1.A1_at | 9.87E−07 | 0.3406 | −0.6734 | 10.55 | gb: CF632730 |
| | | | | | DB_XREF = gi: 37390982 |
| Zm.5766.1.S1_x_at | 1E−06 | 0.3403 | −0.3844 | 5.842 | gb: BG840404 |
| | | | | | DB_XREF = gi: 14242680 |
| Zm.15977.1.S1_at | 1.17E−06 | 0.3368 | 0.08845 | −0.8911 | gb: AY108613.1 |
| | | | | | DB_XREF = gi: 21211748 |
| Zm.3913.1.A1_at | 1.24E−06 | 0.3355 | 0.1163 | −0.4099 | gb: CF000034 |
| | | | | | DB_XREF = gi: 32860352 |
| Zm.303.1.S1_at | 1.3E−06 | 0.3346 | −0.07128 | 2.002 | gb: AF236373.1 |
| | | | | | DB_XREF = gi: 7716465 |
| Zm.4332.1.A1_at | 1.36E−06 | 0.3336 | −0.3654 | 6.262 | gb: AI711854 |
| | | | | | DB_XREF = gi: 5005792 |
| Zm.9376.1.A1_at | 1.41E−06 | 0.3326 | 0.09554 | −0.3578 | gb: BM332576 |
| | | | | | DB_XREF = gi: 18162737 |
| Zm.1423.1.A1_at | 1.46E−06 | 0.3319 | −0.0643 | 1.871 | gb: CF047935 |
| | | | | | DB_XREF = gi: 32943116 |
| Zm.1792.1.A1_at | 1.49E−06 | 0.3314 | 0.06852 | 0.04595 | gb: AY107188.1 |
| | | | | | DB_XREF = gi: 21210266 |
| Zm.17540.1.A1_at | 1.51E−06 | 0.3311 | −0.07019 | 1.93 | gb: CO525036 |
| | | | | | DB_XREF = gi: 50329910 |
| Zm.3561.1.A1_at | 1.52E−06 | 0.3311 | −0.6223 | 9.644 | gb: CK826673 |
| | | | | | DB_XREF = gi: 44900128 |
| ZmAffx.566.1.A1_at | 1.62E−06 | 0.3297 | −0.07933 | 1.337 | gb: AI714636 |
| | | | | | DB_XREF = gi: 5018443 |
| Zm.5597.1.A1_at | 1.63E−06 | 0.3295 | −0.2103 | 3.985 | gb: AI629497 |
| | | | | | DB_XREF = gi: 4680827 |
| Zm.13082.1.S1_a_at | 1.68E−06 | 0.3288 | −0.2151 | 3.969 | gb: CD438478 |
| | | | | | DB_XREF = gi: 31354121 |
| Zm.6216.1.S1_at | 1.69E−06 | 0.3287 | −0.04754 | 1.586 | gb: CO531189 |
| | | | | | DB_XREF = gi: 50336063 |
| Zm.2742.1.A1_at | 1.72E−06 | 0.3283 | −0.1419 | 3.028 | gb: AY111235.1 |
| | | | | | DB_XREF = gi: 21215825 |
| Zm.1559.1.S1_at | 1.72E−06 | 0.3282 | −0.07846 | 1.413 | gb: BF729152 |
| | | | | | DB_XREF = gi: 12058302 |
| Zm.3154.1.A1_at | 1.74E−06 | 0.328 | −0.03944 | 1.529 | gb: BM333548 |
| | | | | | DB_XREF = gi: 18163709 |
| Zm.3357.1.A1_at | 1.75E−06 | 0.3279 | 0.08751 | −0.1318 | gb: BM347858 |
| | | | | | DB_XREF = gi: 18172470 |
| Zm.2924.1.A1_a_at | 1.8E−06 | 0.3273 | −0.05843 | 1.786 | gb: BM349722 |
| | | | | | DB_XREF = gi: 18174334 |
| Zm.10301.1.A1_at | 1.86E−06 | 0.3265 | 0.1287 | −0.5513 | gb: BU050993 |
| | | | | | DB_XREF = gi: 22491070 |
| Zm.5992.1.A1_at | 1.87E−06 | 0.3264 | 0.07232 | 0.08961 | gb: AY108021.1 |
| | | | | | DB_XREF = gi: 21211099 |
| Zm.13693.1.S1_at | 1.87E−06 | 0.3264 | −0.1718 | 3.323 | gb: AY106770.1 |
| | | | | | DB_XREF = gi: 21209848 |
| Zm.6117.1.A1_at | 1.89E−06 | 0.3262 | −0.05436 | 1.737 | gb: BM074413 |
| | | | | | DB_XREF = gi: 16919905 |
| Zm.8911.1.A1_at | 2.03E−06 | 0.3246 | −0.2179 | 4.077 | gb: BM350783 |
| | | | | | DB_XREF = gi: 18175488 |
| Zm.7595.1.A1_at | 2.11E−06 | 0.3237 | −0.05045 | 1.648 | gb: CD437071 |
| | | | | | DB_XREF = gi: 31352714 |
| Zm.2424.1.A1_at | 2.28E−06 | 0.3219 | −0.3084 | 5.458 | gb: BG841655 |
| | | | | | DB_XREF = gi: 14243883 |
| Zm.2391.1.A1_at | 2.44E−06 | 0.3204 | −0.3225 | 5.482 | gb: CK826632 |
| | | | | | DB_XREF = gi: 44900087 |
| Zm.2455.1.A1_at | 2.47E−06 | 0.3201 | −0.09311 | 2.332 | gb: BM416746 |
| | | | | | DB_XREF = gi: 18383546 |
| Zm.12934.1.A1_a_at | 2.55E−06 | 0.3194 | −0.3145 | 4.903 | gb: AY106367.1 |
| | | | | | DB_XREF = gi: 21209445 |
| Zm.13266.2.S1_at | 2.6E−06 | 0.3189 | −0.2755 | 4.818 | gb: CO533594 |
| | | | | | DB_XREF = gi: 50338468 |
| Zm.9364.1.A1_at | 2.63E−06 | 0.3187 | 0.1468 | −0.7177 | gb: BM334062 |
| | | | | | DB_XREF = gi: 18164223 |
| Zm.6293.1.A1_at | 2.68E−06 | 0.3182 | −0.08441 | 2.061 | gb: CF038760 |
| | | | | | DB_XREF = gi: 32933948 |
| Zm.2530.1.A1_at | 2.71E−06 | 0.318 | −0.1539 | 3.168 | gb: CF637153 |
| | | | | | DB_XREF = gi: 37399642 |
| Zm.8204.1.A1_at | 2.8E−06 | 0.3172 | −0.07345 | 2.051 | gb: BM073273 |
| | | | | | DB_XREF = gi: 16917409 |
| Zm.843.1.A1_a_at | 2.81E−06 | 0.3172 | 0.06446 | 0.1415 | gb: AY111573.1 |
| | | | | | DB_XREF = gi: 21216163 |
| Zm.13288.1.S1_at | 2.82E−06 | 0.3171 | −0.07191 | 1.268 | gb: CA826847 |
| | | | | | DB_XREF = gi: 26455264 |
| Zm.19018.1.A1_at | 2.87E−06 | 0.3167 | −0.05674 | 1.775 | gb: CO532922 |
| | | | | | DB_XREF = gi: 50337796 |
| Zm.14036.1.S1_at | 2.89E−06 | 0.3165 | −0.05461 | 0.846 | gb: X55388.1 |
| | | | | | DB_XREF = gi: 22270 |
| Zm.13248.1.S1_at | 2.98E−06 | 0.3158 | −0.04989 | 0.7365 | gb: Y09301.1 |
| | | | | | DB_XREF = gi: 3851330 |
| Zm.14272.2.A1_at | 3.07E−06 | 0.3151 | 0.1132 | −0.5078 | gb: D10622.1 |
| | | | | | DB_XREF = gi: 217961 |
| Zm.14318.1.A1_at | 3.33E−06 | 0.3133 | 0.1184 | −0.4017 | gb: AY104313.1 |
| | | | | | DB_XREF = gi: 21207391 |
| Zm.19303.1.S1_at | 3.4E−06 | 0.3128 | 0.04973 | 0.3873 | gb: CA829102 |
| | | | | | DB_XREF = gi: 26457519 |
| ZmAffx.909.1.S1_at | 3.54E−06 | 0.3119 | −0.1389 | 2.793 | gb: AI770947 |
| | | | | | DB_XREF = gi: 5268983 |
| Zm.2293.1.A1_at | 3.65E−06 | 0.3112 | −0.3914 | 5.735 | gb: AW331208 |
| | | | | | DB_XREF = gi: 6827565 |
| Zm.3796.1.A1_at | 3.66E−06 | 0.3111 | −0.1047 | 2.305 | gb: BG836961 |
| | | | | | DB_XREF = gi: 14203284 |
| Zm.6560.1.S1_a_at | 3.95E−06 | 0.3094 | −0.1021 | 2.428 | gb: Z29518.1 |
| | | | | | DB_XREF = gi: 575959 |
| Zm.6560.1.S1_at | 4.13E−06 | 0.3083 | −0.5382 | 9.188 | gb: Z29518.1 |
| | | | | | DB_XREF = gi: 575959 |
| ZmAffx.667.1.A1_at | 4.19E−06 | 0.308 | −0.1973 | 3.638 | gb: AI734359 |
| | | | | | DB_XREF = gi: 5055472 |
| Zm.9931.1.A1_at | 4.36E−06 | 0.3071 | −0.2746 | 4.617 | gb: BM339241 |
| | | | | | DB_XREF = gi: 18169401 |
| Zm.11852.1.A1_x_at | 4.54E−06 | 0.3062 | 0.1797 | −1.23 | gb: CF013366 |
| | | | | | DB_XREF = gi: 32908553 |
| Zm.520.1.S1_x_at | 4.74E−06 | 0.3052 | 0.1057 | −0.5001 | gb: AF200528.1 |
| | | | | | DB_XREF = gi: 9622879 |
| Zm.16977.1.S1_at | 4.76E−06 | 0.3051 | −0.04535 | 1.634 | gb: AB102956.1 |
| | | | | | DB_XREF = gi: 38347685 |
| Zm.16227.1.A1_at | 4.77E−06 | 0.305 | −0.2137 | 4.017 | gb: BI180294 |
| | | | | | DB_XREF = gi: 14646105 |
| Zm.5379.1.S1_at | 4.91E−06 | 0.3043 | 0.4236 | −3.132 | gb: AI621513 |
| | | | | | DB_XREF = gi: 4630639 |
| Zm.17720.1.A1_at | 4.93E−06 | 0.3042 | −0.08202 | 1.488 | gb: BM340967 |
| | | | | | DB_XREF = gi: 18171127 |
| Zm.588.1.S1_at | 5.14E−06 | 0.3033 | 0.06464 | 0.1791 | gb: AF142322.1 |
| | | | | | DB_XREF = gi: 4927258 |
| Zm.18033.1.A1_at | 5.17E−06 | 0.3031 | −0.08471 | 2.06 | gb: BM080835 |
| | | | | | DB_XREF = gi: 16927766 |
| Zm.663.1.S1_at | 5.22E−06 | 0.3029 | −0.178 | 3.527 | gb: AF318075.1 |
| | | | | | DB_XREF = gi: 14091009 |
| Zm.16513.1.A1_at | 5.27E−06 | 0.3027 | −0.07343 | 1.845 | gb: CF634462 |
| | | | | | DB_XREF = gi: 37394377 |
| Zm.17307.1.S1_at | 5.53E−06 | 0.3016 | 0.06901 | −0.101 | gb: CK367910 |
| | | | | | DB_XREF = gi: 40333840 |
| Zm.13719.1.A1_at | 5.64E−06 | 0.3011 | −0.04963 | 1.62 | gb: AY106357.1 |
| | | | | | DB_XREF = gi: 21209435 |
| Zm.1611.1.A1_at | 5.7E−06 | 0.3009 | −0.09719 | 2.327 | gb: AW787466 |
| | | | | | DB_XREF = gi: 7844244 |
| Zm.6251.1.A1_at | 5.77E−06 | 0.3006 | −0.05725 | 1.778 | gb: CD434479 |
| | | | | | DB_XREF = gi: 31350122 |
| Zm.16854.1.S1_at | 6.1E−06 | 0.2993 | −0.08796 | 2.166 | gb: CF674957 |
| | | | | | DB_XREF = gi: 37621904 |
| Zm.7731.1.A1_at | 6.19E−06 | 0.299 | 0.0859 | −0.1337 | gb: AI612464 |
| | | | | | DB_XREF = gi: 4621631 |
| Zm.7074.1.A1_at | 6.21E−06 | 0.2989 | 0.09015 | −0.1237 | gb: CF634632 |
| | | | | | DB_XREF = gi: 37394712 |
| Zm.8376.1.S1_at | 6.34E−06 | 0.2984 | −0.07696 | 1.936 | gb: BM073880 |
| | | | | | DB_XREF = gi: 16918753 |
| Zm.14497.8.A1_x_at | 6.36E−06 | 0.2983 | 0.06997 | 0.1062 | gb: CO527469 |
| | | | | | DB_XREF = gi: 50332343 |
| Zm.14590.1.A1_x_at | 6.39E−06 | 0.2982 | −0.1306 | 2.728 | gb: AY110683.1 |
| | | | | | DB_XREF = gi: 21215273 |
| Zm.15293.1.S1_a_at | 6.49E−06 | 0.2978 | −0.1162 | 2.534 | gb: AF232008.2 |
| | | | | | DB_XREF = gi: 9313026 |
| Zm.15282.1.A1_at | 6.52E−06 | 0.2977 | −0.1326 | 2.786 | gb: BM382478 |
| | | | | | DB_XREF = gi: 18181268 |
| Zm.520.1.S1_at | 6.67E−06 | 0.2972 | 0.1149 | −0.623 | gb: AF200528.1 |
| | | | | | DB_XREF = gi: 9622879 |
| Zm.10553.1.A1_at | 6.93E−06 | 0.2963 | −0.2323 | 4.09 | gb: CD441187 |
| | | | | | DB_XREF = gi: 31356830 |
| Zm.3428.1.A1_at | 7.38E−06 | 0.2948 | −0.1968 | 3.706 | gb: AI964613 |
| | | | | | DB_XREF = gi: 5757326 |
| ZmAffx.1083.1.A1_at | 7.6E−06 | 0.2942 | −0.09468 | 2.276 | gb: AI974922 |
| | | | | | DB_XREF = gi: 5777303 |
| Zm.6997.1.A1_at | 7.72E−06 | 0.2938 | 0.045 | 0.4419 | gb: BG874061 |
| | | | | | DB_XREF = gi: 14245479 |
| Zm.16489.1.S1_at | 7.76E−06 | 0.2937 | 0.06034 | 0.2686 | gb: CF637893 |
| | | | | | DB_XREF = gi: 37401062 |
| Zm.5851.3.A1_at | 7.91E−06 | 0.2932 | −0.4542 | 7.864 | gb: AY104012.1 |
| | | | | | DB_XREF = gi: 21207090 |
| Zm.19019.1.A1_at | 8.06E−06 | 0.2928 | −0.06012 | 1.716 | gb: BM080703 |
| | | | | | DB_XREF = gi: 16927634 |
| Zm.4880.1.S1_at | 8.19E−06 | 0.2924 | −0.0599 | 1.721 | gb: CF627543 |
| | | | | | DB_XREF = gi: 37381330 |
| Zm.3243.1.A1_at | 8.21E−06 | 0.2924 | 0.08508 | −0.1167 | gb: AY105697.1 |
| | | | | | DB_XREF = gi: 21208775 |
| Zm.19022.1.S1_at | 8.43E−06 | 0.2917 | −0.246 | 3.664 | gb: CO526898 |
| | | | | | DB_XREF = gi: 50331772 |
| Zm.13991.1.S1_at | 8.5E−06 | 0.2915 | 0.07005 | 0.1974 | gb: AW424608 |
| | | | | | DB_XREF = gi: 6952540 |
| Zm.9867.1.A1_at | 8.51E−06 | 0.2915 | 0.3098 | −3.067 | gb: AY106142.1 |
| | | | | | DB_XREF = gi: 21209220 |
| Zm.6480.2.S1_a_at | 8.6E−06 | 0.2912 | 0.04572 | 0.403 | gb: AI065715 |
| | | | | | DB_XREF = gi: 30052426 |
| Zm.6931.1.S1_a_at | 9.14E−06 | 0.2898 | −0.09601 | 2.355 | gb: AY588275.1 |
| | | | | | DB_XREF = gi: 46560601 |
| Zm.12942.1.A1_at | 9.16E−06 | 0.2898 | −0.5247 | 7.489 | gb: CA402151 |
| | | | | | DB_XREF = gi: 24767006 |
| Zm.889.2.S1_at | 9.29E−06 | 0.2894 | −0.6597 | 10.97 | gb: CD439290 |
| | | | | | DB_XREF = gi: 31354933 |
| Zm.6816.1.A1_at | 9.86E−06 | 0.288 | 0.0469 | 0.3894 | gb: AY104584.1 |
| | | | | | DB_XREF = gi: 21207662 |

TABLE 23
Maize Plot Yield Data

| | Grain yield/lb | | |
|---|---|---|---|
| | per plot² | | |
| Hybrid¹ | Plot 1 | Plot 2 | Mean |

Training dataset

| | | | |
|---|---|---|---|
| B97 × B73 | 15.42 | 12.60 | 14.01 |
| CML228 × B73 | 15.11 | 15.23 | 15.17 |
| B73 × CML69 | 13.12 | 12.75 | 12.94 |
| B73 × CML247 | 13.95 | 14.35 | 14.15 |
| B73 × CML277 | 12.29 | 13.49 | 12.89 |
| B73 × CML322 | 10.20 | 11.72 | 10.96 |
| CML333 × B73 | 12.88 | 12.76 | 12.82 |
| CML52 × B73 | 13.97 | 14.99 | 14.48 |
| B73 × IL14H | 9.43 | 7.06 | 8.24 |
| B73 × Ki11 | 12.28 | 13.69 | 12.98 |
| Ky21 × B73 | 11.82 | 12.43 | 12.13 |
| B73 × M37W | 13.88 | 13.80 | 13.84 |
| B73 × Mo17 | 12.99 | 10.10 | 11.55 |
| B73 × Mo18W | 14.51 | 14.19 | 14.35 |
| NC350 × B73 | 18.27 | 19.43 | 18.85 |
| B73 × NC358 | 14.41 | 13.11 | 13.76 |
| Oh43 × B73 | 11.83 | 12.11 | 11.97 |
| P39 × B73 | 5.84 | 7.07 | 6.45 |
| B73 × Tx303 | 10.25 | 13.42 | 11.83 |
| Tzi8 B73 | 12.82 | 14.21 | 13.51 |

Test dataset

| | | | |
|---|---|---|---|
| B73 × CML103 | 14.16 | 14.86 | 14.51 |
| B73 × Hp301 | 8.06 | 9.92 | 8.99 |
| B73 × Ki3 | 12.14 | 14.15 | 13.15 |
| B73 × OH7B | 11.94 | 11.17 | 11.55 |

¹Maternal parent listed first
²Corrected to 15% moisture

### Program 1

```
job ‘kondara br-0 heterosis work’
output [width=132]1
variate [nvalues=22810]sec1,sec2,sec3,sec4,sec5,sec6,sec7,sec8,sec9,\
      DK22,DKLD,DKSD,DB22,DBLD,DBSD,DBH22,DBHLD,DBHSD,DKH22,DKHLD,DKHSD,
\
      HBK22,HBKLD,HBKSD,KBH22,KBHLD,KBHSD,D_K22,D_KLD,D_KSD,H22,HLD,HSD,
\
      BDK22,BDKLD,BDKSD,HB22,HBLD,HBSD,HK22,HKLD,HKSD,B_K22,B_KLD,B_KSD,
\
      r22kb,rldkb,rsdkb,r22bk,rldbk,rsdbk,KHB22,KHBLD,KHBSD,BHK22,BHKLD,
BHKSD,\
      KDB22,KDBLD,KDBSD,BDK22,BDKLD,BDKSD,H22h,HLDh,HSDh,H22l,HLDl,HSDl,
A,B,C,\
      b_k22,b_kLD,b_kSD,K_H22h,K_HLDh,K_HSDh,B_H22h,B_HLDh,B_HSDh,\
      HB22l,HBLDl,HBSDl,HB22h,HBLDh,HBSDh,\
      HK22l,HKLDl,HKSDl,HK22h,HKLDh,HKSDh
variate [values=1...22810]gene
“*********************************READ BASIC EXPRESSION
DATA*************************”
open ‘x:\\daves\\reciprocals\\hk 22k.txt’;ch=2
read [ch=2;print=e,s;serial=n]h22,hld,hsd,k22,kld,ksd,b22,bld,bsd
close ch=2
“ INITIAL SEED FOR RANDOM NUMBER GENERATION
      ”
scalar int,x,y
scalar [value=54321]a
    &  [value=78656]b
    &  [value=17345]c
output [width=132]1
```

| | |
|---|---|
| “ OPEN OUTPUT FILE | ” |

open ‘x:\\daves\\reciprocals\\hk 22k.out’;ch=3;width=132;filetype=o
scalar [value=12345]a
scalar [value=*]miss
scalar [value=1]int

| | |
|---|---|
| “ CALCULATES COMPARISONS FOR THREEOFOLD DIFFERENCES | ” |
| “************************************* ratio of K : B | |
| *****************************” | |
| calc r22kb=k22/b22 | |
| & rldkb=kld/bld | |
| & rsdkb=ksd/bsd | |
| “************************************* ratio of B : K | |
| *****************************” | |
| & r22bk=b22/k22 | |
| & rldbk=bld/kld | |
| & rsdbk=bsd/ksd | |
| “************************************* ratio of H : K | |
| *****************************” | |
| & r22hk=h22/k22 | |
| & rldhk=hld/kld | |
| & rsdhk=hsd/ksd | |
| “************************************* ratio of H : B | |
| *****************************” | |
| & r22hb=h22/b22 | |
| & rldhb=hld/bld | |
| & rsdhb=hsd/bsd | |
| for k=1...22810 | |
| “************************************* B = H (within 2) | |
| *****************************” | |
| for | |
| i=r22hb,rldhb,rsdhb;j=A,B,C;m=b22,bld,bsd;n=h22,hld,hsd;o=HB22l,HBLDl,HB | |
| SDl;p=HB22h, HBLDh, HBSDh | |
| if ((elem(i;k).gt.0.5).and.(elem(i;k).lt.2)) | |
| calc elem(j;k)=int | |
| else | |
| calc elem(j;k)=miss | |
| endif | |
| calc x=elem(m;k) | |
| & y=elem(n;k) | |

| | |
|---|---|
| “ LOWEST VALUE OF B OR H | ” |

```
            if (y.gt.x).and.(elem(j;k).eq.1)
                        calc elem(o;k)=x
                  elsif (x.gt.y).and.(elem(j;k).eq.1)
                        calc elem(o;k)=y
                  else
                        calc elem(o;k)=miss
            endif
```

| | |
|---|---|
| “ HIGHEST VALUE OF B OR H | ” |

```
            if (x.gt.y).and.(elem(j;k).eq.1)
                        calc elem(p;k)=x
                  elsif (y.gt.x).and.(elem(j;k).eq.1)
                        calc elem(p;k)=y
                  else
                        calc elem(p;k)=miss
            endif
      endfor
“*************************************  K = H (within
2)*****************************”
      for
i=r22hk,rldhk,rsdhk;j=A,B,C;m=k22,kld,ksd;n=h22,hld,hsd;o=HK22l,HKLDl,HK
SDl;p=HK22h,HKLDh,HKSDh
            if ((elem(i;k).gt.0.5).and.(elem(i;k).lt.2))
                  calc elem(j;k)=int
                        else
                  calc elem(j;k)=miss
            endif
                  calc x=elem(m;k)
                    &  y=elem(n;k)
```

| | |
|---|---|
| “ LOWEST VALUE OF K OR H | ” |

```
            if (x.lt.y).and.(elem(j;k).eq.1)
                        calc elem(o;k)=x
                  elsif (y.lt.x).and.(elem(j;k).eq.1)
                        calc elem(o;k)=y
                  else
                        calc elem(o;k)=miss
            endif
```

| | |
|---|---|
| “ HIGHEST VALUE OF K OR H | ” |

```
            if (x.gt.y).and.(elem(j;k).eq.1)
                        calc elem(p;k)=x
                  elsif (y.gt.x).and.(elem(j;k).eg.1)
                        calc elem(p;k)=y
                  else
                        calc elem(p;k)=miss
            endif
      endfor
“************************************* K = B (within 2)
*****************************”
      for i=r22kb,rldkb,rsdkb;j=A,B,C;m=k22,kld,ksd;n=b22,bld,bsd
            if ((elem(i;k).gt.0.5).and.(elem(i;k).lt.2))
                  calc elem(j;k)=int
                        else
                  calc elem(j;k)=miss
            endif
      endfor
“*********************************K = B (highest & lowest
values)********************”
      for
i=r22kb,rldkb,rsdkb;j=A,B,C;m=k22,kld,ksd;n=b22,bld,bsd;o=B_K22,B_KLD,B_{—}
KSD;p=b_k22,b_kLD,b_kSD
                  calc x=elem(m;k)
                    &  y=elem(n;k)
            if (x.gt.y)
                        calc elem(o;k)=x
                  else
                        calc elem(o;k)=y
            endif
            if (x.lt.y)
                        calc elem(p;k)=x
                  else
                        calc elem(p;k)=y
            endif
      endfor
endfor
“************************************ratio of H : (K = B)  high
values**************”
calc H22h=h22/B_K22
  &  HLDh=hld/B_KLD
  &  HSDh=hsd/B_KSD
“*************************************ratio of H : (K = B)  low
values***************”
calc H22l=h22/b_k22
  &  HLDl=hld/b_kLD
  &  HSDl=hsd/b_kSD
“***********************************ratio of K : (B = H)
****************************”
calc KDB22=k22/HB22h
  &  KDBLD=kld/HBLDh
  &  KDBSD=ksd/HBSDh
“************************************ratio of B : (K =
H)****************************”
calc BDK22=b22/HK22h
  &  BDKLD=bld/HKLDh
  &  BDKSD=bsd/HKSDh
“************************************ratio of (K = H − low values) : B
************”
calc KHB22=HK22l/b22
  &  KHBLD=HKLDl/bld
  &  KHBSD=HKSDl/bsd
“*************************************ratio of (B = H) :
K***************************”
calc BHK22=HB22l/k22
  &  BHKLD=HBLDl/kld
  &  BHKSD=HBSDl/ksd
“***********************************************************************
*************”
for k=1...22810
“***********************  SEC 1 ---- K>BR-0
      ********************************”
      if
(elem(r22kb;k).gt.2).and.(elem(rldkb;k).gt.2).and.(elem(rsdkb;k).gt.2)
            calc elem(sec1;k)=int
                  else
            calc elem(sec1;k)=miss
      endif
“***********************SEC 2 ---- BR-0>K
      *********************************”
      if
(elem(r22bk;k).gt.2).and.(elem(rldbk;k).gt.2).and.(elem(rsdbk;k).gt.2)
            calc elem(sec2;k)=int
                  else
            calc elem(sec2;k)=miss
      endif
“***********************SEC 3 ---- K AND H > B (BUT K = H)
      *****************”
      if
(elem(KHB22;k).gt.2).and.(elem(KHBLD;k).gt.2).and.(elem(KHBSD;k).gt.2)
            calc elem(sec3;k)=int
                  else
            calc elem(sec3;k)=miss
      endif
“***********************SEC 4 ---- B AND H > K (BUT B = H)
      *******************”
      if
(elem(BHK22;k).gt.2).and.(elem(BHKLD;k).gt.2).and.(elem(BHKSD;k).gt.2)
            calc elem(sec4;k)=int
                  else
            calc elem(sec4;k)=miss
      endif
“***********************SEC 5 K > B and H (BUT B = H)
      ************************”
      if
(elem(KDB22;k).gt.2).and.(elem(KDBLD;k).gt.2).and.(elem(KDBSD;k).gt.2)
            calc elem(sec5;k)=int
                  else
            calc elem(sec5;k)=miss
      endif
“***********************SEC 6 ---- B > K and H (BUT K = H)
      ************************”
      if
(elem(BDK22;k).gt.2).and.(elem(BDKLD;k).gt.2).and.(elem(BDKSD;k).gt.2)
            calc elem(sec6;k)=int
                  else
            calc elem(sec6;k)=miss
      endif
“***********************SEC 7 ---- H > B and
K*********************************”
      if
(elem(H22h;k).gt.2).and.(elem(HLDh;k).gt.2).and.(elem(HSDh;k).gt.2)
            calc elem(sec7;k)=int
                  else
            calc elem(sec7;k)=miss
      endif
“***********************SEC 8 ---- H < B and
K************************************”
      if
(elem(H22l;k).lt.0.5).and.(elem(HLDl;k).lt.0.5).and.(elem(HSDl;k).lt.0.5
)
            calc elem(sec8;k)=int
                  else
            calc elem(sec8;k)=miss
      endif
endfor
“***********************************************************************
*************”
for i=sec1,sec2,sec3,sec4,sec5,sec6,sec7,sec8;\
      j=No1,No2,No3,No4,No5,No6,No7,No8;\
      k=N1,N2,N3,N4,N5,N6,N7,N8;\
      l=mv1,mv2,mv3,mv4,mv5,mv6,mv7,mv8
            calc k=nvalues(i)
              &  l=nmv(i)
              &  j=k−l
endfor
print No1,No2,No3,No4,No5,No6,No7,No8
print [ch=3;iprint=*;rlprint=*;clprint=*]No1,No2,No3,No4,No5,No6,No7,No8
endfor
stop
```

### Program 2

```
job ‘kondara br-0 heterosis work’
output [width=132]1
variate [nvalues=22810]sec1,sec2,sec3,sec4,sec5,sec6,sec7,sec8,sec9,\
      DK22,DKLD,DKSD,DB22,DBLD,DBSD,DBH22,DBHLD,DBHSD,DKH22,DKHLD,DKHSD,
\
      HBK22,HBKLD,HBKSD,KBH22,KBHLD,KBHSD,D_K22,D_KLD,D_KSD,H22,HLD,HSD,
\
      BDK22,BDKLD,BDKSD,HB22,HBLD,HBSD,HK22,HKLD,HKSD,B_K22,B_KLD,B_KSD,
\
      r22kb,rldkb,rsdkb,r22bk,rldbk,rsdbk,KHB22,KHBLD,KHBSD,BHK22,BHKLD,
BHKSD,\
      KDB22,KDBLD,KDBSD,BDK22,BDKLD,BDKSD,H22h,HLDh,HSDh,H22l,HLDl,HSDl,
A,B,C,\
      b_k22,b_kLD,b_kSD,K_H22h,K_HLDh,K_HSDh,B_H22h,B_HLDh,B_HSDh,\
      HB22l,HBLDl,HBSDl,HB22h,HBLDh,HBSDh,\
      HK22l,HKLDl,HKSDl,HK22h,HKLDh,HKSDh
variate [values=1...22810]gene
“*******************************READ BASIC EXPRESSION
DATA***************************”
open ‘x:\\daves\\reciprocals\\hk 22k.txt’;ch=2
read [ch=2;print=e,s;serial=n]h22,hld,hsd,k22,kld,ksd,b22,bld,bsd
close ch=2
“ INITIAL SEED FOR RANDOM NUMBER GENERATION
      ”
scalar int,x,y
scalar [value=54321]a
    &  [value=78656]b
    &  [value=17345]c
output [width=132]1
```

| | |
|---|---|
| “ OPEN OUTPUT FILE | ” |

```
    open ‘x:\\daves\\reciprocals\\hk 22k.out’;ch=3;width=132;filetype=o
    scalar [value=16598]a
  scalar [value=*]miss
    scalar [value=1]int
  for [ntimes=250] “START OF LOOP FOR BOOTSTRAPPING”
  “ RANDOMISES ALL NINE VARIATES  ”
  for i=b22,h22,k22,bld,hld,hsd,bsd,kld,ksd;\
      j=b22,h22,k22,bld,hld,hsd,bsd,kld,ksd
            calc a=a+1
            calc xx=urand(a;22810)
            calc j=sort(i;xx)
  end for
```

| | |
|---|---|
| “ CALCULATES COMPARISONS FOR THREEOFOLD DIFFERENCES | ” |

```
“**********************************ratio of K : B
*****************************”
calc r22kb=k22/b22
  &  rldkb=kld/bld
  &  rsdkb=ksd/bsd
“**********************************ratio of B : K
*****************************”
  &  r22bk=b22/k22
  &  rldbk=bld/kld
  &  rsdbk=bsd/ksd
“***********************************ratio of H : K
*****************************”
  &  r22hk=h22/k22
  &  rldhk=hld/kld
  &  rsdhk=hsd/ksd
“********************************** ratio of H : B
*****************************”
  &  r22hb=h22/b22
  &  rldhb=hld/bld
  &  rsdhb=hsd/bsd
for k=1...22810
“********************************* B = H (within 2)
*****************************”
      for
i=r22hb,rldhb,rsdhb;j=A,B,C;m=b22,bld,bsd;n=h22,hld,hsd;o=HB22l,HBLDl,HBSDl
;p=HB22h,HBLDh,HBSDh
            if ((elem(i;k).gt.0.5).and.(elem(i;k).lt.2))
                  calc elem(j;k)=int
                        else
                  calc elem(j;k)=miss
            endif
                  calc x=elem(m;k)
                    &  y=elem(n;k)
```

| | |
|---|---|
| “ LOWEST VALUE OF B OR H | ” |

```
            if (y.gt.x).and.(elem(j;k).eq.1)
                        calc elem(o;k)=x
                  elsif (x.gt.y).and.(elem(j;k).eq.1)
                        calc elem(o;k)=y
                  else
                        calc elem(o;k)=miss
            endif
```

| | |
|---|---|
| “ HIGHEST VALUE OF B OR H | ” |

```
            if (x.gt.y).and.(elem(j;k).eq.1)
                        calc elem(p;k)=x
                  elsif (y.gt.x).and.(elem(j;k).eq.1)
                        calc elem(p;k)=y
                  else
                        calc elem(p;k)=miss
            endif
      endfor
“*********************************K = H (within 2)
*****************************”
      for
i=r22hk,rldhk,rsdhk; j=A,B,C;m=k22,kld,ksd;n=h22,hld,hsd;o=HK22l,HKLDl,HKSDl
;p=HK22h,HKLDh,HKSDh
            if ((elem(i;k).gt.0.5).and.(elem(i;k).lt.2))
                  calc elem(j;k)=int
                        else
                  calc elem(j;k)=miss
            endif
                  calc x=elem(m;k)
                    &  y=elem(n;k)
```

| | |
|---|---|
| “ LOWEST VALUE OF K OR H | ” |

```
            if (x.lt.y).and.(elem(j;k).eq.1)
                        calc elem(o;k)=x
                  elsif (y.lt.x).and.(elem(j;k).eq.1)
                        calc elem(o;k)=y
                  else
                        calc elem(o;k)=miss
            endif
```

| | |
|---|---|
| “ HIGHEST VALUE OF K OR H | ” |

```
            if (x.gt.y).and.(elem(j;k).eq.1)
                        calc elem(p;k)=x
                  elsif (y.gt.x).and.(elem(j;k).eq.1)
                        calc elem(p;k)=y
                  else
                        calc elem(p;k)=miss
            endif
      endfor
“************************************K = B (within 2)
*****************************”
      for i=r22kb,rldkb,rsdkb;j=A,B,C;m=k22,kld,ksd;n=22,bld,bsd
            if ((elem(i;k).gt.0.5).and.(elem(i;k).lt.2))
                  calc elem(j;k)=int
                        else
                  calc elem(j;k)=miss
            endif
      endfor
“**********************************K = B (highest & lowest
values)*******************”
      for
i=r22kb,rldkb,rsdkb;j=A,B,C;m=k22,kld,ksd;n=b22,bld,bsd;o=B_K22,B_KLD,B_{—}
KSD;p=b_k22,b_kLD,b_kSD
                  calc x=elem(m;k)
                    &  y=elem(n;k)
            if (x.gt.y)
                        calc elem(o;k)=x
                  else
                        calc elem(o;k)=y
            endif
            if (x.lt.y)
                        calc elem(p;k)=x
                  else
                        calc elem(p;k)=y
            endif
      endfor
endfor
“***********************************ratio of H : (K = B)  high values
**************”
calc H22h=h22/B_K22
  &  HLDh=hld/B_KLD
  &  HSDh=hsd/B_KSD
“************************************ratio of H : (K = B)  low
values***************”
calc H22l=h22/b_k22
  &  HLDl=hld/b_kLD
  &  HSDl=hsd/b_kSD
“***********************************ratio of K : (B = H)
****************************”
calc KDB22=k22/HB22h
  &  KDBLD=kld/HBLDh
  &  KDBSD=ksd/HBSDh
“***********************************ratio of B : (K = H)
****************************”
calc BDK22=b22/HK22h
  &  BDKLD=bld/HKLDh
  &  BDKSD=bsd/HKSDh
“***********************************ratio of (K = H − low values) : B
************”
calc KHB22=HK22l/b22
  &  KHBLD=HKLDl/bld
  &  KHBSD=HKSDl/bsd
“************************************ratio of (B = H) : K
***************************”
calc BHK22=HB22l/k22
  &  BHKLD=HBLDl/kld
  &  BHKSD=HBSDl/ksd
“***********************************************************************
*************”
for k=1...22810
“***********************  SEC 1 ---- K>BR-0
      ********************************”
      if
(elem(r22kb;k).gt.2).and.(elem(rldkb;k).gt.2).and.(elem(rsdkb;k).gt.2)
            calc elem(sec1;k)=int
                  else
            calc elem(sec1;k)=miss
      endif
“***********************SEC 2 ---- BR-0>K
      *********************************”
      if
(elem(r22bk;k).gt.2).and.(elem(rldbk;k).gt.2).and.(elem(rsdbk;k).gt.2)
            calc elem(sec2;k)=int
                  else
            calc elem(sec2;k)=miss
      endif
“**********************SEC 3 ---- K AND H > B (BUT K = H)
      ******************”
      if
(elem(KHB22;k).gt.2).and.(elem(KHBLD;k).gt.2).and.(elem(KHBSD;k).gt.2)
            calc elem(sec3;k)=int
                  else
            calc elem(sec3;k)=miss
      endif
“**********************SEC 4 ---- B AND H > K (BUT B = H)
      *******************”
      if
(elem(BHK22;k).gt.2).and.(elem(BHKLD;k).gt.2).and.(elem(BHKSD;k).gt.2)
            calc elem(sec4;k)=int
                  else
            calc elem(sec4;k)=miss
      endif
“***********************SEC 5 ---- K > B and H (BUT B = H)
      *********************”
      if
(elem(KDB22;k).gt.2).and.(elem(KDBLD;k).gt.2).and.(elem(KDBSD;k).gt.2)
            calc elem(sec5;k)=int
                  else
            calc elem(sec5;k)=miss
      endif
“*********************  SEC 6 ---- B > K and H (BUT K = H)
      ************************”
      if
(elem(BDK22;k).gt.2).and.(elem(BDKLD;k).gt.2).and.(elem(BDKSD;k).gt.2)
            calc elem(sec6;k)=int
                  else
            calc elem(sec6;k)=miss
      endif
“*********************  SEC 7 ---- H > B and K
*********************************”
      if
(elem(H22h;k).gt.2).and.(elem(HLDh;k).gt.2).and.(elem(HSDh;k).gt.2)
            calc elem(sec7;k)=int
                  else
            calc elem(sec7;k)=miss
      endif
“***********************SEC 8 ---- H < B and K
************************************”
      if
(elem(H221;k).lt.0.5).and.(elem(HLDl;k).lt.0.5).and.(elem(HSDl;k).lt.0.5
)
            calc elem(sec8;k)=int
                  else
            calc elem(sec8;k)=miss
      endif
endfor
“***********************************************************************
*************”
for i=sec1,sec2,sec3,sec4,sec5,sec6,sec7,sec8;\
      j=No1,No2,No3,No4,No5,No6,No7,No8;\
      k=N1,N2,N3,N4,N5,N6,N7,N8;\
      l=mv1,mv2,mv3,mv4,mv5,mv6,mv7,mv8
            calc k=nvalues(i)
              &  l=nmv(i)
              &  j=k−l
endfor
print No1,No2,No3,No4,No5,No6,No7,No8
endfor
stop
```

### Program 3

```
job ‘correlation & linear regression analysis of expression data for 30
22k chips hybrid‘
“ MID PARENT ADVANTAGE  ”
set [diagnostic=fault]
unit [32]
output [width=132]1
open ‘x:\\daves\\linreg\\all 32 hybs data.txt’;channel=2;width=250
open ‘x:\\daves\\linreg\\fprob 32 hybs lin
midp.out’;channel=3;filetype=o
variate
values=220.29,147.22,242.86,188.79,125.42,97.38,123.46,76.92,
104.48,103.61,
      270.27,200.00,137.50,184.62,127.50,66.10,110.53,97.50,
      121.26,138.46,63.53,124.56,103.23,108.33,128.74,122.89,
      94.38,158.14,230.95,143.75,248.10,186.21]mpadv
scalar [value=45454]a
for [ntimes=22810]
read [ch=2;print=*;serial=n]exp
model exp
fit [print=*]mpadv
rkeep exp;meandev=resms;tmeandev=totms;tdf=df
```

| | |
|---|---|
| calc totss=totms*31 | “= number of genotypes-1” |
| & resss=resms*30 | “= number of genotypes-2” |

```
  &  regms=(totss-resss)/1
  &  regvr=regms/resms
  &  fprob=1−(clf(regvr;1;30))
print [ch=3;iprint=*;squash=y] fprob,df
                  endfor
close ch=2
stop
```

### Program 4

```
job ‘correlation & linear regression analysis of expression data for 30
22k chips hybrid’
“ MID PARENT ADVANTAGE  ”
set [diagnostic=fault]
unit [32]
output [width=132]1
open ‘x:\\daves\\linreg\\all 32 hybs data.txt’;channel=2;width=250
open ‘x:\\daves\\linreg\\fprob 32 hybs lin midpA
boot.out’;channel=2;filetype=o
&  ‘x:\\daves\\linreg\\fprob 32 hybs lin midpB
boot.out’;channel=3;filetype=o
&  ‘x:\\daves\\linreg\\fprob 32 hybs lin midpC
boot.out’;channel=4;filetype=o
&  ‘x:\\daves\\linreg\\fprob 32 hybs lin midpD
boot.out’;channel=5;filetype=o
variate
values=220.29,147.22,242.86,188.79,125.42,97.38,123.46,76.92,
104.48,103.61,
      270.27,200.00,137.50,184.62,127.50,66.10,110.53,97.50,121.26,
      138.46,63.53,124.56,103.23,108.33,128.74,122.89,94.38,158.14,
      230.95,143.75,248.10,186.21]mpadv
scalar [value=89849]a
for [ntimes=6000]
      read [ch=2;print=*;serial=n]exp
            for [ntimes1000]
                  calc a=a+1
                  calc y=urand(a;32)
                    & pex=sort(exp;y)
                        model pex
                        fit [print=*]mpadv
                        rkeep pex;meandev=resms;tmeandev=totms
```

| | |
|---|---|
| calc totss=totms*31 | “= number of |
| genotypes-1” | |
| & resss=resms*30 | “= number of |
| genotypes-2” | |

```
                          &  regms=(totss-resss)/1
                          &  regvr=regms/resms
                          &  fprob=1−(clf(regvr;1;30))
                  print [ch=2;iprint=*;squash=yfprob
                              endfor
      print [ch=2;iprint=*;squash=y]‘:’
                  endfor
for [ntimes=6000]
      read [ch=2;print=*;serial=n]exp
      for [ntimes=1000]
                  calc a=a+1
                  calc y=urand(a;32)
                    &  pex=sort(exp;y)
                        model pex
                        fit [print=*]mpadv
                        rkeep pex;meandev=resms;tmeandev=totms
```

| | |
|---|---|
| calc totss=totms*31 | “= number of |
| genotypes-1” | |
| & resss=resms*30 | “= number of |
| genotypes-2” | |

```
                          &  regms=(totss-resss)/1
                          &  regvr=regms/resms
                          &  fprob=1−(clf(regvr;1;30))
print [ch=3;iprint=*;squash=y] fprob
                        endfor
                  print [ch=3;iprint=*;squash=y]‘:’
endfor
for [ntimes=6000]
      read [ch=2;print=*;serial=n]exp
      for [ntimes=1000]
                  calc a=a+1
                  calc y=urand(a;32)
                    &  pex=sort(exp;y)
                        model pex
                        fit [print=*]mpadv
                        rkeep pex;meandev=resms;tmeandev=totms
```

| | |
|---|---|
| calc totss=totms*31 | “= number of |
| genotypes-1” | |
| & resss=resms*30 | “= number of |
| genotypes-2” | |

```
                          &  regms=(totss-resss)/1
                          &  regvr=regms/resms
                          &  fprob=1−(clf(regvr;1;30))
print [ch=4;iprint=*;squash=y]fprob
                        endfor
                  print [ch=4;iprint=*;squash=y]‘:’
                  endfor
for [ntimes=4810]
      read [ch=2;print=*;serial=n]exp
      for [ntimes=1000]
                  calc a=a+1
                  calc y=urand(a;32)
                    &  pex=sort(exp;y)
                        model pex
                        fit [print=*]mpadv
                        rkeep pex;meandev=resms;tmeandev=totms
```

| | |
|---|---|
| calc totss=totms*31 | “= number of |
| genotypes-1” | |
| & resss=resms*30 | “= number of |
| genotypes-2” | |

```
                          &  regms=(totss-resss)/1
                          &  regvr=regms/resms
                          &  fprob=1−(clf(regvr;1;30))
print [ch=5;iprint=*;squash=y]fprob
                        endfor
                  print [ch=5;iprint=*;squash=y]‘:’
endfor
close ch=2
close ch=3
close ch=4
close ch=5
stop
```

### Program 5

```
job ‘BOOTSTRAP of linear regression analysis of expression data for 32
hybrid 22k chips ’
“  MID PARENT ADVANTAGE  ”
open  ‘x:\\daves\\linreg\\fprob 32 hybs lin midpA boot.out’;channel=2
&  ‘x:\\daves\\linreg\\fprob 32 hybs lin midpB boot.out’;channel=3
&  ‘x:\\daves\\linreg\\fprob 32 hybs lin midpC boot.out’;channel=4
&  ‘x:\\daves\\linreg\\fprob 32 hybs lin midpD boot.out’;channel=5
for [ntimes=6000]
read [ch=2;print=*;serial=y]coeff
sort [dir=d]coeff;bootstrap
calc p05minus=elem(bootstrap;950)
  & p01minus=elem(bootstrap;990)
  & p001minus=elem(bootstrap;999)
print [iprint=*;squash=y]p05minus,p01minus,p001minus
endfor
close ch=2
for [ntimes=6000]
read [ch=3;print=*;serial=y]coeff
sort [dir=d]coeff;bootstrap
calc p05minus=elem(bootstrap;950)
  & p01minus=elem(bootstrap;990)
  & p001minus=elem(bootstrap;999)
print [iprint=*;squash=y]p05minus,p01minus,p01minus
endfor
close ch=3
for [ntimes=6000]
read [ch=4;print=*;serial=y]coeff
sort [dir=d]coeff;bootstrap
calc p05minus=elem(bootstrap;950)
  & p01minus=elem(bootstrap;990)
  & p001minus=elem(bootstrap;999)
print [iprint=*;squash=y]p05minus,p01minus,p001minus
endfor
close ch=4
for [ntimes=4810]
read [ch=5;print=*;serial=y]coeff
sort [dir=d]coeff;bootstrap
calc p05minus=elem(bootstrap;950)
  & p01minus=elem(bootstrap;990)
  & p001minus=elem(bootstrap;999)
print [iprint=*;squash=y]p05minus,p01minus,p001minus
endfor
close ch=5
stop
```

### GenStat Programme 1˜Basic Regression Programme

```
job ‘Basic Regression Programme’
“  ORDER OF ORIGINAL DATA
          Ag-0 P1 Ag-0 P2 Ag-0 P3 BR-0 P1 Br-0 P2 Br-0 P3 Col-0 P1 Ct-1
P1 Ct-1 P2 Ct-1 P3 Cvi-0 P1 Cvi-0 P2 Cvi-0 P3
      Ga-0 P1 Gy-0 P1 Gy-0 P2 Gy-0 P3 Kondara P1 Kondara P2 Kondara P3
Mz-0 P1Mz-0 P2 Mz-0 P3 Nok-2 P1
      Sorbo P1  Ts-5 P1  Wt-5 P1  ms1  1  ms1  2  ms1  3  ms1  4  ms1
5 ”  “DATA ORDER IS OPTIONAL”
“  Data Input Files ”
set [diagnostic=fault]
unit [32] “NUMBER OF GENECHIPS”
output [width=132]1
open ‘x:\\daves\\linreg\\all 32 hybs data.txt’;channel=2;width=250
      “FILE WITH EXPRESSION DATA ”
open ‘x:\\daves\\linreg\\fprob 32 hybs lin
midp.out’;channel=3;filetype=o “OUTPUT FILE”
variate [values=220.29,147.22,242.86,188.79,125.42,97.38,123.46,
      76.92,104.48,103.61,270.27,200.00,137.50,184.62,\
      127.50,66.10,110.53,97.50,121.26,138.46,63.53,124.56,103.23,108.33,
128.74,122.89,94.38,158.14,\
          230.95,143.75,248.10,186.21]mpadv “TRAIT DATA”
scalar [value=45454]a
for [ntimes=22810] “NUMBER OF GENES”
read [ch=2;print=*;serial=n]exp
model exp
fit [print=*]mpadv
rkeep exp;meandev=resms;tmeandev=totms;tdf=df;“est=fd”
                      “Use to calculate Rsq Slope and Intercept”
“scalar intcpt,slope
```

| | |
|---|---|
| equate | [oldform=!(1,−1)]fd;intcpt |
| & | [oldform=!(−1,1)]fd;slope” |

“Regression Model”

| | |
|---|---|
| calc totss=totms*31 | “= number of GeneChips −1” |
| & resss=resms*30 | “= number of GeneChips −2” |

```
  & regms=(totss−resss)/1
  & regvr=regms/resms
  & fprob=1−(clf(regvr;1;30))“= number of GeneChips −2”
print
[ch=3;iprint=*;squash=y]“resms,totms,regms,resss,totss,regvr,“fprob,df,”
rsq,slope,intcpt” “OUTPUT OPTIONS”
endfor
close ch=2
stop
```

### GenStat Programme 2˜Basic Prediction Regression Programme

```
job ‘Basic Prediction Regression Programme’
set [diagnostic=fault]
unit [33]
output [width=250]1
open ‘x:\\Heterosis\\daves\\Predict\\MPH sept05\\BPH pred\\maleparhet
0.1% genes.txt’;channel=2;width=250 “INPUT FILE ”
open ‘x:\\Heterosis\\daves\\Predict\\MPH sept05\\BPH pred\\maleparhet
0.1% genes.out’;channel=3;filetype=o  “OUTPUT FILE ”
variate
[values=97.70,97.70,97.70,130.90,130.90,130.90,103.44,103.44,
103.44,138.89,\
    138.89,138.89,96.18,96.18,141.41,141.41,156.36,156.36,145.77,
145.77,150.80,\
    150.80,150.80,282.42,282.42,385.39,385.39,430.10,430.10,
430.10,205.71,205.71,\
      205.71]mpadv “TRAIT DATA”
scalar [value=68342]a
for [ntimes=706]“Number of Genes”
read [ch=2;print=*;serial=n]exp
model exp
fit [print=*]mpadv
rkeep exp;meandev=resms;tmeandev=totms;tdf=df
```

| | |
|---|---|
| calc totss=totms*32 | “= number of genotypes-1” |
| & resss=resms*31 | “= number of genotypes-2” |

```
  & regms=(totss−resss)/1
  & regvr=regms/resms
  & fprob=1−(clf(regvr;1;31))“= number of genotypes-2”
predict
[print=*;prediction=bin]mpadv;levels=!(95,105,115,125,135,145,155,
165,175,185,195,250,350,450 )“BINS, COVERING RANGE OF DATA”
```

| | |
|---|---|
| print | [ch=3;iprint=*;clprint=*;rlprint=*]bin |
| & | [ch=3;iprint=*;clprint=*]‘:’ |

endfor
close ch=2
stop

### GenStat Programme 3˜Prediction Extraction Programme

```
job ‘Prediction Extraction Programme  ’
“  MID PARENT ADVANTAGE  ”
set [diagnostic=fault]
variate
[values=95,105,115,125,135,145,155,165,175,185,195,250,350,
450]mpadv
“BIN DATA FROM PREDICTION REGRESSION PROGRAMME”
variate [values=*]miss
scalar [value=0]gene,Estimate
output [width=200]1
open ‘x:\\Heterosis\\daves\\predict\\MPH sept05\\BPH pred\\KasLLSha
MalepredprobesSept05_0.1%.txt’;channel=2;width=500  “file with
test parent data”
open ‘x:\\Heterosis\\daves\\Predict\\MPH sept05\\BPH pred\\maleparhet
0.1% genes.out’;channel=3“file with calibration data”
calc y=0
  & z=1
for [ntimes=2118] “Number of test genes X Number of Parents”
  calc y=y+1
  if y.eq.z
      read [ch=3;print*;serial=n]bin “ 11 bins = 11 values”
      calc z=z+3 “No of test parents”
      print ‘:’
  endif
      read [ch=2;print=*;serial=n]exp
          model mpadv
          fit [print=*]bin
          rkeep mpadv;meandev=resms;tmeandev=totms;tdf=df
```

| | |
|---|---|
| calc totss=totms*10 | “= number of genotypes- |
| 1” | |
| & resss=resms*9 | “= number of genotypes- |

```
2”
            & regms=(totss−resss)/1
            & regvr=regms/resms
            & fprob=1−(clf(regvr;1;9))“= number of genotypes-2”
          predict [print=*;prediction=estimate]bin;levels=exp
“should be scalar == or restricted variate”
if (estimate.lt.50) “FOR CAPPED PREDICTION, THIS IS THE LOWER
CAP”
    calc Estimate=miss
elsif (estimate.gt.455)“FOR CAPPED PREDICTION, THIS IS THE
UPPER CAP”
    calc Estimate=miss
else
    calc Estimate=estimate
endif
          calc gene=gene+1
          print
[iprint=*;rlprint=*;squash=y]gene,Estimate,estimate
endfor
close ch=2
stop
```

### GenStat Programme 4˜Basic Best Predictor Programme

```
job ‘Basic Best Predictor Programme’
text
[values=B73×B97,CML103,CML228,CML247,CML277,CML322,
              CML333,CML52,IL14H,\Ki11,Ky21,M37W,Mo18W,
NC350,NC358,Oh43,P39,Tx303,Tzi8]l “Name of Accessions”
  & [values=‘chip 1’,‘chip 2’]c “Number of Replicates”
```

| | |
|---|---|
| factor | [labels=l]line |
| & | [labels=c]chip |

factor gene
open ‘X:\\Heterosis\\daves\\Predictive gene id\\prediction
data.dat’;ch=2 “Input File”
read [ch=2;print=*;serial=n]gene,raw,line,chip,actual;frep=l,*,l,l,*
calc delta=raw-actual
& ratio=raw/actual
tabulate [class=gene;print=*]delta;means=Delta;nobs=number;var=t3
calc se_delta=sqrt(t3)/sqrt(number)
tabulate [class=gene;print=*]ratio;means=Ratio;var=t7
calc se_ratio=sqrt(t7)/sqrt(number)
print number,Delta,se_delta,Ratio,se_ratio;fieldwidth=20;dec=0,2,2,3,4
stop

### GenStat Programme 5˜Basic Linear Regression Bootstrapping Programme

```
job ‘Basic Linear Regression Bootstrapping Programme’
“  Data Input Files ”
set [diagnostic=fault]
unit [32]“NUMBER OF GENECHIPS”
output [width=132]1
open ‘x:\\daves\\linreg\\all 32 hybs data.txt’;channel=2;width=250
“FILE WITH EXPRESSION DATA ”
open ‘x:\\daves\\linreg\\fprob 32 hybs lin midpA
boot.out’;channel=2;filetype=o “OUTPUT FILES ”
&  ‘x:\\daves\\linreg\\fprob 32 hybs lin midpB
boot.out’;channel=3;filetype=o
&  ‘x:\\daves\\linreg\\fprob 32 hybs lin midpC
boot.out’;channel=4;filetype=o
&  ‘x:\\daves\\linreg\\fprob 32 hybs lin midpD
boot.out’;channel=5;filetype=o
variate
[values=220.29,147.22,242.86,188.79,125.42,97.38,123.46,76.92,104.48,
103.61,270.27,200.00,137.50,184.62,\
  127.50,66.10,110.53,97.50,121.26,138.46,63.53,124.56,103.23,108.33,
128.74,122.89,94.38,158.14,\
    230.95,143.75,248.10,186.21]mpadv “TRAIT DATA”
scalar [value=89849]a “SEED NUMBER”
for [ntimes=6000]“NUMBER OF GENES TO ANALYSE IN THIS
SECTION”
  read [ch=2;print=*;serial=n]exp
    for [ntimes=1000]“NUMBER OF RANDOMISATIONS”
      calc a=a+1
      calc y=urand(a;32)“NUMBER OF GENECHIPS TO
      RANDOMISE”
        & pex=sort(exp;y)
        model pex
        fit [print=*]mpadv
        rkeep pex;meandev=resms;tmeandev=totms
```

| | |
|---|---|
| calc totss=totms*31 | “= number of |
| genotypes-1” | |
| & resss=resms*30 | “= number of |

```
genotypes-2”
          & regms=(totss-resss)/1
          & regvr=regms/resms
          & fprob=1−(clf(regvr;1;30)) “= number of
genotypes-2”
      print
[ch=2;iprint=*;squash=y]“resms,totms,regms,resss,totss,regvr,”fprob
    endfor
  print [ch=2;iprint=*;squash=y]‘:’
endfor
for [ntimes=6000] “NUMBER OF GENES TO ANALYSE IN THIS
SECTION“
  read [ch=2;print=*;serial=n]exp
  for [ntimes=1000]“NUMBER OF RANDOMISATIONS”
      calc a=a+1
      calc y=urand(a;32)“NUMBER OF GENECHIPS TO
      RANDOMISE”
        & pex=sort(exp;y)
        model pex
        fit [print=*]mpadv
        rkeep pex;meandev=resms;tmeandev=totms
```

| | |
|---|---|
| calc totss=totms*31 | “= number of |
| genotypes-1” | |
| & resss=resms*30 | “= number of |

```
genotypes-2”
          & regms=(totss−resss)/1
          & regvr=regms/resms
          & fprob=1−(clf(regvr;1;30))“= number of
genotypes-2”
print
[ch=3;iprint=*;squash=y]“resms,totms,regms,resss,totss,regvr,”fprob
  endfor
      print [ch=3;iprint=*;squash=y]‘:’
endfor
for [ntimes=6000]“NUMBER OF GENES TO ANALYSE IN THIS
SECTION”
  read [ch=2;print=*;serial=n]exp
  for [ntimes=1000]“NUMBER OF RANDOMISATIONS”
        calc a=a+1
        calc y=urand(a;32)“NUMBER OF GENECHIPS TO
      RANDOMISE”
        & pex=sort(exp;y)
        model pex
        fit [print=*]mpadv
        rkeep pex;meandev=resms;tmeandev=totms
```

| | |
|---|---|
| calc totss=totms*31 | “= number of |
| genotypes-1” | |
| & resss=resms*30 | “= number of |

```
genotypes-2”
          & regms=(totss−resss)/1
          & regvr=regms/resms
          & fprob=1−(clf(regvr;1;30))“= number of
genotypes-2”
print
[ch=4;iprint=*;squash=y]“resms,totms,regms,resss,totss,regvr,”fprob
  endfor
      print [ch=4;iprint*;squash=y]‘:’
endfor
for [ntimes=4810]“NUMBER OF GENES TO ANALYSE IN THIS
SECTION”
  read [ch=2;print=*;serial=n]exp
  for [ntimes=1000]“NUMBER OF RANDOMISATIONS”
      calc a=a+1
      calc y=urand(a;32)“NUMBER OF GENECHIPS TO
      RANDOMISE”
        & pex=sort(exp;y)
        model pex
        fit [print=*]mpadv
        rkeep pex;meandev=resms;tmeandev=totms
```

| | |
|---|---|
| calc totss=totms*31 | “= number of |
| genotypes-1” | |
| & resss=resms*30 | “= number of |

```
genotypes-2”
          & regms=(totss−resss)/1
          & regvr=regms/resms
          & fprob=1−(clf(regvr;1;30))“= number of
genotypes-2”
print
[ch=5;iprint=*;squash=y]“resms,totms,regms,resss,totss,regvr,”fprob
  endfor
      print [ch=5;iprint=*;squash=y]‘:’
endfor
close ch=2
close ch=3
close ch=4
close ch=5
stop
```

### GenStat Programme 6˜Basic Linear Regression Bootstrapping Data Extraction Programme

```
job ‘Basic Linear Regression Bootstrapping Data Extraction Programme ’
“  DATA INPUT FILES ”
open ‘x:\\daves\\linreg\\fprob 32 hybs lin midpA boot.out’;channel=2
“INPUT FILES”
&  ‘x:\\daves\\linreg\\fprob 32 hybs lin midpB boot.out ’;channel=3
&  ‘x:\\daves\\linreg\\fprob 32 hybs lin midpC boot.out’;channel=4
&  ‘x:\\daves\\linreg\\fprob 32 hybs lin midpD boot.out’;channel=5
for [ntimes=6000]  “FIRST INPUT FILE NUMBER OF GENES”
read [ch=2;print=*;serial=y]coeff
sort [dir=a]coeff;bootstrap
calc p05plus=elem(bootstrap;50)
  & p01plus=elem(bootstrap;10)
  & p001plus=elem(bootstrap;1)
print [iprint=*;squash=y]p05plus,p01plus,p001plus “Extracts 5, 1 and
0.1% Significance levels”
endfor
close ch=2
for [ntimes=6000] “SECOND INPUT FILE NUMBER OF GENES”
read [ch=3;print=*;serial=y]coeff
sort [dir=a]coeff;bootstrap
calc p05plus=elem(bootstrap;50)
  & p01plus=elem(bootstrap;10)
  & p001plus=elem(bootstrap;1)
print [iprint=*;squash=y]p05plus,p01plus,p001plus
endfor
close ch=3
for [ntimes=6000] “THIRD INPUT FILE NUMBER OF GENES”
read [ch=4;print=*;serial=y]coeff
sort [dir=a]coeff;bootstrap
calc p05plus=elem(bootstrap;50)
  & p01plus=elem(bootstrap;10)
  & p001plus=elem(bootstrap;1)
print [iprint=*;squash=y]p05plus,p01plus,p001plus
print
[iprint=*;squash=y]“p05plus,p01plus,p001plus,”p05minus,p01minus,
p001minus
endfor
close ch=4
12 for [ntimes=4810] “FOURTH INPUT FILE NUMBER OF GENES”
read [ch=5;print=*;serial=y]coeff
sort [dir=a]coeff;bootstrap
calc p05plus=elem(bootstrap;50)
  & p01plus=elem(bootstrap;10)
  & p001plus=elem(bootstrap;1)
print [iprint=*;squash=y]p05plus,p01plus,p001plus
endfor
close ch=5
stop
```

### GenStat Programme 7˜Basic Transcriptome Remodelling Programme

```
job ‘Basic Transcriptome Remodelling Programme ’
output [width=132]1
variate [nvalues=22810]sec1,sec2,sec3,sec4,sec5,sec6,sec7,sec8,sec9,\
  DK22,DKLD,DKSD,DB22,DBLD,DBSD,DBH22,DBHLD,DBHSD,DKH22,DKHLD,DKHSD,
\
  HBK22,HBKLD,HBKSD,KBH22,KBHLD,KBHSD,D_K22,D_KLD,D_KSD,H22,HLD,HSD,
\
  BDK22,BDKLD,BDKSD,HB22,HBLD,HBSD,HK22,HKLD,HKSD,B_K22,B_KLD,B_KSD,
\
  r22kb,rldkb,rsdkb,r22bk,rldbk,rsdbk,KHB22,KHBLD,KHBSD,BHK22,BHKLD,
BHKSD,\
  KDB22,KDBLD,KDBSD,BDK22,BDKLD,BDKSD,H22h,HLDh,HSDh,H22l,HLDl,HSDl,
A,B,C,\
  b_k22,b_kLD,b_kSD,K_H22h,K_HLDh,K_HSDh,B_H22h,B_HLDh,B_HSDh,\
  HB22l,HBLDl,HBSDl,HB22h,HBLDh,HBSDh,\
  HK22l,HKLDl,HKSDl,HK22h,HKLDh,HKSDh “FILE IDENTIFIERS-IGNORE”
variate [values=1...22810]gene
“*********************************  READ BASIC EXPRESSION DATA
******************************”
open ‘x:\\daves\\reciprocals\\hb 22k.txt’;ch=2 “INPUT FILE”
read [ch=2;print=e,s;serial=n]h22,hld,hsd,k22,kld,ksd,b22,bld,bsd
close ch=2
“  INITIAL SEED FOR RANDOM NUMBER GENERATION
  ”
scalar int,x,y
```

| | |
|---|---|
| scalar | [value=54321]a |
| & | [value=78656]b |
| & | [value=17345]c |

output [width=132]1
“ OPEN OUTPUT FILE
”
open ‘x:\\daves\\reciprocals\\hk 22k.out’;ch=3;width=132;filetype=o
“OUTPUT FILE”

| | |
|---|---|
| scalar | [value=12345]a |
| scalar | [value=*]miss |
| scalar | [value=1]int |

```
“  CALCULATES COMPARISONS FOR THREEOFOLD DIFFERENCES ”
“*************************************  ratio of K : B
*****************************”
calc r22kb=k22/b22
& rldkb=kld/bld
& rsdkb=ksd/bsd
“*************************************  ratio of B : K
*****************************”
& r22bk=b22/k22
& rldbk=bld/kld
& rsdbk=bsd/ksd
“*************************************  ratio of H : K
*****************************”
& r22hk=h22/k22
& rldhk=hld/kld
& rsdhk=hsd/ksd
“*************************************  ratio of H : B
*****************************”
& r22hb=h22/b22
& rldhb=hld/bld
& rsdhb=hsd/bsd
for k=1...22810
“*************************************  B = H (within 2)
*****************************”
  for
i=r22hb,rldhb,rsdhb;j=A,B,C;m=b22,bld,bsd;n=h22,hld,hsd;o=HB22l,HBLDl,HBSDl
;p=HB22h,HBLDh,HBSDh
    if ((elem(i;k).gt.0.5).and.(elem(i;k).lt.2)) “SETS FOLD
LEVELS”
      calc elem(j;k)=int
        else
      calc elem(j;k)=miss
    endif
      calc x=elem(m;k)
        & y=elem(n;k)
“  LOWEST VALUE OF B OR H  ”
    if (y.gt.x).and.(elem(j;k).eq.1)
        calc elem(o;k)=x
      elsif (x.gt.y).and.(elem(j;k).eq.1)
        calc elem(o;k)=y
      else
        calc elem(o;k)=miss
    endif
“  HIGHEST VALUE OF B OR H  ”
    if (x.gt.y).and.(elem(j;k).eq.1)
        calc elem(p;k)=x
      elsif (y.gt.x).and.(elem(j;k).eq.1)
        calc elem(p;k)=y
      else
        calc elem(p;k)=miss
    endif
  endfor
“*************************************  K = H (within 2)
*****************************”
  for
i=r22hk,rldhk,rsdhk;j=A,B,C;m=k22,kld,ksd;n=h22,hld,hsd;o=HK22l,HKLDl,HKSDl
;p=HK22h,HKLDh,HKSDh
    if ((elem(i;k).gt.0.5).and.(elem(i;k).lt.2))
      calc elem(j;k)=int
        else
      calc elem(j;k)=miss
    endif
      calc x=elem(m;k)
        & y=elem(n;k)
“  LOWEST VALUE OF K OR H  ”
    if (x.lt.y).and.(elem(j;k).eq.1)
        calc elem(o;k)=x
      elsif (y.lt.x).and.(elem(j;k).eq.1)
        calc elem(o;k)=y
      else
        calc elem(o;k)=miss
    endif
“  HIGHEST VALUE OF K OR H  ”
    if (x.gt.y).and.(elem(j;k).eq.1)
        calc elem(p;k)=x
      elsif (y.gt.x).and.(elem(j;k).eq.1)
        calc elem(p;k)=y
      else
        calc elem(p;k)=miss
    endif
  endfor
“*************************************  K = B (within 2)
*****************************”
  for i=r22kb,rldkb,rsdkb;j=A,B,C;m=k22,kld,ksd;n=b22,bld,bsd
    if ((elem(i;k).gt.0.5).and.(elem(i;k).lt.2))
      calc elem(j;k)=int
        else
      calc elem(j;k)=miss
    endif
  endfor
“*************************************  K = B (highest & lowest values)
*************************”
  for
i=r22kb,rldkb,rsdkb;j=A,B,C;m=k22,kld,ksd;n=b22,bld,bsd;o=B_K22,B_KLD,B_{—}
KSD;p=b_k22,b_kLD,b_kSD
      calc x=elem(m;k)
        & y=elem(n;k)
    if (x.gt.y)
        calc elem(o;k)=x
      else
        calc elem(o;k)=y
    endif
    if (x.lt.y)
        calc elem(p;k)=x
      else
        calc elem(p;k)=y
    endif
  endfor
endfor
“*************************************  ratio of H : (K = B) high
values  **************”
calc H22h=h22/B_K22
  & HLDh=hld/B_KLD
  & HSDh=hsd/B_KSD
“*************************************  ratio of H : (K = B) low
values  ***************”
calc H22l=h22/b_k22
  & HLDl=hld/b_kLD
  & HSDl=hsd/b_kSD
“*************************************  ratio of K : (B = H)
****************************”
calc KDB22=k22/HB22h
  & KDBLD=kld/HBLDh
  & KDBSD=ksd/HBSDh
“*************************************  ratio of B : (K = H)
****************************”
calc BDK22=b22/HK22h
  & BDKLD=bld/HKLDh
  & BDKSD=bsd/HKSDh
“*************************************  ratio of (K = H − low values) :
B  ************”
calc KHB22=HK22l/b22
  & KHBLD=HKLDl/bld
  & KHBSD=HKSDl/bsd
“*************************************  ratio of (B = H) : K
****************************”
calc BHK22=HB22l/k22
  & BHKLD=HBLDl/kld
  & BHKSD=HBSDl/ksd
“***********************************************************************
****************”
for k=1...22810
“***********************  SEC 1 ---- K>BR-0
  ********************************”
  if
(elem(r22kb;k).gt.2).and.(elem(rldkb;k).gt.2).and.(elem(rsdkb;k).gt.2)
    calc elem(sec1;k)=int
      else
    calc elem(sec1;k)=miss
  endif
“***********************  SEC 2 ---- BR-0>K
  *********************************”
  if
(elem(r22bk;k).gt.2).and.(elem(rldbk;k).gt.2).and.(elem(rsdbk;k).gt.2)
    calc elem(sec2;k)=int
      else
    calc elem(sec2;k)=miss
  endif
“***********************  SEC 3 ---- K AND H > B (BUT K = H)
  ******************”
  if
(elem(KHB22;k).gt.2).and.(elem(KHBLD;k).gt.2).and.(elem(KHBSD;k).gt.2)
    calc elem(sec3;k)=int
      else
    calc elem(sec3;k)=miss
  endif
“***********************  SEC 4 ---- B AND H > K (BUT B = H)
  *******************”
  if
(elem(BHK22;k).gt.2).and.(elem(BHKLD;k).gt.2).and.(elem(BHKSD;k).gt.2)
    calc elem(sec4;k)=int
      else
    calc elem(sec4;k)=miss
  endif
“***********************  SEC 5 ---- K > B and H (BUT B = H)
  *********************”
  if
(elem(KDB22;k).gt.2).and.(elem(KDBLD;k).gt.2).and.(elem(KDBSD;k).gt.2)
    calc elem(sec5;k)=int
      else
    calc elem(sec5;k)=miss
  endif
“***********************  SEC 6 ---- B > K and H (BUT K = H)
  ************************”
  if
(elem(BDK22;k).gt.2).and.(elem(BDKLD;k).gt.2).and.(elem(BDKSD;k).gt.2)
    calc elem(sec6;k)=int
      else
    calc elem(sec6;k)=miss
  endif
“***********************  SEC 7 ---- H > B and K
  *********************************”
  if
(elem(H22h;k).gt.2).and.(elem(HLDh;k).gt.2).and.(elem(HSDh;k).gt.2)
    calc elem(sec7;k)=int
      else
    calc elem(sec7;k)=miss
  endif
“***********************  SEC 8 ---- H < B and K
  ************************************”
  if
(elem(H22l;k).lt.0.5).and.(elem(HLDl;k).lt.0.5).and.(elem(HSDl.k).lt.0.5)
    calc elem(sec8;k)=int
      else
    calc elem(sec8;k)=miss
  endif
endfor
“***********************************************************************
******************************”
print gene,sec1,sec2,sec3,sec4,sec5,sec6,sec7,sec8
for i=sec1,sec2,sec3,sec4,sec5,sec6,sec7,sec8;\
  j=No1,No2,No3,No4,No5,No6,No7,No8;\
  k=N1,N2,N3,N4,N5,N6,N7,N8;\
  l=mv1,mv2,mv3,mv4,mv5,mv6,mv7,mv8
    calc k=nvalues(i)
      & l=nmv(i)
      & j=k−l
endfor
print No1,No2,No3,No4,No5,No6,No7,No8
stop
```

### GenStat Programme 8˜Dominance Pattern Programme

```
job ‘Dominance Pattern Programme’
scalar AG1M,AG1,AG2M,AG2,AG3M,AG3,CT1M,CT1,CT2M,CT2,CT3M,CT3,\
  CV1M,CV1,CV2M,CV2,CV3M,CV3,GY1M,GY1,GY2M,GY2,GY3M,GY3,K1M,\
  K1,K2M,K2,K3M,K3,MZ1M,MZ1,MZ2M,MZ2,MZ3M,MZ3,BK1M,BK1,BK2M,\
  BK2,BK3M,BK3,KB1M,KB1,KB2M,KB2,KB3M,KB3 “genotypes
names/bins for calculations”
```

| | |
|---|---|
| scalar [value=48]a | “starting value |
| for equate directive” | |
| & [value=12345]seed | “seed value for |
| randomisation” | |
| & [value=*]miss | “missing value” |
| & | |

```
[value=0]AGEQ,AGGT,AGLT,CTEQ,CTGT,CTLT,CVEQ,CVGT,CVLT,GYEQ,GYGT,GYLT,\
    KEQ,KGT,KLT,MZEQ,MZGT,MZLT,BKEQ,BKGT,BKLT,KBEQ,KBGT,KBLT
  “scalars for total signifiant genes”
variate  [nvalues=48]gene
  &  [nvalues=22810]AG,CT,CV,GY,K,MZ,BK,KB
  &
[nvalues=3]eqAG,gtAG,ltAG,eqCT,gtCT,ltCT,eqCV,gtCV,ltCV,eqGY,gtGY,ltGY,\
    eqK,gtK,ltK,eqMZ,gtMZ,ltMZ,eqBK,gtBK,ltBK,eqKB,gtKB,ltKB
output [width=400]1
“  OPEN OUTPUT FILE
  ”
open ‘x:\\daves\\Dominance method\\dom 2
fold.out’;ch=3;width=300;filetype=o “OUTPUT FILE”
open ‘x:\\daves\\Dominance method\\Expression datab.txt’;ch=2;width=500
“INPUT FILE”
read [ch=2;print=e,s;serial=n]EXP
close ch=2
```

| | |
|---|---|
| for i=1...22810 | “reads through |
| data gene by gene” | |
| calc a=a−48 | “incremnets data” |
| equate [oldformat=!(a,48)]EXP;gene | “puts data in one |

```
variate per gene”
  “randomises variate for subsequent calculations
  calc nege=rand(gene;seed)”
“places data for 1 gene at a time into variate bins”
  for
geno=AG1M,AG1,AG2M,AG2,AG3M,AG3,CT1M,CT1,CT2M,CT2,CT3M,CT3,CV1M,CV1,CV2M,
CV2,CV3M,CV3,\
  GY1M,GY1,GY2M,GY2,GY3M,GY3,K1M,K1,K2M,K2,K3M,K3,MZ1M,MZ1,MZ2M,MZ2,
NZ3M,MZ3,BK1M,BK1,\
    BK2M,BK2,BK3M,BK3,KB1M,KB1,KB2M,KB2,KB3M,KB3;\
    j=1...48
    calc geno=elem(gene;j)
  endfor
“calculation of ratios”
  for
genom=AG1M,AG2M,AG3M,CT1M,CT2M,CT3M,CV1M,CV2M,CV3M,GY1M,GY2M,GY3M,K1M,\
    K2M,K3M,MZ1M,MZ2M,MZ3M,BK1M,BK2M,BK3M,KB1M,KB2M,KB3M;\
    genoh=AG1,AG2,AG3,CT1,CT2,CT3,CV1,CV2,CV3,GY1,GY2,GY3,\
    K1,K2,K3,MZ1,MZ2,MZ3,BK1,BK2,BK3,KB1,KB2,KB3;\
ratio=rAG1,rAG2,rAG3,rCT1,rCT2,rCT3,rCV1,rCV2,rCV3,rGY1,rGY2,rGY3,\
    rK1,rK2,rK3,rMZ1,rMZ2,rMZ3,rBK1,rBK2,rBK3,rKB1,rKB2,rKB3;\
hEQmp=eqAG,eqAG,eqAG,eqCT,eqCT,eqCT,eqCV,eqCV,eqCV,eqGY,eqGY,eqGY,\
    eqK,eqK,eqK,eqMZ,eqMZ,eqMZ,eqBK,eqBK,eqBK,eqKB,eqKB,eqKB;\
hGTmp=gtAG,gtAG,gtAG,gtCT,gtCT,gtCT,gtCV,gtCV,gtCV,gtGY,gtGY,gtGY,\
    gtK,gtK,gtK,gtMZ,gtMZ,gtMZ,gtBK,gtBK,gtBK,gtKB,gtKB,gtKB;\
hLTmp=ltAG,ltAG,ltAG,ltCT,ltCT,ltCT,ltCV,ltCV,ltCV,ltGY,ltGY,ltGY,\
    ltK,ltK,ltK,ltMZ,ltMZ,ltMZ,ltBK,ltBK,ltBK,ltKB,ltKB,ltKB;\
    k=1,2,3,1,2,3,1,2,3,1,2,3,1,2,3,1,2,3,1,2,3,1,2,3
      calc ratio=genoh/genom “calculates
ratios”
        calc heqmp=miss
          & hgtmp=miss  “sets default flag
values”
          & hltmp=miss
```

| | |
|---|---|
| if (ratio.ge.0.5).and.(ratio.le.2) | “SETS FOLD LEVEL” |
| calc heqmp=1 | |
| elsif (ratio.gt.2) | “SETS UPPER FOLD LEVEL” |
| calc hgtmp=1 | |
| elsif (ratio.lt.0.5) | “SETS LOWER FOLD LEVEL” |

```
        calc hltmp=1
      else
        calc heqmp=miss
          & hgtmp=miss
          & hltmp=miss
    endif
          calc elem(hEQmp;k)=heqmp
            & elem(hGTmp;k)=hgtmp
            & elem(hLTmp;k)=hltmp
  endfor
    for
X=eqAG,gtAG,ltAG,eqCT,gtCT,ltCT,eqCV,gtCV,ltCV,eqGY,gtGY,ltGY,\
  eqK,gtK,ltK,eqMZ,gtMZ,ltMZ,eqBK,gtBK,ltBK,eqKB,gtKB,ltKB;\
Y=AGeq,AGgt,AGlt,CTeq,CTgt,CTlt,CVeq,CVgt,CVlt,GYeq,GYgt,GYlt,\
  Keq,Kgt,Klt,MZeq,MZgt,MZlt,BKeq,BKgt,BKlt,KBeq,KBgt,KBlt;\
Z=AGEQ,AGGT,AGLT,CTEQ,CTGT,CTLT,CVEQ,CVGT,CVLT,GYEQ,GYGT,GYLT,\
  KEQ,KGT,KLT,MZEQ,MZGT,MZLT,BKEQ,BKGT,BKLT,KBEQ,KBGT,KBLT
        calc Y=sum(X)
          if Y.eq.3
            calc Y=1
          else
            calc Y=0
          endif
        calc Z=Z+Y
    endfor
  print
[ch=3;iprint=*;squash=y]AGeq,AGgt,AGlt,CTeq,CTgt,CTlt,CVeq,CVgt,CVlt,GYeq,
GYgt,GYlt,\
  Keq,Kgt,Klt,MZeq,MZgt,MZlt,BKeq,BKgt,BKlt,KBeq,KBgt,KBlt;fieldwidth=8;
dec=0
endfor
stop
```

### GenStat Programme 9˜Dominance Permutation Programme

```
job ‘Dominance Permutation Programme’
scalar AG1M,AG1,AG2M,AG2,AG3M,AG3,CT1M,CT1,CT2M,CT2,CT3M,CT3,\
  CV1M,CV1,CV2M,CV2,CV3M,CV3,GY1M,GY1,GY2M,GY2,GY3M,GY3,K1M,\
  K1,K2M,K2,K3M,K3,MZ1M,MZ1,MZ2M,MZ2,MZ3M,MZ3,BK1M,BK1,BK2M,\
```

| | |
|---|---|
| BK2,BK3M,BK3,KB1M,KB1,KB2M,KB2,KB3M,KB3 | “genotypes |
| names/bins for calculations” | |
| scalar [value=48]a | “starting value |
| for equate directive” | |
| & [value=12345]seed | “seed value for |
| randomisation” | |

```
  &
[value=0]AGEQ,AGGT,AGLT,CTEQ,CTGT,CTLT,CVEQ,CVGT,CVLT,GYEQ,GYGT,GYLT,\
    KEQ,KGT,KLT,MZEQ,MZGT,MZLT,BKEQ,BKGT,BKLT,KBEQ,KBGT,KBLT
  “scalars for total signifiant genes”
variate [nvalues=48]gene
  & [nvalues=22810]AG,CT,CV,GY,K,MZ,BK,KB
  &
[nvalues=3]eqAG,gtAG,ltAG,eqCT,gtCT,ltCT,eqCV,gtCV,ltCV,eqGY,gtGY,ltGY,\
    eqK,gtK,ltK,eqMZ,gtMZ,ltMZ,eqBK,gtBK,ltBK,eqKB,gtKB,ltKB
output [width=400]1
“  OPEN OUTPUT FILE
”
open ‘x:\\daves\\Dominance
method\\domperm.out’;ch=3;width=300;filetype=o “OUTPUT FILE”
open ‘x:\\daves\\Dominance method\\Expression datab.txt’;ch=2;width=500
“INPUT FILE”
read [ch=2;print=e,s;serial=n]EXP
close ch=2
```

| | |
|---|---|
| for [ntimes=1000] | “NUMBER OF |
| PERMUTATIONS” | |
| calc seed=seed+1 | |
| for [ntimes=22810] “NUMBER OF GENES” | |

“***********************************************************************
***********”
calc a=a−48

| | |
|---|---|
| equate [oldformat=!(a,48)]EXP;gene | “puts data |

```
in one variate per gene”
    “randomises variate for subsequent calculations”
    calc y=urand(seed;48)
      & nege=sort(gene;y)
  “places data for 1 gene at a time into variate bins”
    for
geno=AG1M,AG1,AG2M,AG2,AG3M,AG3,CT1M,CT1,CT2M,CT2,CT3M,CT3,CV1M,CV1,CV2M
,CV2,CV3M,CV3,\
    GY1M,GY1,GY2M,GY2,GY3M,GY3,K1M,K1,K2M,K2,K3M,K3,MZ1M,MZ1,MZ2M,MZ2,
MZ3M,MZ3,BK1M,BK1,\
      BK2M,BK2,BK3M,BK3,KB1M,KB1,KB2M,KB2,KB3M,KB3;\
      j=1...48
      calc geno=elem(nege;j)
    endfor
“***********************************************************************
********”
  “calculation of ratios”
    for
genom=AG1M,AG2M,AG3M,CT1M,CT2M,CT3M,CV1M,CV2M,CV3M,GY1M,GY2M,GY3M,K1M,\
      K2M,K3M,MZ1M,MZ2M,MZ3M,BK1M,BK2M,BK3M,KB1M,KB2M,KB3M;\
      genoh=AG1,AG2,AG3,CT1,CT2,CT3,CV1,CV2,CV3,GY1,GY2,GY3,\
    K1,K2,K3,MZ1,MZ2,MZ3,BK1,BK2,BK3,KB1,KB2,KB3;\
ratio=rAG1,rAG2,rAG3,rCT1,rCT2,rCT3,rCV1,rCV2,rCV3,rGY1,rGY2,rGY3,\
  rK1,rK2,rK3,rMZ1,rMZ2,rMZ3,rBK1,rBK2,rBK3,rKB1,rKB2,rKB3;\
hEQmp=eqAG,eqAG,eqAG,eqCT,eqCT,eqCT,eqCV,eqCV,eqCV,eqGY,eqGY,eqGY,\
  eqK,eqK,eqK,eqMZ,eqMZ,eqMZ,eqBK,eqBK,eqBK,eqKB,eqKB,eqKB;\
hGTmp=gtAG,gtAG,gtAG,gtCT,gtCT,gtCT,gtCV,gtCV,gtCV,gtGY,gtGY,gtGY,\
  gtK,gtK,gtK,gtMZ,gtMZ,gtMZ,gtBK,gtBK,gtBK,gtKB,gtKB,gtKB;\
hLTmp=ltAG,ltAG,ltAG,ltCT,ltCT,ltCT,ltCV,ltCV,ltCV,ltGY,ltGY,ltGY,\
  ltK,ltK,ltK,ltMZ,ltMZ,ltMZ,ltBK,ltBK,ltBK,ltKB,ltKB,ltKB;\
      k=1,2,3,1,2,3,1,2,3,1,2,3,1,2,3,1,2,3,1,2,3,1,2,3
        calc ratio=genoh/genom “calculates
ratios”
          calc heqmp=0
```

| | |
|---|---|
| & hgtmp=0 | “sets |
| default flag values” | |
| & hltmp=0 | |
| if (ratio.le.2.0).and.(ratio.ge.0.5) | “SETS FOLD |
| LEVEL” | |
| calc heqmp=1 | |
| elsif (ratio.gt.2.0) | “SETS UPPER |
| FOLD LEVEL” | |
| calc hgtmp=1 | |
| elsif (ratio.lt.0.5) | “SETS LOWER |

```
FOLD LEVEL”
          calc hltmp=1
        else
          calc heqmp=0
            & hgtmp=0
            & hltmp=0
      endif
          calc elem(hEQmp;k)=heqmp
            & elem(hGTmp;k)=hgtmp
            & elem(hLTmp;k)=hltmp
    endfor
      for
X=eqAG,gtAG,ltAG,eqCT,gtCT,ltCT,eqCV,gtCV,ltCV,eqGY,gtGY,ltGY,\
  eqK,gtK,ltK,eqMZ,gtMZ,ltMZ,eqBK,gtBK,ltBK,eqKB,gtKB,ltKB;\
Y=AGeq,AGgt,AGlt,CTeq,CTgt,CTlt,CVeq,CVgt,CVlt,GYeq,GYgt,GYlt,\
  Keq,Kgt,Klt,MZeq,MZgt,MZlt,BKeq,BKgt,BKlt,KBeq,KBgt,KBlt;\
Z=AGEQ,AGGT,AGLT,CTEQ,CTGT,CTLT,CVEQ,CVGT,CVLT,GYEQ,GYGT,GYLT,\
  KEQ,KGT,KLT,MZEQ,MZGT,MZLT,BKEQ,BKGT,BKLT,KBEQ,KBGT,KBLT
          calc Y=sum(X)
            if Y.eq.3
              calc Y=1
            else
              calc Y=0
            endif
          calc Z=Z+Y
      endfor
  endfor
  print
[ch=3;iprint=*;squash=y]AGEQ,AGGT,AGLT,CTEQ,CTGT,CTLT,CVEQ,CVGT,CVLT,GYEQ
,GYGT,GYLT,\
  KEQ,KGT,KLT,MZEQ,MZGT,MZLT,BKEQ,BKGT,BKLT,KBEQ,KBGT,KBLT;fieldwidth
=8; dec=0
  for list=AGEQ,AGGT,AGLT,CTEQ,CTGT,CTLT,CVEQ,CVGT,CVLT,GYEQ,GYGT,GYLT,\
    KEQ,KGT,KLT,MZEQ,MZGT,MZLT,BKEQ,BKGT,BKLT,KBEQ,KBGT,KBLT
      calc list=0
  endfor
endfor
stop
```

### GenStat Programme 10˜Transcriptome Remodelling Bootstrap Programme

```
job ‘Transcriptome Remodelling Bootstrap Programme’
output [width=132]1
variate [nvalues=22810]sec1,sec2,sec3,sec4,sec5,sec6,sec7,sec8,sec9,\
  DK22,DKLD,DKSD,DB22,DBLD,DBSD,DBH22,DBHLD,DBHSD,DKH22,DKHLD,DKHSD,
\
  HBK22,HBKLD,HBKSD,KBH22,KBHLD,KBHSD,D_K22,D_KLD,D_KSD,H22,HLD,HSD,
\
  BDK22,BDKLD,BDKSD,HB22,HBLD,HBSD,HK22,HKLD,HKSD,B_K22,B_KLD,B_KSD,
\
  r22kb,rldkb,rsdkb,r22bk,rldbk,rsdbk,KHB22,KHBLD,KHBSD,BHK22,BHKLD,
BHKSD,\
  KDB22,KDBLD,KDBSD,BDK22,BDKLD,BDKSD,H22h,HLDh,HSDh,H22l,HLDl,HSDl,
A,B,C,\
  b_k22,b_kLD,b_kSD,K_H22h,K_HLDh,K_HSDh,B_H22h,B_HLDh,B_HSDh,\
  HB22l,HBLDl,HBSDl,HB22h,HBLDh,HBSDh,\
  HK22l,HKLDl,HKSDl,HK22h,HKLDh,HKSDh  “FILE IDENTIFIERS-IGNORE”
variate [values=1...22810]gene
“*********************************  READ BASIC EXPRESSION DATA
******************************”
open ‘x:\\daves\\reciprocals\\hb 22k.txt’;ch=2 “INPUT FILE”
read [ch=2;print=e,s;serial=n]h22,hld,hsd,k22,kld,ksd,b22,bld,bsd
close ch=2
“  INITIAL SEED FOR RANDOM NUMBER GENERATION
  ”
scalar int,x,y
```

| | |
|---|---|
| scalar | [value=54321]a |
| & | [value=78656]b |
| & | [value=17345]c |
| output | [width=132]1 |

```
“  OPEN OUTPUT FILE
  ”
    open ‘x:\\daves\\reciprocals\\hb 22k.out’;ch=3;width=132;filetype=o
“OUTPUT FILE”
    scalar [value=17589]a
  scalar [value=*]miss
    scalar [value=1]int
      “START OF LOOP FOR BOOTSTRAPPING”
    for [ntimes=1000] “NUMBER OF RANDOMISATIONS”
    “  RANDOMISES ALL NINE VARIATES  ”
```

| | |
|---|---|
| for | i=b22,h22,k22,bld,hld,hsd,bsd,kld,ksd;\ |
| | j=b22,h22,k22,bld,hld,hsd,bsd,kld,ksd |

```
    calc a=a+1
    calc xx=urand(a;22810)“NUMBER OF GENES”
    calc j=sort(i;xx)
    endfor
“  CALCULATES COMPARISONS FOR THREEOFOLD DIFFERENCES  ”
“*************************************  ratio of K : B
*****************************”
calc r22kb=k22/b22
    & rldkb=kld/bld
    & rsdkb=ksd/bsd
“*************************************  ratio of B : K
*****************************”
    & r22bk=b22/k22
    & rldbk=bld/kld
    & rsdbk=bsd/ksd
“*************************************  ratio of H : K
*****************************”
    & r22hk=h22/k22
    & rldhk=hld/kld
    & rsdhk=hsd/ksd
“*************************************  ratio of H : B
*****************************”
    & r22hb=h22/b22
    & rldhb=hld/bld
    & rsdhb=hsd/bsd
for k=1...22810
“*************************************  B = H (within 2)
*****************************”
  for
i=r22hb,rldhb,rsdhb;j=A,B,C;m=b22,bld,bsd;n=h22,hld,hsd;o=HB22l,HBLDl,HBSDl;
p=HB22h,HBLDh,HBSDh
    if ((elem(i;k).gt.0.5).and.(elem(i;k).lt.2))“SETS FOLD
LEVELS”
      calc elem(j;k)=int
        else
      calc elem(j;k)=miss
    endif
      calc x=elem(m;k)
        & y=elem(n;k)
“  LOWEST VALUE OF B OR H  ”
    if (y.gt.x).and.(elem(j;k).eq.1)
        calc elem(o;k)=x
      elsif (x.gt.y).and.(elem(j;k).eq.1)
        calc elem(o;k)=y
      else
        calc elem(o;k)=miss
    endif
“  HIGHEST VALUE OF B OR H  ”
    if (x.gt.y).and.(elem(j;k).eq.1)
        calc elem(p;k)=x
      elsif (y.gt.x).and.(elem(j;k).eq.1)
        calc elem(p;k)=y
      else
        calc elem(p;k)=miss
    endif
  endfor
“*************************************  K = H (within 2)
*****************************”
    for
i=r22hk,rldhk,rsdhk;j=A,B,C;m=k22,kld,ksd;n=h22,hld,hsd,o=HK22l,HKLDl,HKSDl;
p=HK22h,HKLDh,HKSDh
    if ((elem(i;k).gt.0.5).and.(elem(i;k).lt.2))
      calc elem(j;k)=int
        else
      calc elem(j;k)=miss
    endif
      calc x=elem(m;k)
        & y=elem(n;k)
“  LOWEST VALUE OF K OR H  ”
    if (x.lt.y).and.(elem(j;k).eq.1)
        calc elem(o;k)=x
      elsif (y.lt.x).and.(elem(j;k).eq.1)
        calc elem(o;k)=y
      else
        calc elem(o;k)=miss
    endif
“  HIGHEST VALUE OF K OR H  ”
    if (x.gt.y).and.(elem(j;k).eq.1)
        calc elem(p;k)=x
      elsif (y.gt.x).and.(elem(j;k).eq.1)
        calc elem(p;k)=y
      else
        calc elem(p;k)=miss
    endif
  endfor
“*************************************  K = B (within 2)
*****************************”
  for i=r22kb,rldkb,rsdkb;j=A,B,C;m=k22,kld,ksd;n=b22,bld,bsd
    if ((elem(i;k).gt.0.5).and.(elem(i;k).lt.2))
      calc elem(j;k)=int
        else
      calc elem(j;k)=miss
    endif
  endfor
“*************************************  K = B (highest & lowest values)
*************************”
  for
i=r22kb,rldkb,rsdkb;j=A,B,C;m=k22,kld,ksd;n=b22,bld,bsd;o=B_K22,B_KLD,B_{—KSD;}
p=b_k22,b_kLD,b_kSD
      calc x=elem(m;k)
        & y=elem(n;k)
    if (x.gt.y)
        calc elem(o;k)=x
      else
        calc elem(o;k)=y
    endif
    if (x.lt.y)
        calc elem(p;k)=x
      else
        calc elem(p;k)=y
    endif
  endfor
endfor
“*************************************  ratio of H : (K = B) high
values  **************”
calc H22h=h22/B_K22
    & HLDh=hld/B_KLD
    & HSDh=hsd/B_KSD
“*************************************  ratio of H : (K = B) low
values  ***************”
calc H22l=h22/b_k22
    & HLDl=hld/b_kLD
    & HSDl=hsd/b_kSD
“*************************************  ratio of K : (B = H)
****************************”
calc KDB22=k22/HB22h
    & KDBLD=kld/HBLDh
    & KDBSD=ksd/HBSDh
“*************************************  ratio of B : (K = H)
****************************”
calc BDK22=b22/HK22h
    & BDKLD=bld/HKLDh
    & BDKSD=bsd/HKSDh
“*************************************  ratio of (K = H − low values) :
B  ************”
calc KHB22=HK22l/b22
    & KHBLD=HKLDl/bld
    & KHBSD=HKSDl/bsd
“*************************************  ratio of (B = H) : K
****************************”
calc BHK22=HB22l/k22
    & BHKLD=HBLDl/kld
    & BHKSD=HBSDl/ksd
“***********************************************************************
****************”
for k=1...22810
“***********************  SEC 1 ---- K>BR-0
  ********************************”
  if
(elem(r22kb;k).gt.2).and.(elem(rldkb;k).gt.2).and.(elem(rsdkb;k).gt.2)
    calc elem(sec1;k)=int
      else
    calc elem(sec1;k)=miss
  endif
“***********************  SEC 2 ---- BR-0>K
  *********************************”
  if
(elem(r22bk;k).gt.2).and.(elem(rldbk;k).gt.2).and.(elem(rsdbk;k).gt.2)
    calc elem(sec2;k)=int
      else
    calc elem(sec2;k)=miss
  endif
“***********************  SEC 3 ---- K AND H > B (BUT K = H)
  ******************”
  if
(elem(KHB22;k).gt.2).and.(elem(KHBLD;k).gt.2).and.(elem(KHBSD;k).gt.2)
    calc elem(sec3;k)=int
      else
    calc elem(sec3;k)=miss
  endif
“***********************  SEC 4 ---- B AND H > K (BUT B = H)
  *******************”
  if
(elem(BHK22;k).gt.2).and.(elem(BHKLD;k).gt.2).and.(elem(BHKSD;k).gt.2)
    calc elem(sec4;k)=int
      else
    calc elem(sec4;k)=miss
  endif
“***********************  SEC 5 ---- K > B and H (BUT B = H)
  *********************”
  if
(elem(KDB22;k).gt.2).and.(elem(KDBLD;k).gt.2).and.(elem(KDBSD;k).gt.2)
    calc elem(sec5;k)=int
      else
    calc elem(sec5;k)=miss
  endif
“***********************  SEC 6 ---- B > K and H (BUT K = H)
  ************************”
  if
(elem(BDK22;k).gt.2).and.(elem(BDKLD;k).gt.2).and.(elem(BDKSD.k).gt.2)
    calc elem(sec6;k)=int
      else
    calc elem(sec6;k)=miss
  endif
“***********************  SEC 7 ---- H > B and K
  *********************************”
  if
(elem(H22h;k).gt.2).and.(elem(HLDh;k).gt.2).and.(elem(HSDh;k).gt.2)
    calc elem(sec7;k)=int
      else
    calc elem(sec7;k)=miss
  endif
“***********************  SEC 8 ---- H < B and K
  ************************************”
  if
(elem(H22l;k).lt.0.5).and.(elem(HLDl;k).lt.0.5).and.(elem(HSDl;k).lt.0.5)
    calc elem(sec8;k)=int
      else
    calc elem(sec8;k)=miss
  endif
endfor
“***********************************************************************
******************************”
“print gene,sec1,sec2,sec3,sec4,sec5,sec6,sec7,sec8”
for i=sec1,sec2,sec3,sec4,sec5,sec6,sec7,sec8;\
  j=No1.No2,No3,No4,No5,No6,No7,No8;\
  k=N1,N2,N3,N4,N5,N6,N7,N8;\
  l=mv1,mv2,mv3,mv4,mv5,mv6,mv7,mv8
    calc k=nvalues (i)
      & l=nmv(i)
      & j=k−l
endfor
print No1,No2,No3,No4,No5,No6,No7,No8
endfor
stop
```

### REFERENCES

1 R. H. Moll, W. S. Salhuana, H. F. Robinson, Crop Sci 2, 197 (1962).
2 J. H. Xiao, J. M. Li, L. P. Yuan, S. D. Tanksley, Genetics 140, 745 (1995)
3 M. A. Kosba, Beitr Trop Landwirtsch Veterinarmed 16, 187 (1978)
4 K. E. Gregory, L. V. Cundiff, R. M. Koch, J. Anim Sci. 70, 2366 (1992)
5 G. H. Shull, Am Breed Assoc 4, 296 (1908)
6 D. E. Comings, J. P. MacMurray, Molecular Genetics and Metabolism 71, 19 (2000)
7 Meyer, R. C., et al. 2004 Plant Physiol. 134: 1813-1823
8 Piepho, Hans-Peter (2005) Genetics 171:359-364
9 Stuber, C. W., et al. (1992) Genetics 132:823-839
10 C. B. Davenport, Science 28, 454 (1908)
11 E. M. East, Reports of the Connecticut agricultural experiment station for years 1907-1908 419 (1908).
12 J. B. Hollick, V. L. Chandler, Genetics 150, 891 (1998)
13 D. A. Fasoula, V. A. Fasoula, Plant Breeding Reviews 14, 89 (1997)
14 J. P. Hua et al., Proceedings of the National Academy of Sciences of the United States of America 100, 2574 (2003)
15 S. W. Omholt, E. Plahte, L. Oyehaug, K. F. Xiang, Genetics 155, 969 (2000)
16 Duvick, D. N. (1999). Genetic diversity and heterosis. In: Coors, C. G. and Pandey, S. (Eds.) Genetics and exploitation of heterosis in crops. American Society of Agronomy, Madison 293-304
17 Melchinger, A. E. 1999 Genetic diversity and heterosis. In: Coors, C. G. and Pandey, S. (Eds.) Genetics and exploitation of heterosis in crops. American Society of Agronomy, Madison 99-1118.
18 Moll, R. H., et al. 1965. Genetics 52 139-144.
19 Stokes, D., et al. Euphytica in press. 2007
20 Melchinger, A. E., et al. (1990) TAG Theoretical and Applied Genetics (Historical Archive) 80:488-496
21 Xiao, J., et al. (1996) TAG Theoretical and Applied Genetics 92: 637-643
22 Fabrizius, M. A., et al. (1998). Crop Science 38:1108-1112.
23 L. Z. Xiong, G. P. Yang, C. G. Xu, Q. F. Zhang, M. A. S. Maroof, Molecular Breeding 4, 129 (1998)
24 Q. X. Sun, Z. F. Ni, Z. Y. Liu, Euphytica 106, 117 (1999)
25 Z. Ni, Q. Sun, Z. Liu, L. Wu, X. Wang, Molecular and General Genetics 263, 934 (2000)
26 L. M. Wu, Z. F. Ni, F. R. Meng, Z. Lin, Q. X. Sun, Molecular Genetics and Genomics 270, 281 (2003)
27 Auger et al. Genetics 169:389-397 2005
28 Sun, Q. X., et al. 2004 Plant Science 166, 651-657
29 M. Guo et al., Plant Cell 16, 1707 (2004) Vuylsteke et al. Genetics 171:1267-1275 2005
31 Kliebenstein et al. Genetics 172:1179-1189 February 2006
32 Kirst et al. Genetics 169:2295-2303 2005
33 Paux et al. New Phytologist 167:89-100 2005
34 H. Kacser, J. A. Burns, Genetics 97, 639 (1981)
35 Langton, Smith & Edmondson 1990 Euphytica 49(1):15-23
36 L. M EJNARTOWICZ Silvae Genetica 48, 2 (1999) Pg 100-103
37 Cassady, J. P., Young, L. D., and Leymaster, K. A. (2002) J. Anim Sci. 80, 2286-2302
38 Gama, L. T., et al. (1991). J. Anim Sci. 69, 2727-2743
39 Bradford G E, Burfening P J, Cartwright T C. J. Anim Sci 1989
40 Marks H L. Poult Sci 1995 November; 74(11):1730-44
41 S. Einum and I. A. Fleming (1997) 50 (3) Journal of Fish Biology 634-651
42 Peyman and Ulman, Chemical Reviews, 90:543-584, (1990)
43 Crooke, Ann. Rev. Pharmacol. Toxicol., 32:329-376, (1992)
44 John et al, PLoS Biology, 11(2), 1862-1879, 2004
45 Myers (2003) Nature Biotechnology 21:324-328
46 Shinagawa et al., Genes and Dev., 17, 1340-5, 2003
47 Fire A, et al., 1998 Nature 391:806-811
48 Fire, A. Trends Genet. 15, 358-363 (1999)
49 Sharp, P. A. RNA interference 2001. Genes Dev. 15, 485-490 (2001)
50 Hammond, S. M., et al., Nature Rev. Genet. 2, 110-1119 (2001)
51 Tuschl, T. Chem. Biochem. 2, 239-245 (2001)
52 Hamilton, A. et al., Science 286, 950-952 (1999)
53 Hammond, S. M., et al., Nature 404, 293-296 (2000)
54 Zamore, P. D., et al., Cell 101, 25-33 (2000)
55 Bernstein, E., et al., Nature 409, 363-366 (2001)
56 Elbashir, S. M., et al., Genes Dev. 15, 188-200 (2001)
57 WO0129058
58 WO9932619
59 Elbashir S M, et al., 2001 Nature 411:494-498
60 Marschall, et al. Cellular and Molecular Neurobiology, 1994. 14(5): 523
61 Hasselhoff, Nature 334: 585 (1988) and Cech, J. Amer. Med. Assn., 260: 3030 (1988)
62 AGI, Nature 408, 796 (2000).
63 T. Zhu, X. Wang, Plant Physiol. 124, 1472 (2000)
64 R. Meyer, O. Törjék, C. Müssig, M. Lück, T. Altmann, paper presented at the Signals, Sensing and Plant Primary Metabolism 2nd Symposium. Potsdam, Germany, 2003)
65 S. Barth, A. K. Busimi, H. F. Utz, A. E. Melchinger, Heredity 91, 36 (2003)
66 M. Guo, M. A. Rupe, O. N. Danilevskaya, X. F. Yang, Z. H. Hut, Plant Journal 36, 30 (2003)
67 Sakamoto, A., et al. 2003 Plant Cell 15 2042-2057.
68 Schmid, M., et al. Nature Genetics 37 501-506 2005.
69 Tian, D., et al. Nature 423 74-77 2003
70 GenStat for Windows. Seventh Edition (7.1.0.198). 2005. Oxford, Lawes Agricultural Trust. Ref Type: Computer Program
71 C. M. O'Neill, I. Bancroft, The Plant Journal 23, 233 (2000)
72 Liu, K., et al. (2003). Genetics 165 2117-2128.

1. A method of predicting the magnitude of a trait in a plant or animal; comprising
determining transcript abundances of a gene or a set of genes in the plant or animal, wherein transcript abundances of the gene or set of genes in the plant or animal transcriptome correlate with the trait; and
thereby predicting the trait in the plant or animal.

2. A method according to claim 1, comprising earlier steps of
analysing the transcriptome of a population of plants or animals;
measuring the trait in plants or animals in the population; and
identifying a correlation between transcript abundances of a gene or set of genes in the plant or animal transcriptomes and the trait in the plants or animals.

3. A method according to claim 1, wherein the plant or animal is a hybrid.

4. A method according to claim 3, wherein the trait is heterosis.

5. A method according to claim 4, wherein the heterosis is heterosis for yield.

6. A method according to claim 1, wherein the plant or animal is inbred or recombinant.

7. A method according to claim 4, wherein the method is for predicting the magnitude of heterosis and the gene or set of genes comprises At1g67500 or At5g45500 or orthologues thereof and/or a gene or set of genes selected from the genes shown in Table 1 or Table 19, or orthologues thereof.

8-12. (canceled)

13. A method according to claim 1, comprising determining transcript abundance of a gene or set of genes in the plant or animal wherein the trait is not yet determinable from the phenotype of the plant or animal.

14-15. (canceled)

16. A method according to claim 1, wherein the method is for predicting a trait in a plant and wherein the plant a crop plant.

17. A method according to claim 16, wherein the crop plant is maize.

18. A method comprising increasing the magnitude of heterosis in a hybrid, by:
(i) upregulating expression in the hybrid of a gene or set of genes whose transcript abundance in hybrids correlates positively with the magnitude of heterosis, wherein the gene or set of genes comprises a gene or set of genes selected from the positively correlating genes shown in Table 1 and/or Table 19A, or orthologues thereof; and/or
(ii) downregulating expression in the hybrid of a gene or set of genes whose transcript abundance in hybrids correlates negatively with the magnitude of heterosis, wherein the gene or set of genes comprises a gene or set of genes selected from At1g67500, At5g45500 and/or the negatively correlating genes shown in Table 1 and/or Table 19B, or orthologues thereof.

19-21. (canceled)

22. A method of increasing a trait in a plant, by:
(i) upregulating expression in the plant of a gene or set of genes whose transcript abundance in plants correlates positively with the trait, wherein:
the trait is flowering time and wherein the gene or set of genes comprises a gene or set of genes selected from the genes listed in Table 3A or Table 4A, or orthologues thereof;
the trait is seed oil content and wherein the gene or set of genes comprises a gene or set of genes selected from the genes listed in Table 6A, or orthologues thereof;
the trait is ratio of 18:2/18:1 fatty acids in seed oil, wherein the gene or set of genes comprises a gene or set of genes selected from the genes listed in Table 7A, or orthologues thereof;
the trait is ratio of 18:3/18:1 fatty acids in seed oil, wherein the gene or set of genes comprises a gene or set of genes selected from the genes shown in Table 8A, or orthologues thereof;
the trait is ratio of 18:3/18:2 fatty acids in seed oil, wherein the gene or set of genes comprises a gene or set of genes selected from the genes shown in Table 9A, or orthologues thereof;
the trait is ratio of 20C+22C/16C+18C fatty acids in seed oil, wherein the gene or set of genes comprises a gene or set of genes selected from the genes shown in Table 10A, or orthologues thereof;
the trait is ratio of polyunsaturated/monounsaturated+saturated 18C fatty acids in seed oil, wherein the gene or set of genes comprises a gene or set of genes selected from the genes shown in Table 12A, or orthologues thereof;
the trait is % 16:0 fatty acid in seed oil, wherein the gene or set of genes comprises a gene or set of genes selected from the genes shown in Table 14A, or orthologues thereof;
the trait is % 18:1 fatty acid in seed oil, wherein the gene or set of genes comprises a gene or set of genes selected from the genes shown in Table 15A, or orthologues thereof;
the trait is % 18:2 fatty acid in seed oil, wherein the gene or set of genes comprises a gene or set of genes selected from the genes shown in Table 16A, or orthologues thereof;
the trait is % 18:3 fatty acid in seed oil, wherein the gene or set of genes comprises a gene or set of genes selected from the genes shown in Table 17A, or orthologues thereof; or
the trait is yield, and wherein the gene or set of genes comprises a gene or set of genes selected from the genes shown in Table 20A, or orthologues thereof;
or
(ii) upregulating expression in the plant of a gene or set of genes whose transcript abundance in plants correlates positively with the trait, wherein:
the trait is flowering time and wherein the gene or set of genes comprises a gene or set of genes selected from the genes listed in Table 3B or Table 4B, or orthologues thereof;
the trait is seed oil content and wherein the gene or set of genes comprises a gene or set of genes selected from the genes listed in Table 6B, or orthologues thereof;
the trait is ratio of 18:2/18:1 fatty acids in seed oil, wherein the gene or set of genes comprises a gene or set of genes selected from the genes listed in Table 7B, or orthologues thereof;
the trait is ratio of 18:3/18:1 fatty acids in seed oil, wherein the gene or set of genes comprises a gene or set of genes selected from the shown in Table 8B, or orthologues thereof;
the trait is ratio of 18:3/18:2 fatty acids in seed oil, wherein the gene or set of genes comprises a gene or set of genes selected from the genes shown in Table 9B, or orthologues thereof;
the trait is ratio of 20C+22C/16C+18C fatty acids in seed oil, wherein the gene or set of genes comprises a gene or set of genes selected from the genes shown in Table 10B, or orthologues thereof;
the trait is ratio of polyunsaturated/monounsaturated+saturated 18C fatty acids in seed oil, wherein the gene or set of genes comprises a gene or set of genes selected from the genes shown in Table 12B, or orthologues thereof;
the trait is % 16:0 fatty acid in seed oil, wherein the gene or set of genes comprises a gene or set of genes selected from the genes shown in Table 14B, or orthologues thereof;
the trait is % 18:1 fatty acid in seed oil, wherein the gene or set of genes comprises a gene or set of genes selected from the genes shown in Table 15B, or orthologues thereof;
the trait is % 18:2 fatty acid in seed oil, wherein the gene or set of genes comprises a gene or set of genes selected from the genes shown in Table 16B, or orthologues thereof;
the trait is % 18:3 fatty acid in seed oil, wherein the gene or set of genes comprises a gene or set of genes selected from the genes shown in Table 17B, or orthologues thereof; or
the trait is yield, and wherein the gene or set of genes comprises a gene or set of genes selected from the genes shown in Table 20B, or orthologues thereof.

23. (canceled)

24. A method of predicting a trait in a hybrid, wherein the hybrid is a cross between a first plant or animal and a second plant or animal; comprising
determining the transcript abundance of a gene or set of genes in the second plant or animal, wherein transcript abundance of the gene or the genes in the set of genes correlates with the trait in a population of hybrids produced by crossing the first plant or animal with different plants or animals; and
thereby predicting the trait in the hybrid.

25. A method according to claim 24, comprising earlier steps of:
analysing transcriptomes of plants or animals in a population of plants or animals;
determining a trait in a population of hybrids, wherein each hybrid in the population is a cross between a first plant or animal and a plant or animal selected from the population of plants or animals; and
identifying a correlation between transcript abundance of a gene or set of genes in the population of plants or animals and the trait in the population of hybrids.

26. A method according to claim 24, wherein the hybrid is a maize hybrid cross between a first maize plant and a second maize plant.

27-31. (canceled)

32. A method comprising:
determining the transcript abundance of a gene or set of genes in plants or animals, wherein the transcript abundances of the gene or the genes in the set of genes in plants or animals correlate with a trait in hybrid crosses between a first plant or animal and other plants or animals;
selecting one of the plants or animals on the basis of said correlation; and
selecting a hybrid that has already been produced or producing a hybrid cross between the selected plant or animal and the said first plant or animal.

33. A method according to claim 32, wherein the plants are maize and wherein a maize hybrid cross is produced.

34-43. (canceled)

44. A method comprising:
analysing the transcriptomes of hybrids in a population of hybrids;
determining heterosis or other trait of hybrids in the population; and
identifying a correlation between transcript abundance of a gene or set of genes in the hybrid transcriptomes and heterosis or other trait in the hybrids.

45. A method for determining hybrids to be grown or tested in yield or performance trials which comprises determining transcript abundance from vegetative phase plants or pre-adolescent animals.

46. A method according to claim 45, wherein the hybrids are maize hybrids.

47. A method which comprises analyzing the transcriptome of hybrids or inbred or recombinant plants or animals, said method comprising:
(i) identifying genes involved in the manifestation of heterosis and other traits in hybrids; and, optionally,
(ii) predicting and producing hybrid plants or animals of improved heterosis and other traits by selecting plants or animals for breeding, wherein the plants or animals exhibit enhanced transcriptome characteristics with respect to a selected set of genes relevant to the transcriptional regulatory networks present in potential parental breeding partners; and, optionally,
(iii) predicting a range of trait characteristics for plants and animals based on transcriptome characteristics.

48. A method according to claim 47, wherein the hybrids or inbred or recombinant plants are maize.

49. A non-human hybrid produced using the method of claim 47.

50. A subset of genes that retain most of the predictive power of a large set of genes the transcript abundance of which correlates well with a particular characteristic in a hybrid.

51. The subset according to claim 50 which comprises between 10 and 70 genes for prediction of heterosis based on hybrid transcriptomes.

52-54. (canceled)

55. A method for identifying a limited set of genes which comprises iterative testing of the precision of predictions by progressively reducing the numbers of genes in a trait predictive model, and preferentially retaining those with the best correlation of transcript abundance with the trait.

56. A computer program which, when executed by a computer, performs the method of claim 1.

57. (canceled)

58. A computer system having a processor and a display, the processor being operably configured to perform the method of claim 1 and display the results of said method on said display.
